(19) United States
(12) Patent Application Publication
(10) Pub. No.: US 2017/0309841 A1
(43) Pub. Date: Oct. 26, 2017

(54) A PLURALITY OF HOST MATERIALS AND AN ORGANIC ELECTROLUMINESCENCE DEVICE COMPRISING THE SAME

(71) Applicant: Rohm and Haas Electronic Materials Korea Ltd., Cheonan (KR)

(72) Inventors: Hyun Kim, Suwon (KR); Hee-Ryong Kang, Seoul (KR); Doo-Hyeon Moon, Hwaseong (KR); Hyun-Ju Kang, Gwangmyeong (KR); Hong-Yeop Na, Seoul (KR); Bitnari Kim, Cheonan (KR)

(21) Appl. No.: 15/517,534

(22) PCT Filed: Oct. 16, 2015
(86) PCT No.: PCT/KR2015/010975

§ 371 Date: Apr. 7, 2017

(30) Foreign Application Priority Data
Oct. 17, 2014 (KR) 10-2014-0140831

(51) Int. Cl. H01L 51/00; C07D 495/04; C07D 405/14; C07D 409/14; C07D 487/04; C07D 491/048; C07D 403/04; C09K 11/06; C09K 11/02; C07F 7/08; C07F 3/06; C07F 3/00; C07D 519/00; C07D 403/14; C07D 403/10; H01L 51/50

(52) U.S. Cl. CPC H01L 51/0092; C07D 403/10; C07D 495/04; C07D 405/14; C07D 409/14; C07D 487/04; C07D 491/048; H01L 51/0073; H01L 51/0072; H01L 51/0067; H01L 51/0052; C07D 403/04; H01L 51/0094; H01L 51/0085; H01L 51/0077; H01L 51/0074; C09K 11/06; C09K 11/025; C07F 7/0814; C07F 3/06; C07F 3/006; C07D 519/00; C07D 403/14; C09K 2211/1007; C09K 2211/1029; C09K 2211/1033; C09K 2211/1037; C09K 2211/185; H01L 2251/5384; H01L 51/5016

(57) ABSTRACT

The present invention relates to a plurality of host materials and an organic electroluminescent device comprising the same. By comprising a specific combination of a plurality of host compounds, the organic electroluminescent device according to the present invention provides high efficiency and a long lifespan.

### TECHNICAL FIELD

The present invention relates to a plurality of host materials and an organic electroluminescence device comprising the same.

### BACKGROUND ART

An electroluminescence device (EL device) is a self-light-emitting device which has advantages in that it provides a wider viewing angle, a greater contrast ratio, and a faster response time. The first organic EL device was developed by Eastman Kodak, by using small aromatic diamine molecules, and aluminum complexes as materials for forming a light-emitting layer [Appl. Phys. Lett. 51, 913, 1987].

An organic EL device (OLED) is a device changing electrical energy to light by applying electricity to an organic electroluminescent material, and generally has a structure comprising an anode, a cathode, and an organic layer between the anode and the cathode. The organic layer of an organic EL device may be comprised of a hole injection layer, a hole transport layer, an electron blocking layer, a light-emitting layer (which comprises host and dopant materials), an electron buffer layer, a hole blocking layer, an electron transport layer, an electron injection layer, etc., and the materials used for the organic layer are categorized by their functions in hole injection material, hole transport material, electron blocking material, light-emitting material, electron buffer material, hole blocking material, electron transport material, electron injection material, etc. In the organic EL device, due to an application of a voltage, holes are injected from the anode to the light-emitting layer, electrons are injected from the cathode to the light-emitting layer, and excitons of high energies are formed by a recombination of the holes and the electrons. By this energy, luminescent organic compounds reach an excited state, and light emission occurs by emitting light from energy due to the excited state of the luminescent organic compounds returning to a ground state.

The most important factor determining luminous efficiency in an organic EL device is light-emitting materials. A light-emitting material must have high quantum efficiency, high electron and hole mobility, and the formed light-emitting material layer must be uniform and stable. Light-emitting materials are categorized into blue, green, and red light-emitting materials dependent on the color of the light emission, and additionally yellow or orange light-emitting materials. In addition, light-emitting materials can also be categorized into host and dopant materials according to their functions. Recently, the development of an organic EL device providing high efficiency and long lifespan is an urgent issue. In particular, considering EL characteristic requirements for a middle or large-sized panel of OLED, materials showing better characteristics than conventional ones must be urgently developed. The host material, which acts as a solvent in a solid state and transfers energy, needs to have high purity and a molecular weight appropriate for vacuum deposition. Furthermore, the host material needs to have high glass transition temperature and high thermal degradation temperature to achieve thermal stability, high electro-chemical stability to achieve a long lifespan, ease of forming an amorphous thin film, good adhesion to materials of adjacent layers, and non-migration to other layers.

A light-emitting material can be used as a combination of a host and a dopant to improve color purity, luminous efficiency, and stability. Generally, an EL device having excellent characteristics has a structure comprising a light-emitting layer formed by doping a dopant to a host. Since host materials greatly influence the efficiency and lifespan of the EL device when using a dopant/host material system as a light-emitting material, their selection is important.

International Publication No. WO 2013/168688 A1, EP Patent Appl. Laid-Open No. EP 1323808, and International Publication No. WO 2013/112557 A1 disclose an organic electroluminescent device comprising a dopant/host material system. In the references, a carbazole-carbazole structured host or a metal complex compound having a 2^{nd} group metal as a central metal for a host was used. However, the references fail to disclose an organic electroluminescent device using plural hosts comprising a metal complex derivative and a carbazole derivative comprising a nitrogen-containing heteroaryl.

### DISCLOSURE OF THE INVENTION

### Problems to be Solved

The objective of the present invention is to provide an organic electroluminescent device having high efficiency and long lifespan.

### Solution to Problems

The present inventors found that the above objective can be achieved by an organic electroluminescent device comprising at least one light-emitting layer between an anode and a cathode, wherein the light-emitting layer comprises a host and a phosphorescent dopant, the host comprises plural host compounds, at least a first host compound of the plural host compounds is a metal complex derivative represented by the following formula 1, and a second host compound is represented by the following formula 2:

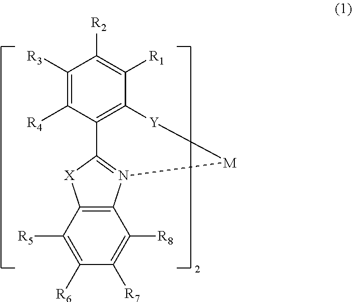

wherein

M represents a divalent metal;

Y represents O or S;

X represents NR₉, O, or S;

R₁ to R₉ each independently represent hydrogen, a halogen, a cyano, a substituted or unsubstituted (C1-C60)alkyl, a substituted or unsubstituted (C3-C60)cycloalkyl, a substituted or unsubstituted (C6-C60)aryl, a substituted or unsubstituted 4- to 60-membered heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted mono- or di- (C6-C30)arylamino; or are linked to each other to form a mono- or polycyclic, (C3-C30) alicyclic or aromatic ring, whose carbon atom(s) may be replaced with at least one hetero atom selected from nitrogen, oxygen, and sulfur;

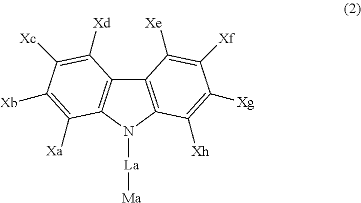

wherein

Ma represents a substituted or unsubstituted 5- to 30-membered nitrogen-containing heteroaryl;

La represents a single bond, a substituted or unsubstituted (C6-C30)arylene, or a substituted or unsubstituted 3- to 30-membered heteroarylene;

Xa to Xh each independently represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted 3- to 30-membered heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted mono- or di- (C6-C30)arylamino; or are linked to each other to form a substituted or unsubstituted mono- or polycyclic, (C3-C30) alicyclic or aromatic ring, whose carbon atom(s) may be replaced with at least one hetero atom selected from nitrogen, oxygen, and sulfur; and

the heteroaryl(ene) contains at least one hetero atom selected from B, N, O, S, Si, and P.

### Effects of the Invention

According to the present invention, an organic electroluminescent device having high efficiency and long lifespan is provided, and a display device or a lighting device using the organic electroluminescent device can be manufactured.

### EMBODIMENTS OF THE INVENTION

Hereinafter, the present invention will be described in detail. However, the following description is intended to explain the invention, and is not meant in any way to restrict the scope of the invention.

The compound represented by formula 1 can be represented by one of the following formulas 3 to 8:

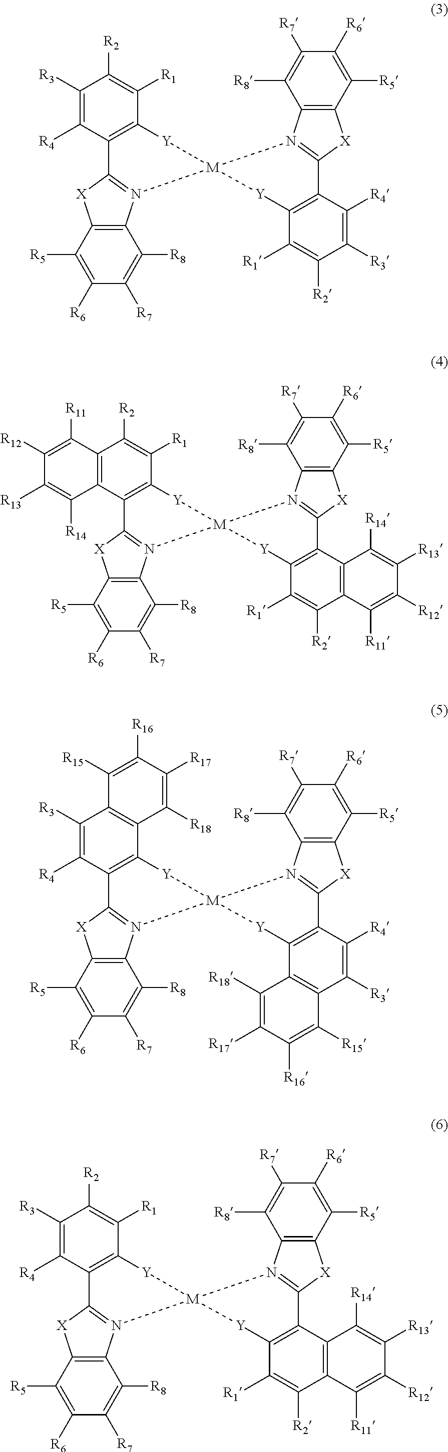

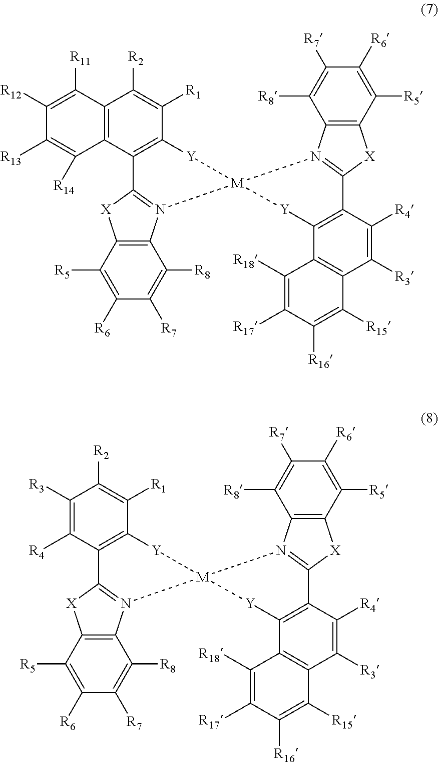

wherein

M, Y, X, and R₁ to R₈ are as defined in formula 1, and

R₁₁ to R₁₈, R₁′ to R₈′, and R₁₁′ to R₁₈^{′} each independently represent hydrogen, a halogen, a cyano, a substituted or unsubstituted (C1-C60)alkyl, a substituted or unsubstituted (C3-C60)cycloalkyl, a substituted or unsubstituted (C6-C60)aryl, a substituted or unsubstituted 4- to 60-membered heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted mono- or di- (C6-C30)arylamino; or are linked to each other to form a mono- or polycyclic, (C3-C30) alicyclic or aromatic ring, whose carbon atom(s) may be replaced with at least one hetero atom selected from nitrogen, oxygen, and sulfur.

In formula 1 above, preferably, M represents Be or Zn, Y represents O, X represents NR₉, O, or S, R₁ to R₉ each independently represent hydrogen, a halogen, a cyano, a substituted or unsubstituted (C1-C30)alkyl, a substituted or unsubstituted (C6-C30)aryl, a substituted or unsubstituted tri(C1-C30)alkylsilyl, a substituted or unsubstituted tri(C6-C30)arylsilyl, or a substituted or unsubstituted di(C6-C30)arylamino; or are linked to each other to form a mono- or polycyclic, (C3-C30) alicyclic or aromatic ring; and more preferably, M represents Be or Zn, Y represents O, X represents NR₉, O, or S, R₁ to R₉ each independently represent hydrogen, a halogen, an unsubstituted (C1-C6)alkyl, a (C6-C15)aryl unsubstituted or substituted with a halogen or a (C1-C6)alkyl, a substituted or unsubstituted tri(C1-C6)alkylsilyl, a substituted or unsubstituted tri(C6-C12)arylsilyl, or a substituted or unsubstituted di(C6-C12)arylamino; or are linked to each other to form a monocyclic, (C3-C12) aromatic ring.

In formula 2 above, La represents a single bond, a substituted or unsubstituted (C6-C30)arylene, or a substituted or unsubstituted 3- to 30-membered heteroarylene; preferably represents a single bond, a substituted or unsubstituted (C6-C12)arylene, or a substituted or unsubstituted 5- to 15-membered heteroarylene; and more preferably represents a single bond, a (C6-C12)arylene unsubstituted or substituted with a tri(C6-C10)arylsilyl or a (C6-C12)aryl, or an unsubstituted 6- to 15-membered heteroarylene.

In addition, La may represent a single bond or a carbazolylene, or can be represented by one of the following formulas 9 to 21:

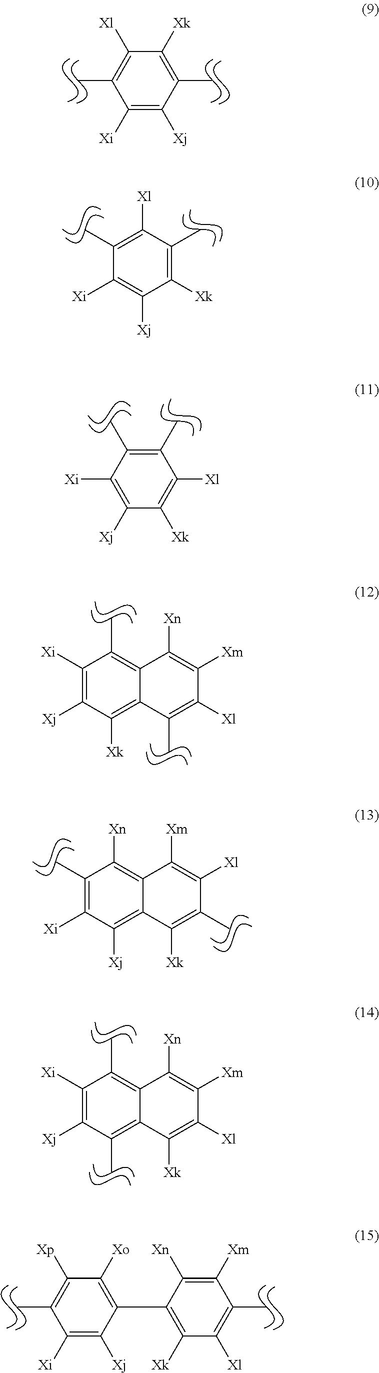

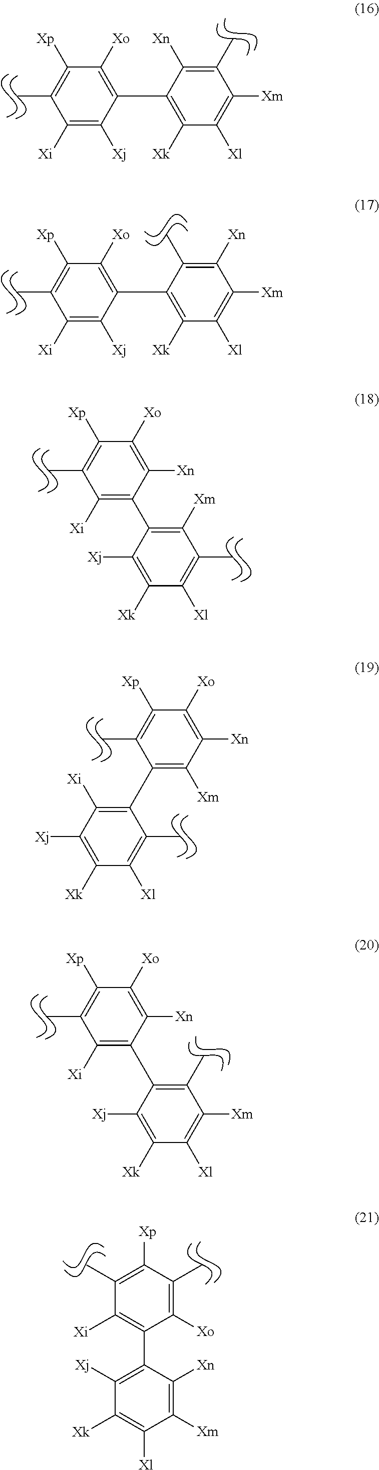

wherein

Xi to Xp each independently represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted 3- to 30-membered heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted mono- or di- (C6-C30)arylamino; or are linked to each other to form a substituted or unsubstituted, mono- or polycyclic, (C3-C30) alicyclic or aromatic ring, whose carbon atom(s) may be replaced with at least one hetero atom selected from nitrogen, oxygen, and sulfur; preferably each independently represent hydrogen, a cyano, a substituted or unsubstituted (C6-C15)aryl, a substituted or unsubstituted 10- to 20-membered heteroaryl, or a substituted or unsubstituted tri(C6-C10)arylsilyl; and more preferably each independently represent hydrogen, a cyano, a (C6-C15)aryl unsubstituted or substituted with a tri(C6-C10)arylsilyl, or a 10- to 20-membered heteroaryl unsubstituted or substituted with a (C6-C15)aryl.

In formula 2 above, Ma represents a substituted or unsubstituted 5- to 30-membered nitrogen-containing heteroaryl; preferably represents a substituted or unsubstituted 6- to 10-membered nitrogen-containing heteroaryl; and more preferably represents a 6- to 10-membered nitrogen-containing heteroaryl substituted with a substituent selected from a group consisting of an unsubstituted (C6-C25)aryl, a (C6-C12)aryl substituted with a cyano, a (C6-C12)aryl substituted with a (C1-C6)alkyl, a (C6-C12)aryl substituted with a tri(C6-C12)arylsilyl, an unsubstituted 6- to 15-membered heteroaryl, and a 6- to 15-membered heteroaryl substituted with a (C6-C12)aryl.

In addition, Ma may represent a monocyclic ring-type heteroaryl such as a substituted or unsubstituted pyrrolyl, a substituted or unsubstituted imidazolyl, a substituted or unsubstituted pyrazolyl, a substituted or unsubstituted triazinyl, a substituted or unsubstituted tetrazinyl, a substituted or unsubstituted triazolyl, a substituted or unsubstituted tetrazolyl, a substituted or unsubstituted pyridyl, a substituted or unsubstituted pyrazinyl, a substituted or unsubstituted pyrimidinyl, a substituted or unsubstituted pyridazinyl, etc., or a fused ring-type heteroaryl such as a substituted or unsubstituted benzimidazolyl, a substituted or unsubstituted isoindolyl, a substituted or unsubstituted indolyl, a substituted or unsubstituted indazolyl, a substituted or unsubstituted benzothiadiazolyl, a substituted or unsubstituted quinolyl, a substituted or unsubstituted isoquinolyl, a substituted or unsubstituted cinnolinyl, a substituted or unsubstituted quinazolinyl, a substituted or unsubstituted naphthyridinyl, a substituted or unsubstituted quinoxalinyl, a substituted or unsubstituted carbazolyl, a substituted or unsubstituted phenanthridinyl, etc. Preferably, Ma may represent a substituted or unsubstituted triazinyl, a substituted or unsubstituted pyrimidinyl, a substituted or unsubstituted pyridyl, a substituted or unsubstituted quinolyl, a substituted or unsubstituted isoquinolyl, a substituted or unsubstituted quinazolinyl, a substituted or unsubstituted naphthyridinyl, or a substituted or unsubstituted quinoxalinyl. In Ma, the substituent of the substituted pyrrolyl, etc., may be a (C6-C25)aryl, a (C6-C12)aryl substituted with a cyano, a (C6-C12)aryl substituted with a (C1-C6)alkyl, a (C6-C12)aryl substituted with a tri(C6-C12)arylsilyl, a cyano, a (C1-C6)alkyl, a tri(C6-C12)arylsilyl, a 6- to 15-membered heteroaryl, or a 6- to 15-membered heteroaryl substituted with a (C6-C12)aryl; and specifically, a cyano, a (C1-C6)alkyl, a phenyl, a biphenyl, a terphenyl, a naphthyl, a phenylnaphthyl, a naphthylphenyl, a diphenylfluorene, a phenanthrenyl, an anthracenyl, a dibenzothiophenyl, a dibenzofuranyl, or a phenylcarbazolyl, unsubstituted or substituted with a cyano, a (C1-C6)alkyl, or a triphenylsilyl.

In formula 2 above, Xa to Xh each independently represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted 3- to 30-membered heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted mono- or di- (C6-C30)arylamino; or are linked to each other to form a substituted or unsubstituted mono- or polycyclic, (C3-C30) alicyclic or aromatic ring, whose carbon atom(s) may be replaced with at least one hetero atom selected from nitrogen, oxygen, and sulfur; and preferably each independently represent hydrogen, a cyano, a substituted or unsubstituted (C6-C15)aryl, a substituted or unsubstituted 10- to 20-membered heteroaryl, or a substituted or unsubstituted tri(C6-C10)arylsilyl; or are linked to each other to form a substituted or unsubstituted mono- or polycyclic, (C6-C20) aromatic ring, whose carbon atom(s) may be replaced with at least one hetero atom selected from nitrogen, oxygen, and sulfur. More preferably, Xa to Xh each independently represent hydrogen; a cyano; a (C6-C15)aryl unsubstituted or substituted with a 10- to 20-membered heteroaryl or a tri(C6-C10)arylsilyl; a 10- to 20-membered heteroaryl unsubstituted or substituted with a (C6-C12)aryl or a cyano(C6-C12)aryl; or an unsubstituted tri(C6-C10)arylsilyl; or are linked to each other to form a substituted or unsubstituted benzene, a substituted or unsubstituted indole, a substituted or unsubstituted benzoindole, a substituted or unsubstituted indene, a substituted or unsubstituted benzofuran, or a substituted or unsubstituted benzothiophene.

Herein, “(C1-C30)alkyl” is meant to be a linear or branched alkyl having 1 to 30 carbon atoms consisting the chain, in which the number of carbon atoms is preferably 1 to 20, more preferably 1 to 10, and includes methyl, ethyl, n-propyl, isopropyl, n-butyl, isobutyl, tert-butyl, etc.; “(C2-C30)alkenyl” is meant to be a linear or branched alkenyl having 2 to 30 carbon atoms consisting the chain, in which the number of carbon atoms is preferably 2 to 20, more preferably 2 to 10, and includes vinyl, 1-propenyl, 2-propenyl, 1-butenyl, 2-butenyl, 3-butenyl, 2-methylbut-2-enyl, etc.; “(C2-C30)alkynyl” is meant to be a linear or branched alkynyl having 2 to 30 carbon atoms consisting the chain, in which the number of carbon atoms is preferably 2 to 20, more preferably 2 to 10, and includes ethynyl, 1-propynyl, 2-propynyl, 1-butynyl, 2-butynyl, 3-butynyl, 1-methylpent-2-ynyl, etc.; “(C3-C30)cycloalkyl” is a mono- or polycyclic hydrocarbon having 3 to 30 ring backbone carbon atoms, in which the number of carbon atoms is preferably 3 to 20, more preferably 3 to 7, and includes cyclopropyl, cyclobutyl, cyclopentyl, cyclohexyl, etc.; “3- to 7- membered heterocycloalkyl” is a cycloalkyl having 3 to 7 ring backbone atoms, preferably 5 to 7, including at least one heteroatom selected from B, N, O, S, Si, and P, preferably O, S, and N, and includes tetrahydrofuran, pyrrolidine, thiolan, tetrahydropyran, etc.; “(C6-C30)aryl(ene)” is a monocyclic or fused ring derived from an aromatic hydrocarbon having 6 to 30 ring backbone carbon atoms, in which the number of carbon atoms is preferably 6 to 20, more preferably 6 to 15, and includes phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, etc.; “3- to 30-membered heteroaryl” is an aryl having 3 to 30 ring backbone atoms, preferably 3 to 20 ring backbone atoms, and more preferably 3 to 15 ring backbone atoms, including at least one, preferably 1 to 4 heteroatoms selected from the group consisting of B, N, O, S, Si, and P; is a monocyclic ring, or a fused ring condensed with at least one benzene ring; may be partially saturated; may be one formed by linking at least one heteroaryl or aryl group to a heteroaryl group via a single bond(s); and includes a monocyclic ring-type heteroaryl including fury!, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl, etc.; “nitrogen-containing 5- to 30-membered heteroaryl” is an aryl having 5 to 30 ring backbone atoms, preferably 5 to 20, and more preferably 5 to 15, including at least one heteroatom, N; is a monocyclic ring, or a fused ring condensed with at least one benzene ring; may be partially saturated; may be one formed by linking at least one heteroaryl or aryl group to a heteroaryl group via a single bond(s); and includes a monocyclic ring-type heteroaryl including pyrrolyl, imidazolyl, pyrazolyl, triazinyl, tetrazinyl, triazolyl, tetrazolyl, pyridyl, pyrazinyl, pyrimidinyl, pyridazinyl, etc., and a fused ring-type heteroaryl including benzimidazolyl, isoindolyl, indolyl, indazolyl, benzothiadiazolyl, quinolyl, isoquinolyl, cinnolinyl, quinazolinyl, quinoxalinyl, carbazolyl, phenanthridinyl, etc. Further, “halogen” includes F, Cl, Br, and I.

Herein, “substituted” in the expression “substituted or unsubstituted” means that a hydrogen atom in a certain functional group is replaced with another atom or group, i.e. a substituent. The substituents of the substituted alkyl(ene), the substituted alkenyl, the substituted alkynyl, the substituted cycloalkyl, the substituted aryl(ene), the substituted heteroaryl, the substituted trialkylsilyl, the substituted triarylsilyl, the substituted dialkylarylsilyl, the substituted alkyldiarylsilyl, the substituted mono- or di-arylamino, or the substituted mono- or polycyclic, (C3-C30) alicyclic or aromatic ring in the formulas each independently are at least one selected from the group consisting of deuterium, a halogen, a cyano, a carboxyl, a nitro, a hydroxyl, a (C1-C30)alkyl, a halo(C1-C30)alkyl, a (C2-C30) alkenyl, a (C2-C30) alkynyl, a (C1-C30)alkoxy, a (C1-C30)alkylthio, a (C3-C30)cycloalkyl, a (C3-C30)cycloalkenyl, a 3- to 7-membered heterocycloalkyl, a (C6-C30)aryloxy, a (C6-C30)arylthio, a 3- to 30-membered heteroaryl unsubstituted or substituted with a (C6-C30)aryl, a (C6-C30)aryl unsubstituted or substituted with a cyano, a 3- to 30-membered heteroaryl, or a tri(C6-C30)arylsilyl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di- (C1-C30)alkylamino, a mono- or di- (C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl, and preferably are at least one selected from the group consisting of a halogen, a cyano, a (C1-C6)alkyl, a 5- to 15-membered heteroaryl unsubstituted or substituted with a (C6-C12)aryl, a (C6-C25)aryl unsubstituted or substituted with a cyano, a (C6-C12)aryl, or a tri(C6-C12)arylsilyl, a tri(C6-C12)arylsilyl, and a (C1-C6)alkyl(C6-C12)aryl. The first host compound represented by formula 1 includes the following compounds, but is not limited thereto:

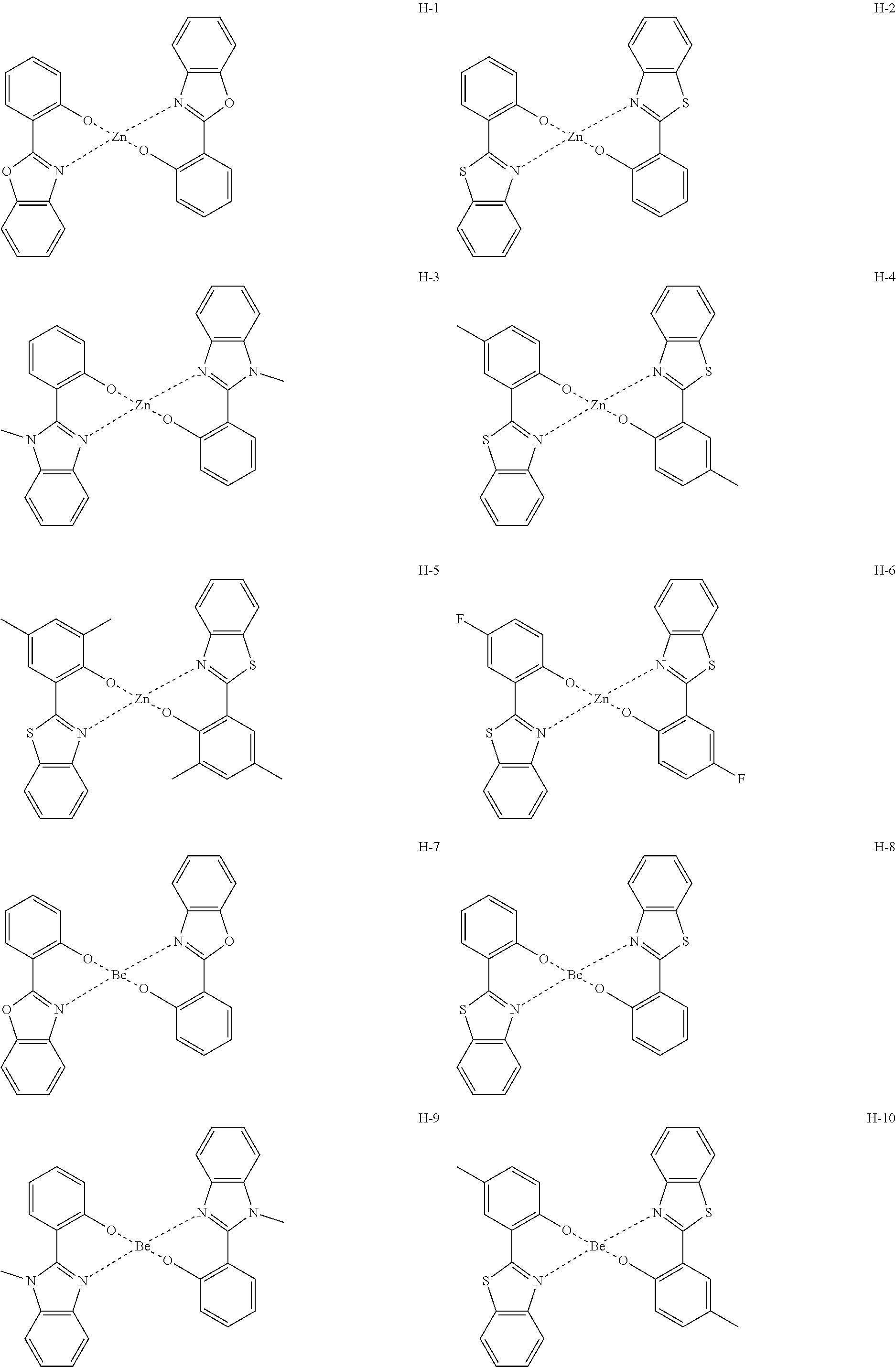

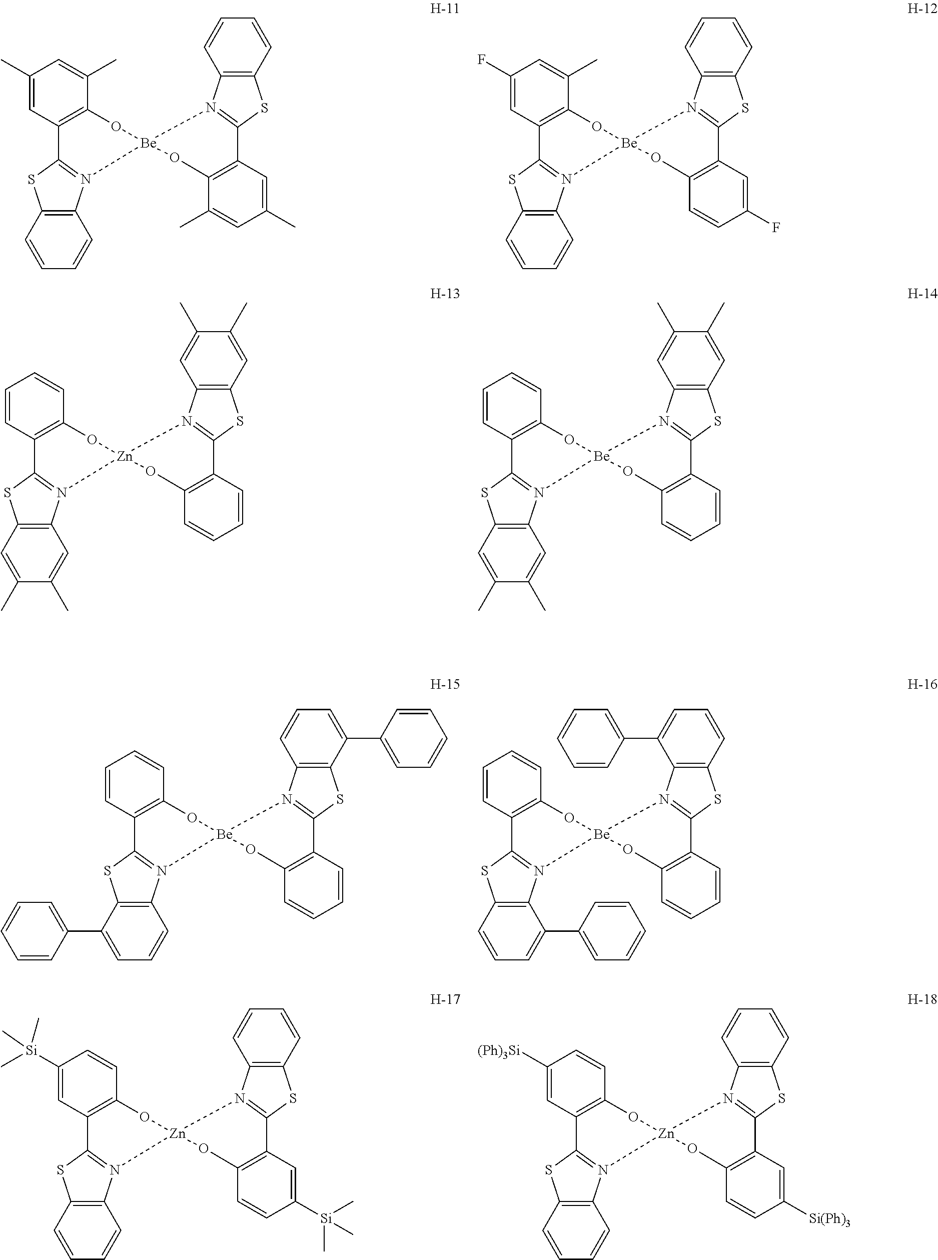

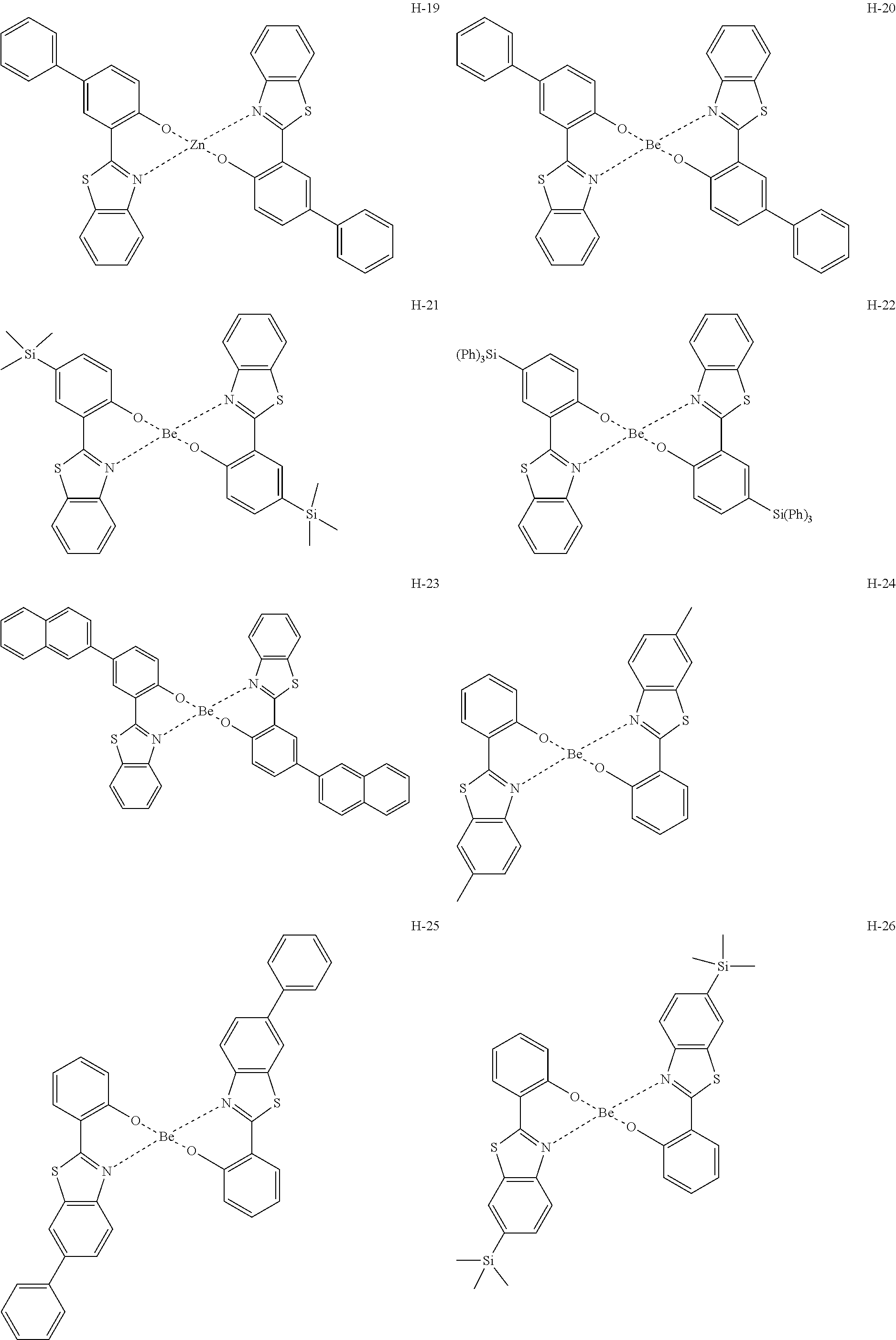

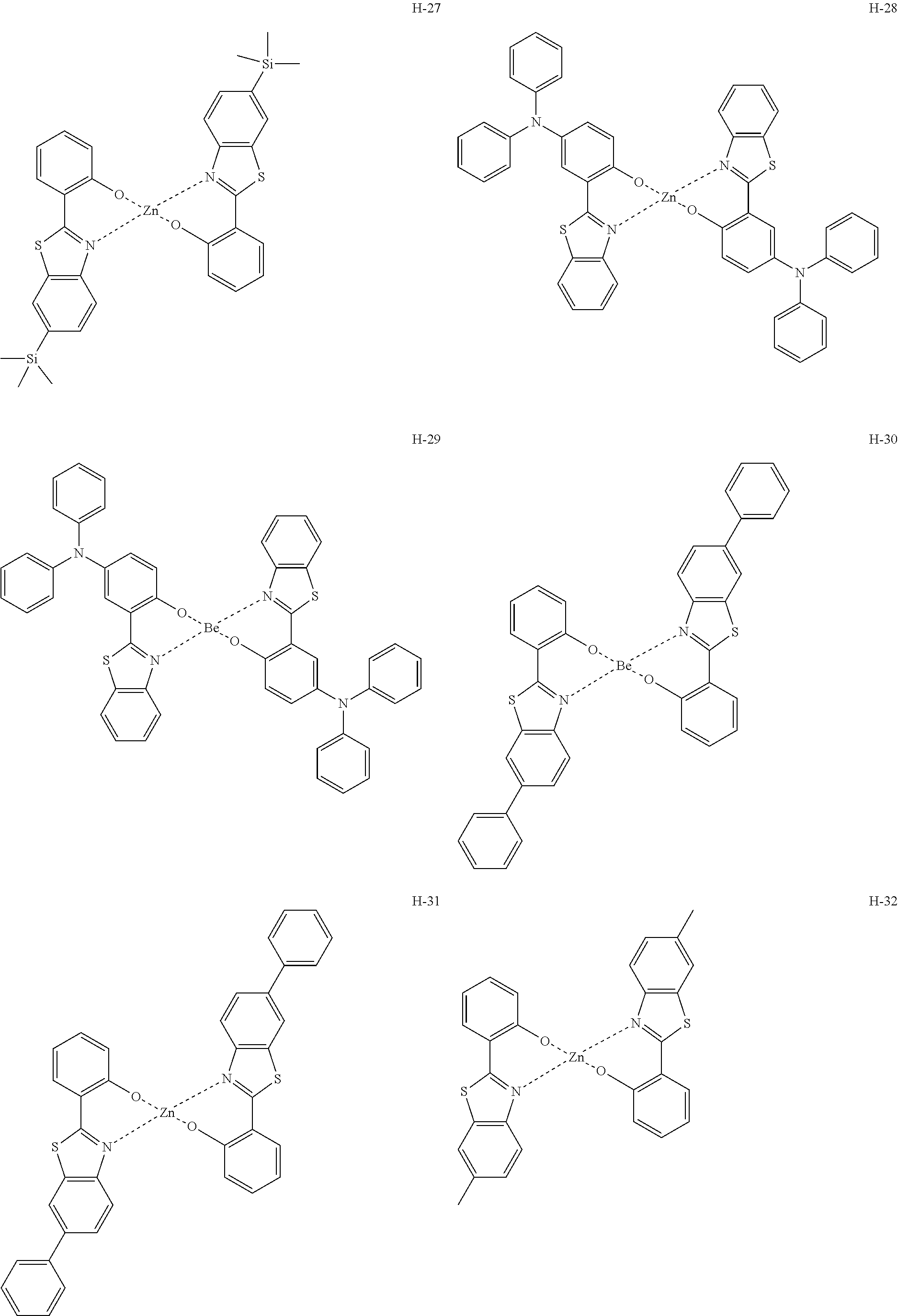

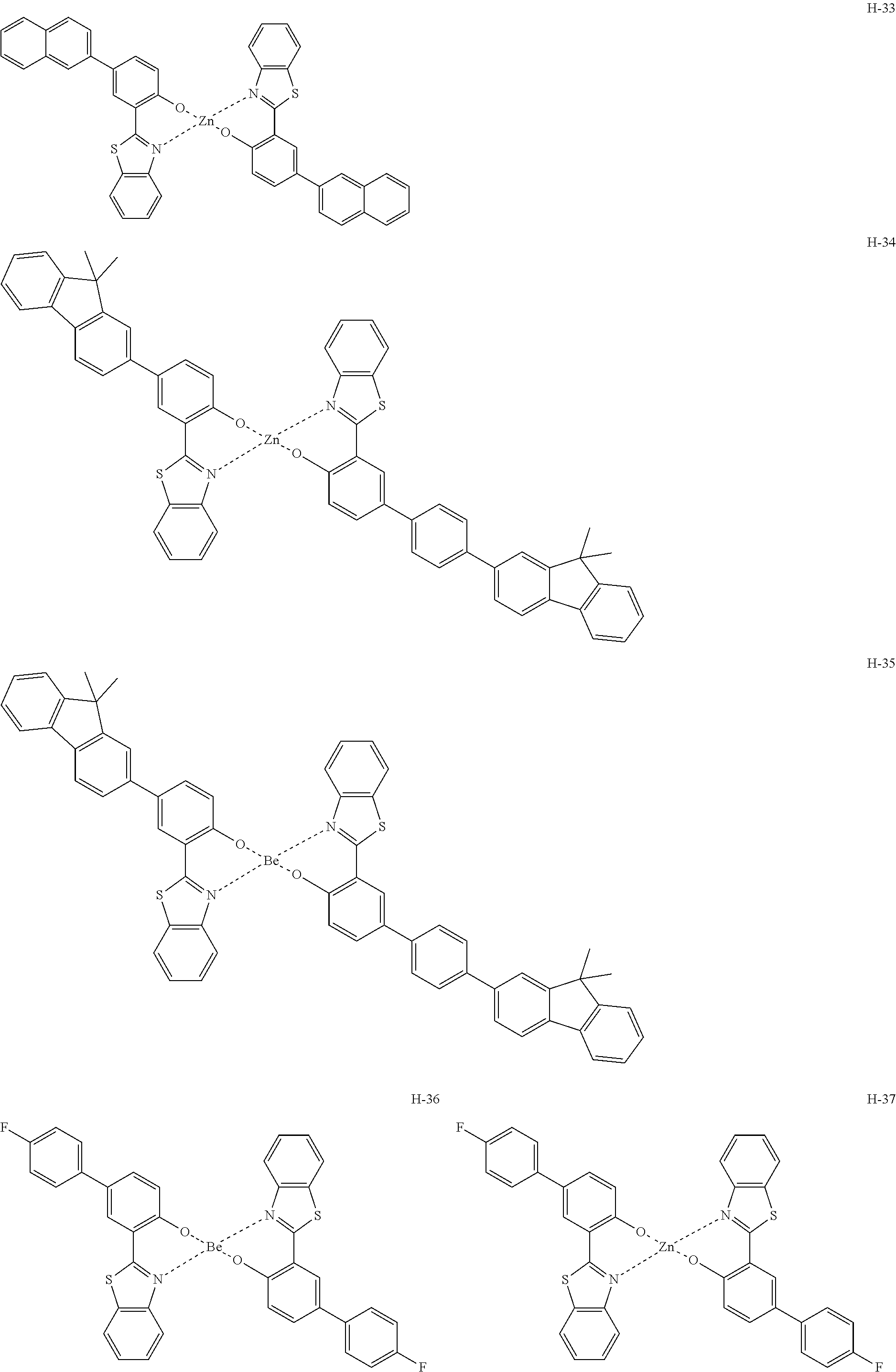

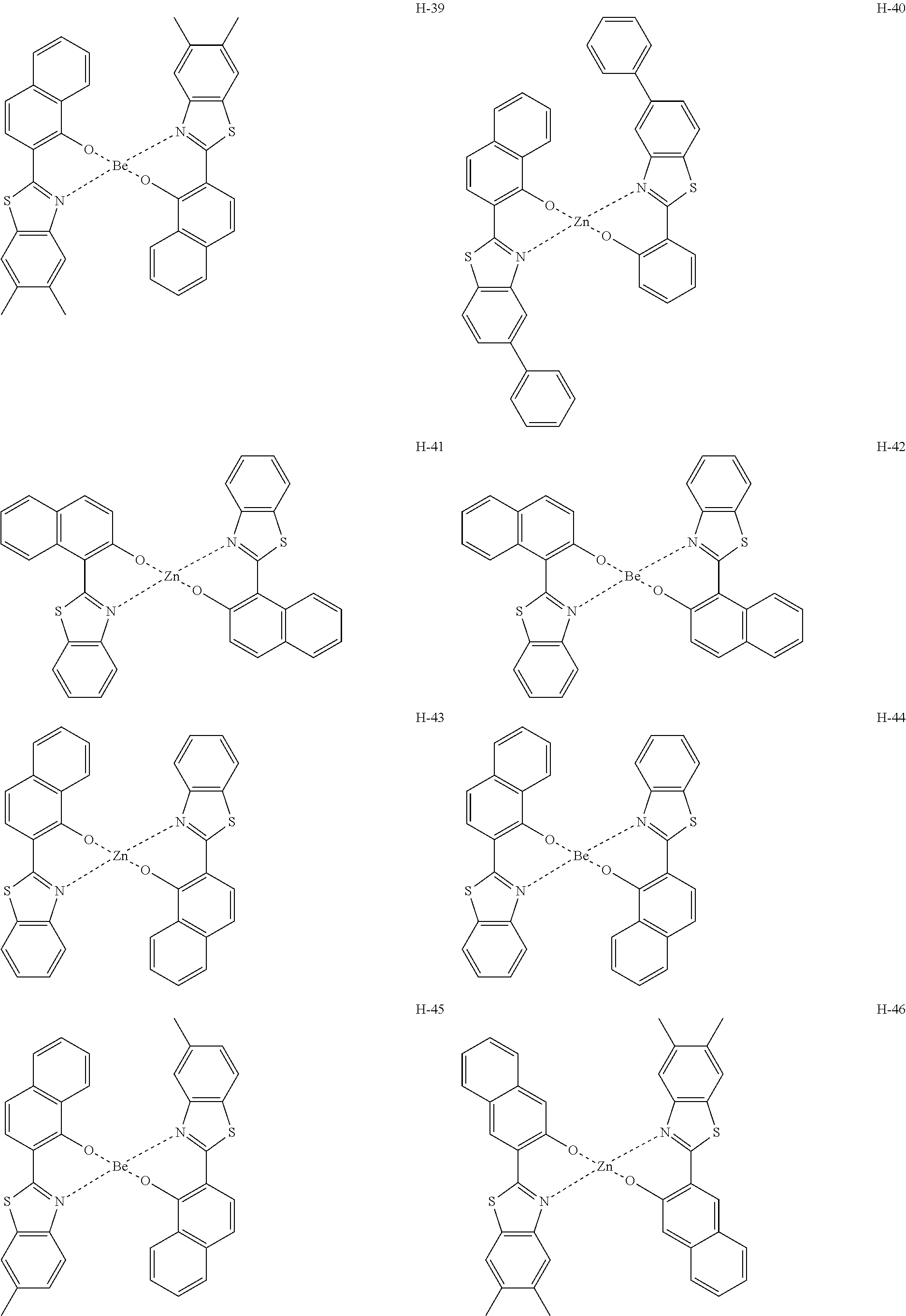

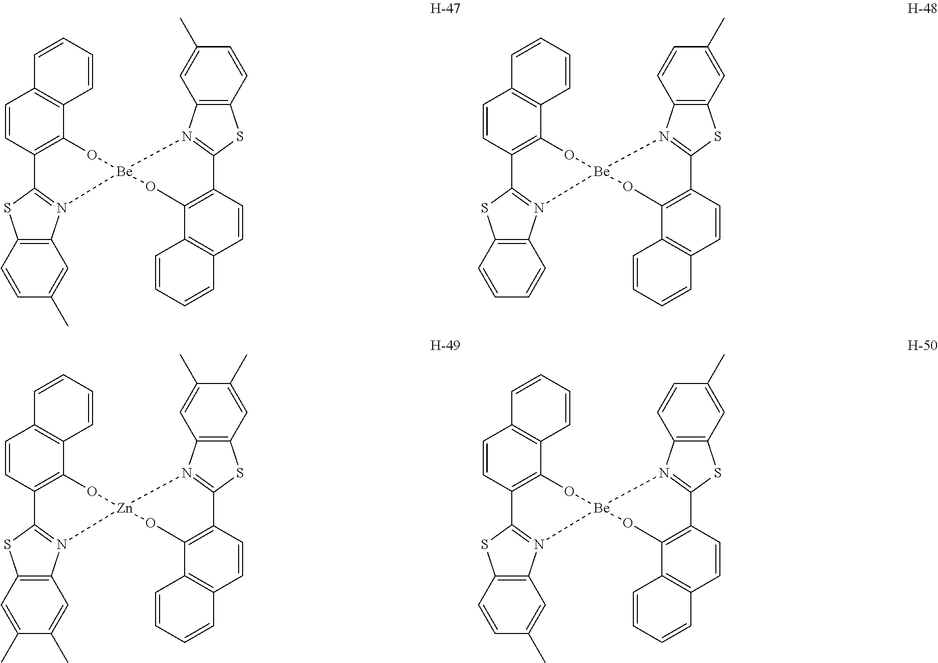

The second host compound represented by formula 2 includes the following compounds, but is not limited thereto:

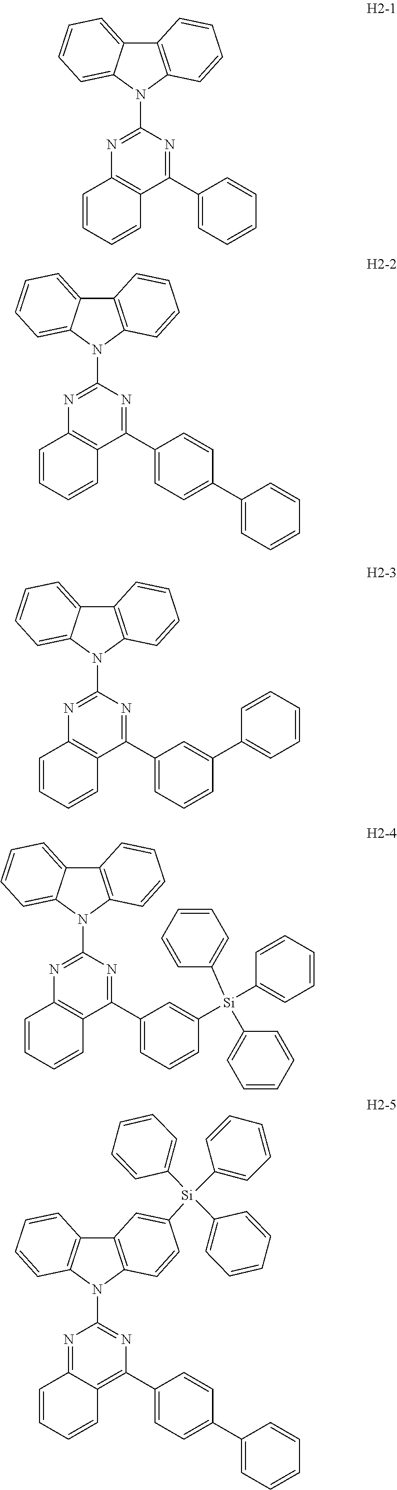

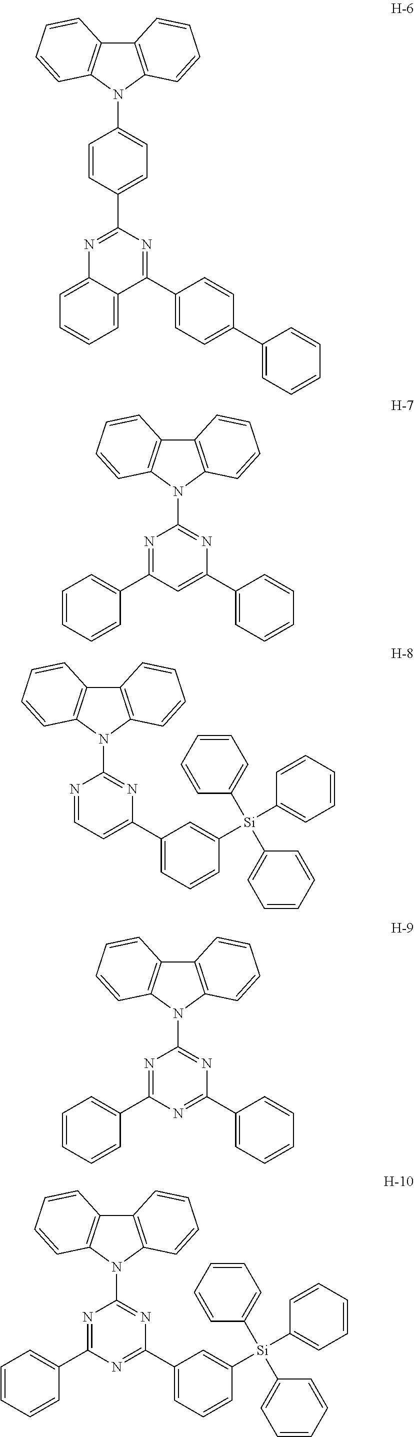

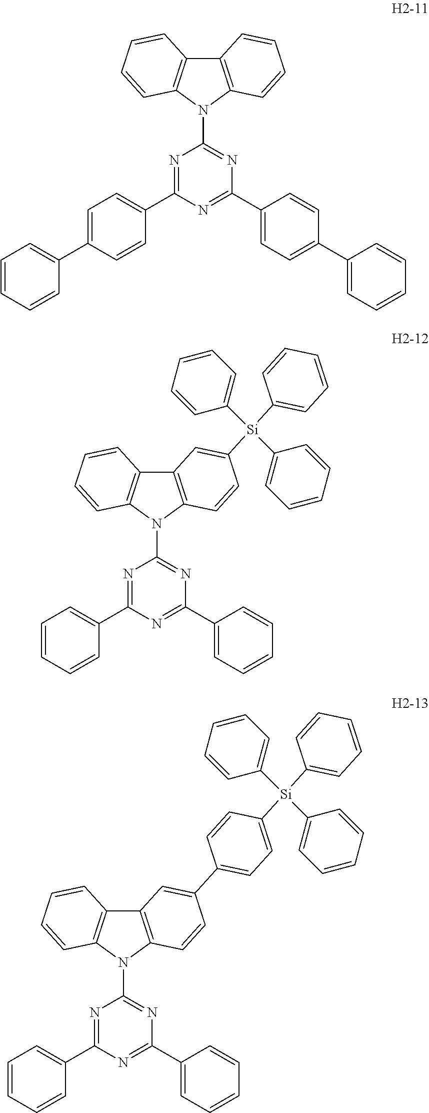

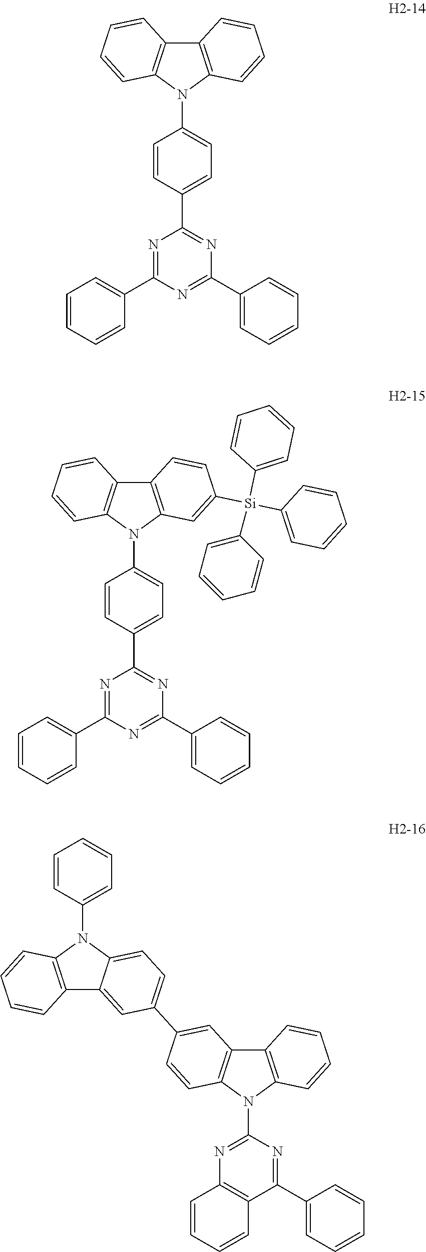 [SMILES: c1ccc(-c2nc(-c3ccccc3)nc(-c3ccc(-n4c5ccccc5c5ccccc54)cc3)n2)cc1 ; c1ccc(-c2nc(-c3ccccc3)nc(-c3ccc(-n4c5ccccc5c5ccc([Si](c6ccccc6)(c6ccccc6)c6ccccc6)cc54)cc3)n2)cc1 ; c1ccc(-c2nc(-n3c4ccccc4c4cc(-c5ccc6c(c5)c5ccccc5n6-c5ccccc5)ccc43)nc3ccccc23)cc1]

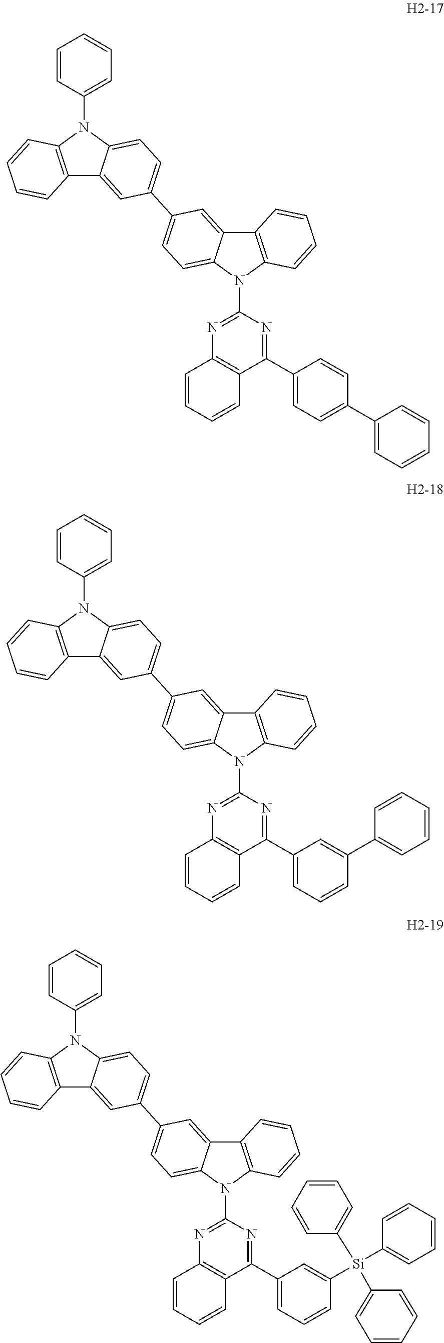 [SMILES: c1ccc(-c2ccc(-c3nc(-n4c5ccccc5c5cc(-c6ccc7c(c6)c6ccccc6n7-c6ccccc6)ccc54)nc4ccccc34)cc2)cc1 ; c1ccc(-c2cccc(-c3nc(-n4c5ccccc5c5cc(-c6ccc7c(c6)c6ccccc6n7-c6ccccc6)ccc54)nc4ccccc34)c2)cc1 ; c1ccc(-n2c3ccccc3c3cc(-c4ccc5c(c4)c4ccccc4n5-c4nc(-c5cccc([Si](c6ccccc6)(c6ccccc6)c6ccccc6)c5)c5ccccc5n4)ccc32)cc1]

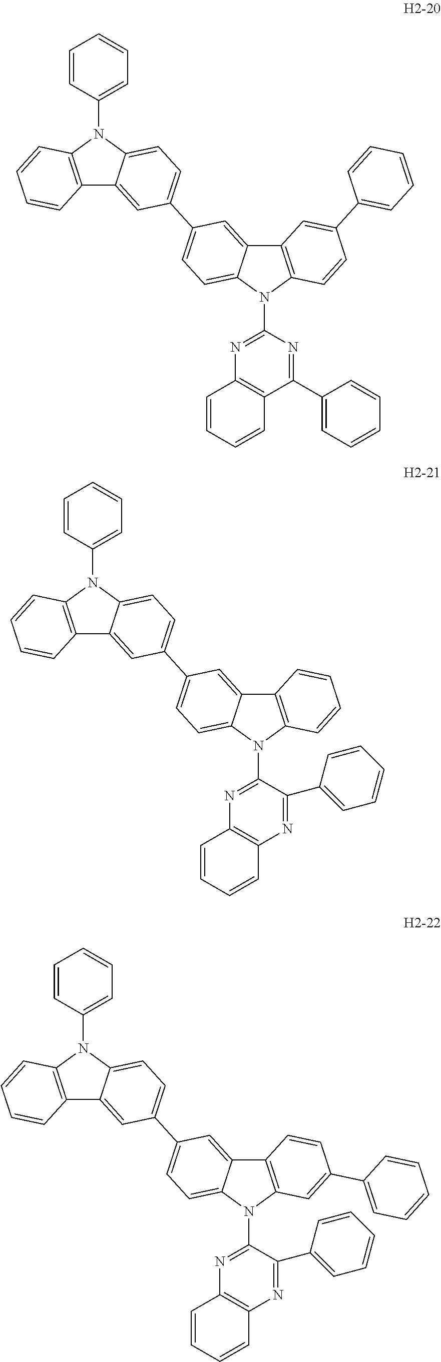 [SMILES: c1ccc(-c2ccc3c(c2)c2cc(-c4ccc5c(c4)c4ccccc4n5-c4ccccc4)ccc2n3-c2nc(-c3ccccc3)c3ccccc3n2)cc1 ; c1ccc(-c2nc3ccccc3nc2-n2c3ccccc3c3cc(-c4ccc5c(c4)c4ccccc4n5-c4ccccc4)ccc32)cc1 ; c1ccc(-c2ccc3c4cc(-c5ccc6c(c5)c5ccccc5n6-c5ccccc5)ccc4n(-c4nc5ccccc5nc4-c4ccccc4)c3c2)cc1]

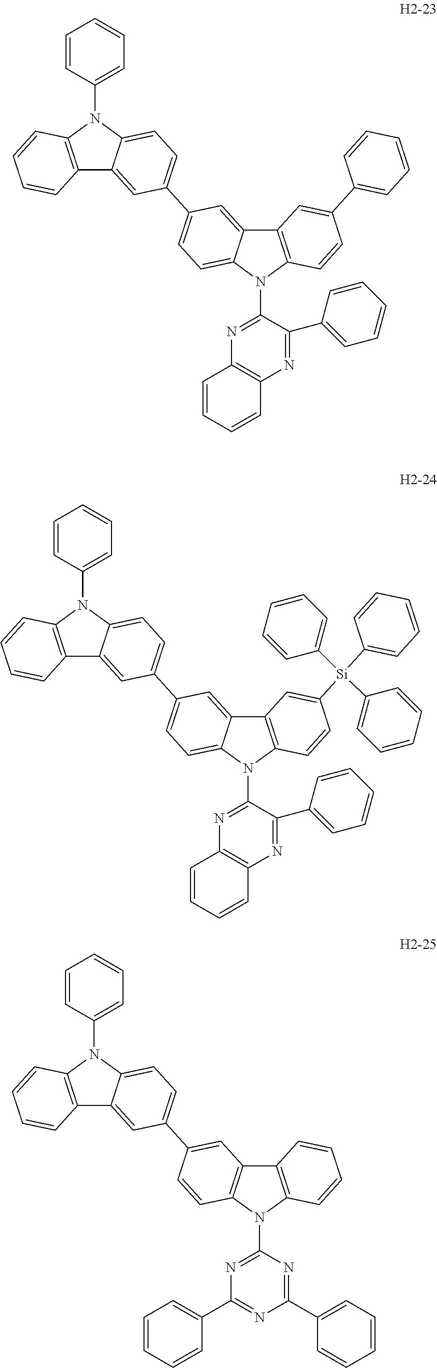 [SMILES: c1ccc(-c2ccc3c(c2)c2cc(-c4ccc5c(c4)c4ccccc4n5-c4ccccc4)ccc2n3-c2nc3ccccc3nc2-c2ccccc2)cc1 ; c1ccc(-c2nc3ccccc3nc2-n2c3ccc(-c4ccc5c(c4)c4ccccc4n5-c4ccccc4)cc3c3cc([Si](c4ccccc4)(c4ccccc4)c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1]

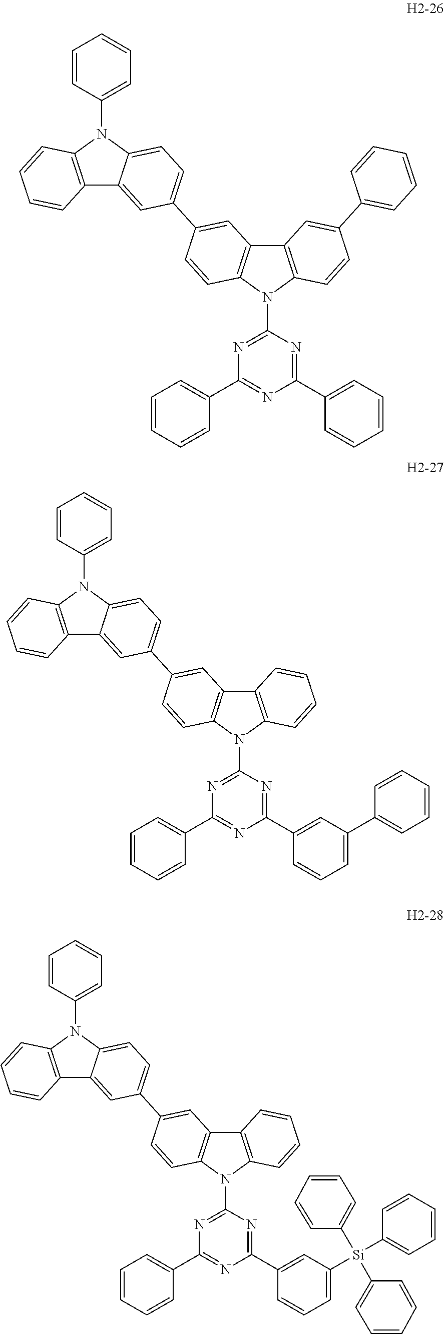 [SMILES: c1ccc(-c2ccc3c(c2)c2cc(-c4ccc5c(c4)c4ccccc4n5-c4ccccc4)ccc2n3-c2nc(-c3ccccc3)nc(-c3ccccc3)n2)cc1 ; c1ccc(-c2cccc(-c3nc(-c4ccccc4)nc(-n4c5ccccc5c5cc(-c6ccc7c(c6)c6ccccc6n7-c6ccccc6)ccc54)n3)c2)cc1 ; c1ccc(-c2nc(-c3cccc([Si](c4ccccc4)(c4ccccc4)c4ccccc4)c3)nc(-n3c4ccccc4c4cc(-c5ccc6c(c5)c5ccccc5n6-c5ccccc5)ccc43)n2)cc1]

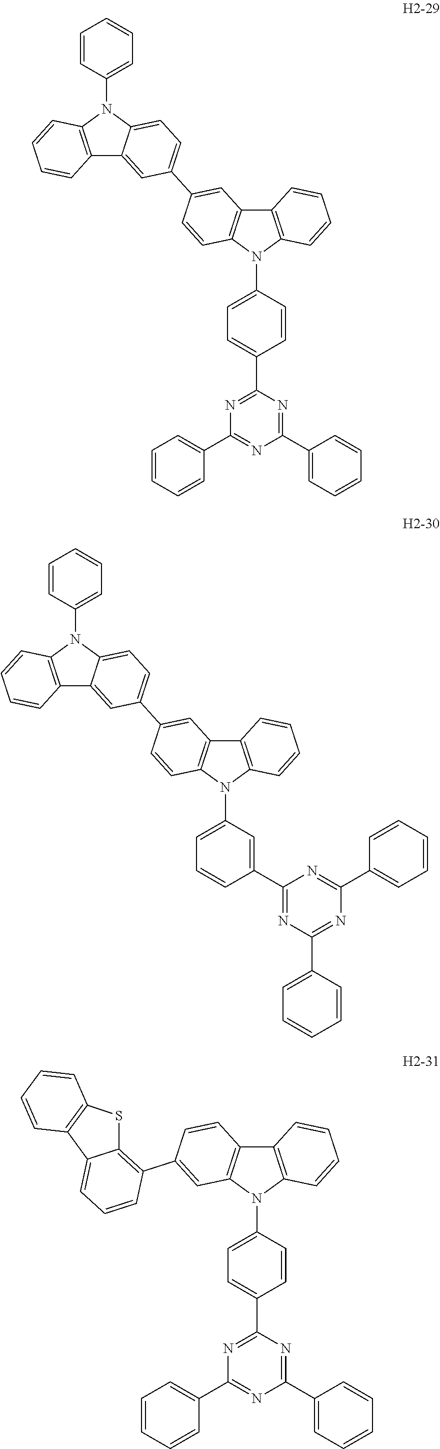 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2nc(-c3ccccc3)nc(-c3ccc(-n4c5ccccc5c5ccc(-c6cccc7c6sc6ccccc67)cc54)cc3)n2)cc1]

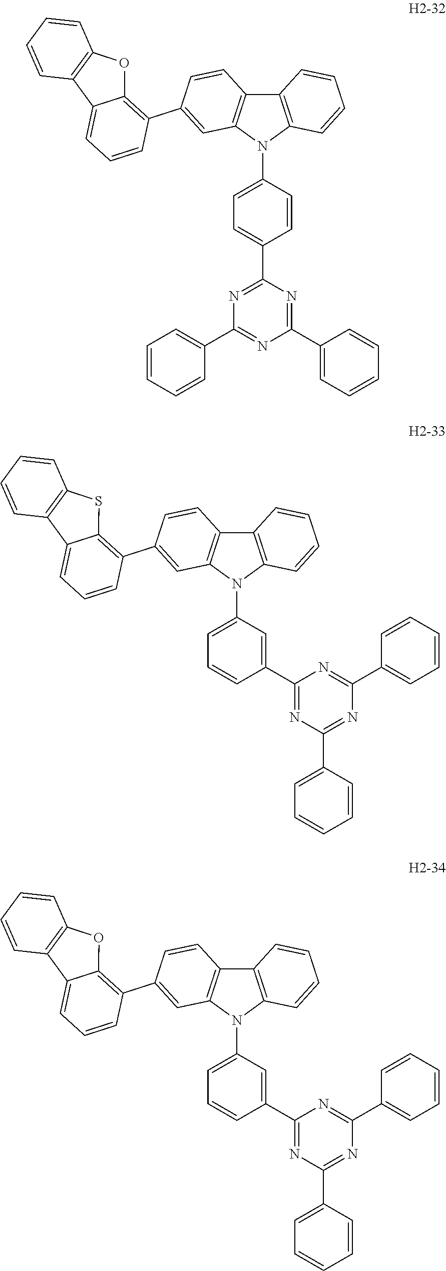 [SMILES: c1ccc(-c2nc(-c3ccccc3)nc(-c3ccc(-n4c5ccccc5c5ccc(-c6cccc7c6oc6ccccc67)cc54)cc3)n2)cc1 ; c1ccc(-c2nc(-c3ccccc3)nc(-c3cccc(-n4c5ccccc5c5ccc(-c6cccc7c6sc6ccccc67)cc54)c3)n2)cc1 ; c1ccc(-c2nc(-c3ccccc3)nc(-c3cccc(-n4c5ccccc5c5ccc(-c6cccc7c6oc6ccccc67)cc54)c3)n2)cc1]

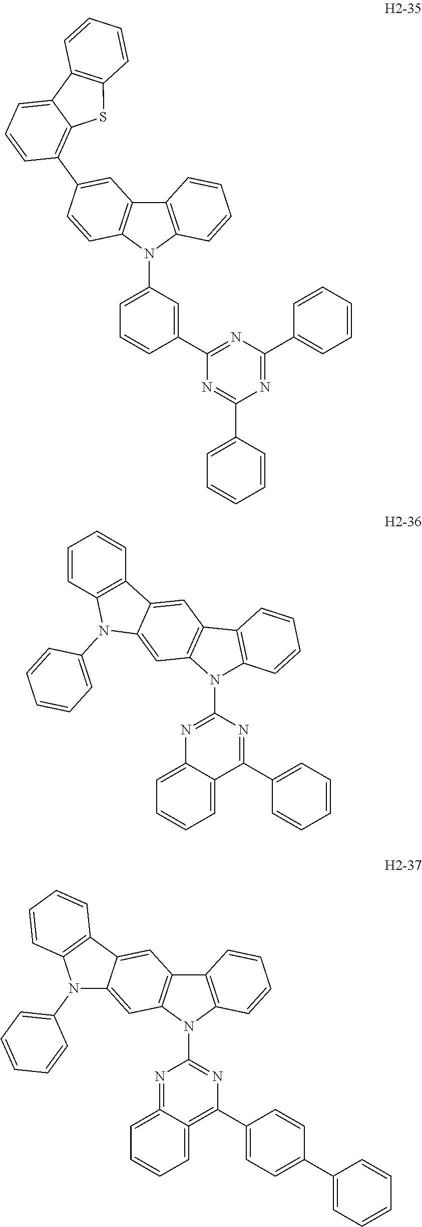

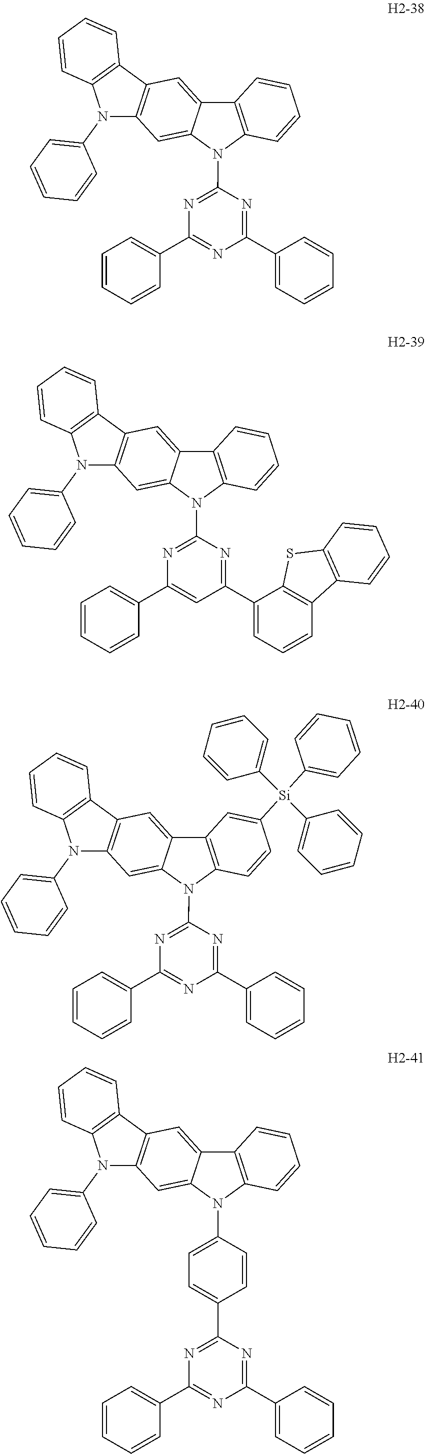

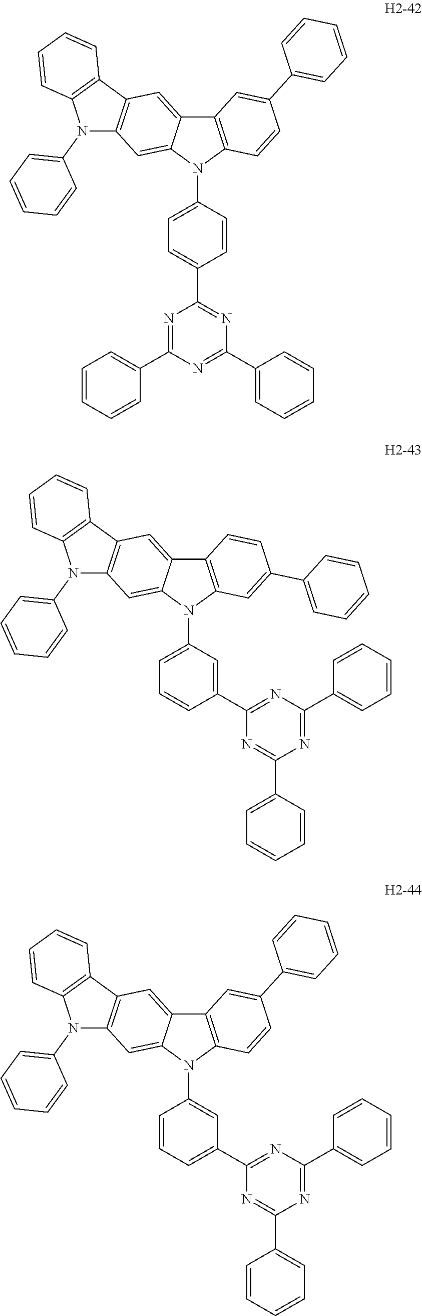 [SMILES: c1ccc(-c2ccc3c(c2)c2cc4c5ccccc5n(-c5ccccc5)c4cc2n3-c2ccc(-c3nc(-c4ccccc4)nc(-c4ccccc4)n3)cc2)cc1 ; c1ccc(-c2ccc3c4cc5c6ccccc6n(-c6ccccc6)c5cc4n(-c4cccc(-c5nc(-c6ccccc6)nc(-c6ccccc6)n5)c4)c3c2)cc1 ; c1ccc(-c2ccc3c(c2)c2cc4c5ccccc5n(-c5ccccc5)c4cc2n3-c2cccc(-c3nc(-c4ccccc4)nc(-c4ccccc4)n3)c2)cc1]

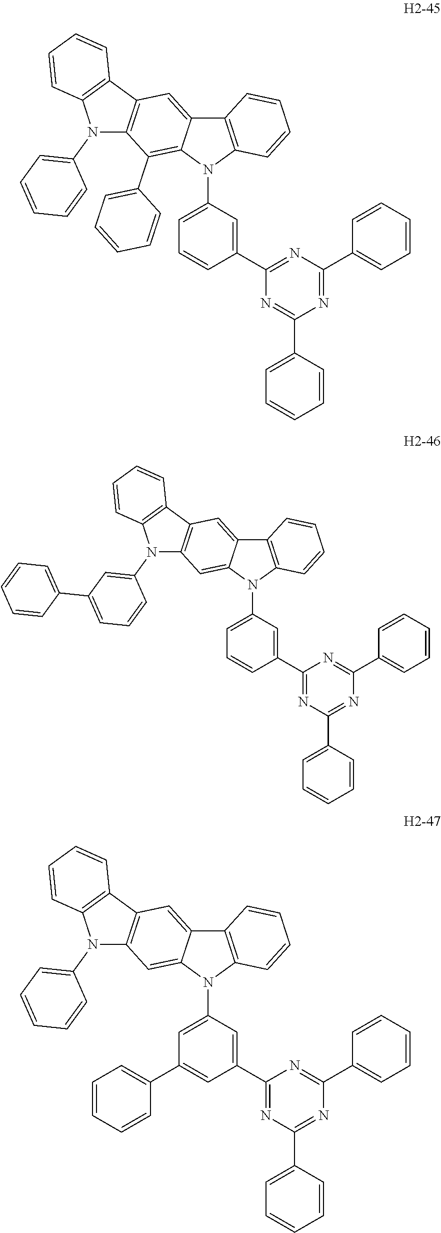 [SMILES: c1ccc(-c2nc(-c3ccccc3)nc(-c3cccc(-n4c5ccccc5c5cc6c7ccccc7n(-c7ccccc7)c6c(-c6ccccc6)c54)c3)n2)cc1 ; c1ccc(-c2cccc(-n3c4ccccc4c4cc5c6ccccc6n(-c6cccc(-c7nc(-c8ccccc8)nc(-c8ccccc8)n7)c6)c5cc43)c2)cc1 ; c1ccc(-c2cc(-c3nc(-c4ccccc4)nc(-c4ccccc4)n3)cc(-n3c4ccccc4c4cc5c6ccccc6n(-c6ccccc6)c5cc43)c2)cc1]

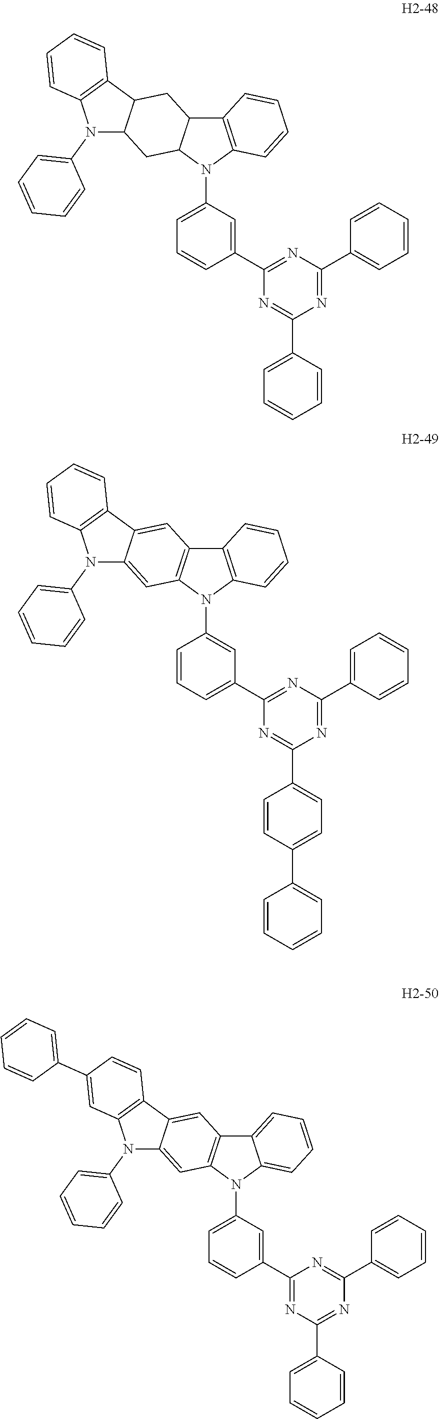 [SMILES: c1ccc(-c2nc(-c3ccccc3)nc(-c3cccc(N4c5ccccc5C5CC6c7ccccc7N(c7ccccc7)C6CC54)c3)n2)cc1 ; c1ccc(-c2ccc(-c3nc(-c4ccccc4)nc(-c4cccc(-n5c6ccccc6c6cc7c8ccccc8n(-c8ccccc8)c7cc65)c4)n3)cc2)cc1 ; c1ccc(-c2ccc3c4cc5c6ccccc6n(-c6cccc(-c7nc(-c8ccccc8)nc(-c8ccccc8)n7)c6)c5cc4n(-c4ccccc4)c3c2)cc1]

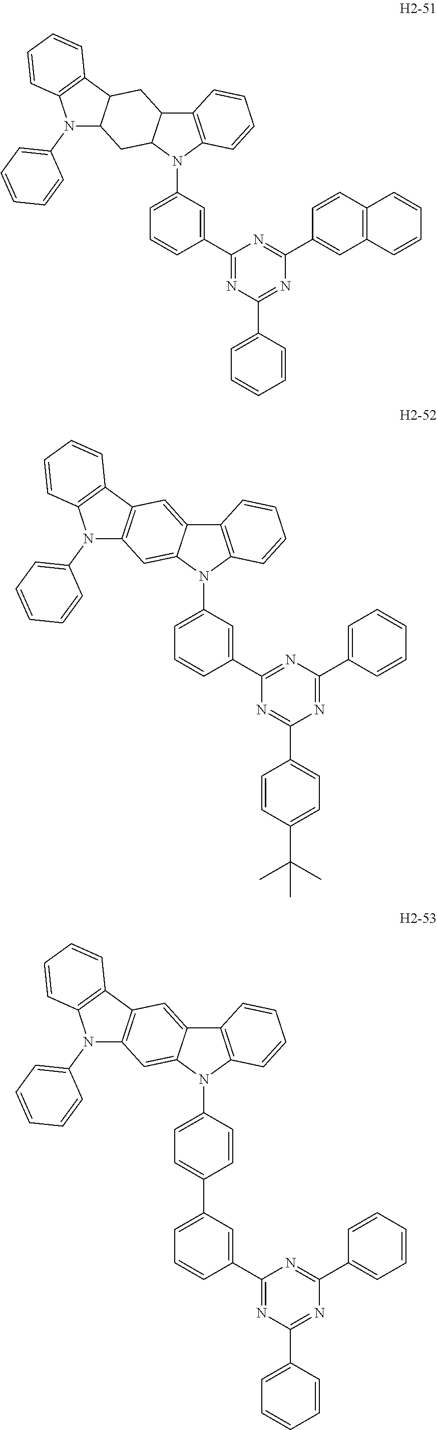 [SMILES: c1ccc(-c2nc(-c3cccc(N4c5ccccc5C5CC6c7ccccc7N(c7ccccc7)C6CC54)c3)nc(-c3ccc4ccccc4c3)n2)cc1 ; CC(C)(C)c1ccc(-c2nc(-c3ccccc3)nc(-c3cccc(-n4c5ccccc5c5cc6c7ccccc7n(-c7ccccc7)c6cc54)c3)n2)cc1 ; c1ccc(-c2nc(-c3ccccc3)nc(-c3cccc(-c4ccc(-n5c6ccccc6c6cc7c8ccccc8n(-c8ccccc8)c7cc65)cc4)c3)n2)cc1]

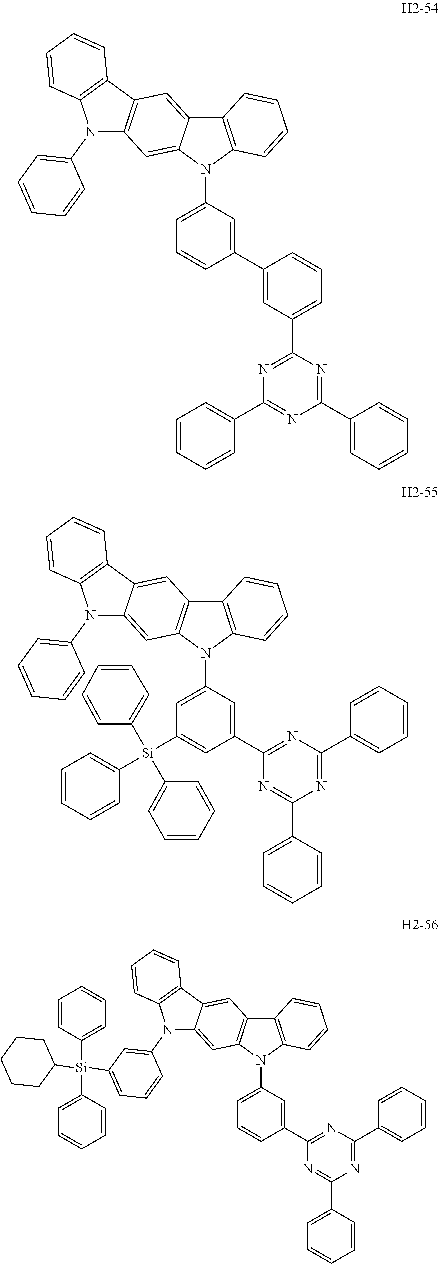 [SMILES: c1ccc(-c2nc(-c3ccccc3)nc(-c3cccc(-c4cccc(-n5c6ccccc6c6cc7c8ccccc8n(-c8ccccc8)c7cc65)c4)c3)n2)cc1 ; c1ccc(-c2nc(-c3ccccc3)nc(-c3cc(-n4c5ccccc5c5cc6c7ccccc7n(-c7ccccc7)c6cc54)cc([Si](c4ccccc4)(c4ccccc4)c4ccccc4)c3)n2)cc1 ; c1ccc(-c2nc(-c3ccccc3)nc(-c3cccc(-n4c5ccccc5c5cc6c7ccccc7n(-c7cccc([Si](c8ccccc8)(c8ccccc8)C8CCCCC8)c7)c6cc54)c3)n2)cc1]

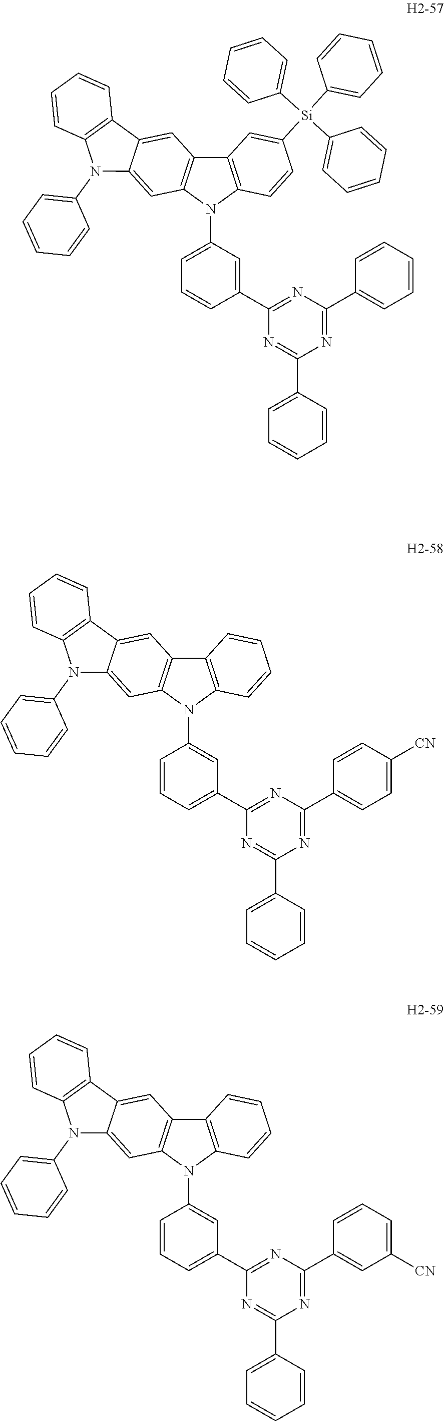 [SMILES: c1ccc(-c2nc(-c3ccccc3)nc(-c3cccc(-n4c5ccc([Si](c6ccccc6)(c6ccccc6)c6ccccc6)cc5c5cc6c7ccccc7n(-c7ccccc7)c6cc54)c3)n2)cc1 ; N#Cc1ccc(-c2nc(-c3ccccc3)nc(-c3cccc(-n4c5ccccc5c5cc6c7ccccc7n(-c7ccccc7)c6cc54)c3)n2)cc1 ; N#Cc1cccc(-c2nc(-c3ccccc3)nc(-c3cccc(-n4c5ccccc5c5cc6c7ccccc7n(-c7ccccc7)c6cc54)c3)n2)c1]

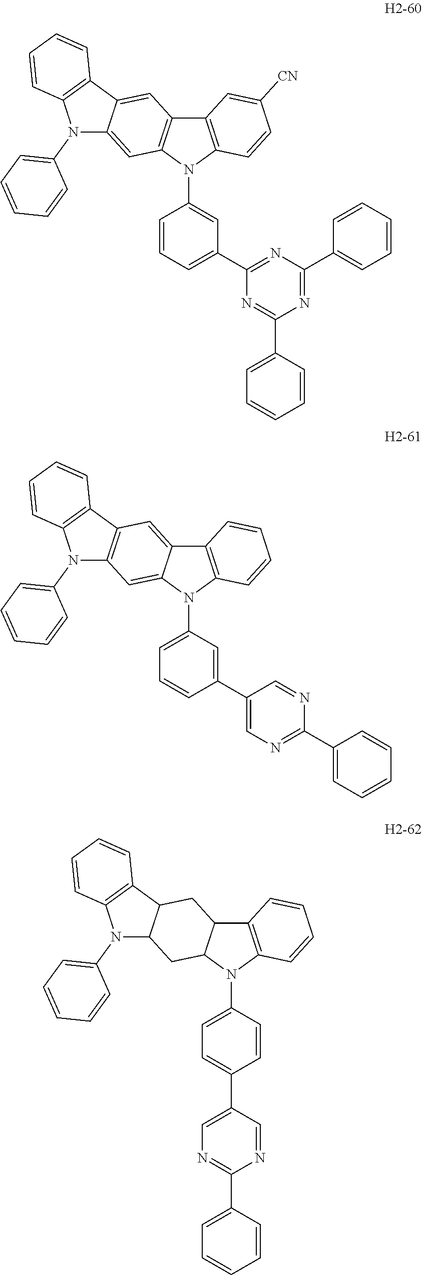 [SMILES: N#Cc1ccc2c(c1)c1cc3c4ccccc4n(-c4ccccc4)c3cc1n2-c1cccc(-c2nc(-c3ccccc3)nc(-c3ccccc3)n2)c1 ; c1ccc(-c2ncc(-c3cccc(-n4c5ccccc5c5cc6c7ccccc7n(-c7ccccc7)c6cc54)c3)cn2)cc1 ; c1ccc(-c2ncc(-c3ccc(N4c5ccccc5C5CC6c7ccccc7N(c7ccccc7)C6CC54)cc3)cn2)cc1]

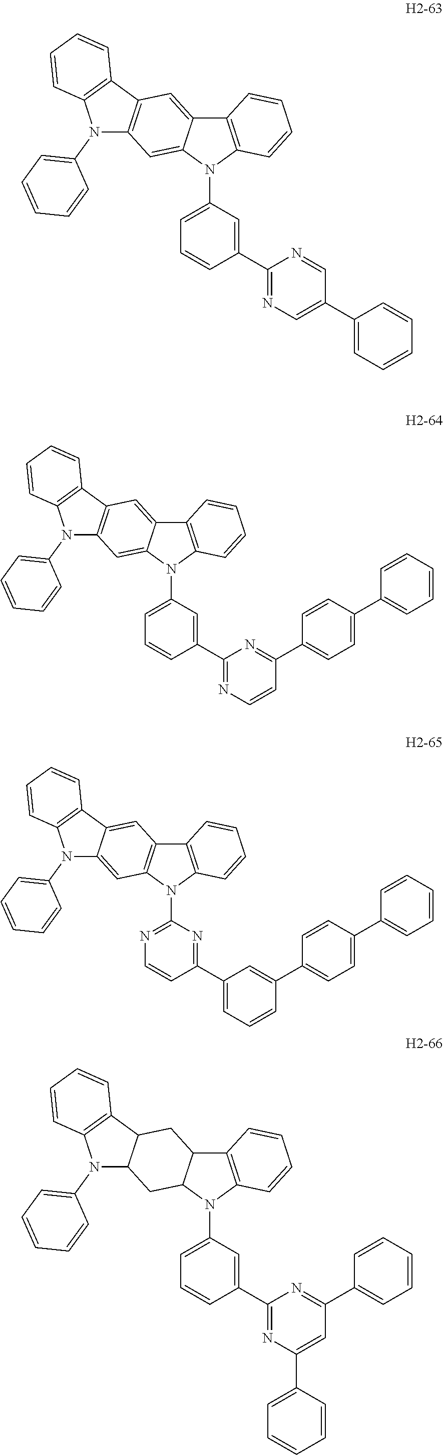 [SMILES: c1ccc(-c2cnc(-c3cccc(-n4c5ccccc5c5cc6c7ccccc7n(-c7ccccc7)c6cc54)c3)nc2)cc1 ; c1ccc(-c2ccc(-c3ccnc(-c4cccc(-n5c6ccccc6c6cc7c8ccccc8n(-c8ccccc8)c7cc65)c4)n3)cc2)cc1 ; c1ccc(-c2ccc(-c3cccc(-c4ccnc(-n5c6ccccc6c6cc7c8ccccc8n(-c8ccccc8)c7cc65)n4)c3)cc2)cc1 ; c1ccc(-c2cc(-c3ccccc3)nc(-c3cccc(N4c5ccccc5C5CC6c7ccccc7N(c7ccccc7)C6CC54)c3)n2)cc1]

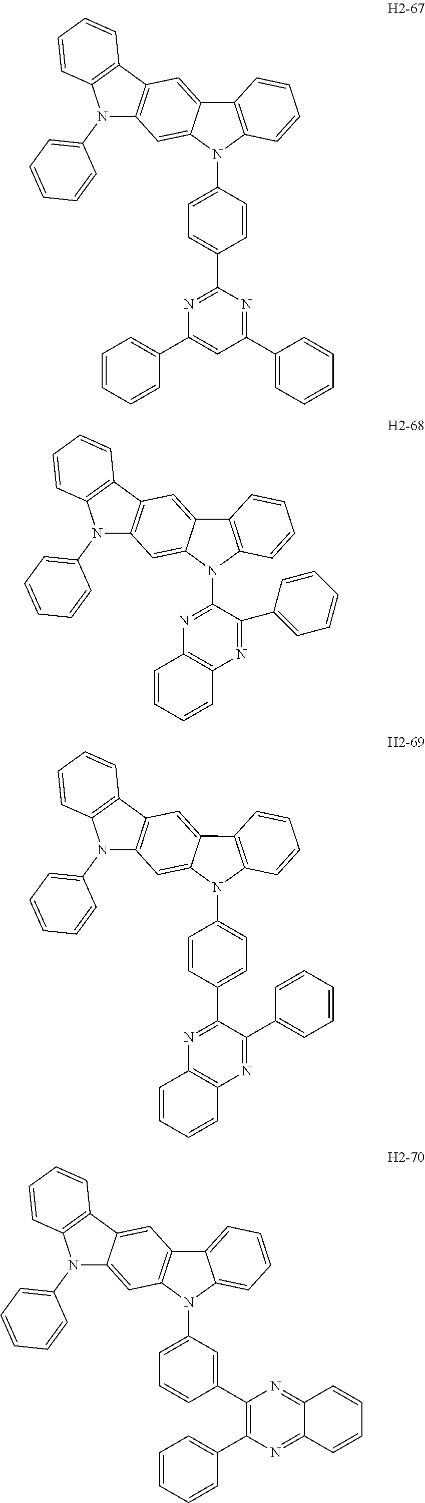 [SMILES: c1ccc(-c2cc(-c3ccccc3)nc(-c3ccc(-n4c5ccccc5c5cc6c7ccccc7n(-c7ccccc7)c6cc54)cc3)n2)cc1 ; c1ccc(-c2nc3ccccc3nc2-n2c3ccccc3c3cc4c5ccccc5n(-c5ccccc5)c4cc32)cc1 ; c1ccc(-c2nc3ccccc3nc2-c2ccc(-n3c4ccccc4c4cc5c6ccccc6n(-c6ccccc6)c5cc43)cc2)cc1 ; c1ccc(-c2nc3ccccc3nc2-c2cccc(-n3c4ccccc4c4cc5c6ccccc6n(-c6ccccc6)c5cc43)c2)cc1]

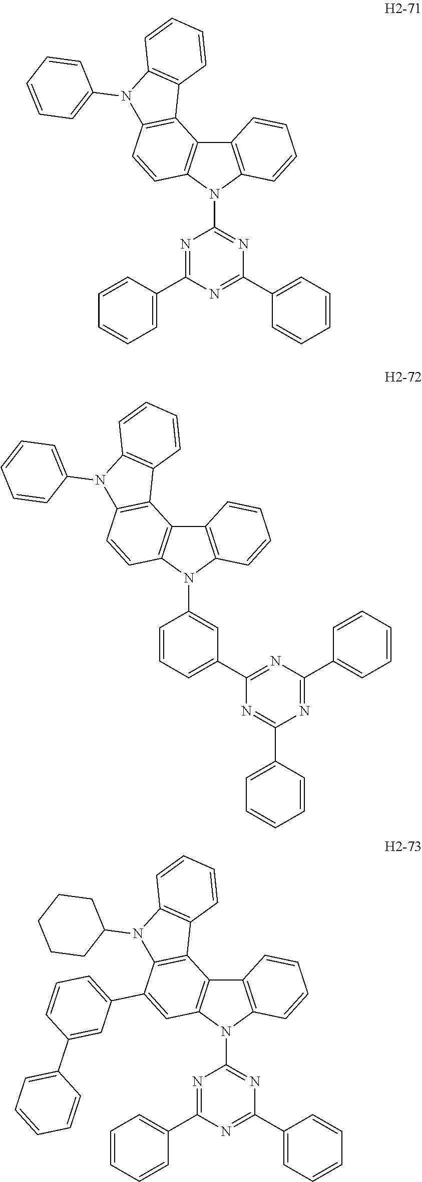 [SMILES: c1ccc(-c2nc(-c3ccccc3)nc(-n3c4ccccc4c4c5c6ccccc6n(-c6ccccc6)c5ccc43)n2)cc1 ; c1ccc(-c2nc(-c3ccccc3)nc(-c3cccc(-n4c5ccccc5c5c6c7ccccc7n(-c7ccccc7)c6ccc54)c3)n2)cc1 ; c1ccc(-c2cccc(-c3cc4c(c5ccccc5n4-c4nc(-c5ccccc5)nc(-c5ccccc5)n4)c4c5ccccc5n(C5CCCCC5)c34)c2)cc1]

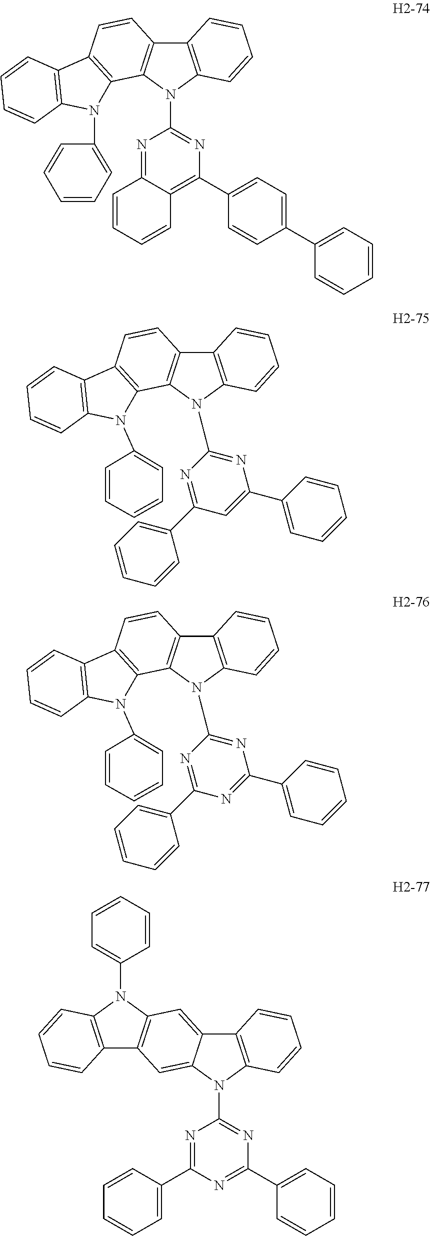

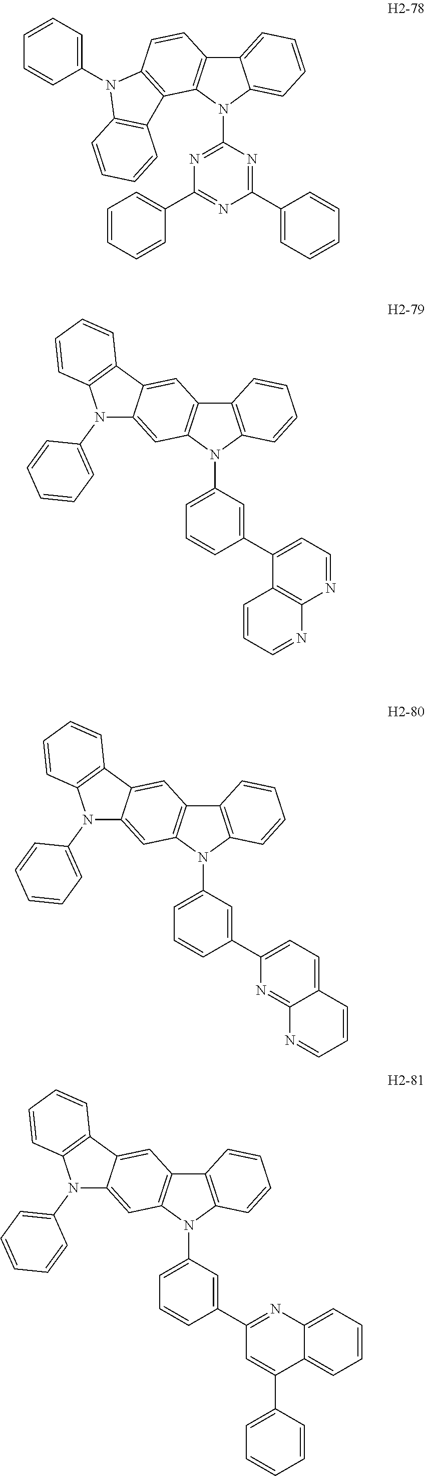 [SMILES: c1ccc(-c2nc(-c3ccccc3)nc(-n3c4ccccc4c4ccc5c(c6ccccc6n5-c5ccccc5)c43)n2)cc1 ; c1ccc(-n2c3ccccc3c3cc4c5ccccc5n(-c5cccc(-c6ccnc7ncccc67)c5)c4cc32)cc1 ; c1ccc(-n2c3ccccc3c3cc4c5ccccc5n(-c5cccc(-c6ccc7cccnc7n6)c5)c4cc32)cc1 ; c1ccc(-c2cc(-c3cccc(-n4c5ccccc5c5cc6c7ccccc7n(-c7ccccc7)c6cc54)c3)nc3ccccc23)cc1]

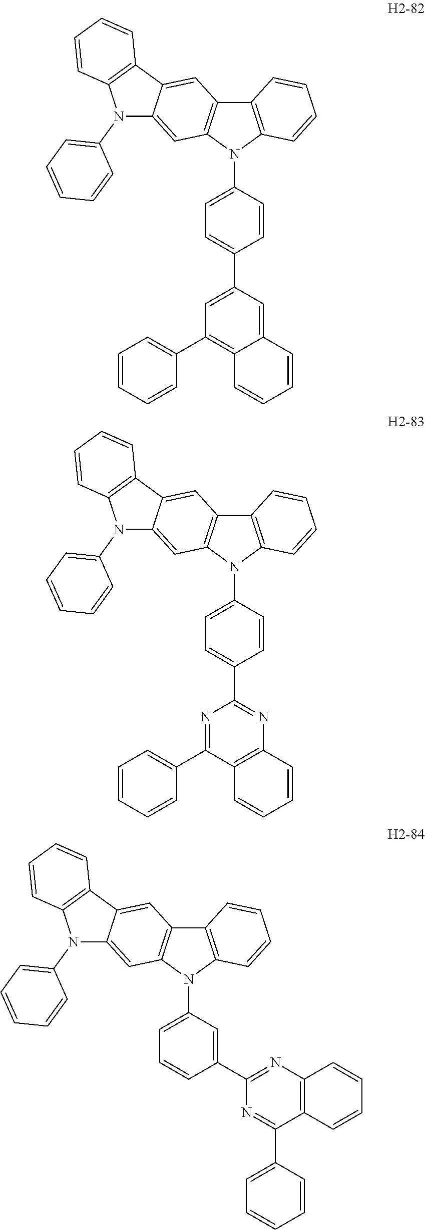

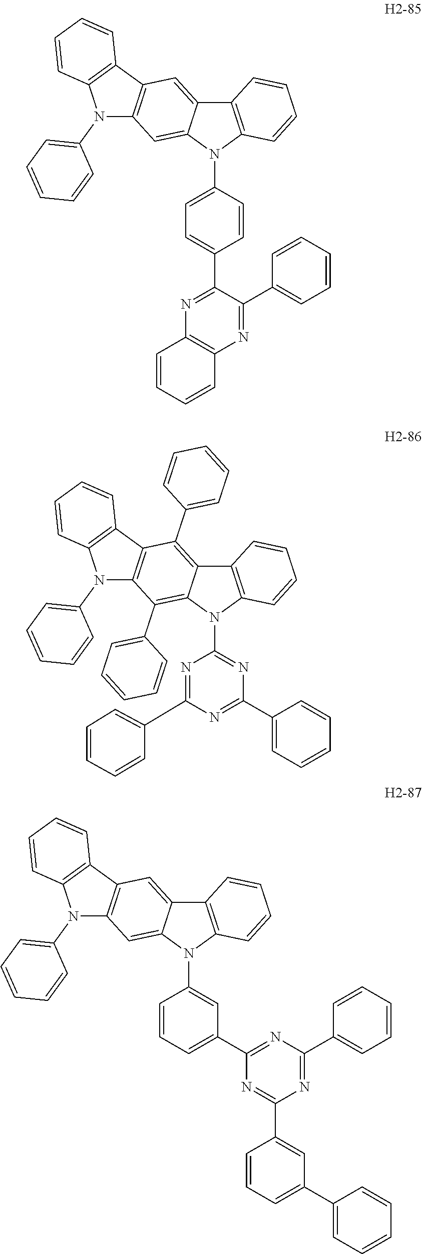 [SMILES: c1ccc(-c2nc3ccccc3nc2-c2ccc(-n3c4ccccc4c4cc5c6ccccc6n(-c6ccccc6)c5cc43)cc2)cc1 ; c1ccc(-c2nc(-c3ccccc3)nc(-n3c4ccccc4c4c(-c5ccccc5)c5c6ccccc6n(-c6ccccc6)c5c(-c5ccccc5)c43)n2)cc1 ; c1ccc(-c2cccc(-c3nc(-c4ccccc4)nc(-c4cccc(-n5c6ccccc6c6cc7c8ccccc8n(-c8ccccc8)c7cc65)c4)n3)c2)cc1]

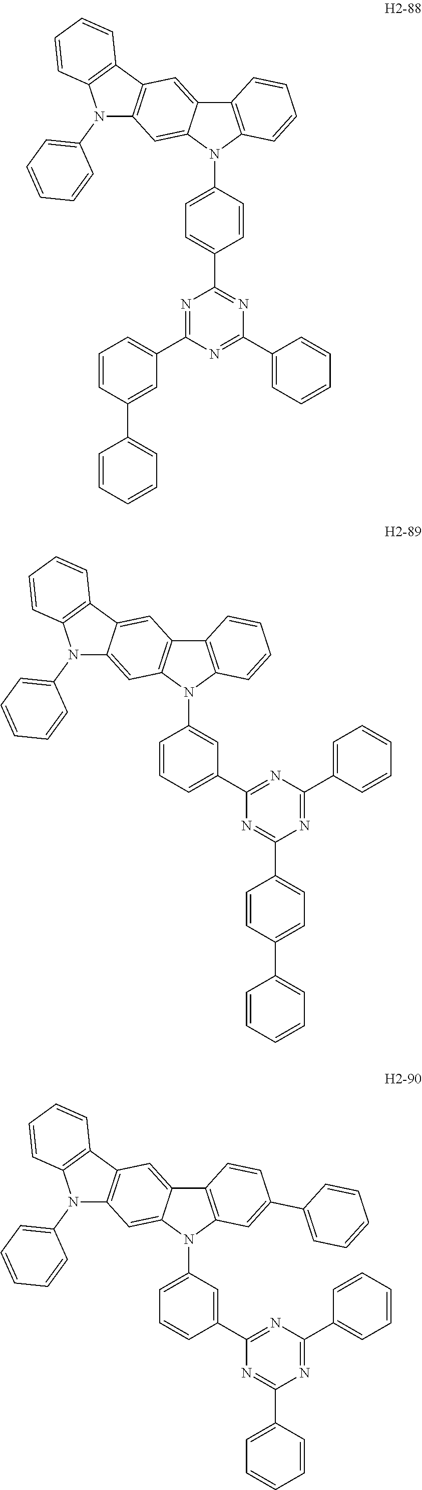 [SMILES: c1ccc(-c2cccc(-c3nc(-c4ccccc4)nc(-c4ccc(-n5c6ccccc6c6cc7c8ccccc8n(-c8ccccc8)c7cc65)cc4)n3)c2)cc1 ; c1ccc(-c2ccc(-c3nc(-c4ccccc4)nc(-c4cccc(-n5c6ccccc6c6cc7c8ccccc8n(-c8ccccc8)c7cc65)c4)n3)cc2)cc1 ; c1ccc(-c2ccc3c4cc5c6ccccc6n(-c6ccccc6)c5cc4n(-c4cccc(-c5nc(-c6ccccc6)nc(-c6ccccc6)n5)c4)c3c2)cc1]

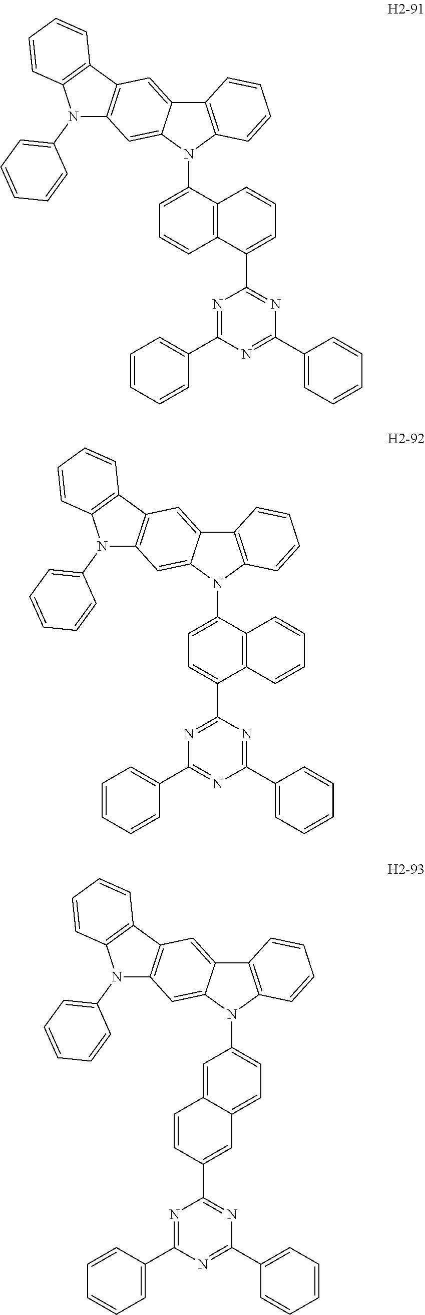 [SMILES: c1ccc(-c2nc(-c3ccccc3)nc(-c3cccc4c(-n5c6ccccc6c6cc7c8ccccc8n(-c8ccccc8)c7cc65)cccc34)n2)cc1 ; c1ccc(-c2nc(-c3ccccc3)nc(-c3ccc(-n4c5ccccc5c5cc6c7ccccc7n(-c7ccccc7)c6cc54)c4ccccc34)n2)cc1 ; c1ccc(-c2nc(-c3ccccc3)nc(-c3ccc4cc(-n5c6ccccc6c6cc7c8ccccc8n(-c8ccccc8)c7cc65)ccc4c3)n2)cc1]

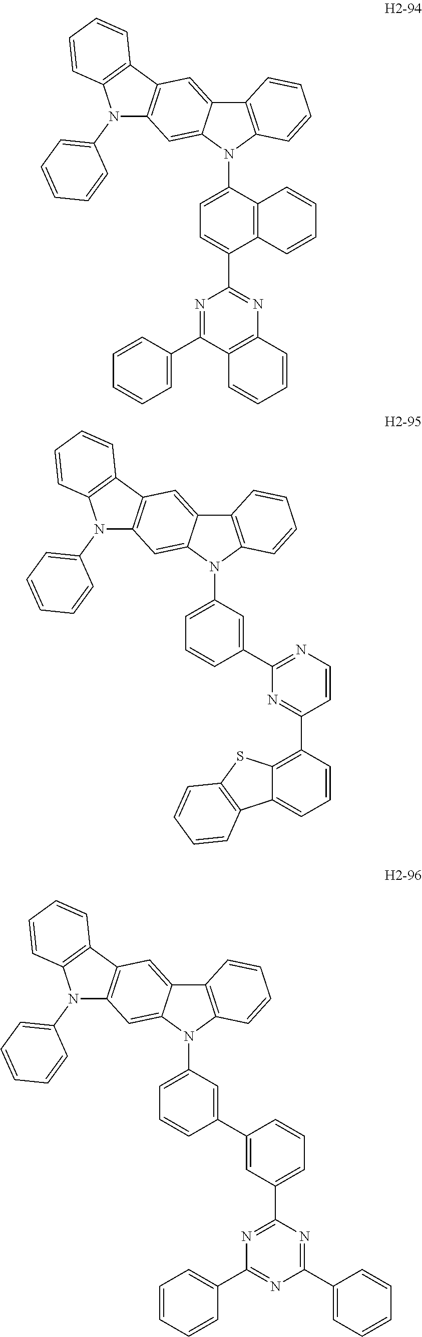 [SMILES: c1ccc(-c2nc(-c3ccc(-n4c5ccccc5c5cc6c7ccccc7n(-c7ccccc7)c6cc54)c4ccccc34)nc3ccccc23)cc1 ; c1ccc(-n2c3ccccc3c3cc4c5ccccc5n(-c5cccc(-c6nccc(-c7cccc8c7sc7ccccc78)n6)c5)c4cc32)cc1 ; c1ccc(-c2nc(-c3ccccc3)nc(-c3cccc(-c4cccc(-n5c6ccccc6c6cc7c8ccccc8n(-c8ccccc8)c7cc65)c4)c3)n2)cc1]

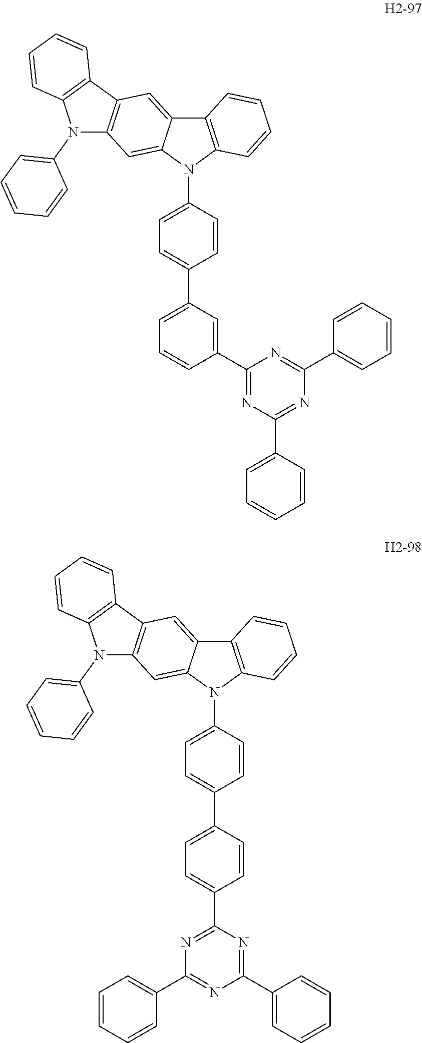 [SMILES: c1ccc(-c2nc(-c3ccccc3)nc(-c3cccc(-c4ccc(-n5c6ccccc6c6cc7c8ccccc8n(-c8ccccc8)c7cc65)cc4)c3)n2)cc1 ; c1ccc(-c2nc(-c3ccccc3)nc(-c3ccc(-c4ccc(-n5c6ccccc6c6cc7c8ccccc8n(-c8ccccc8)c7cc65)cc4)cc3)n2)cc1]

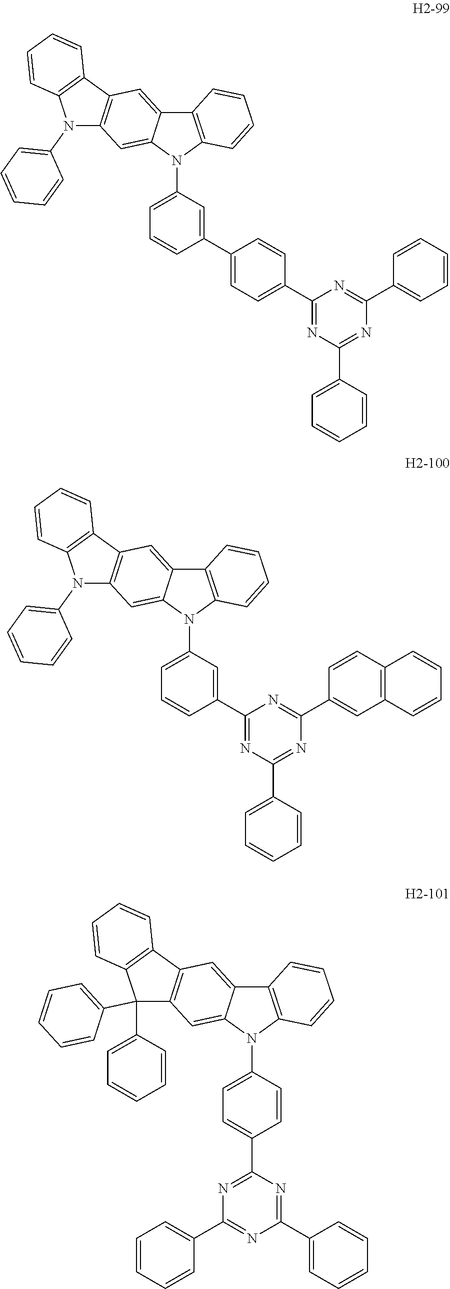 [SMILES: c1ccc(-c2nc(-c3ccccc3)nc(-c3ccc(-c4cccc(-n5c6ccccc6c6cc7c8ccccc8n(-c8ccccc8)c7cc65)c4)cc3)n2)cc1 ; c1ccc(-c2nc(-c3cccc(-n4c5ccccc5c5cc6c7ccccc7n(-c7ccccc7)c6cc54)c3)nc(-c3ccc4ccccc4c3)n2)cc1 ; c1ccc(-c2nc(-c3ccccc3)nc(-c3ccc(-n4c5ccccc5c5cc6c(cc54)C(c4ccccc4)(c4ccccc4)c4ccccc4-6)cc3)n2)cc1]

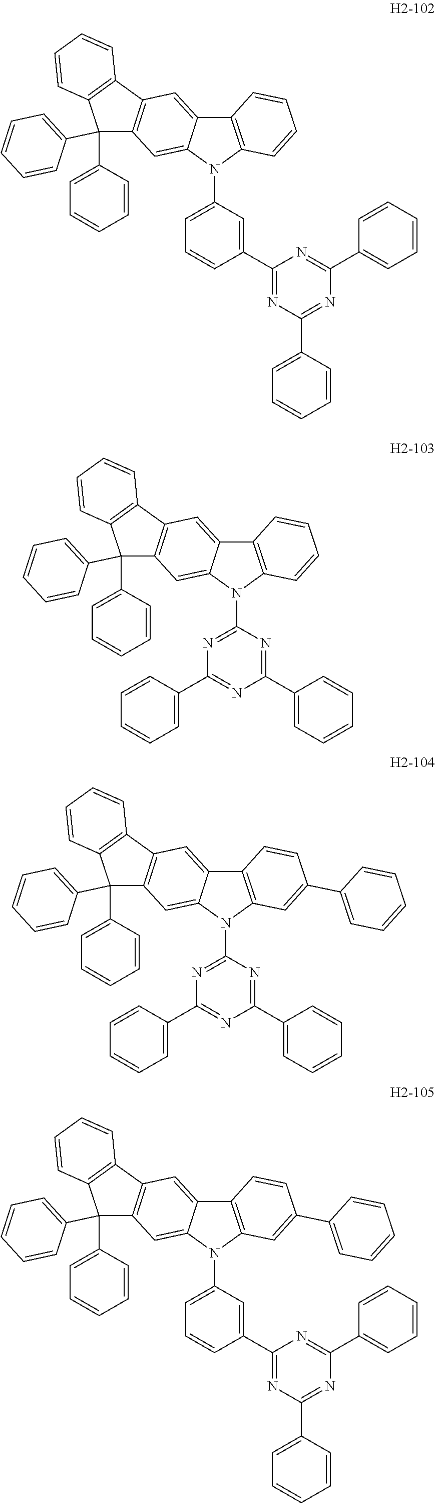 [SMILES: c1ccc(-c2nc(-c3ccccc3)nc(-c3cccc(-n4c5ccccc5c5cc6c(cc54)C(c4ccccc4)(c4ccccc4)c4ccccc4-6)c3)n2)cc1 ; c1ccc(-c2nc(-c3ccccc3)nc(-n3c4ccccc4c4cc5c(cc43)C(c3ccccc3)(c3ccccc3)c3ccccc3-5)n2)cc1 ; c1ccc(-c2ccc3c4cc5c(cc4n(-c4nc(-c6ccccc6)nc(-c6ccccc6)n4)c3c2)C(c2ccccc2)(c2ccccc2)c2ccccc2-5)cc1 ; c1ccc(-c2ccc3c4cc5c(cc4n(-c4cccc(-c6nc(-c7ccccc7)nc(-c7ccccc7)n6)c4)c3c2)C(c2ccccc2)(c2ccccc2)c2ccccc2-5)cc1]

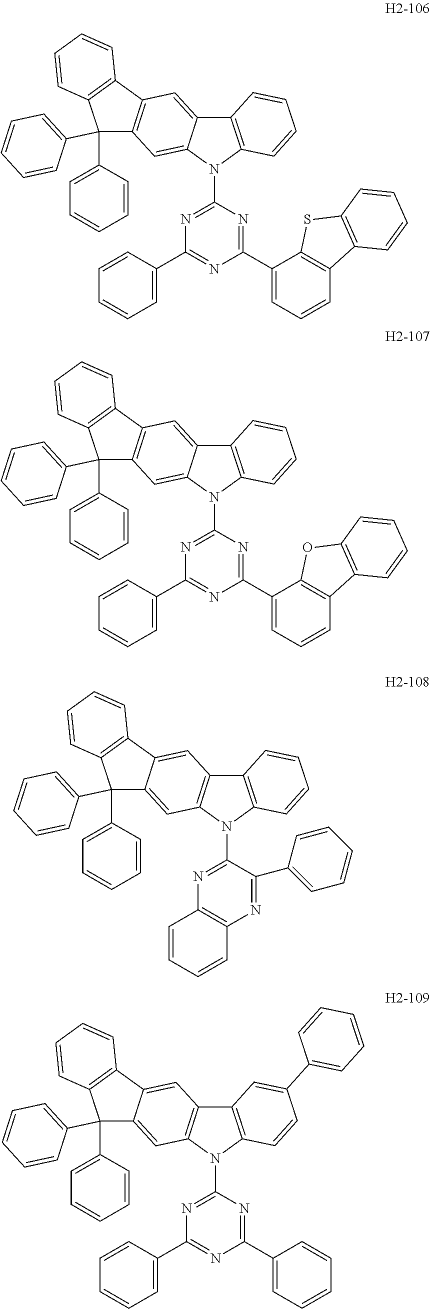 [SMILES: c1ccc(-c2nc(-c3cccc4c3sc3ccccc34)nc(-n3c4ccccc4c4cc5c(cc43)C(c3ccccc3)(c3ccccc3)c3ccccc3-5)n2)cc1 ; c1ccc(-c2nc(-c3cccc4c3oc3ccccc34)nc(-n3c4ccccc4c4cc5c(cc43)C(c3ccccc3)(c3ccccc3)c3ccccc3-5)n2)cc1 ; c1ccc(-c2nc3ccccc3nc2-n2c3ccccc3c3cc4c(cc32)C(c2ccccc2)(c2ccccc2)c2ccccc2-4)cc1 ; c1ccc(-c2ccc3c(c2)c2cc4c(cc2n3-c2nc(-c3ccccc3)nc(-c3ccccc3)n2)C(c2ccccc2)(c2ccccc2)c2ccccc2-4)cc1]

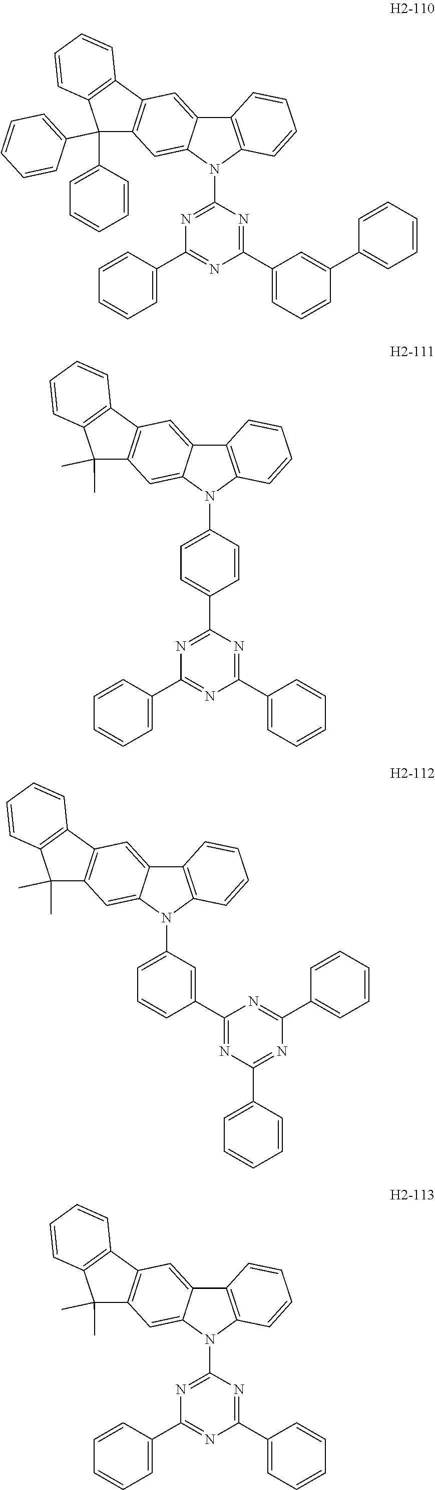 [SMILES: c1ccc(-c2cccc(-c3nc(-c4ccccc4)nc(-n4c5ccccc5c5cc6c(cc54)C(c4ccccc4)(c4ccccc4)c4ccccc4-6)n3)c2)cc1 ; CC1(C)c2ccccc2-c2cc3c4ccccc4n(-c4ccc(-c5nc(-c6ccccc6)nc(-c6ccccc6)n5)cc4)c3cc21 ; CC1(C)c2ccccc2-c2cc3c4ccccc4n(-c4cccc(-c5nc(-c6ccccc6)nc(-c6ccccc6)n5)c4)c3cc21 ; CC1(C)c2ccccc2-c2cc3c4ccccc4n(-c4nc(-c5ccccc5)nc(-c5ccccc5)n4)c3cc21]

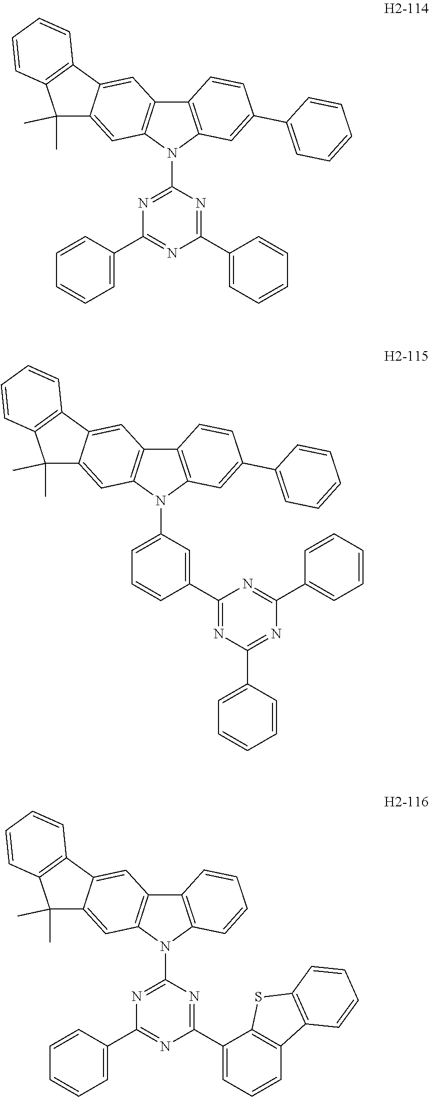 [SMILES: CC1(C)c2ccccc2-c2cc3c4ccc(-c5ccccc5)cc4n(-c4nc(-c5ccccc5)nc(-c5ccccc5)n4)c3cc21 ; CC1(C)c2ccccc2-c2cc3c4ccc(-c5ccccc5)cc4n(-c4cccc(-c5nc(-c6ccccc6)nc(-c6ccccc6)n5)c4)c3cc21 ; CC1(C)c2ccccc2-c2cc3c4ccccc4n(-c4nc(-c5ccccc5)nc(-c5cccc6c5sc5ccccc56)n4)c3cc21]

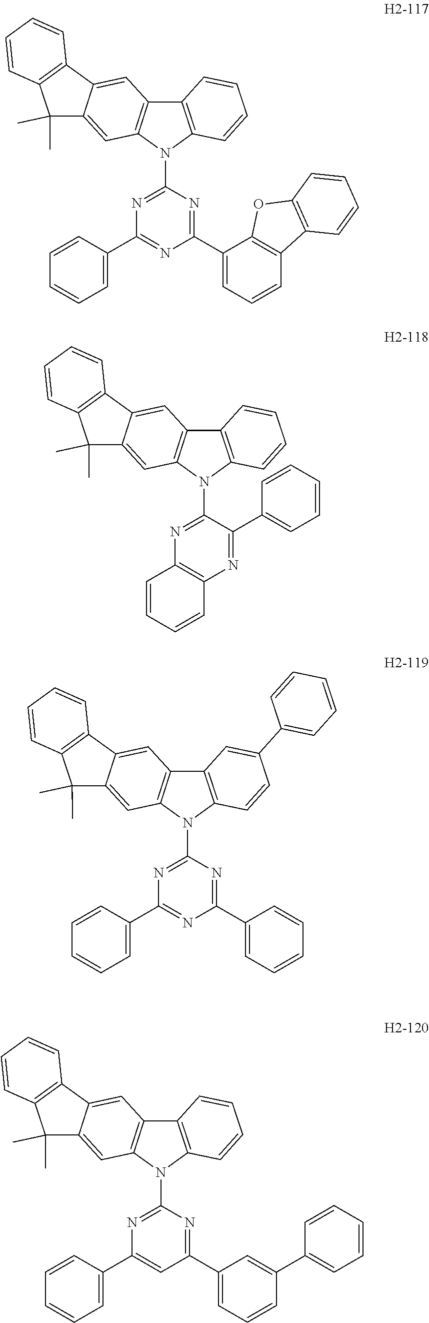

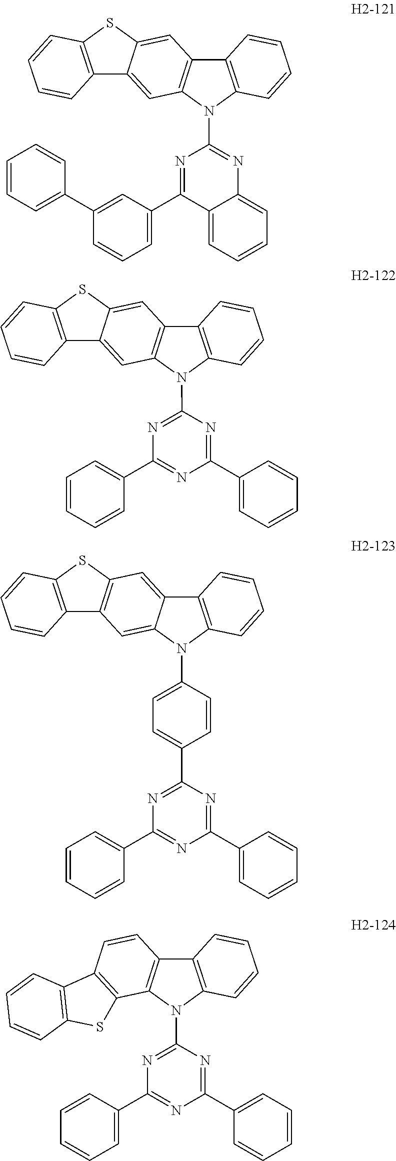

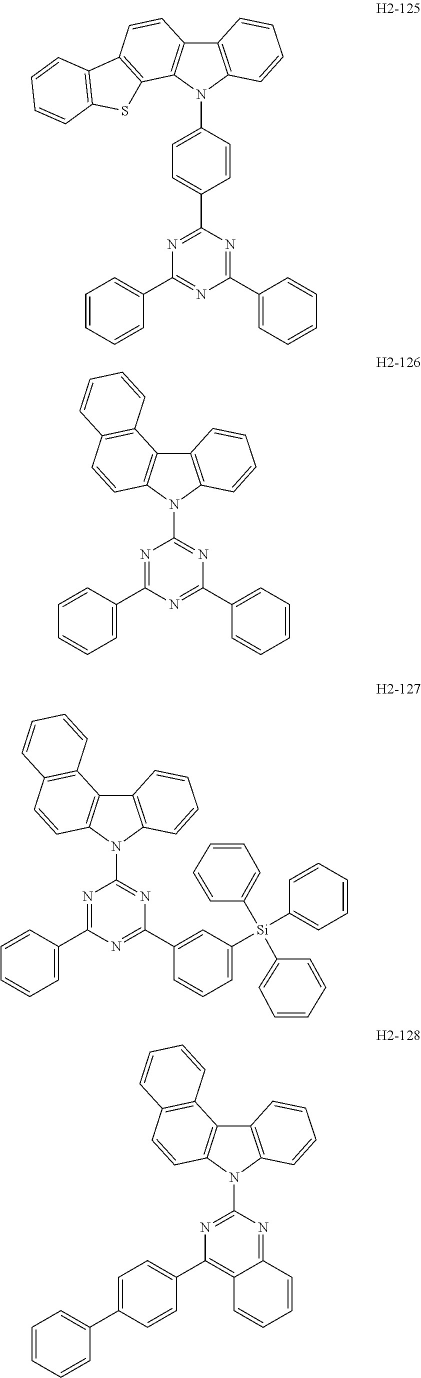

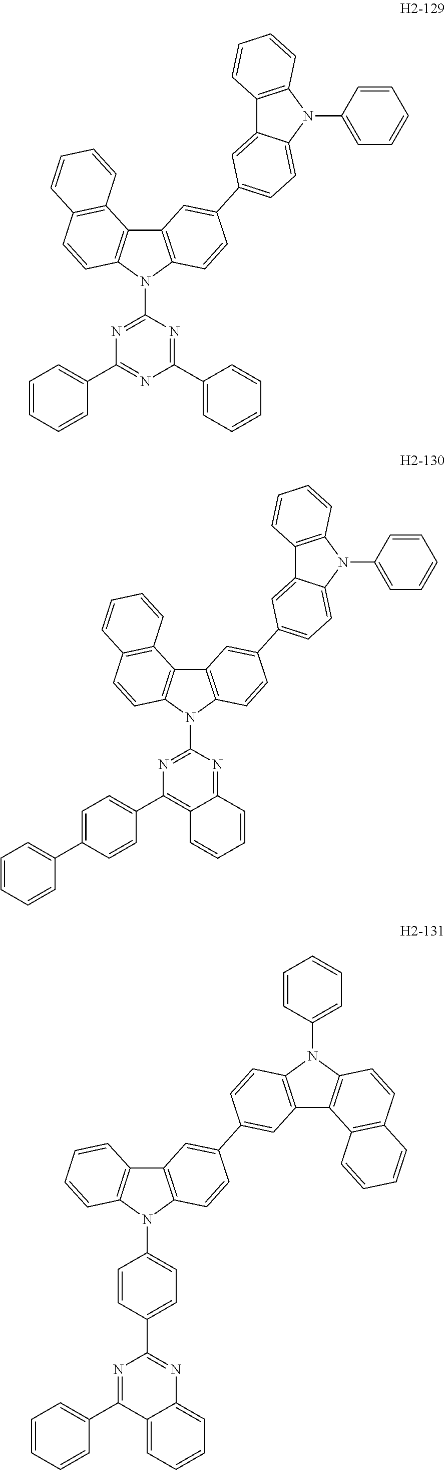 [SMILES: c1ccc(-c2nc(-c3ccccc3)nc(-n3c4ccc(-c5ccc6c(c5)c5ccccc5n6-c5ccccc5)cc4c4c5ccccc5ccc43)n2)cc1 ; c1ccc(-c2ccc(-c3nc(-n4c5ccc(-c6ccc7c(c6)c6ccccc6n7-c6ccccc6)cc5c5c6ccccc6ccc54)nc4ccccc34)cc2)cc1 ; c1ccc(-c2nc(-c3ccc(-n4c5ccccc5c5cc(-c6ccc7c(c6)c6c8ccccc8ccc6n7-c6ccccc6)ccc54)cc3)nc3ccccc23)cc1]

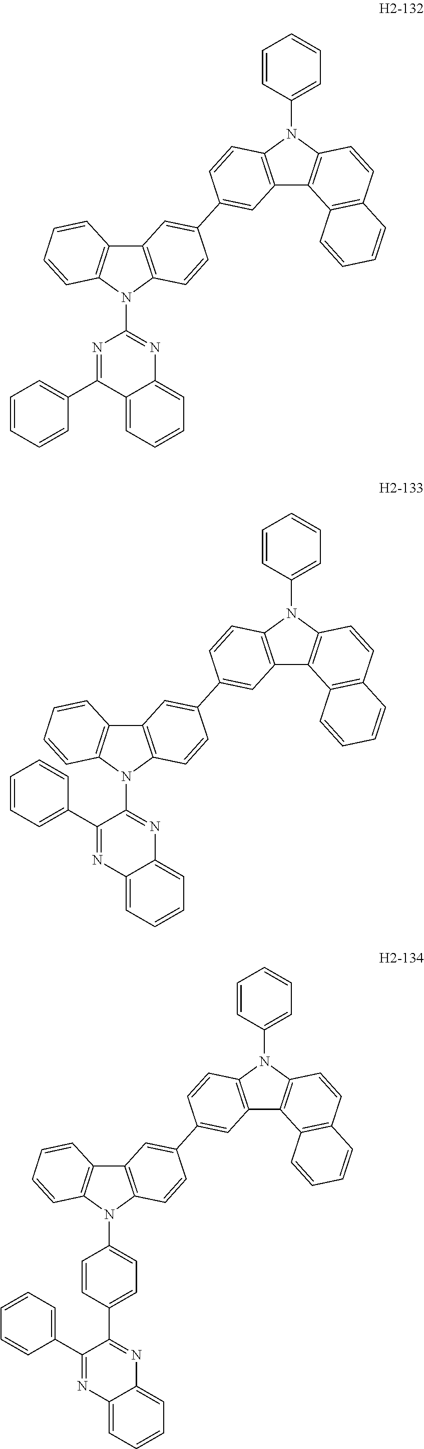 [SMILES: c1ccc(-c2nc(-n3c4ccccc4c4cc(-c5ccc6c(c5)c5c7ccccc7ccc5n6-c5ccccc5)ccc43)nc3ccccc23)cc1 ; c1ccc(-c2nc3ccccc3nc2-n2c3ccccc3c3cc(-c4ccc5c(c4)c4c6ccccc6ccc4n5-c4ccccc4)ccc32)cc1 ; c1ccc(-c2nc3ccccc3nc2-c2ccc(-n3c4ccccc4c4cc(-c5ccc6c(c5)c5c7ccccc7ccc5n6-c5ccccc5)ccc43)cc2)cc1]

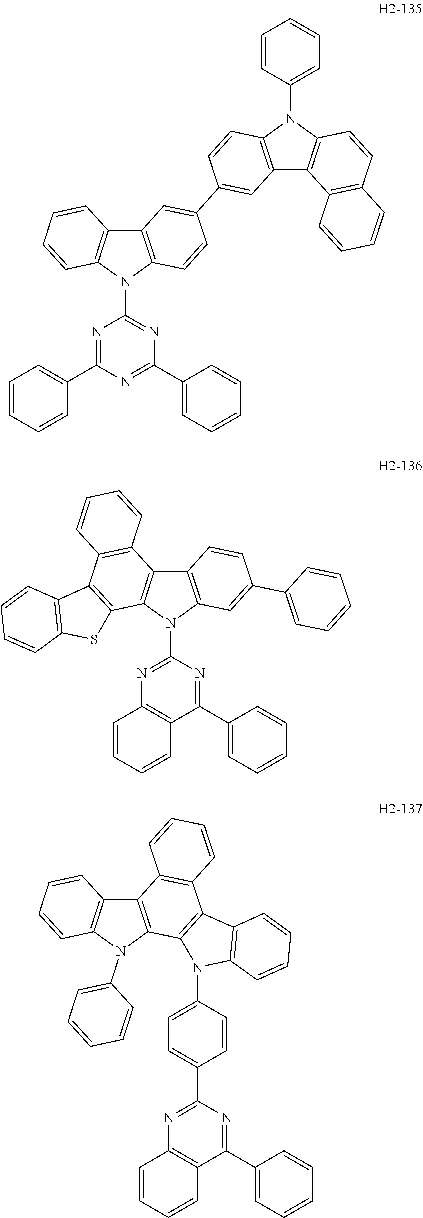 [SMILES: c1ccc(-c2nc(-c3ccccc3)nc(-n3c4ccccc4c4cc(-c5ccc6c(c5)c5c7ccccc7ccc5n6-c5ccccc5)ccc43)n2)cc1 ; c1ccc(-c2ccc3c4c5ccccc5c5c6ccccc6sc5c4n(-c4nc(-c5ccccc5)c5ccccc5n4)c3c2)cc1 ; c1ccc(-c2nc(-c3ccc(-n4c5ccccc5c5c6ccccc6c6c7ccccc7n(-c7ccccc7)c6c54)cc3)nc3ccccc23)cc1]

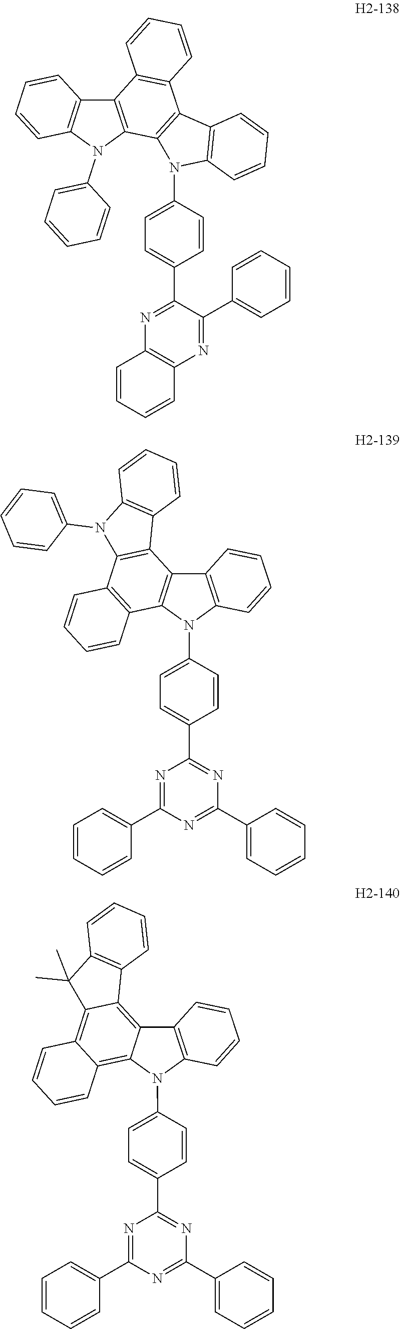

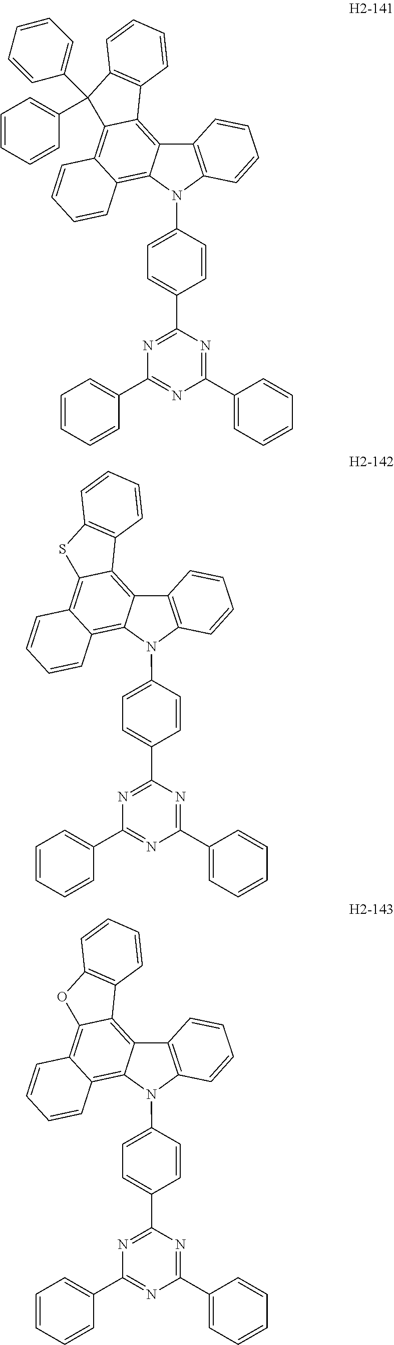

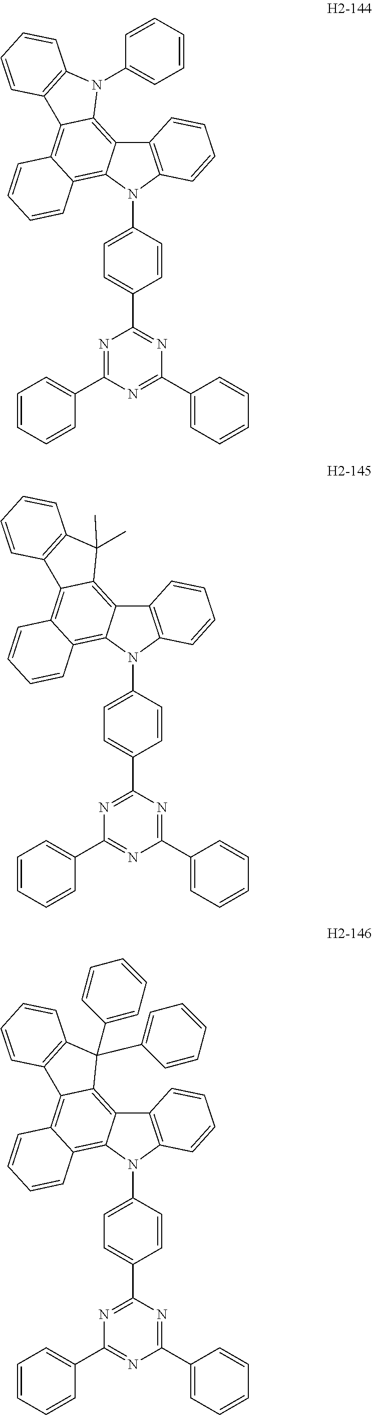 [SMILES: c1ccc(-c2nc(-c3ccccc3)nc(-c3ccc(-n4c5ccccc5c5c4c4ccccc4c4c6ccccc6n(-c6ccccc6)c45)cc3)n2)cc1 ; CC1(C)c2ccccc2-c2c1c1c3ccccc3n(-c3ccc(-c4nc(-c5ccccc5)nc(-c5ccccc5)n4)cc3)c1c1ccccc21 ; c1ccc(-c2nc(-c3ccccc3)nc(-c3ccc(-n4c5ccccc5c5c6c(c7ccccc7c54)-c4ccccc4C6(c4ccccc4)c4ccccc4)cc3)n2)cc1]

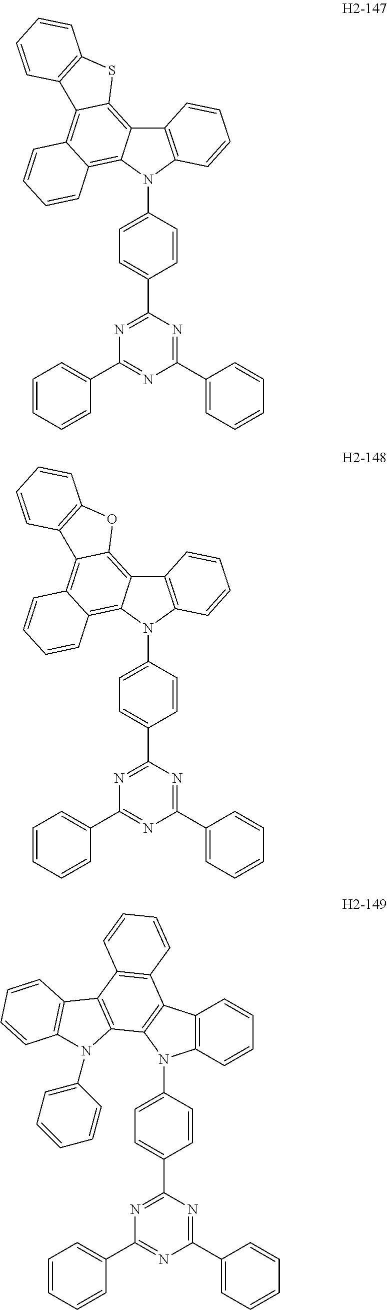

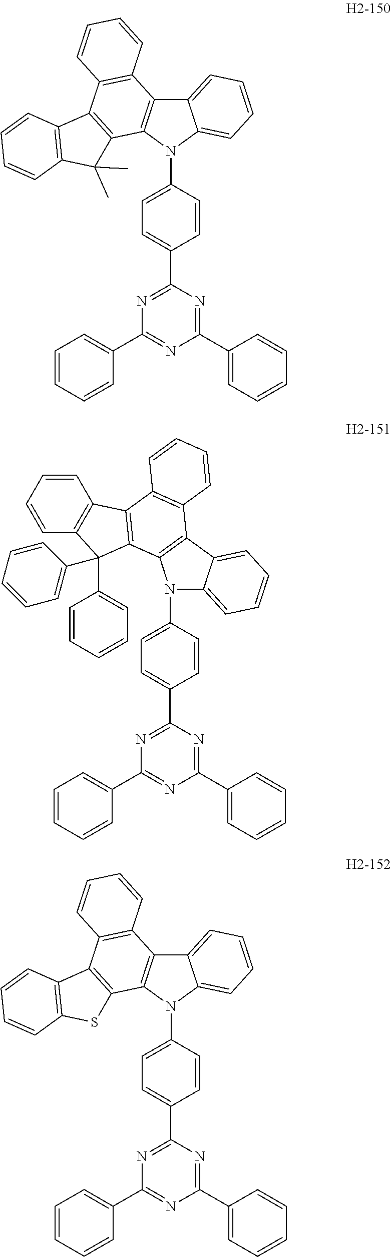

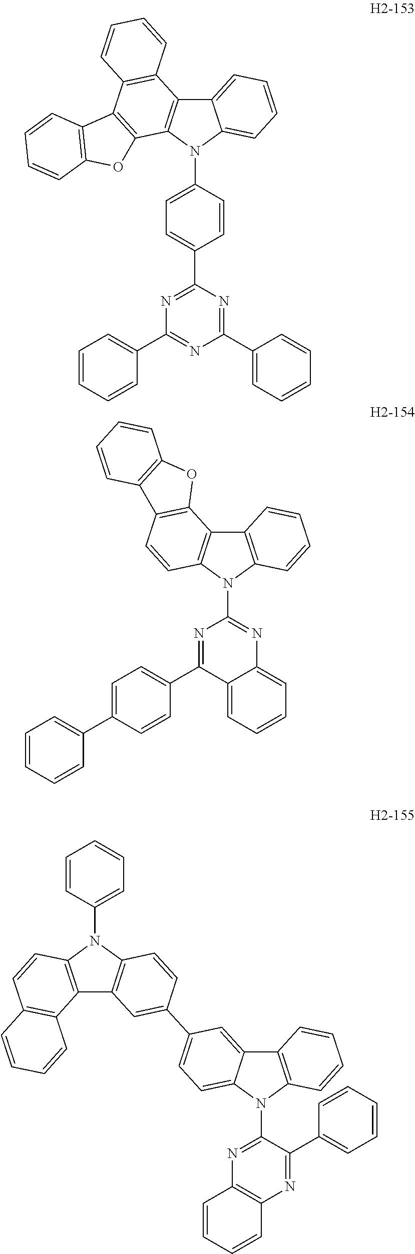

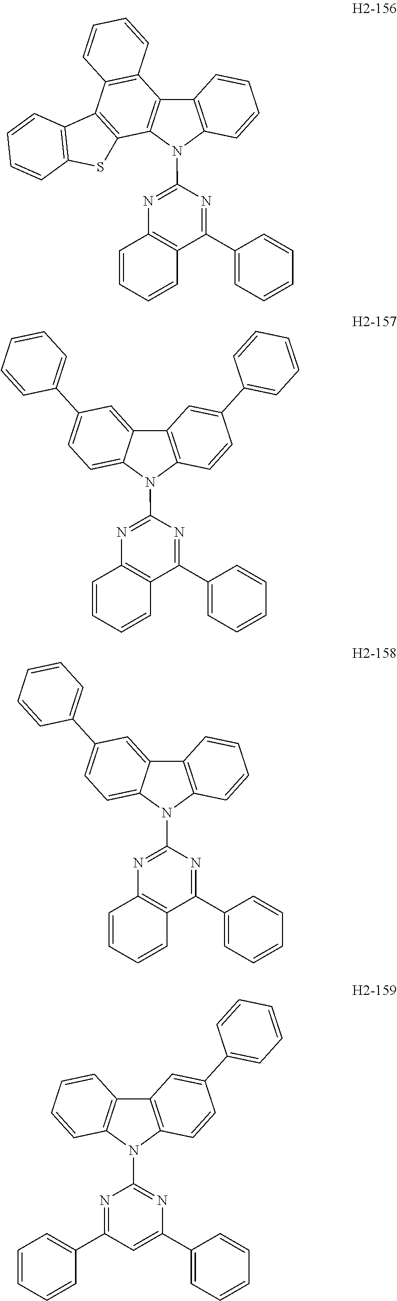

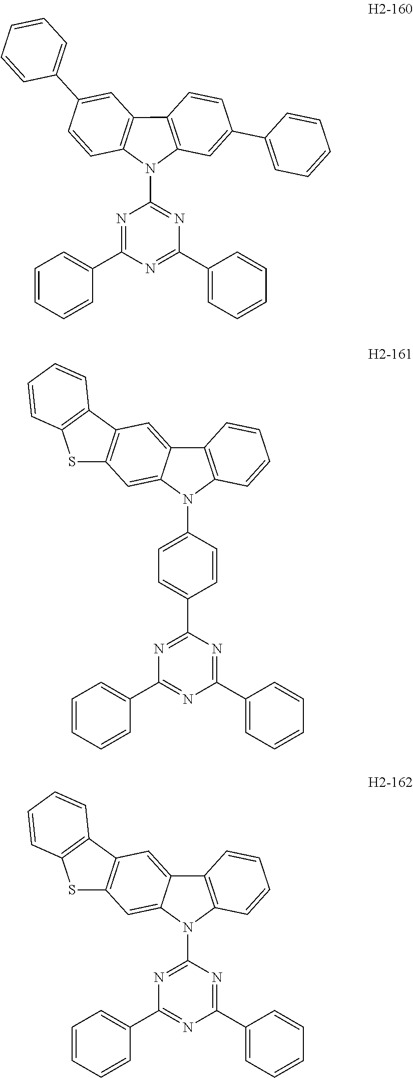

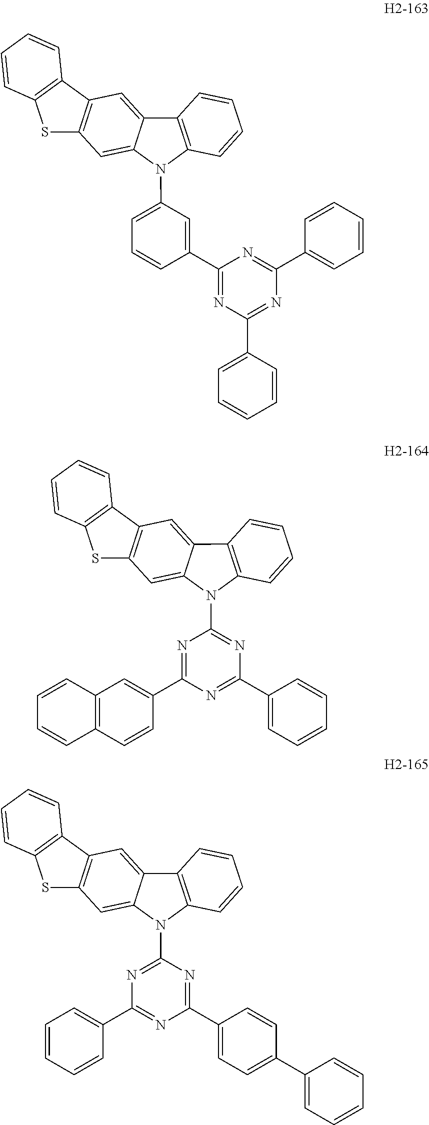 [SMILES: c1ccc(-c2nc(-c3ccccc3)nc(-c3cccc(-n4c5ccccc5c5cc6c(cc54)sc4ccccc46)c3)n2)cc1 ; c1ccc(-c2nc(-c3ccc4ccccc4c3)nc(-n3c4ccccc4c4cc5c(cc43)sc3ccccc35)n2)cc1 ; c1ccc(-c2ccc(-c3nc(-c4ccccc4)nc(-n4c5ccccc5c5cc6c(cc54)sc4ccccc46)n3)cc2)cc1]

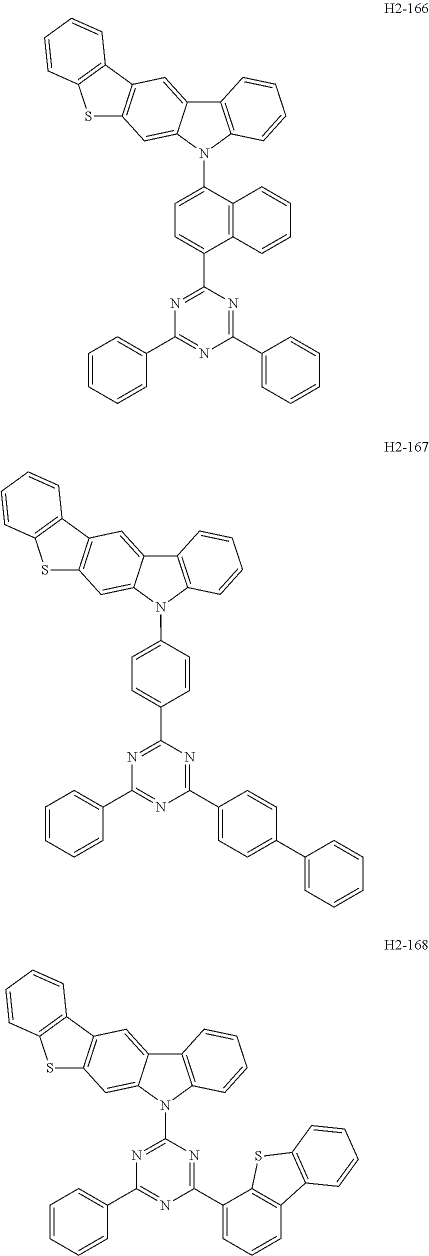 [SMILES: c1ccc(-c2nc(-c3ccccc3)nc(-c3ccc(-n4c5ccccc5c5cc6c(cc54)sc4ccccc46)c4ccccc34)n2)cc1 ; c1ccc(-c2ccc(-c3nc(-c4ccccc4)nc(-c4ccc(-n5c6ccccc6c6cc7c(cc65)sc5ccccc57)cc4)n3)cc2)cc1 ; c1ccc(-c2nc(-c3cccc4c3sc3ccccc34)nc(-n3c4ccccc4c4cc5c(cc43)sc3ccccc35)n2)cc1]

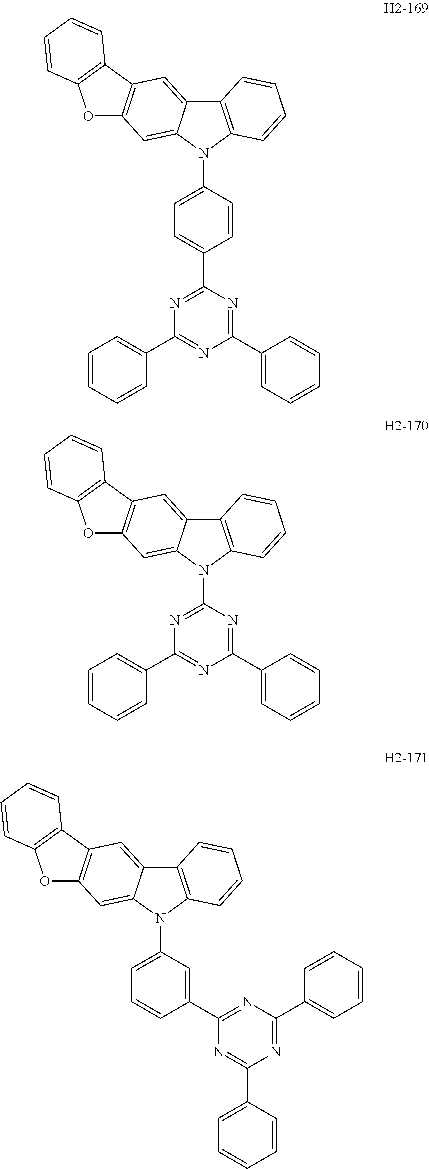 [SMILES: c1ccc(-c2nc(-c3ccccc3)nc(-c3ccc(-n4c5ccccc5c5cc6c(cc54)oc4ccccc46)cc3)n2)cc1 ; c1ccc(-c2nc(-c3ccccc3)nc(-n3c4ccccc4c4cc5c(cc43)oc3ccccc35)n2)cc1 ; c1ccc(-c2nc(-c3ccccc3)nc(-c3cccc(-n4c5ccccc5c5cc6c(cc54)oc4ccccc46)c3)n2)cc1]

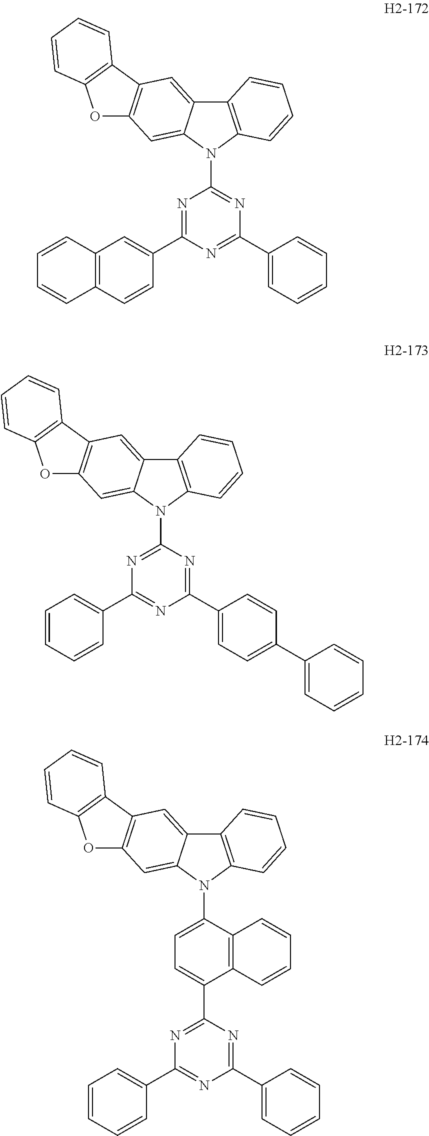 [SMILES: c1ccc(-c2nc(-c3ccc4ccccc4c3)nc(-n3c4ccccc4c4cc5c(cc43)oc3ccccc35)n2)cc1 ; c1ccc(-c2ccc(-c3nc(-c4ccccc4)nc(-n4c5ccccc5c5cc6c(cc54)oc4ccccc46)n3)cc2)cc1 ; c1ccc(-c2nc(-c3ccccc3)nc(-c3ccc(-n4c5ccccc5c5cc6c(cc54)oc4ccccc46)c4ccccc34)n2)cc1]

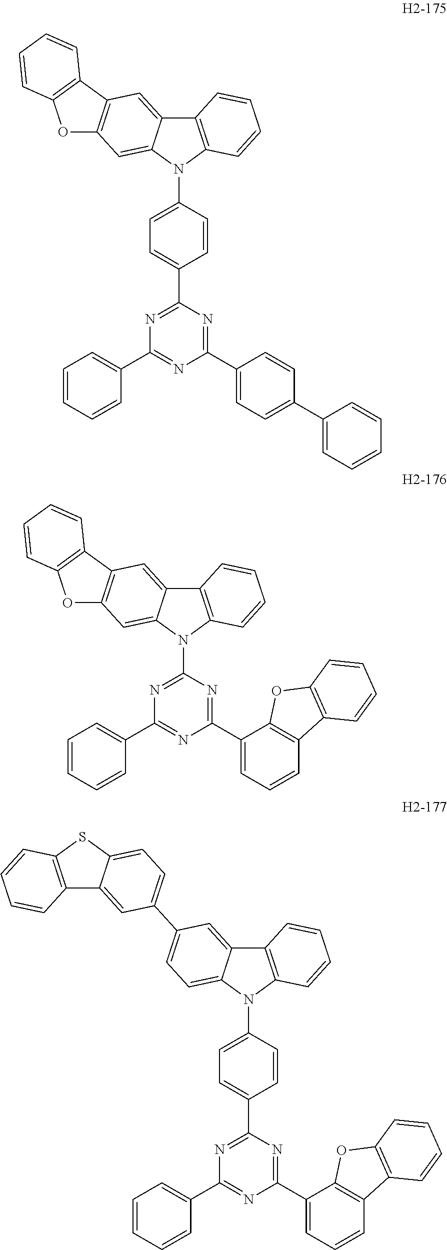 [SMILES: c1ccc(-c2ccc(-c3nc(-c4ccccc4)nc(-c4ccc(-n5c6ccccc6c6cc7c(cc65)oc5ccccc57)cc4)n3)cc2)cc1 ; c1ccc(-c2nc(-c3cccc4c3oc3ccccc34)nc(-n3c4ccccc4c4cc5c(cc43)oc3ccccc35)n2)cc1 ; c1ccc(-c2nc(-c3ccc(-n4c5ccccc5c5cc(-c6ccc7sc8ccccc8c7c6)ccc54)cc3)nc(-c3cccc4c3oc3ccccc34)n2)cc1]

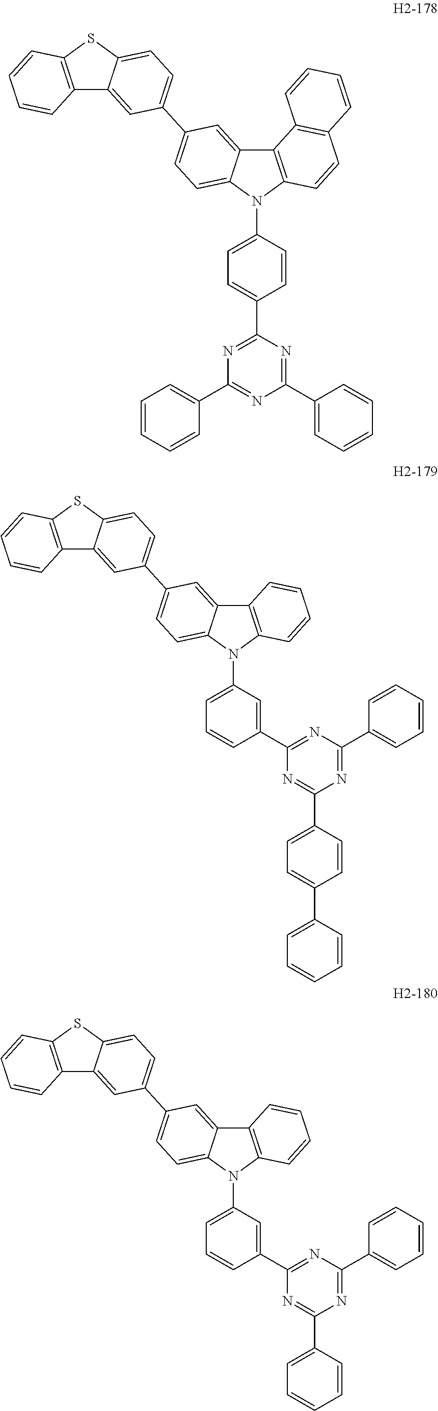 [SMILES: c1ccc(-c2nc(-c3ccccc3)nc(-c3ccc(-n4c5ccc(-c6ccc7sc8ccccc8c7c6)cc5c5c6ccccc6ccc54)cc3)n2)cc1 ; c1ccc(-c2ccc(-c3nc(-c4ccccc4)nc(-c4cccc(-n5c6ccccc6c6cc(-c7ccc8sc9ccccc9c8c7)ccc65)c4)n3)cc2)cc1 ; c1ccc(-c2nc(-c3ccccc3)nc(-c3cccc(-n4c5ccccc5c5cc(-c6ccc7sc8ccccc8c7c6)ccc54)c3)n2)cc1]

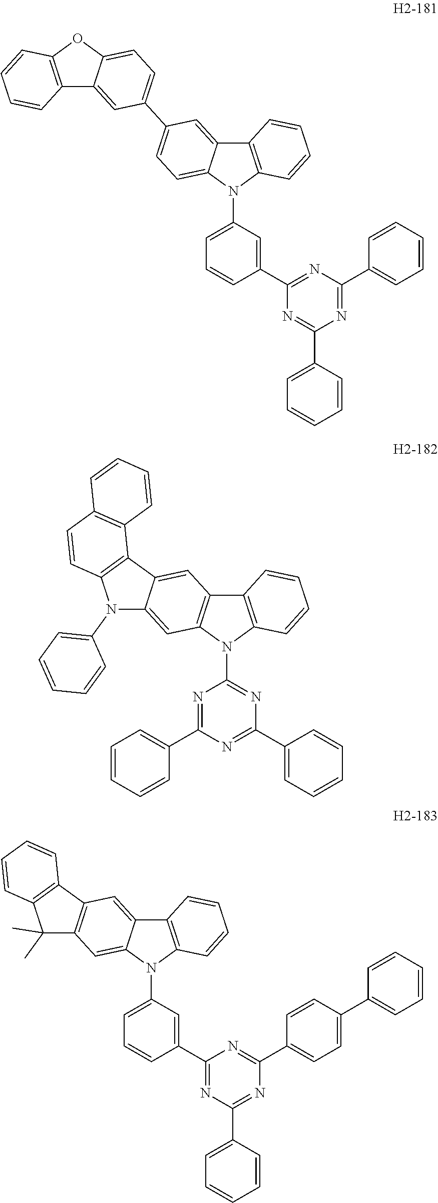 [SMILES: c1ccc(-c2nc(-c3ccccc3)nc(-c3cccc(-n4c5ccccc5c5cc(-c6ccc7oc8ccccc8c7c6)ccc54)c3)n2)cc1 ; c1ccc(-c2nc(-c3ccccc3)nc(-n3c4ccccc4c4cc5c6c7ccccc7ccc6n(-c6ccccc6)c5cc43)n2)cc1 ; CC1(C)c2ccccc2-c2cc3c4ccccc4n(-c4cccc(-c5nc(-c6ccccc6)nc(-c6ccc(-c7ccccc7)cc6)n5)c4)c3cc21]

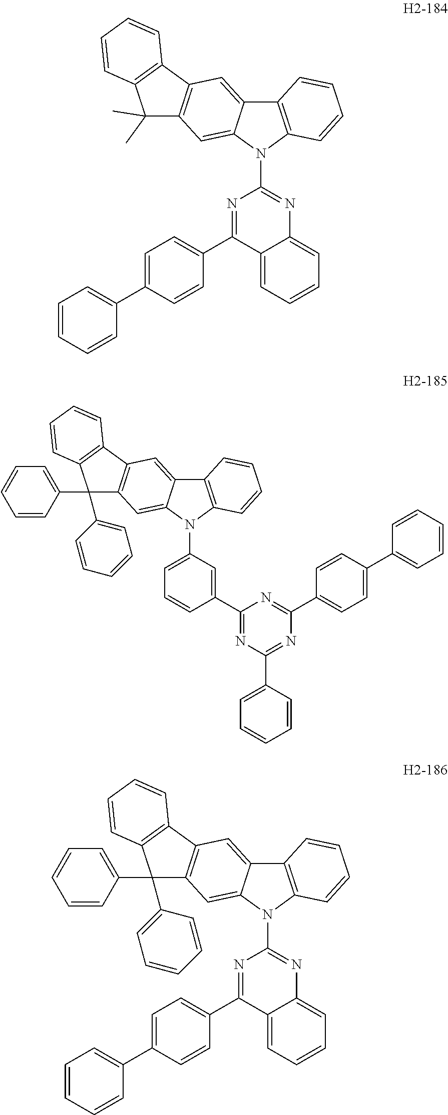 [SMILES: CC1(C)c2ccccc2-c2cc3c4ccccc4n(-c4nc(-c5ccc(-c6ccccc6)cc5)c5ccccc5n4)c3cc21 ; c1ccc(-c2ccc(-c3nc(-c4ccccc4)nc(-c4cccc(-n5c6ccccc6c6cc7c(cc65)C(c5ccccc5)(c5ccccc5)c5ccccc5-7)c4)n3)cc2)cc1 ; c1ccc(-c2ccc(-c3nc(-n4c5ccccc5c5cc6c(cc54)C(c4ccccc4)(c4ccccc4)c4ccccc4-6)nc4ccccc34)cc2)cc1]

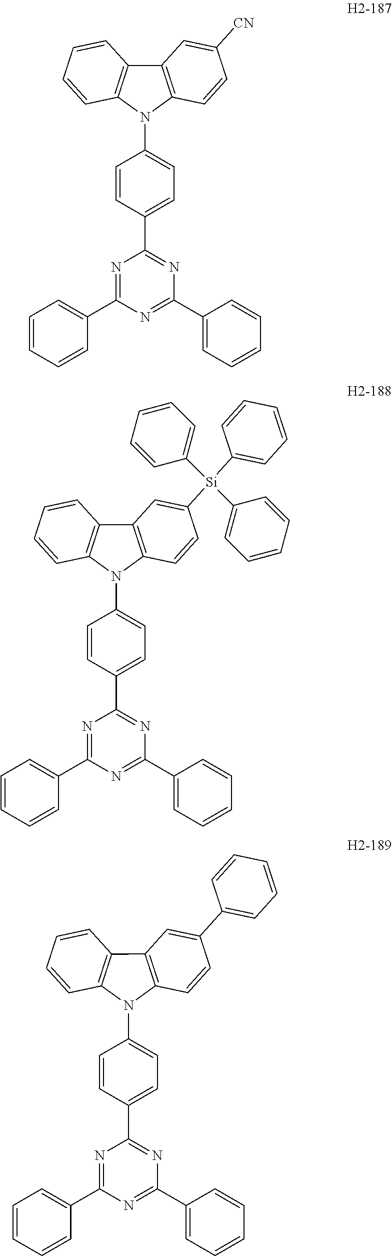

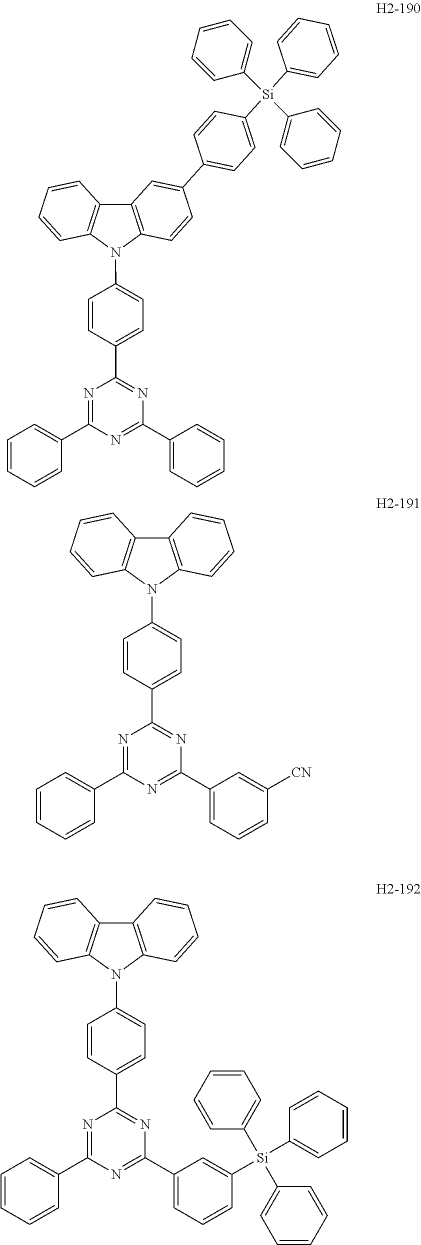

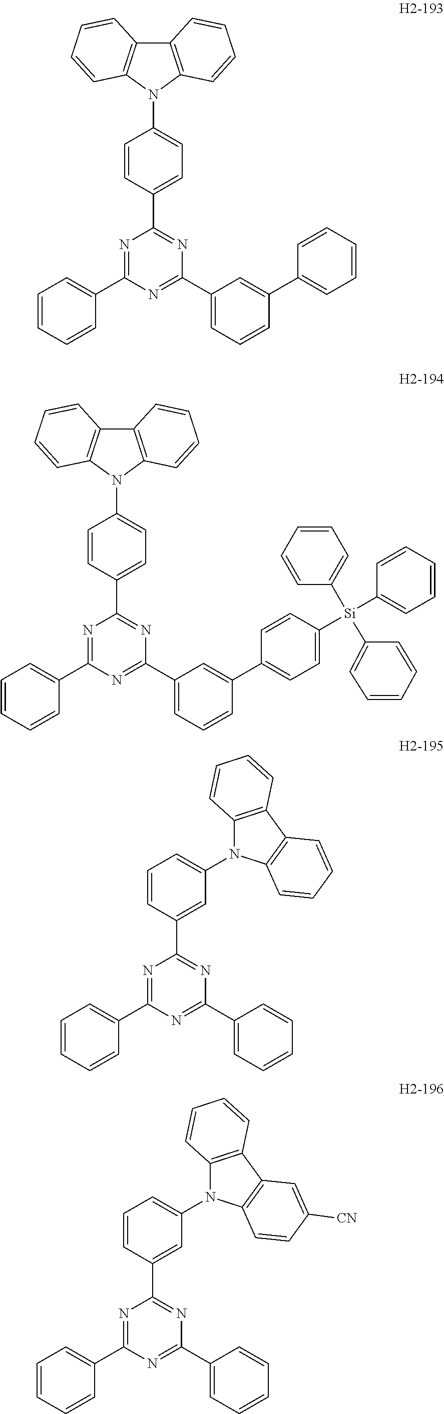

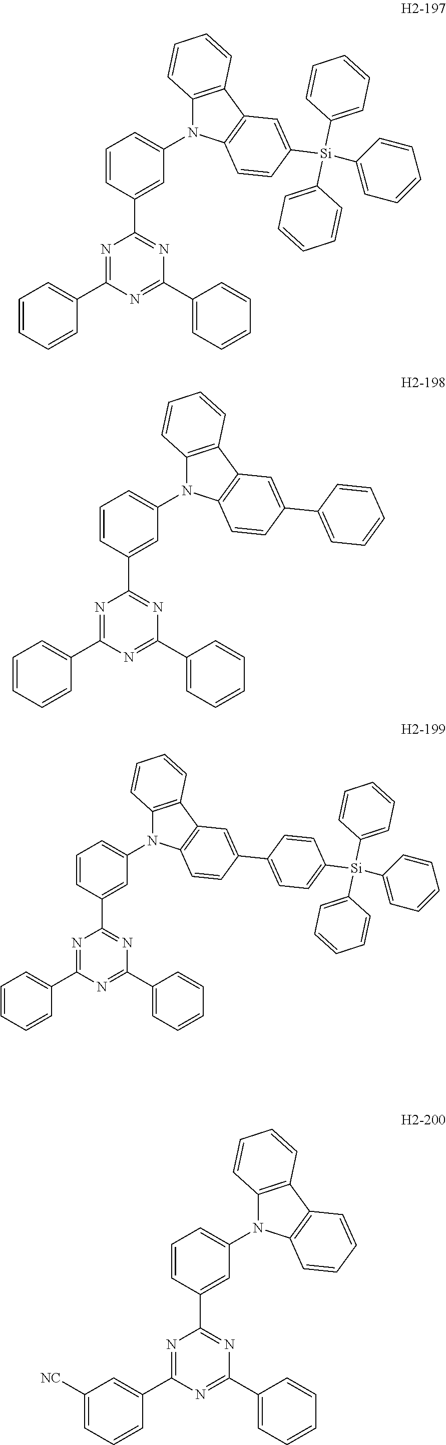 [SMILES: c1ccc(-c2nc(-c3ccccc3)nc(-c3cccc(-n4c5ccccc5c5cc([Si](c6ccccc6)(c6ccccc6)c6ccccc6)ccc54)c3)n2)cc1 ; c1ccc(-c2ccc3c(c2)c2ccccc2n3-c2cccc(-c3nc(-c4ccccc4)nc(-c4ccccc4)n3)c2)cc1 ; c1ccc(-c2nc(-c3ccccc3)nc(-c3cccc(-n4c5ccccc5c5cc(-c6ccc([Si](c7ccccc7)(c7ccccc7)c7ccccc7)cc6)ccc54)c3)n2)cc1 ; N#Cc1cccc(-c2nc(-c3ccccc3)nc(-c3cccc(-n4c5ccccc5c5ccccc54)c3)n2)c1]

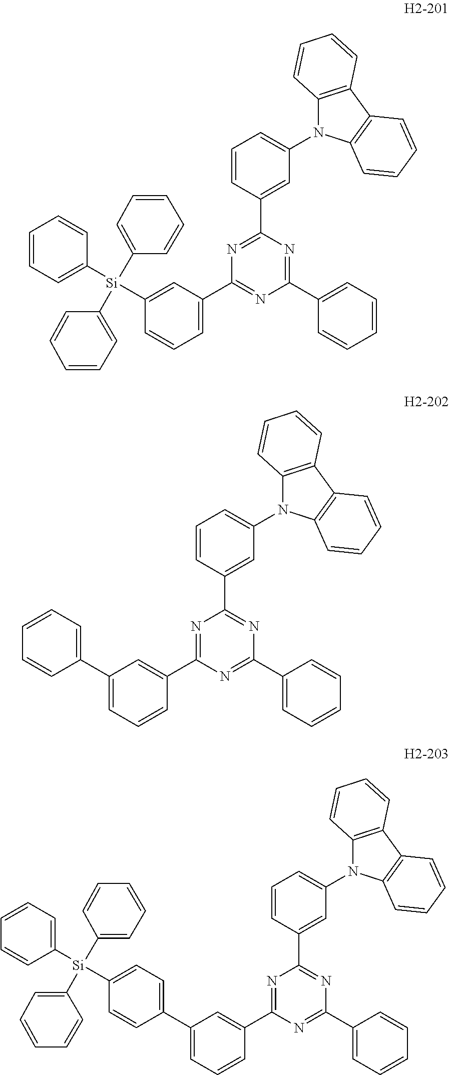 [SMILES: c1ccc(-c2nc(-c3cccc(-n4c5ccccc5c5ccccc54)c3)nc(-c3cccc([Si](c4ccccc4)(c4ccccc4)c4ccccc4)c3)n2)cc1 ; c1ccc(-c2cccc(-c3nc(-c4ccccc4)nc(-c4cccc(-n5c6ccccc6c6ccccc65)c4)n3)c2)cc1 ; c1ccc(-c2nc(-c3cccc(-c4ccc([Si](c5ccccc5)(c5ccccc5)c5ccccc5)cc4)c3)nc(-c3cccc(-n4c5ccccc5c5ccccc54)c3)n2)cc1]

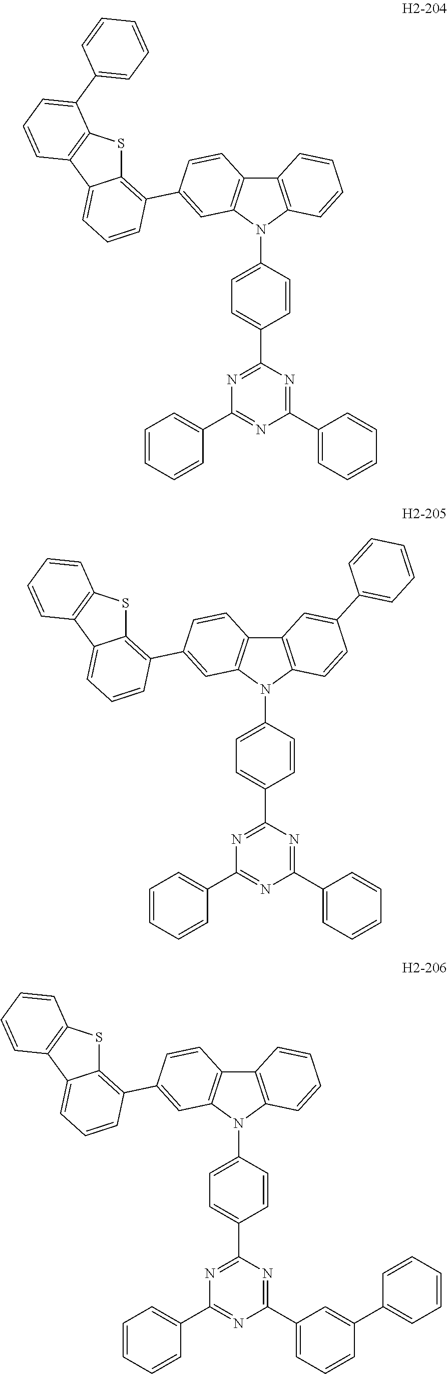 [SMILES: c1ccc(-c2nc(-c3ccccc3)nc(-c3ccc(-n4c5ccccc5c5ccc(-c6cccc7c6sc6c(-c8ccccc8)cccc67)cc54)cc3)n2)cc1 ; c1ccc(-c2ccc3c(c2)c2ccc(-c4cccc5c4sc4ccccc45)cc2n3-c2ccc(-c3nc(-c4ccccc4)nc(-c4ccccc4)n3)cc2)cc1 ; c1ccc(-c2cccc(-c3nc(-c4ccccc4)nc(-c4ccc(-n5c6ccccc6c6ccc(-c7cccc8c7sc7ccccc78)cc65)cc4)n3)c2)cc1]

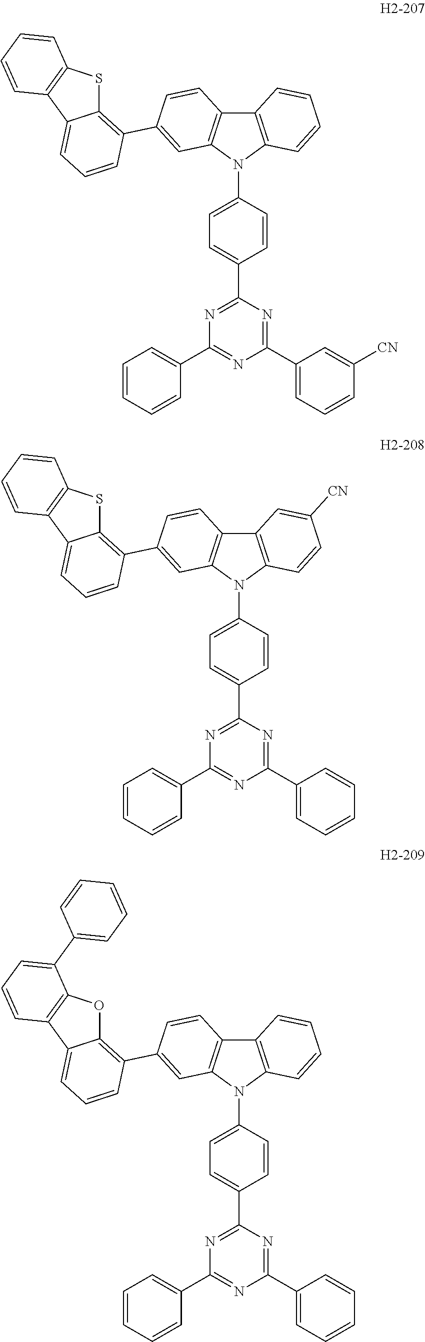 [SMILES: N#Cc1cccc(-c2nc(-c3ccccc3)nc(-c3ccc(-n4c5ccccc5c5ccc(-c6cccc7c6sc6ccccc67)cc54)cc3)n2)c1 ; N#Cc1ccc2c(c1)c1ccc(-c3cccc4c3sc3ccccc34)cc1n2-c1ccc(-c2nc(-c3ccccc3)nc(-c3ccccc3)n2)cc1 ; c1ccc(-c2nc(-c3ccccc3)nc(-c3ccc(-n4c5ccccc5c5ccc(-c6cccc7c6oc6c(-c8ccccc8)cccc67)cc54)cc3)n2)cc1]

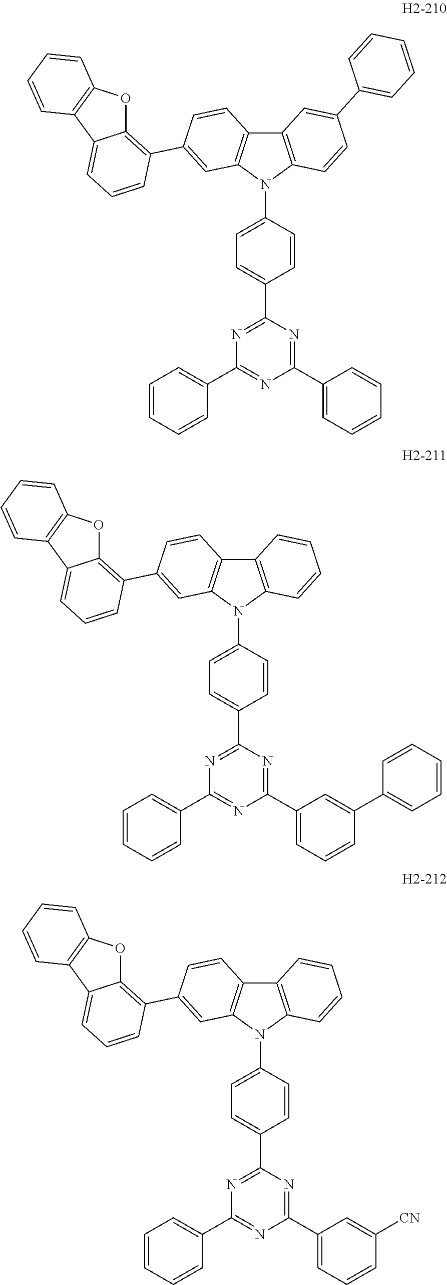 [SMILES: c1ccc(-c2ccc3c(c2)c2ccc(-c4cccc5c4oc4ccccc45)cc2n3-c2ccc(-c3nc(-c4ccccc4)nc(-c4ccccc4)n3)cc2)cc1 ; c1ccc(-c2cccc(-c3nc(-c4ccccc4)nc(-c4ccc(-n5c6ccccc6c6ccc(-c7cccc8c7oc7ccccc78)cc65)cc4)n3)c2)cc1 ; N#Cc1cccc(-c2nc(-c3ccccc3)nc(-c3ccc(-n4c5ccccc5c5ccc(-c6cccc7c6oc6ccccc67)cc54)cc3)n2)c1]

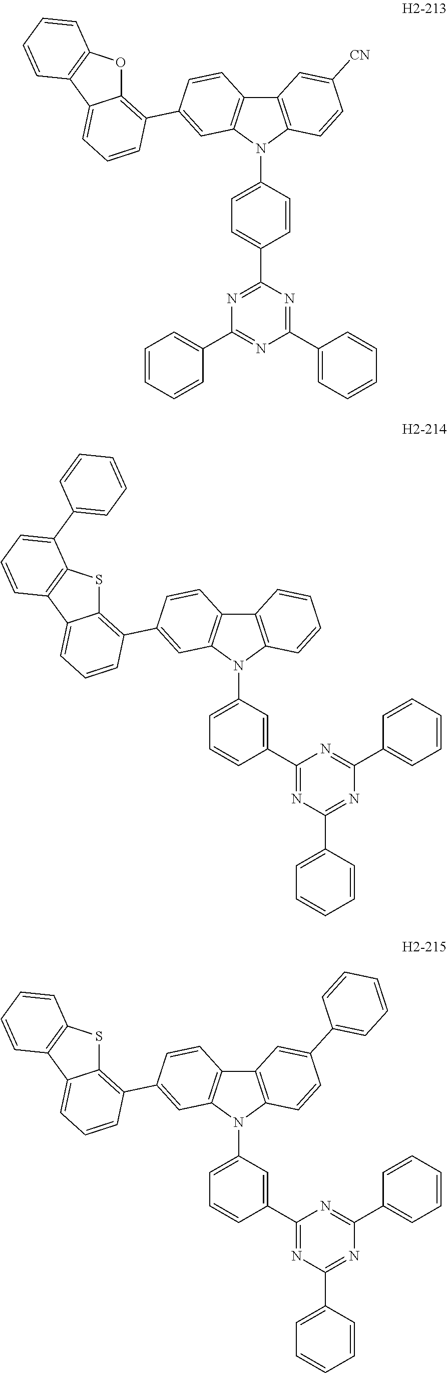 [SMILES: N#Cc1ccc2c(c1)c1ccc(-c3cccc4c3oc3ccccc34)cc1n2-c1ccc(-c2nc(-c3ccccc3)nc(-c3ccccc3)n2)cc1 ; c1ccc(-c2nc(-c3ccccc3)nc(-c3cccc(-n4c5ccccc5c5ccc(-c6cccc7c6sc6c(-c8ccccc8)cccc67)cc54)c3)n2)cc1 ; c1ccc(-c2ccc3c(c2)c2ccc(-c4cccc5c4sc4ccccc45)cc2n3-c2cccc(-c3nc(-c4ccccc4)nc(-c4ccccc4)n3)c2)cc1]

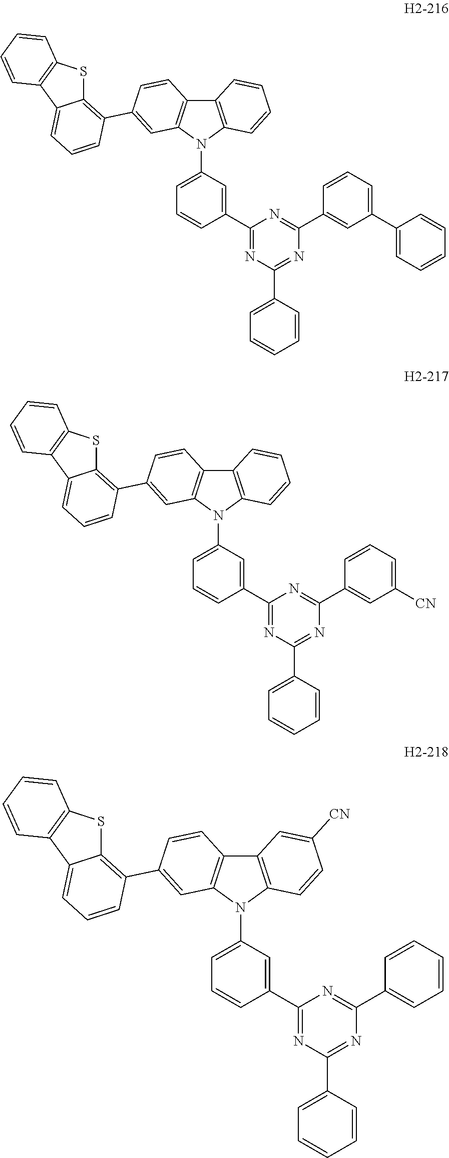 [SMILES: c1ccc(-c2cccc(-c3nc(-c4ccccc4)nc(-c4cccc(-n5c6ccccc6c6ccc(-c7cccc8c7sc7ccccc78)cc65)c4)n3)c2)cc1 ; N#Cc1cccc(-c2nc(-c3ccccc3)nc(-c3cccc(-n4c5ccccc5c5ccc(-c6cccc7c6sc6ccccc67)cc54)c3)n2)c1 ; N#Cc1ccc2c(c1)c1ccc(-c3cccc4c3sc3ccccc34)cc1n2-c1cccc(-c2nc(-c3ccccc3)nc(-c3ccccc3)n2)c1]

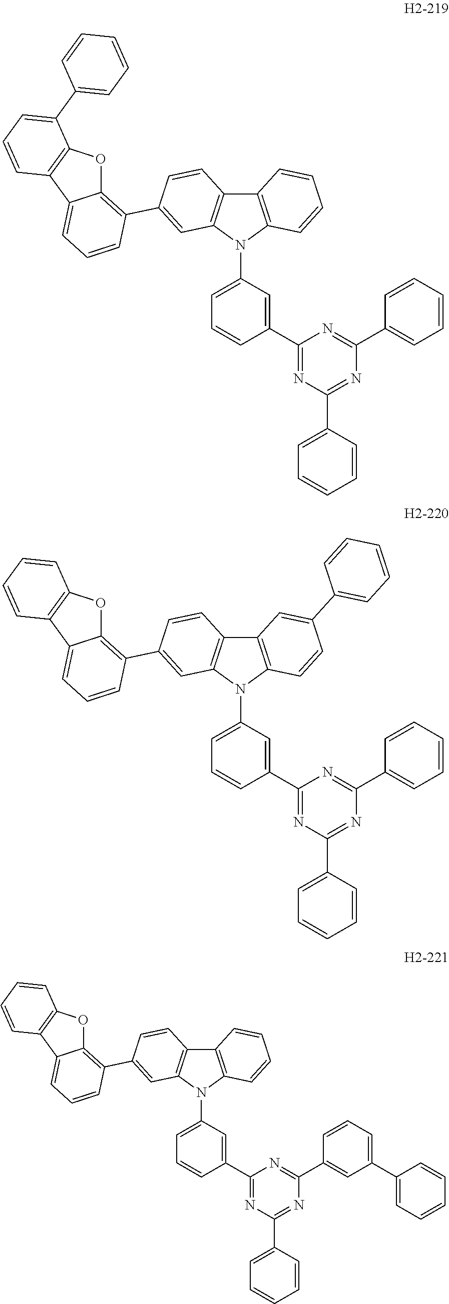 [SMILES: c1ccc(-c2nc(-c3ccccc3)nc(-c3cccc(-n4c5ccccc5c5ccc(-c6cccc7c6oc6c(-c8ccccc8)cccc67)cc54)c3)n2)cc1 ; c1ccc(-c2ccc3c(c2)c2ccc(-c4cccc5c4oc4ccccc45)cc2n3-c2cccc(-c3nc(-c4ccccc4)nc(-c4ccccc4)n3)c2)cc1 ; c1ccc(-c2cccc(-c3nc(-c4ccccc4)nc(-c4cccc(-n5c6ccccc6c6ccc(-c7cccc8c7oc7ccccc78)cc65)c4)n3)c2)cc1]

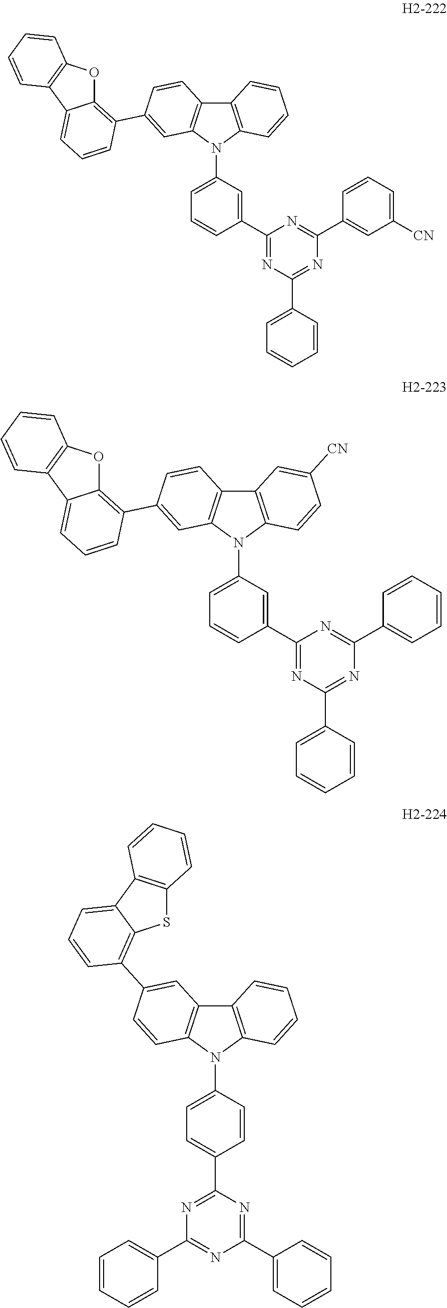 [SMILES: N#Cc1cccc(-c2nc(-c3ccccc3)nc(-c3cccc(-n4c5ccccc5c5ccc(-c6cccc7c6oc6ccccc67)cc54)c3)n2)c1 ; N#Cc1ccc2c(c1)c1ccc(-c3cccc4c3oc3ccccc34)cc1n2-c1cccc(-c2nc(-c3ccccc3)nc(-c3ccccc3)n2)c1 ; c1ccc(-c2nc(-c3ccccc3)nc(-c3ccc(-n4c5ccccc5c5cc(-c6cccc7c6sc6ccccc67)ccc54)cc3)n2)cc1]

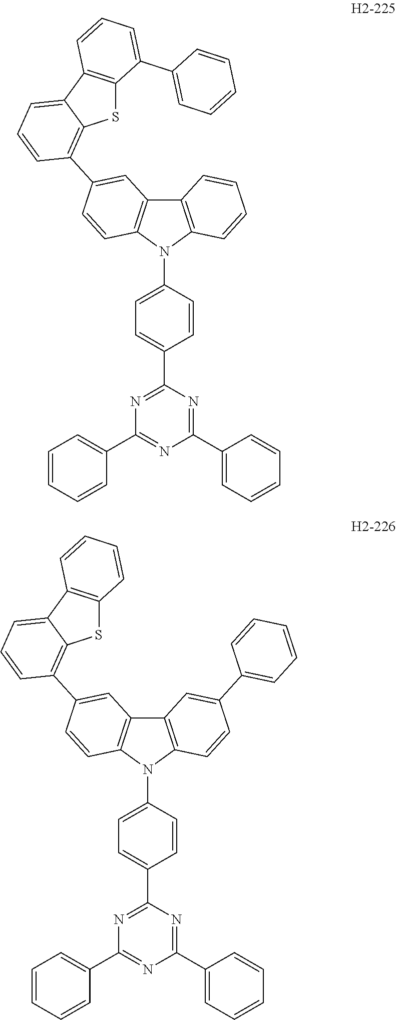

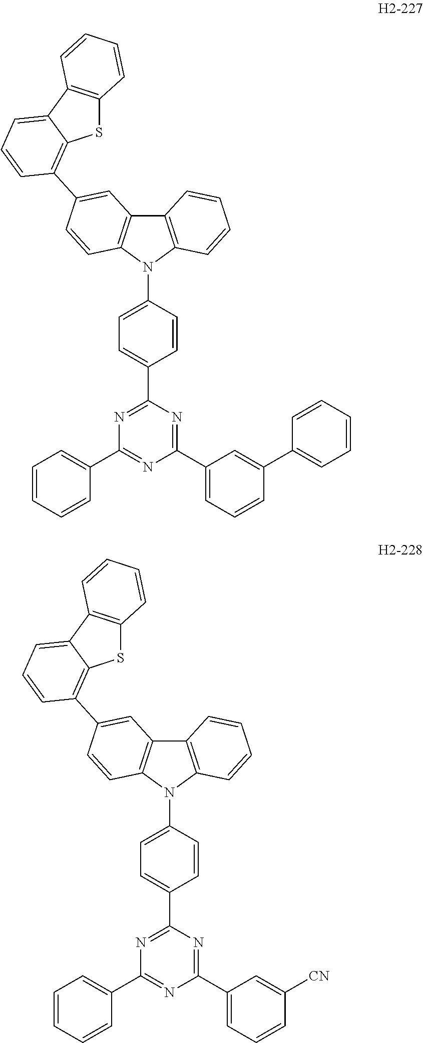 [SMILES: c1ccc(-c2cccc(-c3nc(-c4ccccc4)nc(-c4ccc(-n5c6ccccc6c6cc(-c7cccc8c7sc7ccccc78)ccc65)cc4)n3)c2)cc1 ; N#Cc1cccc(-c2nc(-c3ccccc3)nc(-c3ccc(-n4c5ccccc5c5cc(-c6cccc7c6sc6ccccc67)ccc54)cc3)n2)c1]

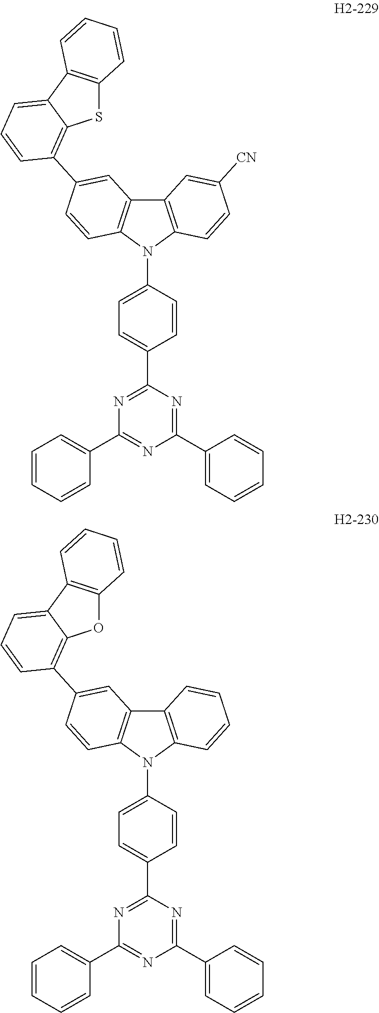

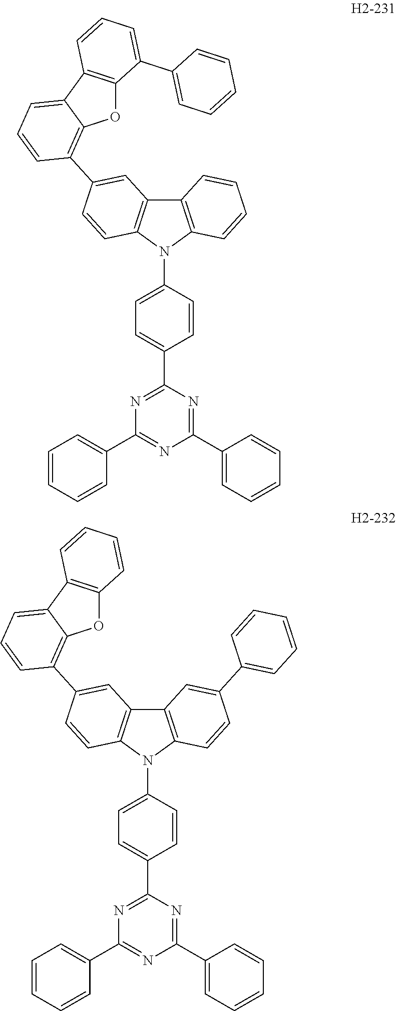

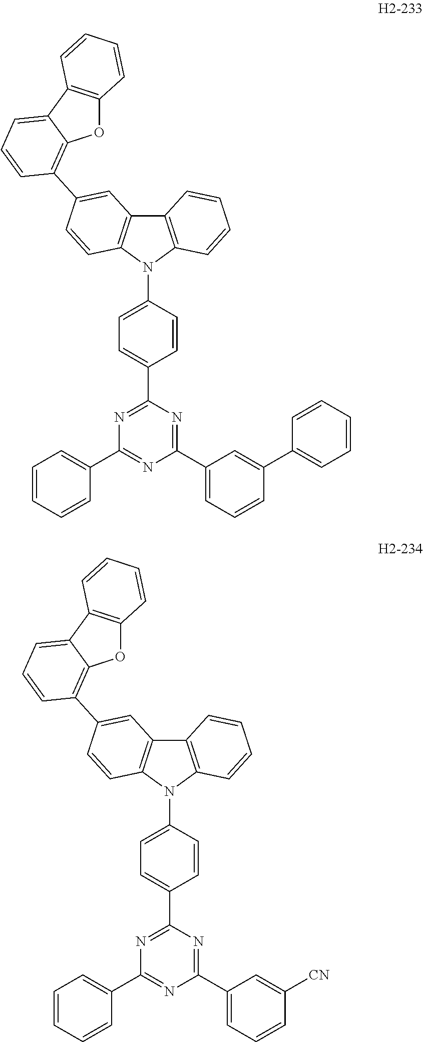 [SMILES: c1ccc(-c2cccc(-c3nc(-c4ccccc4)nc(-c4ccc(-n5c6ccccc6c6cc(-c7cccc8c7oc7ccccc78)ccc65)cc4)n3)c2)cc1 ; N#Cc1cccc(-c2nc(-c3ccccc3)nc(-c3ccc(-n4c5ccccc5c5cc(-c6cccc7c6oc6ccccc67)ccc54)cc3)n2)c1]

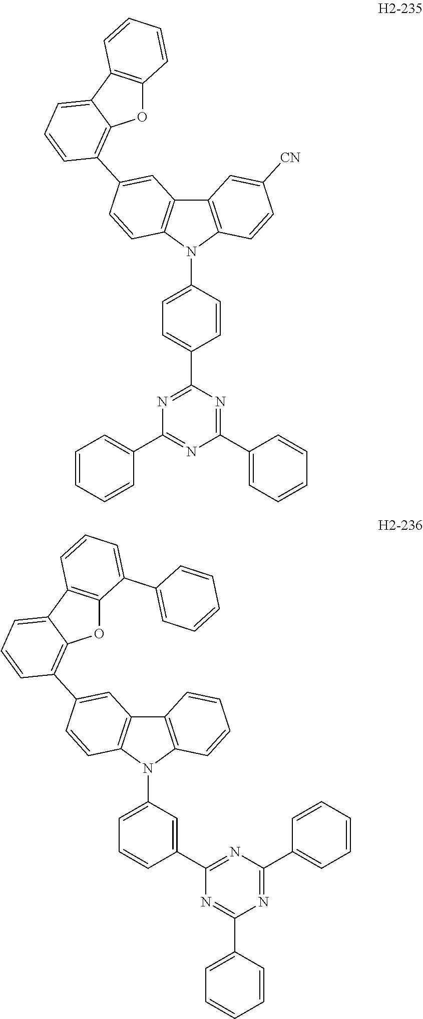

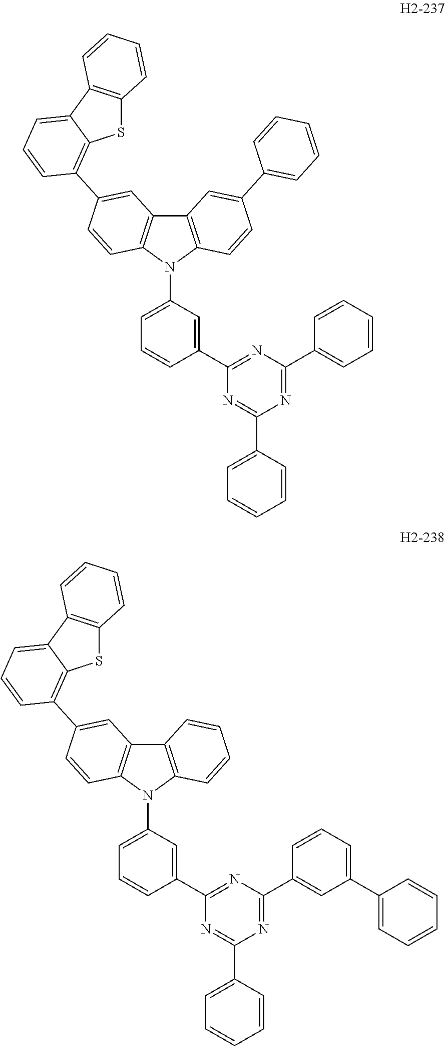 [SMILES: c1ccc(-c2ccc3c(c2)c2cc(-c4cccc5c4sc4ccccc45)ccc2n3-c2cccc(-c3nc(-c4ccccc4)nc(-c4ccccc4)n3)c2)cc1 ; c1ccc(-c2cccc(-c3nc(-c4ccccc4)nc(-c4cccc(-n5c6ccccc6c6cc(-c7cccc8c7sc7ccccc78)ccc65)c4)n3)c2)cc1]

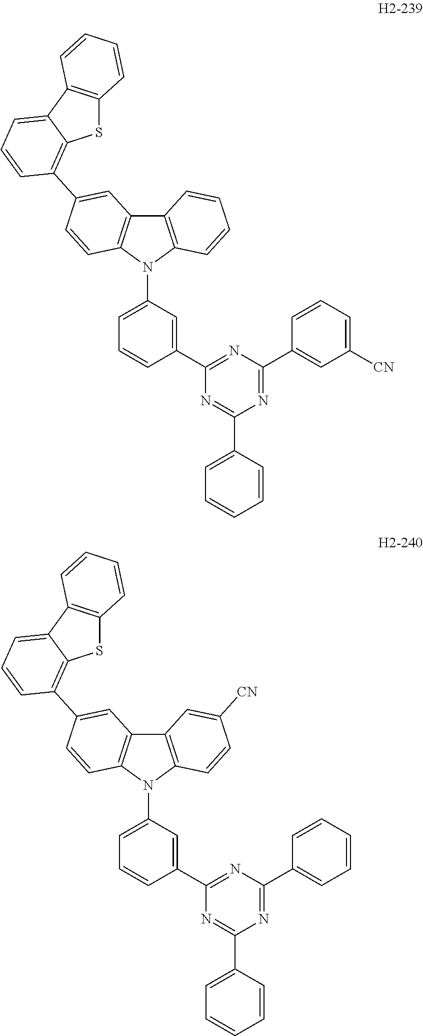 [SMILES: N#Cc1cccc(-c2nc(-c3ccccc3)nc(-c3cccc(-n4c5ccccc5c5cc(-c6cccc7c6sc6ccccc67)ccc54)c3)n2)c1 ; N#Cc1ccc2c(c1)c1cc(-c3cccc4c3sc3ccccc34)ccc1n2-c1cccc(-c2nc(-c3ccccc3)nc(-c3ccccc3)n2)c1]

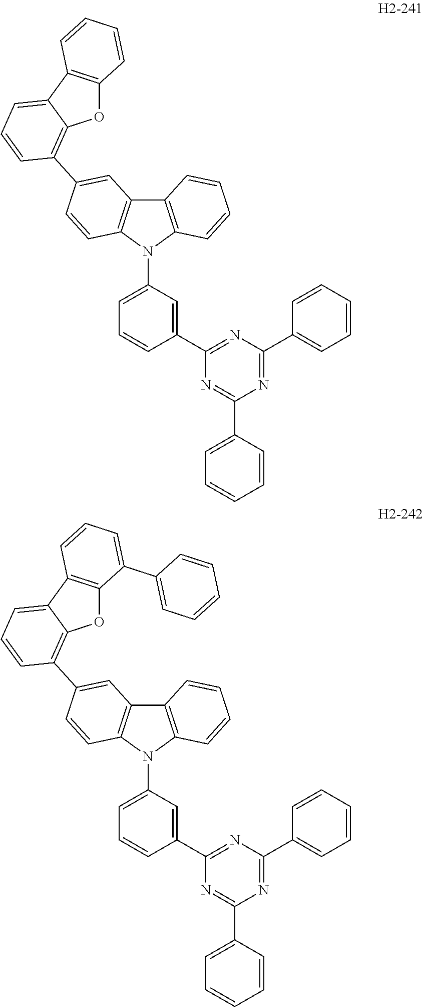 [SMILES: c1ccc(-c2nc(-c3ccccc3)nc(-c3cccc(-n4c5ccccc5c5cc(-c6cccc7c6oc6ccccc67)ccc54)c3)n2)cc1 ; c1ccc(-c2nc(-c3ccccc3)nc(-c3cccc(-n4c5ccccc5c5cc(-c6cccc7c6oc6c(-c8ccccc8)cccc67)ccc54)c3)n2)cc1]

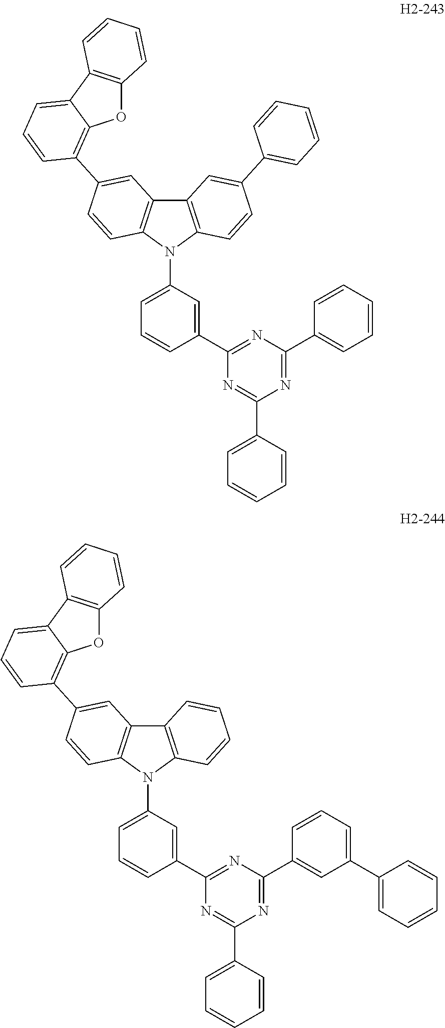 [SMILES: c1ccc(-c2ccc3c(c2)c2cc(-c4cccc5c4oc4ccccc45)ccc2n3-c2cccc(-c3nc(-c4ccccc4)nc(-c4ccccc4)n3)c2)cc1 ; c1ccc(-c2cccc(-c3nc(-c4ccccc4)nc(-c4cccc(-n5c6ccccc6c6cc(-c7cccc8c7oc7ccccc78)ccc65)c4)n3)c2)cc1]

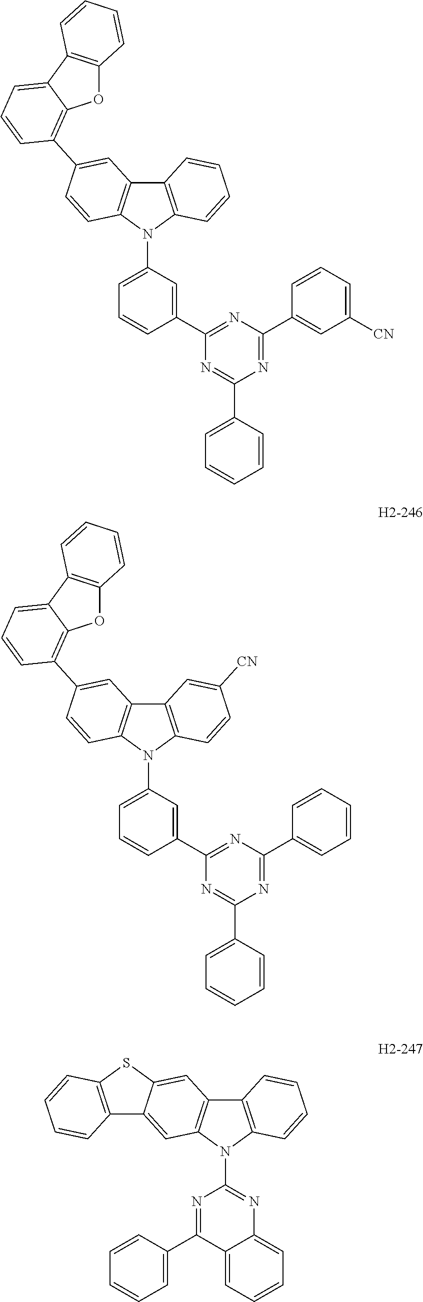 [SMILES: N#Cc1cccc(-c2nc(-c3ccccc3)nc(-c3cccc(-n4c5ccccc5c5cc(-c6cccc7c6oc6ccccc67)ccc54)c3)n2)c1 ; N#Cc1ccc2c(c1)c1cc(-c3cccc4c3oc3ccccc34)ccc1n2-c1cccc(-c2nc(-c3ccccc3)nc(-c3ccccc3)n2)c1 ; c1ccc(-c2nc(-n3c4ccccc4c4cc5sc6ccccc6c5cc43)nc3ccccc23)cc1]

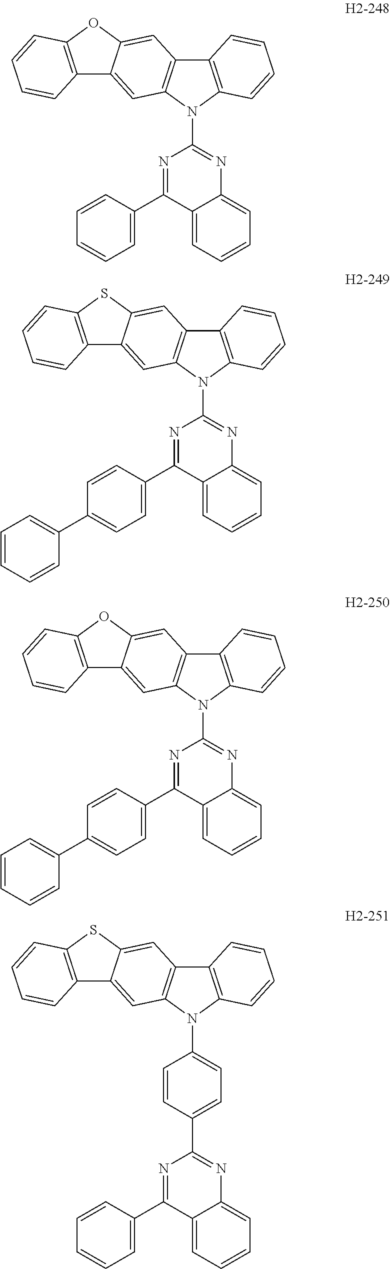

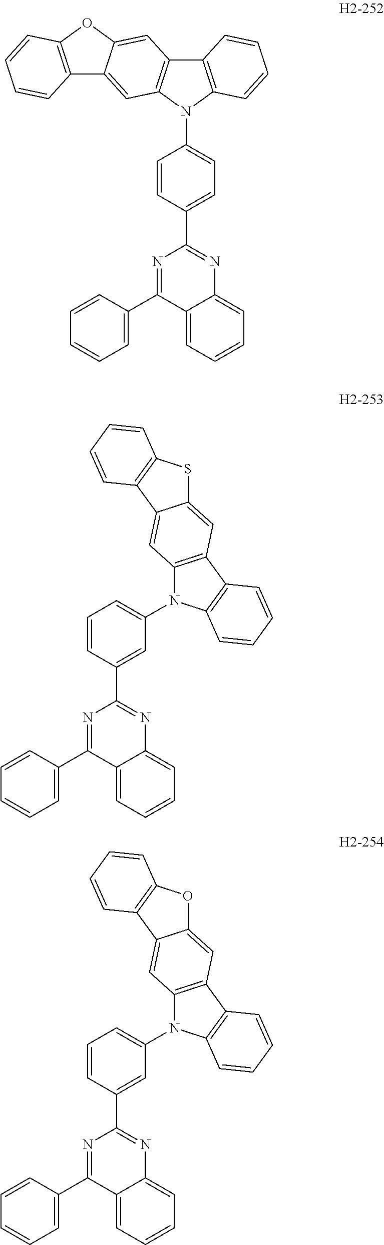

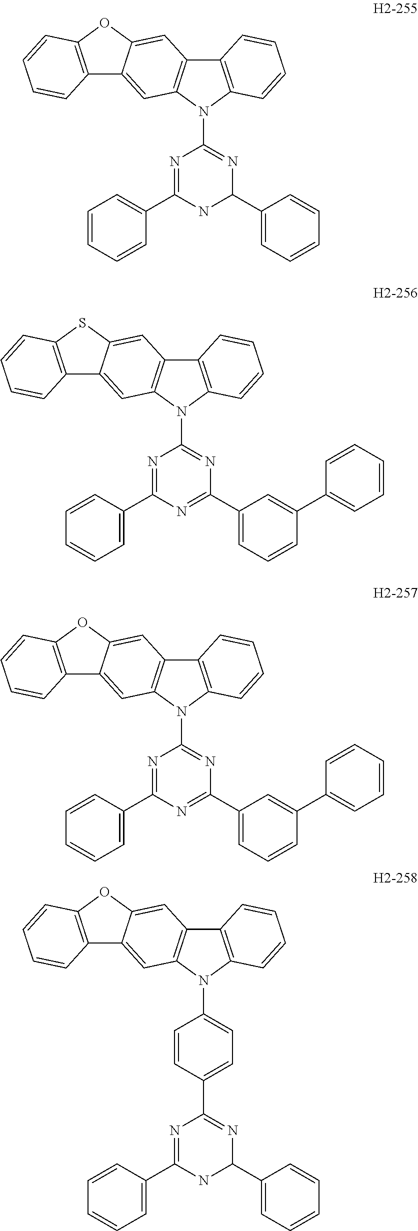

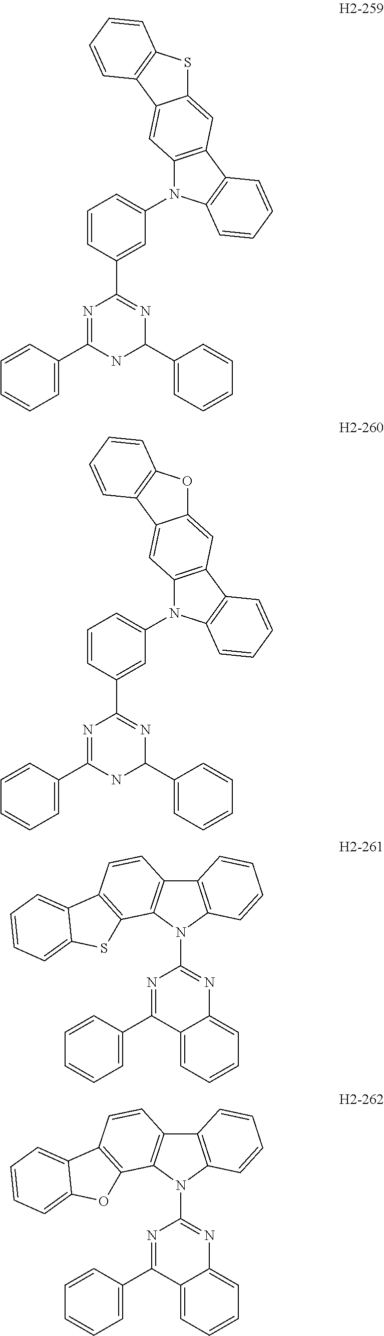

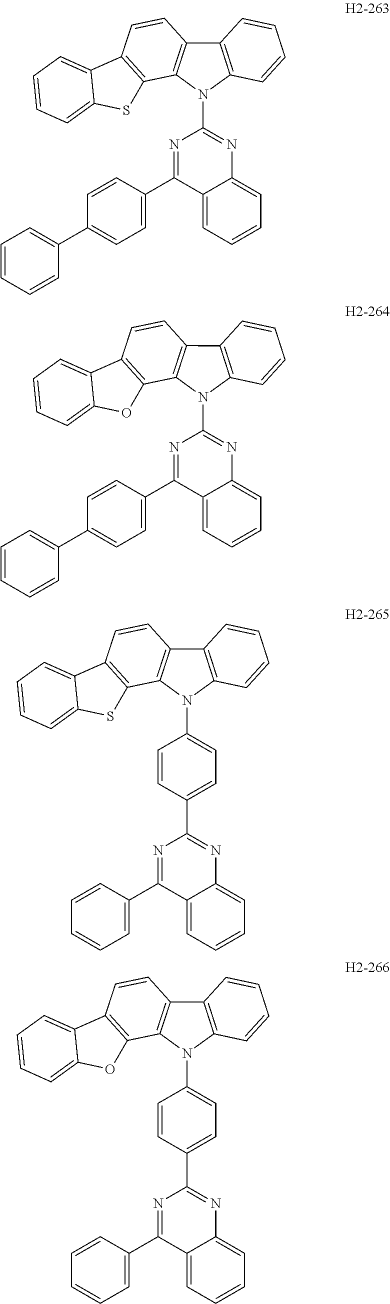

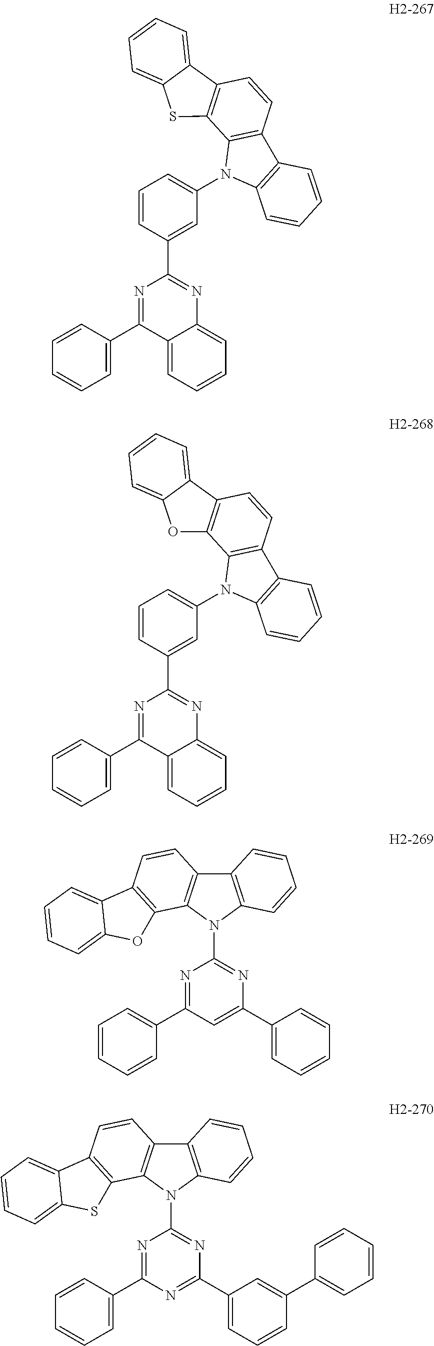

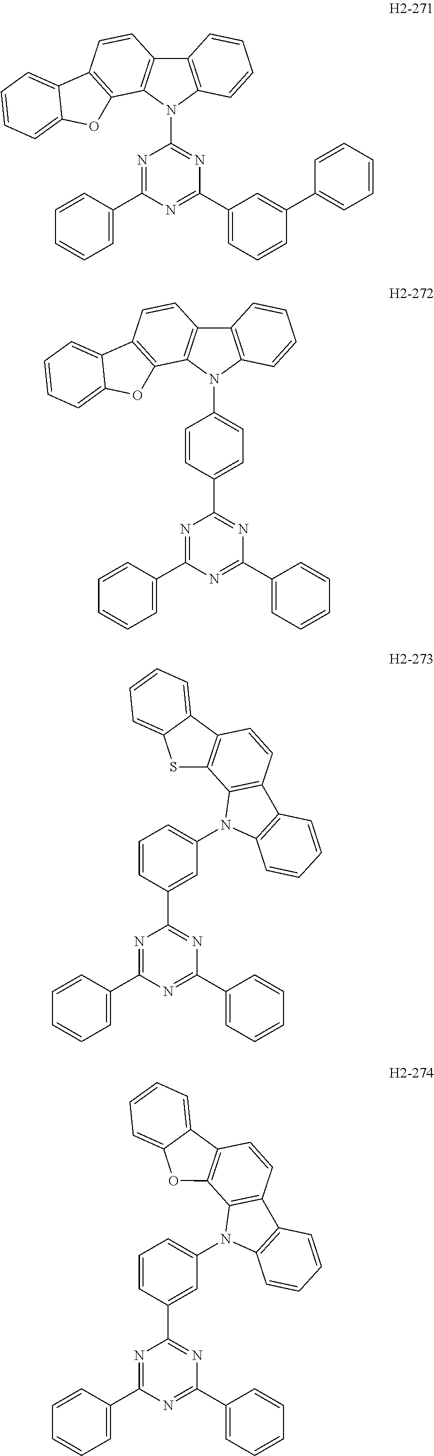

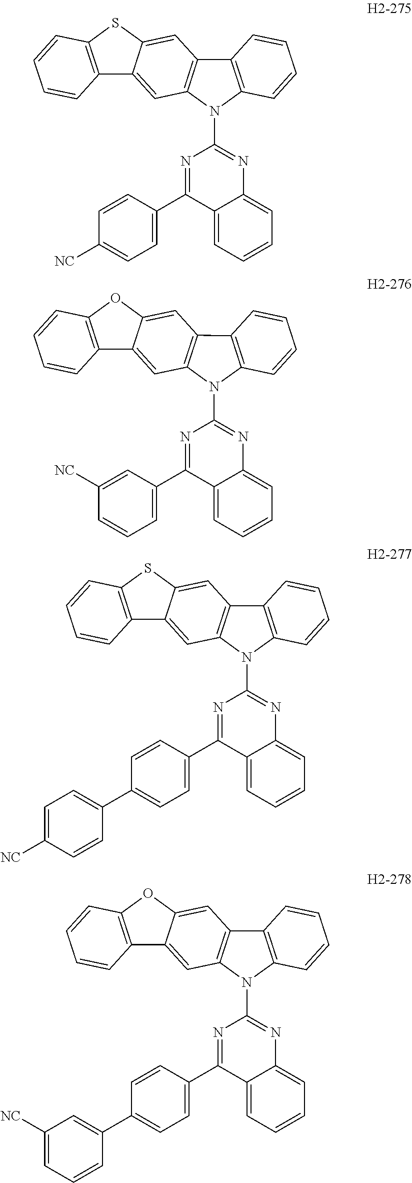

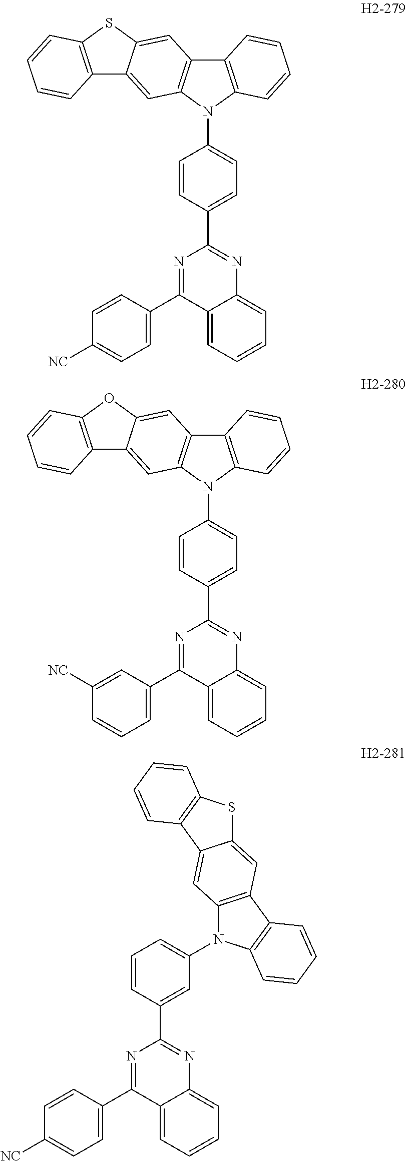

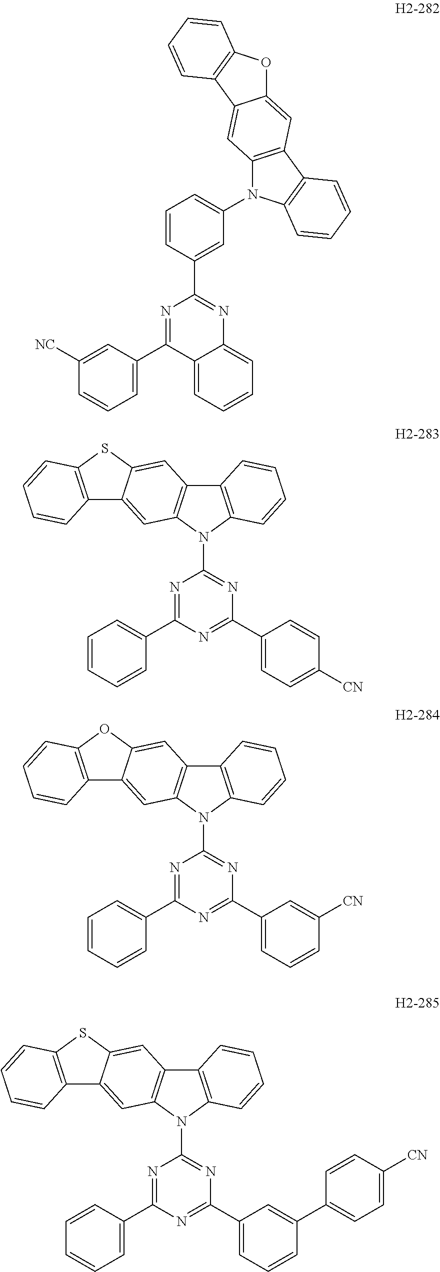

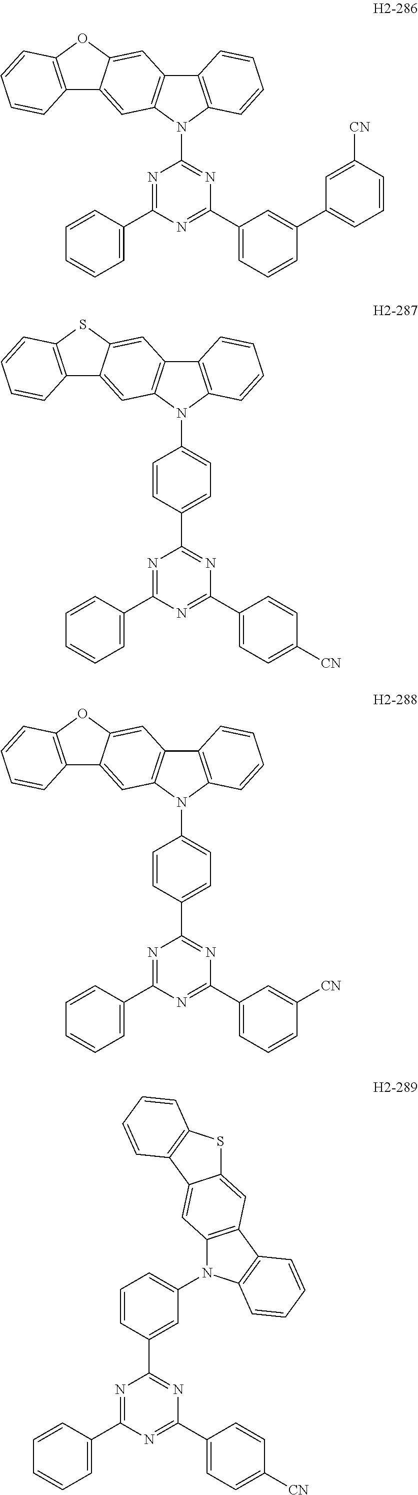 [SMILES: N#Cc1cccc(-c2cccc(-c3nc(-c4ccccc4)nc(-n4c5ccccc5c5cc6oc7ccccc7c6cc54)n3)c2)c1 ; N#Cc1ccc(-c2nc(-c3ccccc3)nc(-c3ccc(-n4c5ccccc5c5cc6sc7ccccc7c6cc54)cc3)n2)cc1 ; N#Cc1cccc(-c2nc(-c3ccccc3)nc(-c3ccc(-n4c5ccccc5c5cc6oc7ccccc7c6cc54)cc3)n2)c1 ; N#Cc1ccc(-c2nc(-c3ccccc3)nc(-c3cccc(-n4c5ccccc5c5cc6sc7ccccc7c6cc54)c3)n2)cc1]

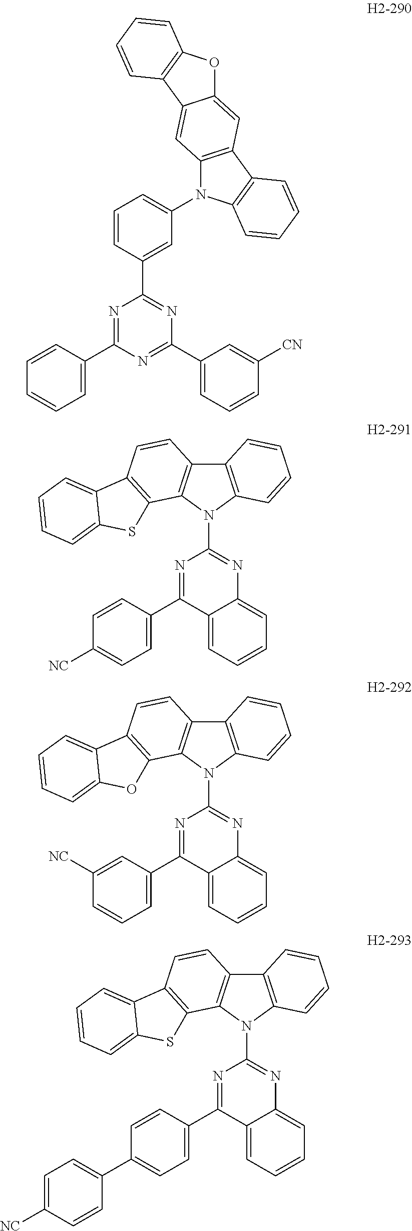

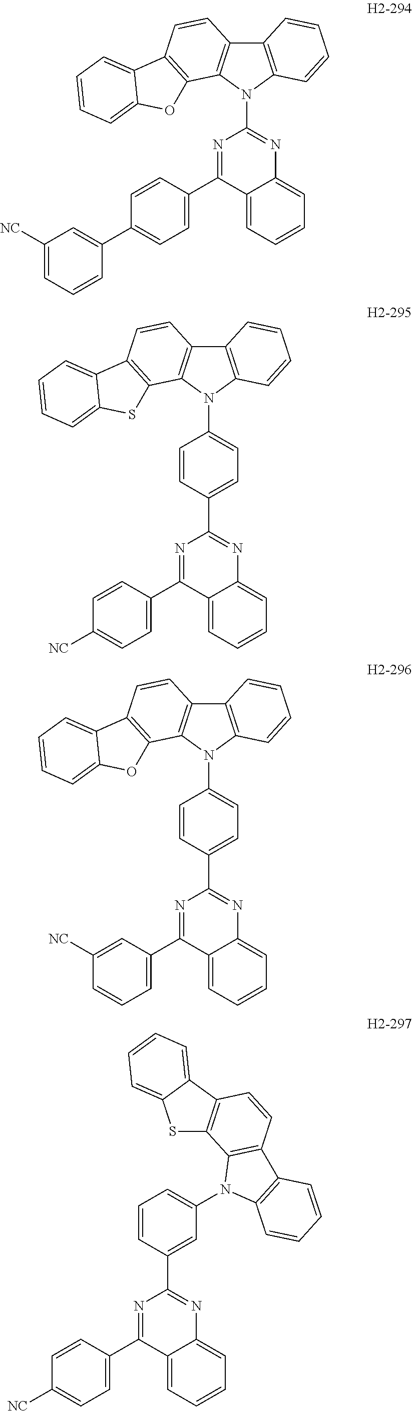

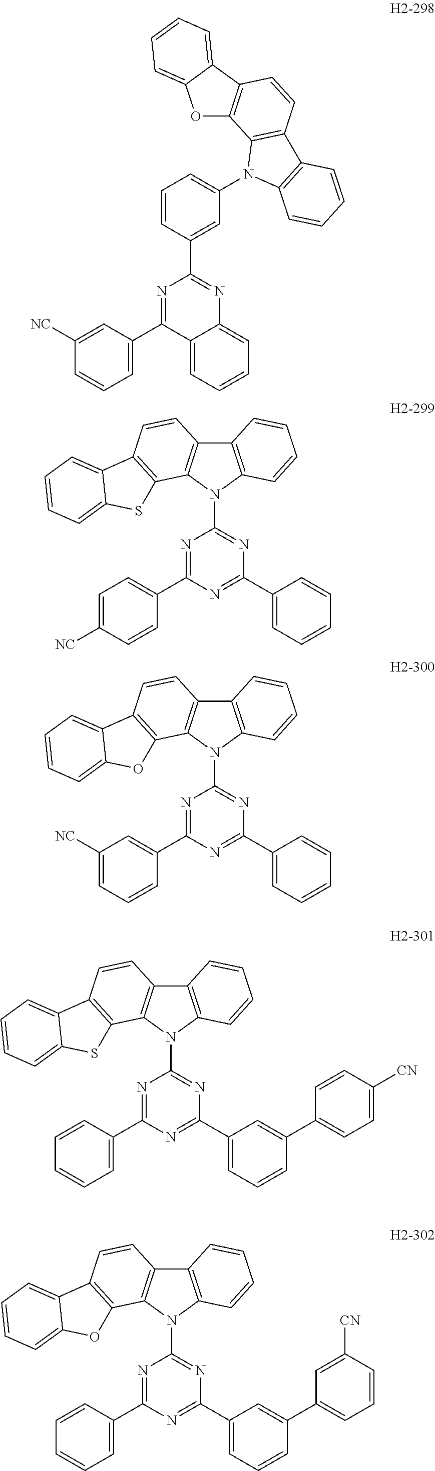

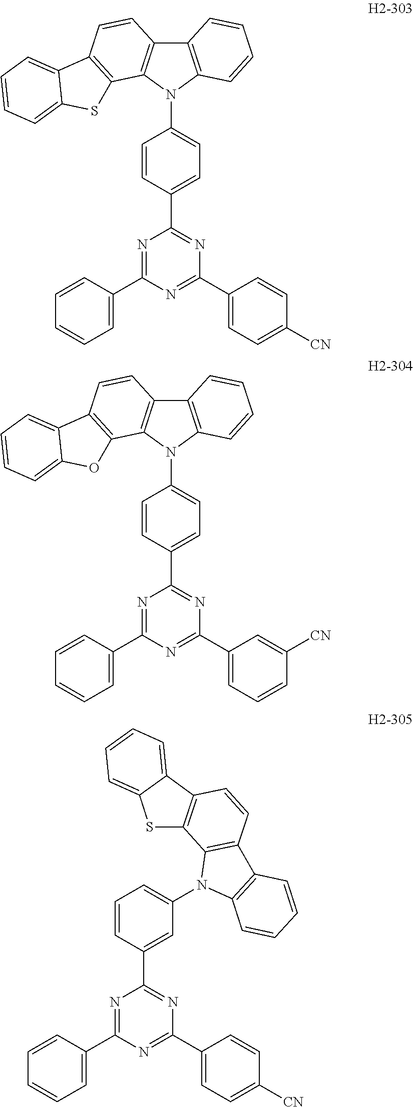

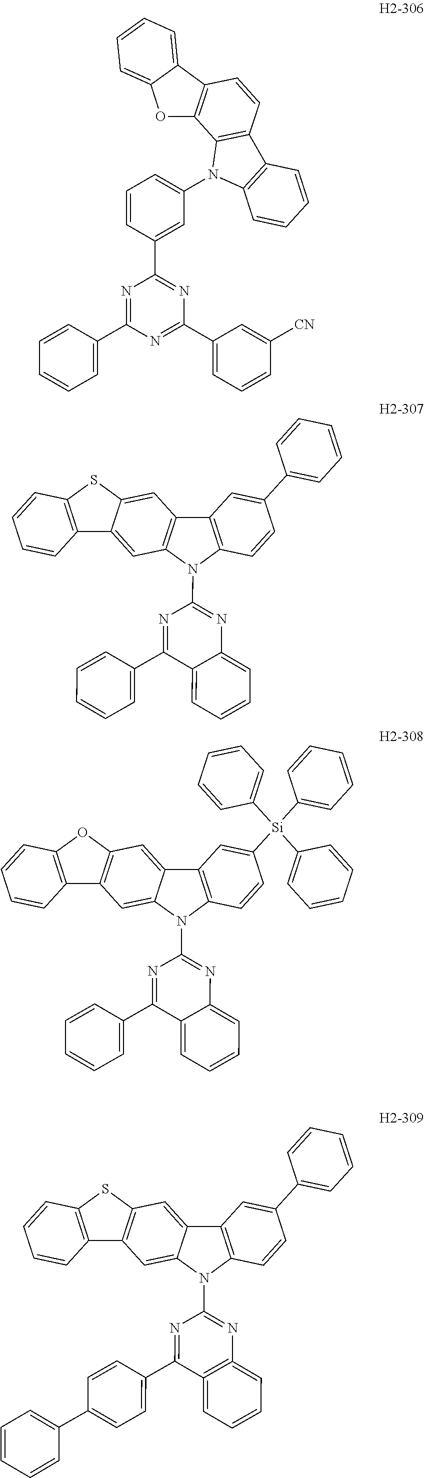

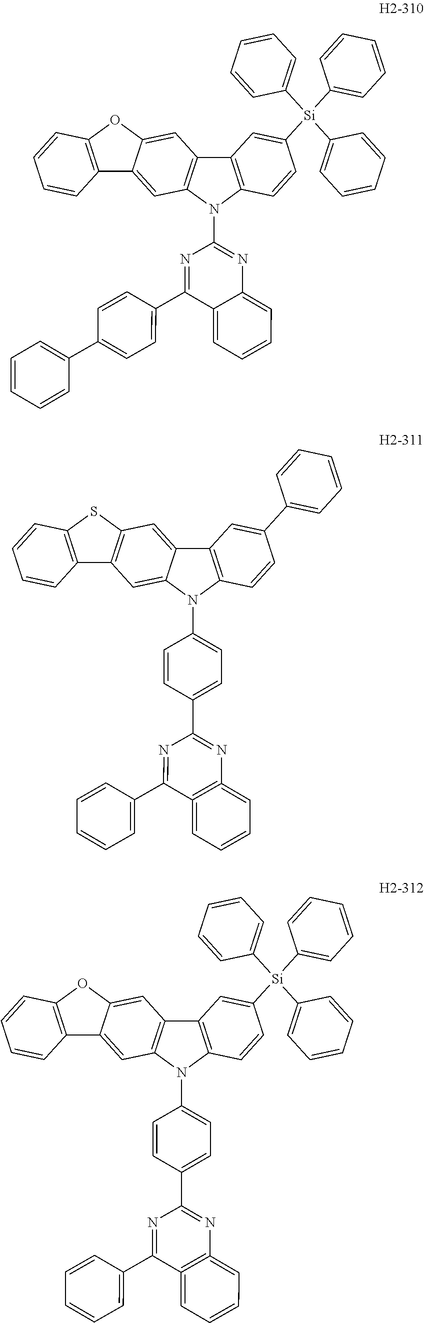

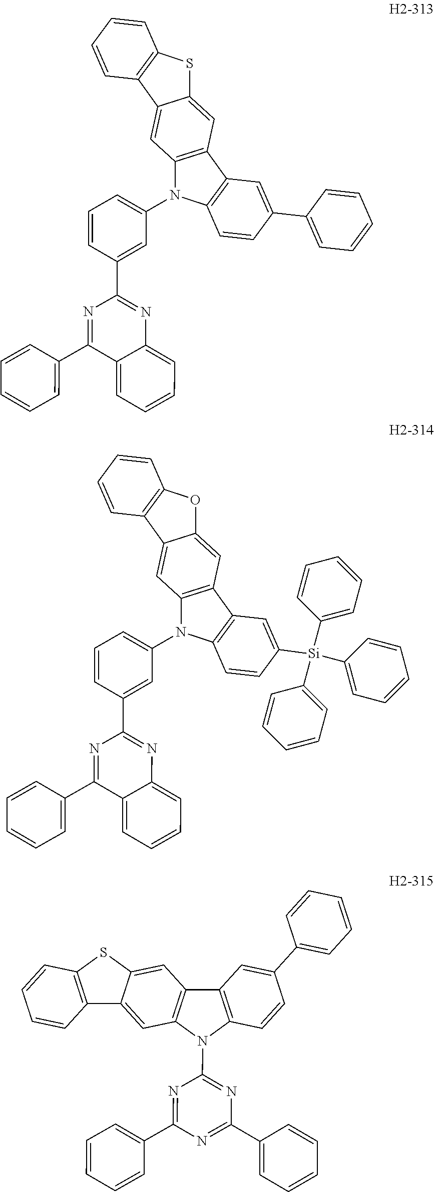 [SMILES: c1ccc(-c2ccc3c(c2)c2cc4sc5ccccc5c4cc2n3-c2cccc(-c3nc(-c4ccccc4)c4ccccc4n3)c2)cc1 ; c1ccc(-c2nc(-c3cccc(-n4c5ccc([Si](c6ccccc6)(c6ccccc6)c6ccccc6)cc5c5cc6oc7ccccc7c6cc54)c3)nc3ccccc23)cc1 ; c1ccc(-c2ccc3c(c2)c2cc4sc5ccccc5c4cc2n3-c2nc(-c3ccccc3)nc(-c3ccccc3)n2)cc1]

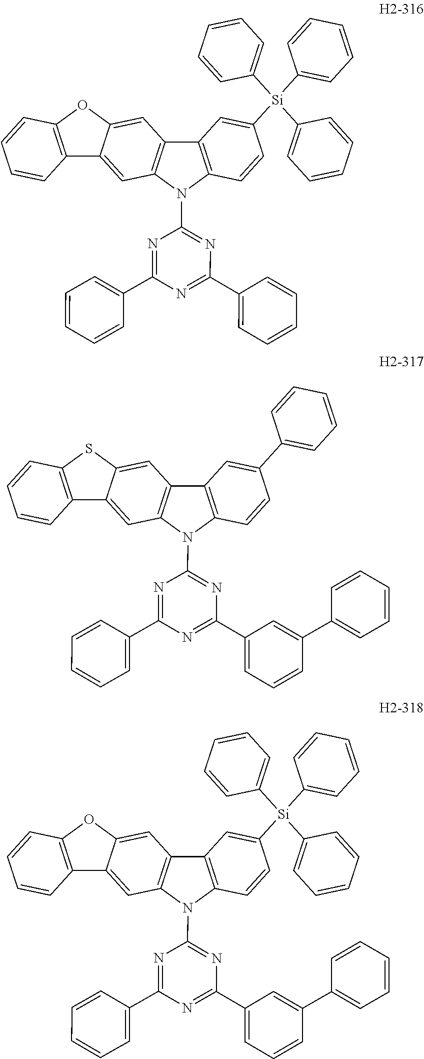 [SMILES: c1ccc(-c2nc(-c3ccccc3)nc(-n3c4ccc([Si](c5ccccc5)(c5ccccc5)c5ccccc5)cc4c4cc5oc6ccccc6c5cc43)n2)cc1 ; c1ccc(-c2cccc(-c3nc(-c4ccccc4)nc(-n4c5ccc(-c6ccccc6)cc5c5cc6sc7ccccc7c6cc54)n3)c2)cc1 ; c1ccc(-c2cccc(-c3nc(-c4ccccc4)nc(-n4c5ccc([Si](c6ccccc6)(c6ccccc6)c6ccccc6)cc5c5cc6oc7ccccc7c6cc54)n3)c2)cc1]

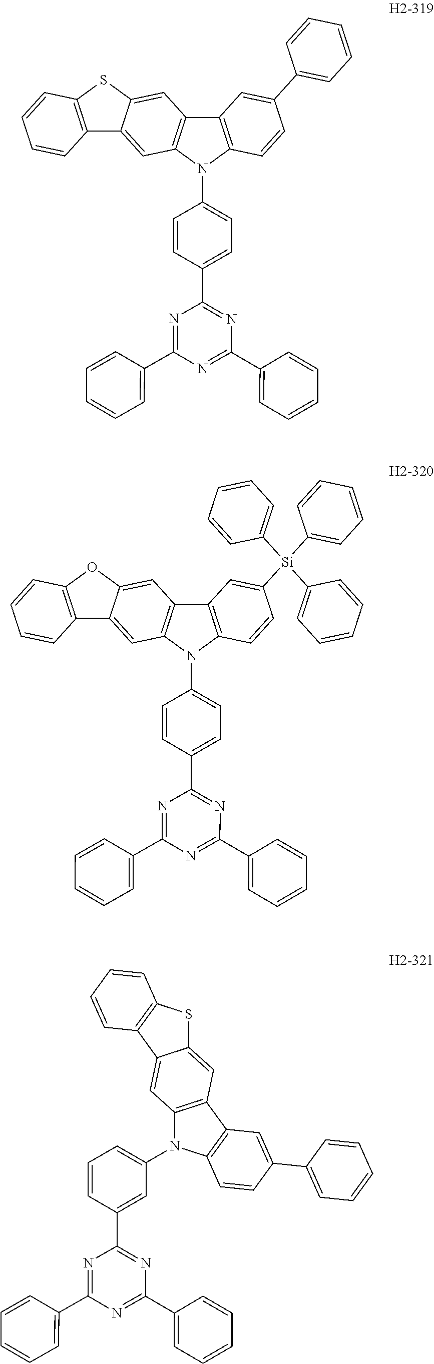 [SMILES: c1ccc(-c2ccc3c(c2)c2cc4sc5ccccc5c4cc2n3-c2ccc(-c3nc(-c4ccccc4)nc(-c4ccccc4)n3)cc2)cc1 ; c1ccc(-c2nc(-c3ccccc3)nc(-c3ccc(-n4c5ccc([Si](c6ccccc6)(c6ccccc6)c6ccccc6)cc5c5cc6oc7ccccc7c6cc54)cc3)n2)cc1 ; c1ccc(-c2ccc3c(c2)c2cc4sc5ccccc5c4cc2n3-c2cccc(-c3nc(-c4ccccc4)nc(-c4ccccc4)n3)c2)cc1]

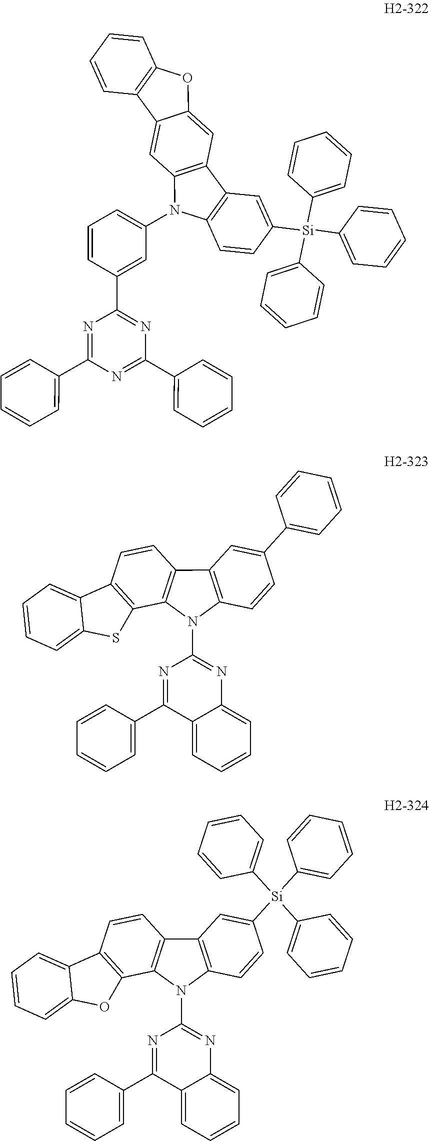 [SMILES: c1ccc(-c2nc(-c3ccccc3)nc(-c3cccc(-n4c5ccc([Si](c6ccccc6)(c6ccccc6)c6ccccc6)cc5c5cc6oc7ccccc7c6cc54)c3)n2)cc1 ; c1ccc(-c2ccc3c(c2)c2ccc4c5ccccc5sc4c2n3-c2nc(-c3ccccc3)c3ccccc3n2)cc1 ; c1ccc(-c2nc(-n3c4ccc([Si](c5ccccc5)(c5ccccc5)c5ccccc5)cc4c4ccc5c6ccccc6oc5c43)nc3ccccc23)cc1]

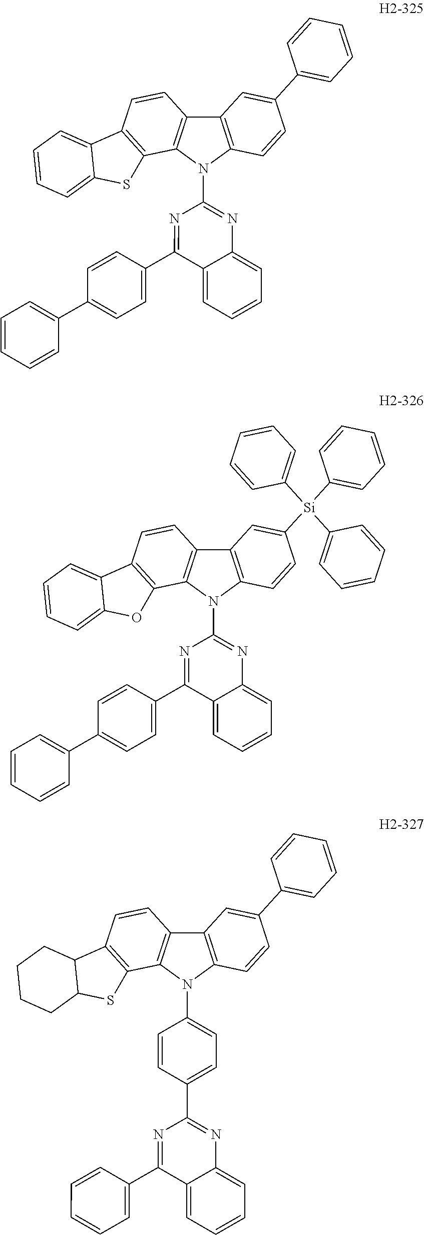 [SMILES: c1ccc(-c2ccc(-c3nc(-n4c5ccc(-c6ccccc6)cc5c5ccc6c7ccccc7sc6c54)nc4ccccc34)cc2)cc1 ; c1ccc(-c2ccc(-c3nc(-n4c5ccc([Si](c6ccccc6)(c6ccccc6)c6ccccc6)cc5c5ccc6c7ccccc7oc6c54)nc4ccccc34)cc2)cc1 ; c1ccc(-c2ccc3c(c2)c2ccc4c(c2n3-c2ccc(-c3nc(-c5ccccc5)c5ccccc5n3)cc2)SC2CCCCC42)cc1]

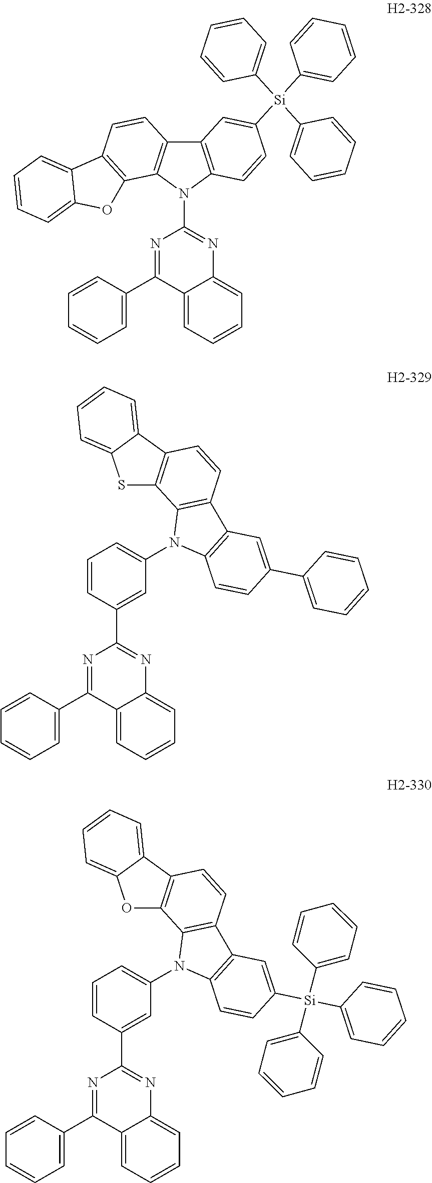

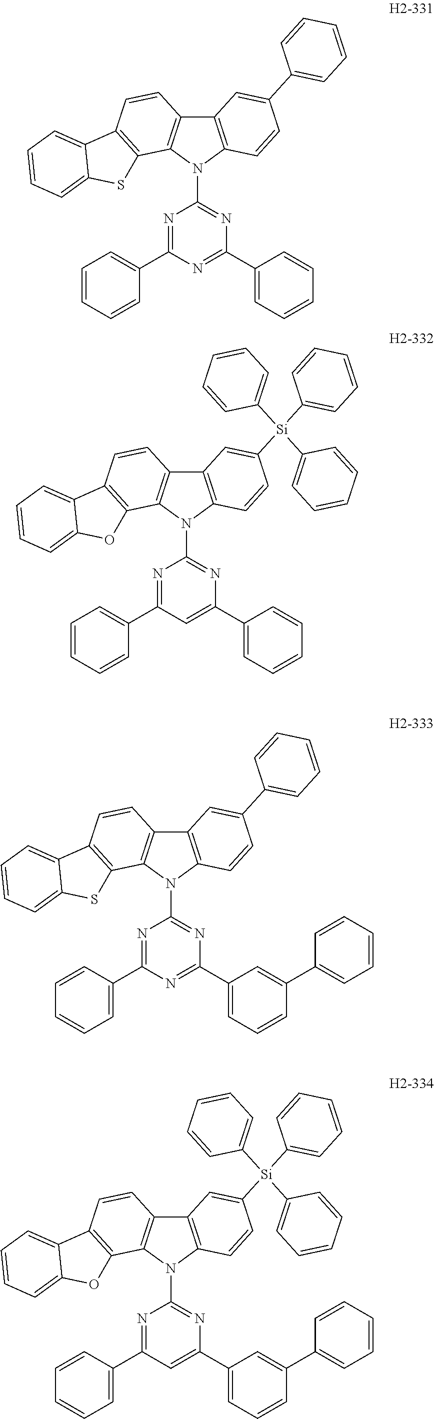

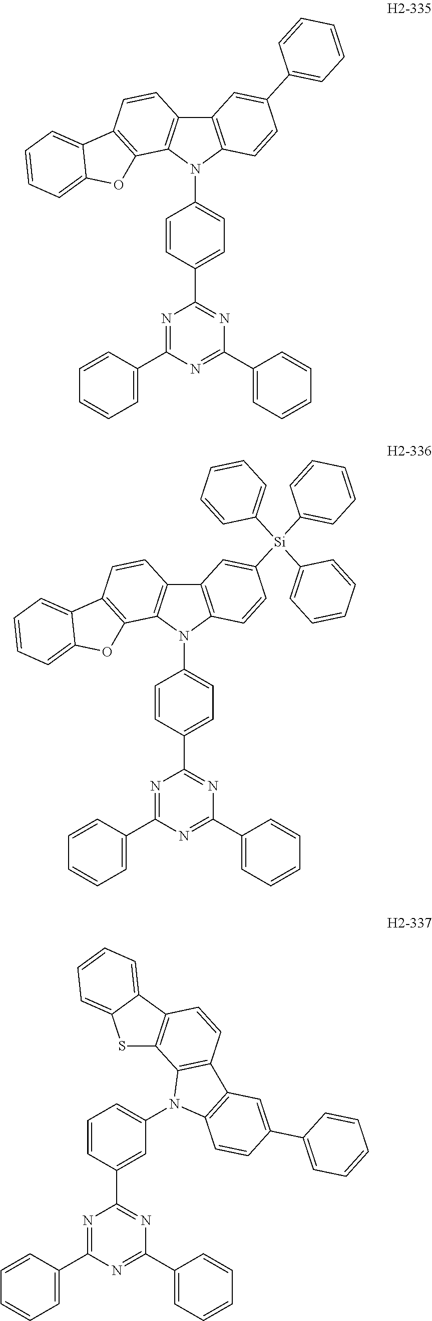 [SMILES: c1ccc(-c2ccc3c(c2)c2ccc4c5ccccc5oc4c2n3-c2ccc(-c3nc(-c4ccccc4)nc(-c4ccccc4)n3)cc2)cc1 ; c1ccc(-c2nc(-c3ccccc3)nc(-c3ccc(-n4c5ccc([Si](c6ccccc6)(c6ccccc6)c6ccccc6)cc5c5ccc6c7ccccc7oc6c54)cc3)n2)cc1 ; c1ccc(-c2ccc3c(c2)c2ccc4c5ccccc5sc4c2n3-c2cccc(-c3nc(-c4ccccc4)nc(-c4ccccc4)n3)c2)cc1]

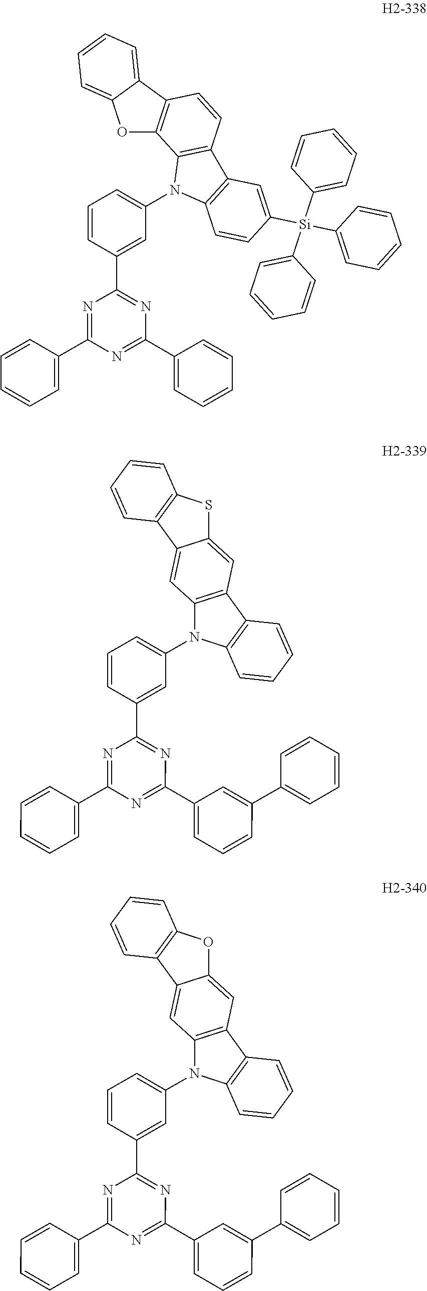 [SMILES: c1ccc(-c2nc(-c3ccccc3)nc(-c3cccc(-n4c5ccc([Si](c6ccccc6)(c6ccccc6)c6ccccc6)cc5c5ccc6c7ccccc7oc6c54)c3)n2)cc1 ; c1ccc(-c2cccc(-c3nc(-c4ccccc4)nc(-c4cccc(-n5c6ccccc6c6cc7sc8ccccc8c7cc65)c4)n3)c2)cc1 ; c1ccc(-c2cccc(-c3nc(-c4ccccc4)nc(-c4cccc(-n5c6ccccc6c6cc7oc8ccccc8c7cc65)c4)n3)c2)cc1]

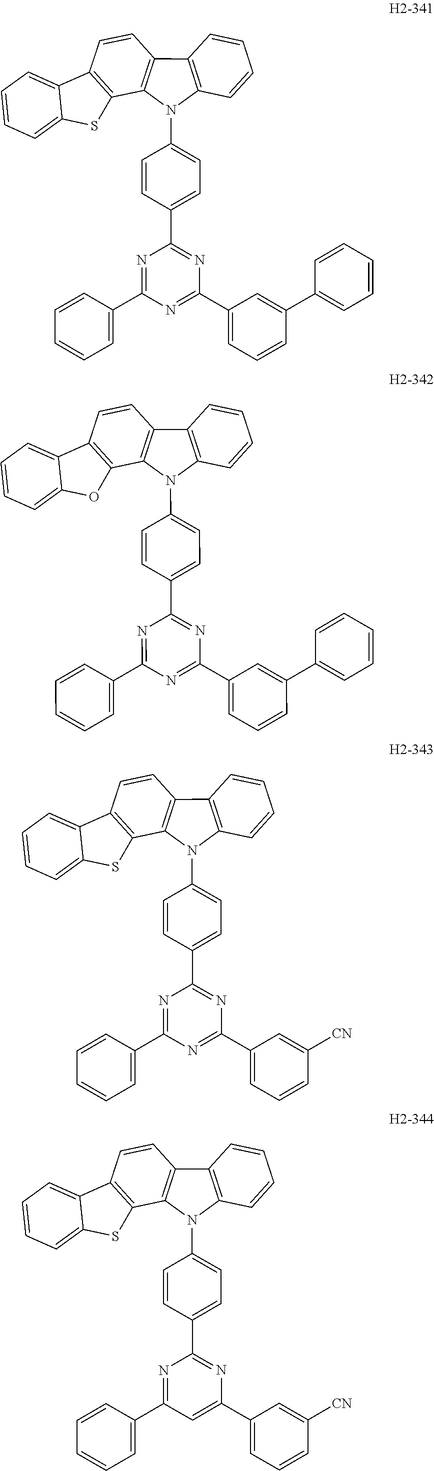 [SMILES: c1ccc(-c2cccc(-c3nc(-c4ccccc4)nc(-c4ccc(-n5c6ccccc6c6ccc7c8ccccc8sc7c65)cc4)n3)c2)cc1 ; c1ccc(-c2cccc(-c3nc(-c4ccccc4)nc(-c4ccc(-n5c6ccccc6c6ccc7c8ccccc8oc7c65)cc4)n3)c2)cc1 ; N#Cc1cccc(-c2nc(-c3ccccc3)nc(-c3ccc(-n4c5ccccc5c5ccc6c7ccccc7sc6c54)cc3)n2)c1 ; N#Cc1cccc(-c2cc(-c3ccccc3)nc(-c3ccc(-n4c5ccccc5c5ccc6c7ccccc7sc6c54)cc3)n2)c1]

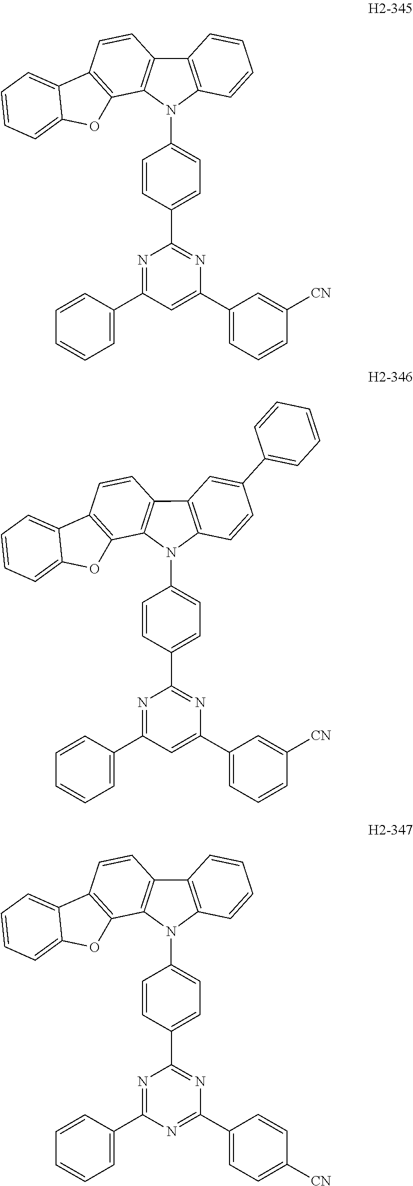

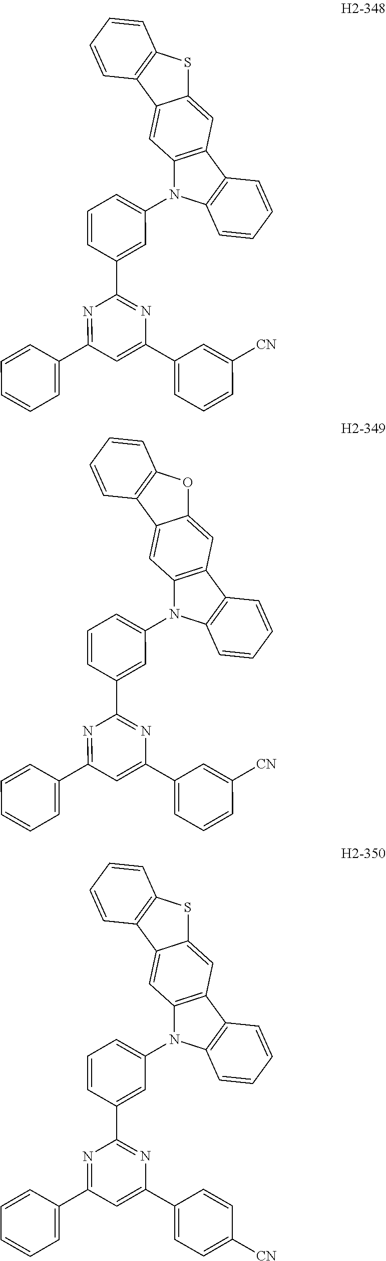 [SMILES: N#Cc1cccc(-c2cc(-c3ccccc3)nc(-c3cccc(-n4c5ccccc5c5cc6sc7ccccc7c6cc54)c3)n2)c1 ; N#Cc1cccc(-c2cc(-c3ccccc3)nc(-c3cccc(-n4c5ccccc5c5cc6oc7ccccc7c6cc54)c3)n2)c1 ; N#Cc1ccc(-c2cc(-c3ccccc3)nc(-c3cccc(-n4c5ccccc5c5cc6sc7ccccc7c6cc54)c3)n2)cc1]

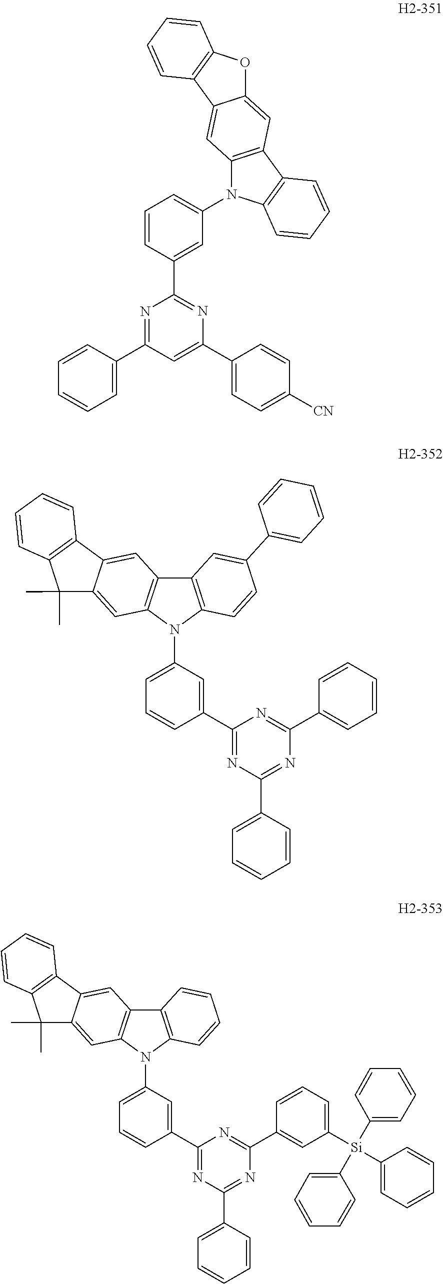 [SMILES: N#Cc1ccc(-c2cc(-c3ccccc3)nc(-c3cccc(-n4c5ccccc5c5cc6oc7ccccc7c6cc54)c3)n2)cc1 ; CC1(C)c2ccccc2-c2cc3c4cc(-c5ccccc5)ccc4n(-c4cccc(-c5nc(-c6ccccc6)nc(-c6ccccc6)n5)c4)c3cc21 ; CC1(C)c2ccccc2-c2cc3c4ccccc4n(-c4cccc(-c5nc(-c6ccccc6)nc(-c6cccc([Si](c7ccccc7)(c7ccccc7)c7ccccc7)c6)n5)c4)c3cc21]

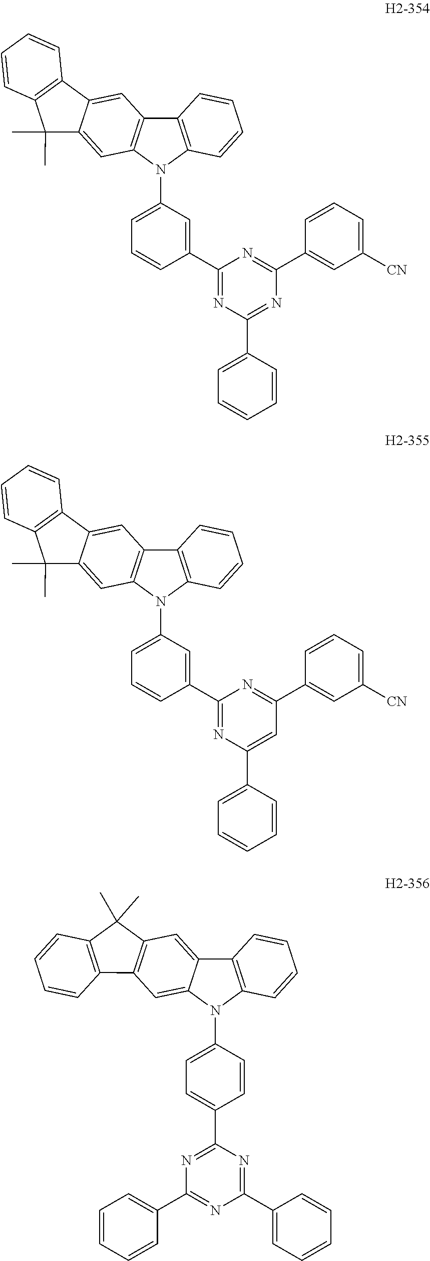 [SMILES: CC1(C)c2ccccc2-c2cc3c4ccccc4n(-c4cccc(-c5nc(-c6ccccc6)nc(-c6cccc(C#N)c6)n5)c4)c3cc21 ; CC1(C)c2ccccc2-c2cc3c4ccccc4n(-c4cccc(-c5nc(-c6ccccc6)cc(-c6cccc(C#N)c6)n5)c4)c3cc21 ; CC1(C)c2ccccc2-c2cc3c(cc21)c1ccccc1n3-c1ccc(-c2nc(-c3ccccc3)nc(-c3ccccc3)n2)cc1]

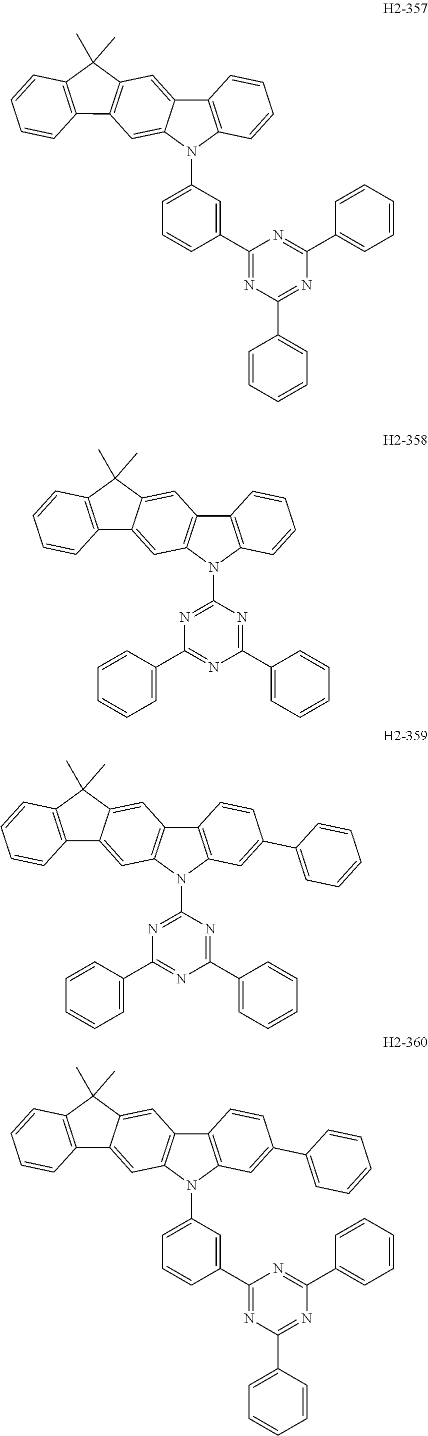

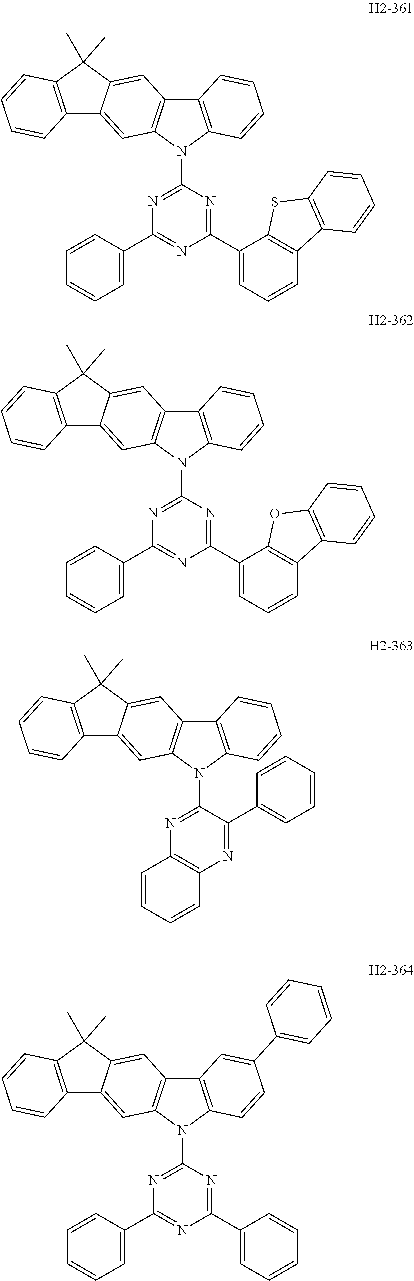

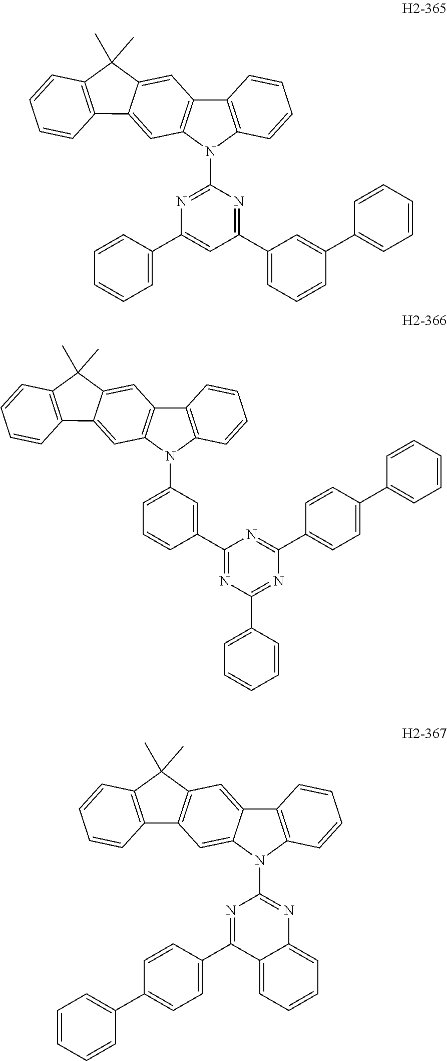 [SMILES: CC1(C)c2ccccc2-c2cc3c(cc21)c1ccccc1n3-c1nc(-c2ccccc2)cc(-c2cccc(-c3ccccc3)c2)n1 ; CC1(C)c2ccccc2-c2cc3c(cc21)c1ccccc1n3-c1cccc(-c2nc(-c3ccccc3)nc(-c3ccc(-c4ccccc4)cc3)n2)c1 ; CC1(C)c2ccccc2-c2cc3c(cc21)c1ccccc1n3-c1nc(-c2ccc(-c3ccccc3)cc2)c2ccccc2n1]

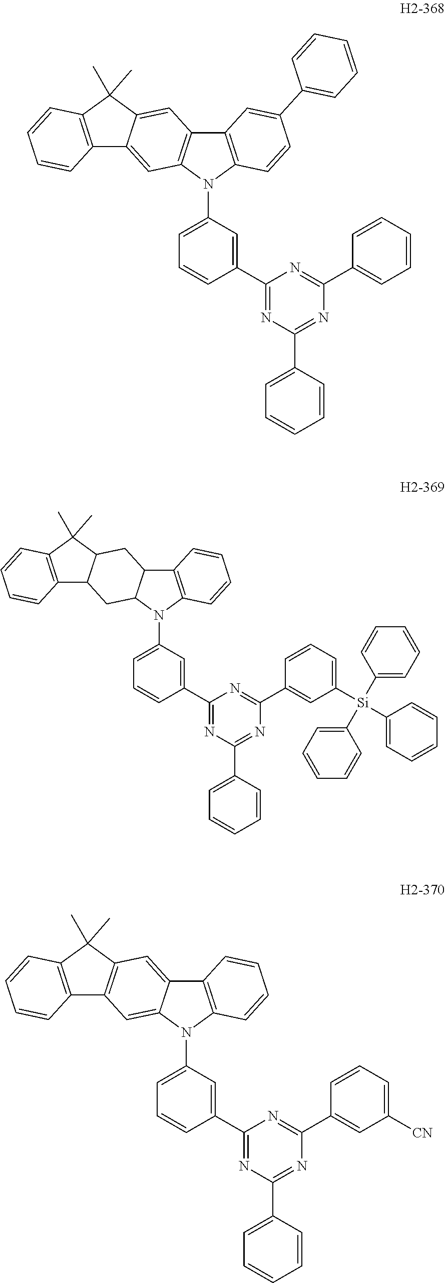 [SMILES: CC1(C)c2ccccc2-c2cc3c(cc21)c1cc(-c2ccccc2)ccc1n3-c1cccc(-c2nc(-c3ccccc3)nc(-c3ccccc3)n2)c1 ; CC1(C)c2ccccc2C2CC3C(CC21)c1ccccc1N3c1cccc(-c2nc(-c3ccccc3)nc(-c3cccc([Si](c4ccccc4)(c4ccccc4)c4ccccc4)c3)n2)c1 ; CC1(C)c2ccccc2-c2cc3c(cc21)c1ccccc1n3-c1cccc(-c2nc(-c3ccccc3)nc(-c3cccc(C#N)c3)n2)c1]

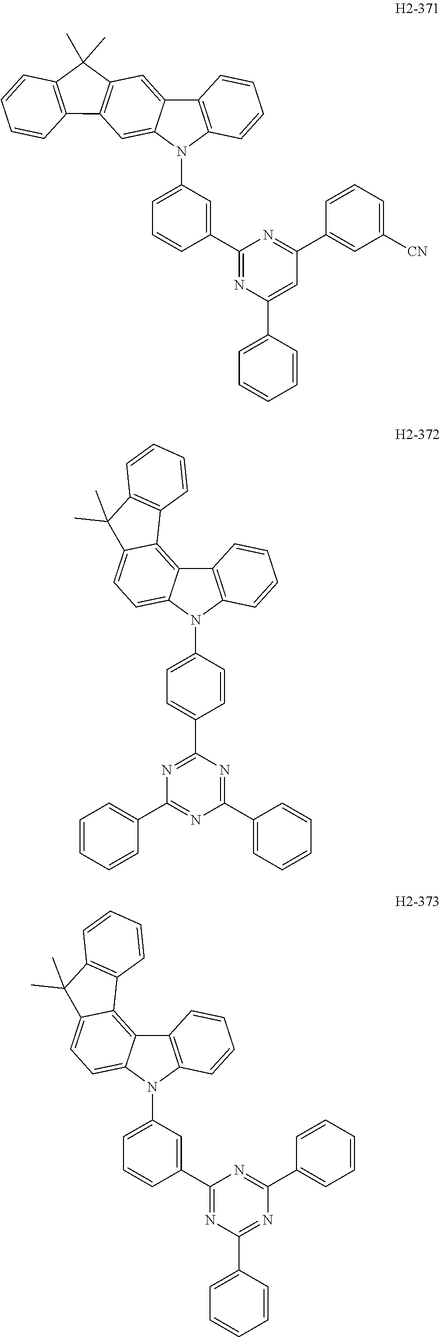

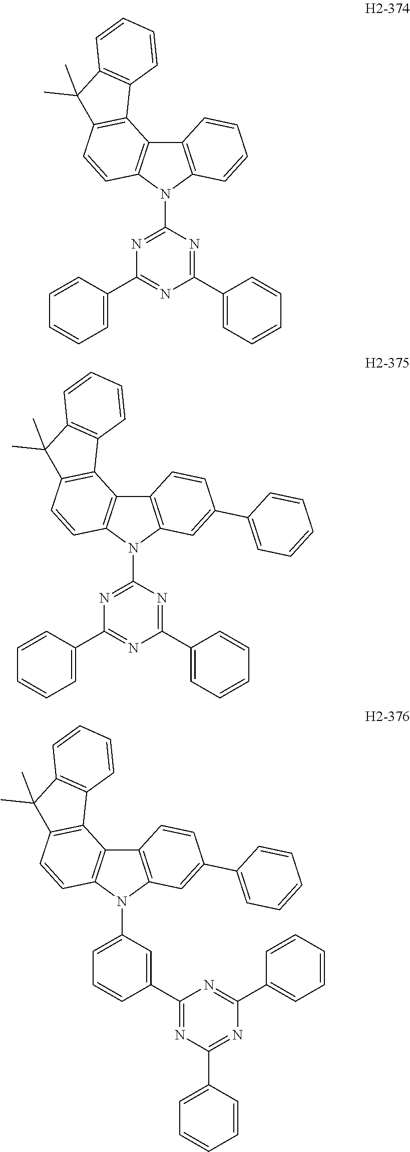

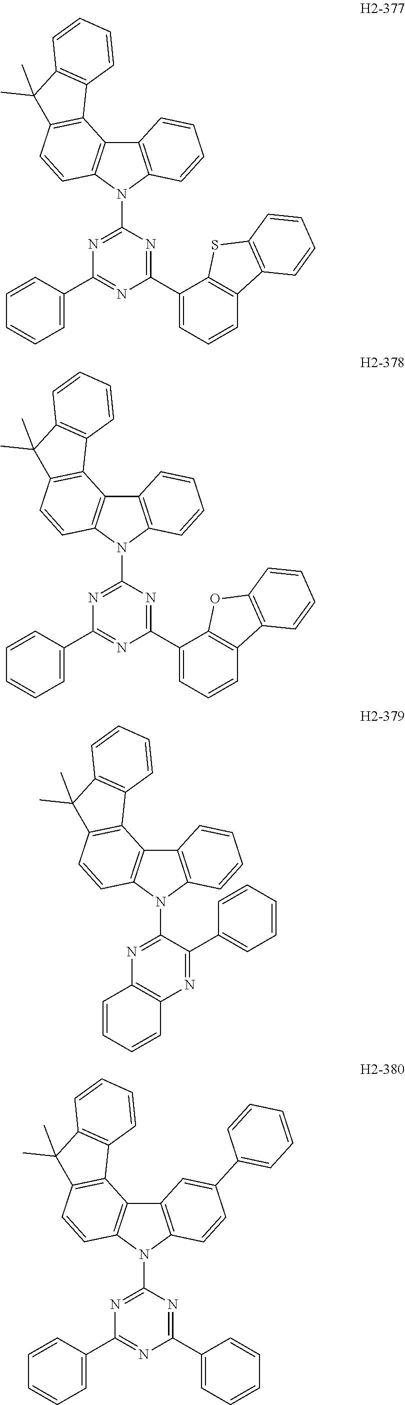

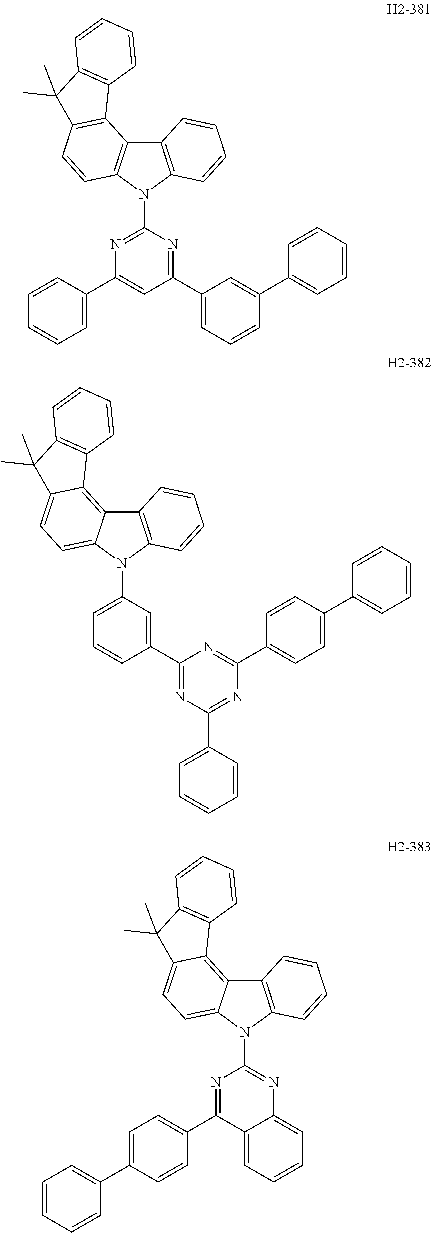

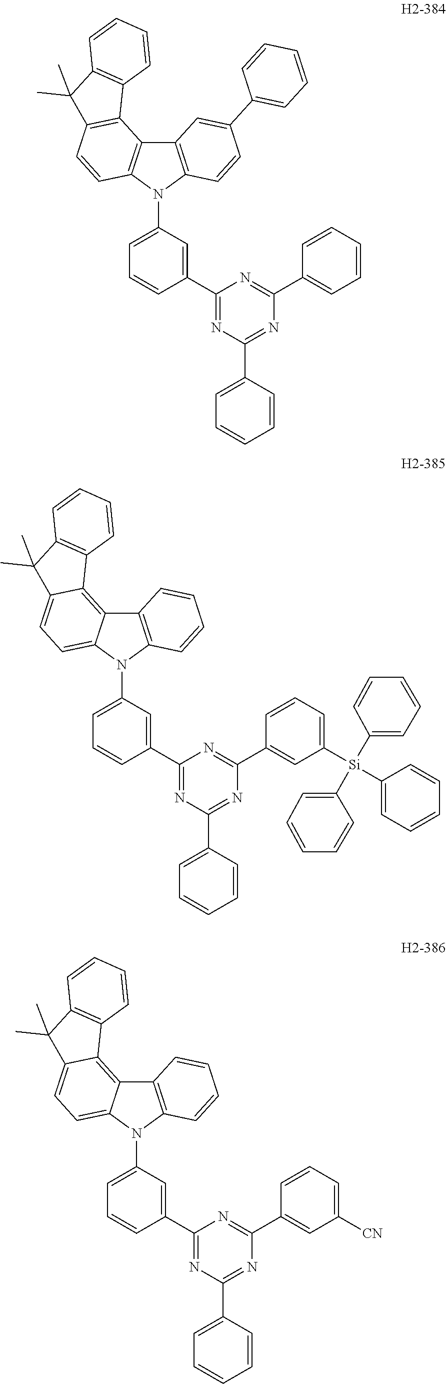 [SMILES: CC1(C)c2ccccc2-c2c1ccc1c2c2cc(-c3ccccc3)ccc2n1-c1cccc(-c2nc(-c3ccccc3)nc(-c3ccccc3)n2)c1 ; CC1(C)c2ccccc2-c2c1ccc1c2c2ccccc2n1-c1cccc(-c2nc(-c3ccccc3)nc(-c3cccc([Si](c4ccccc4)(c4ccccc4)c4ccccc4)c3)n2)c1 ; CC1(C)c2ccccc2-c2c1ccc1c2c2ccccc2n1-c1cccc(-c2nc(-c3ccccc3)nc(-c3cccc(C#N)c3)n2)c1]

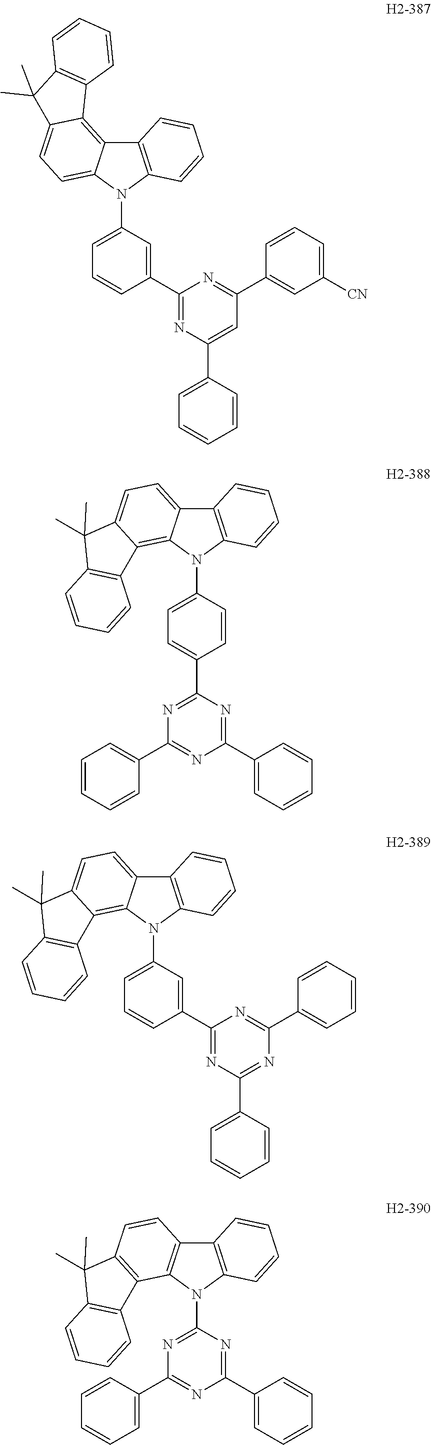

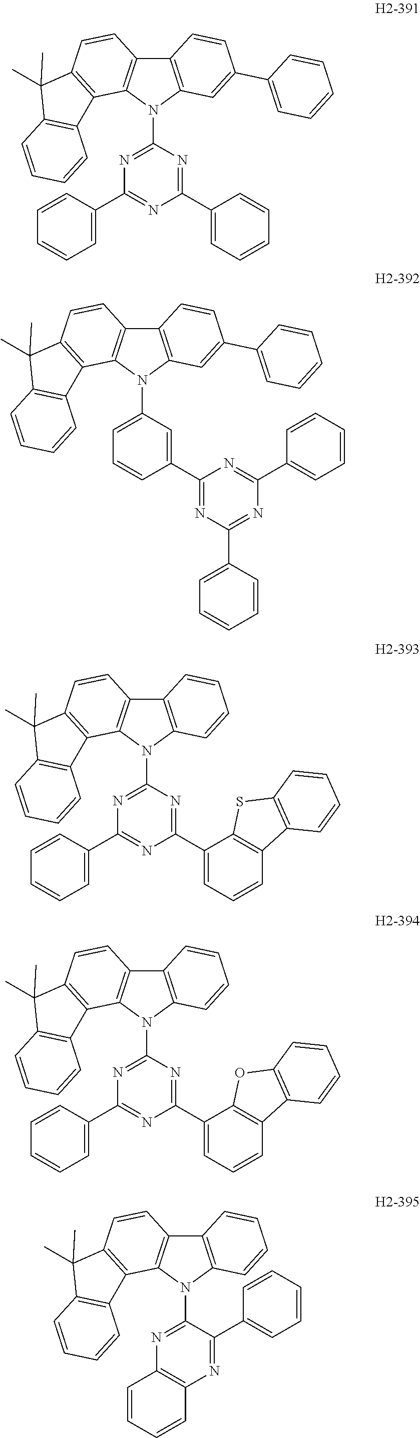

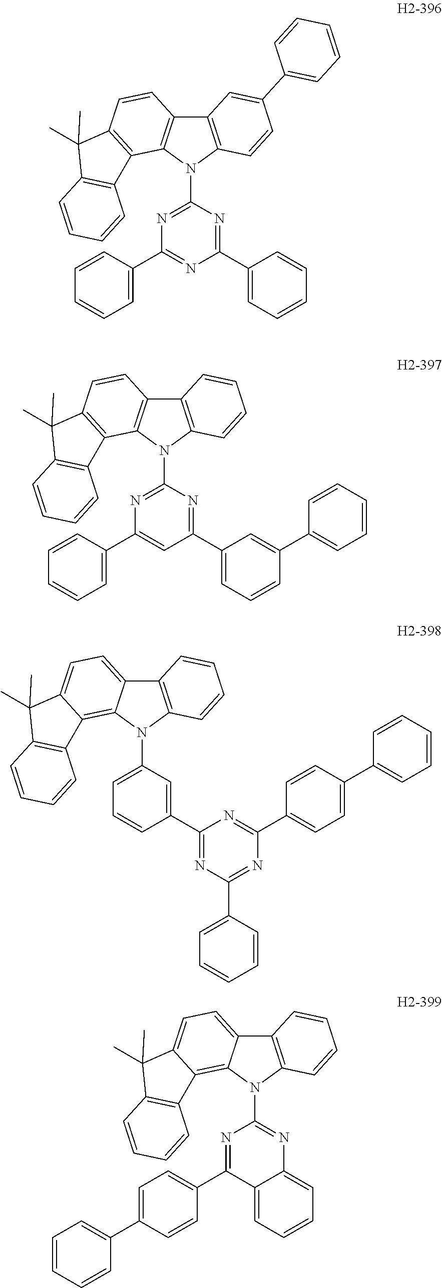

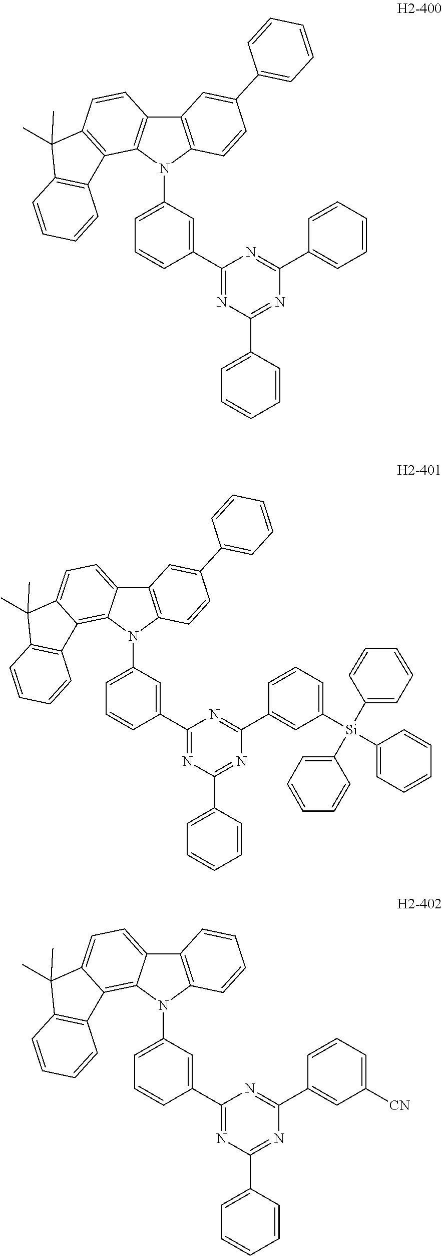 [SMILES: CC1(C)c2ccccc2-c2c1ccc1c3cc(-c4ccccc4)ccc3n(-c3cccc(-c4nc(-c5ccccc5)nc(-c5ccccc5)n4)c3)c21 ; CC1(C)c2ccccc2-c2c1ccc1c3cc(-c4ccccc4)ccc3n(-c3cccc(-c4nc(-c5ccccc5)nc(-c5cccc([Si](c6ccccc6)(c6ccccc6)c6ccccc6)c5)n4)c3)c21 ; CC1(C)c2ccccc2-c2c1ccc1c3ccccc3n(-c3cccc(-c4nc(-c5ccccc5)nc(-c5cccc(C#N)c5)n4)c3)c21]

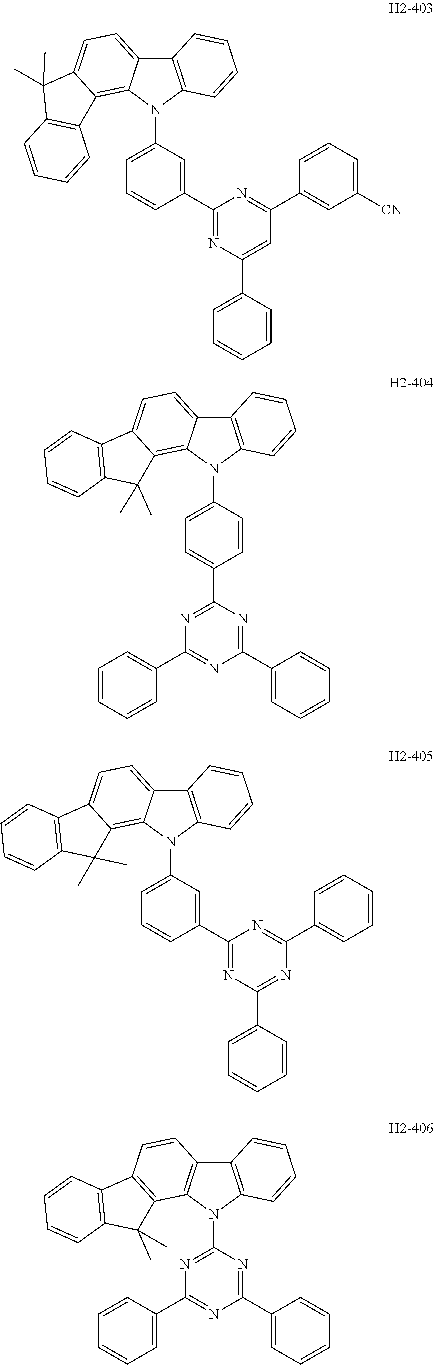

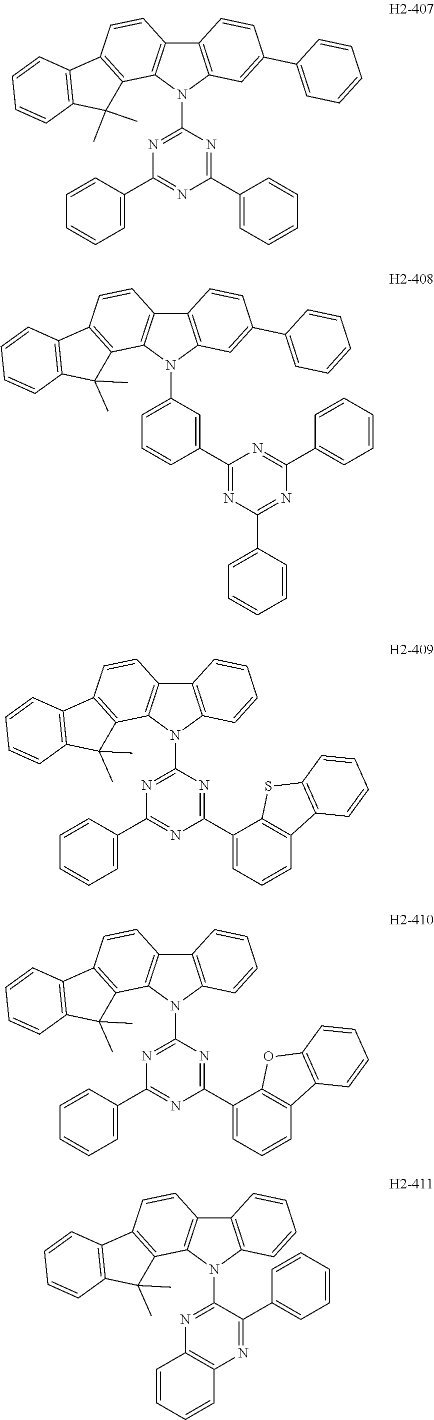

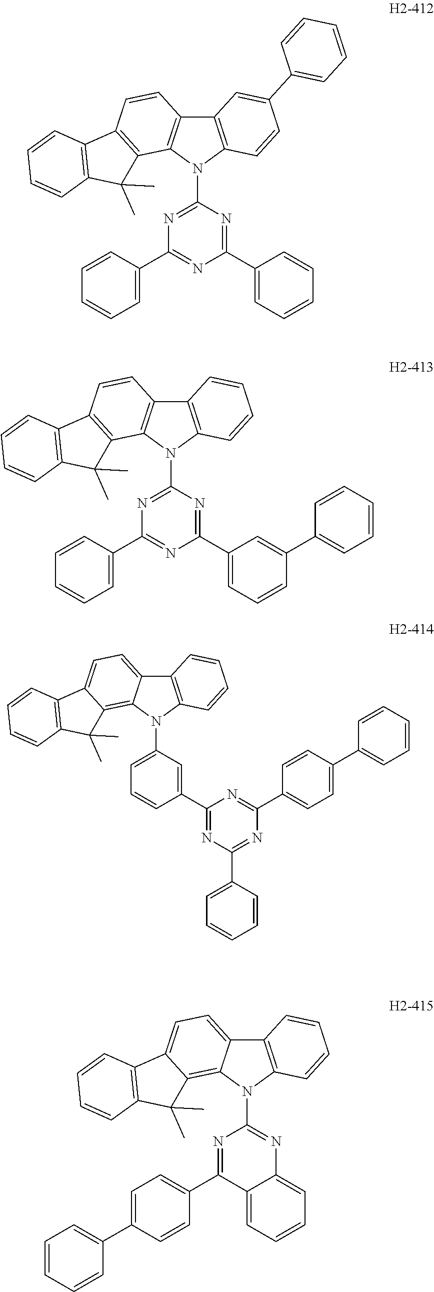 [SMILES: CC1(C)c2ccccc2-c2ccc3c4cc(-c5ccccc5)ccc4n(-c4nc(-c5ccccc5)nc(-c5ccccc5)n4)c3c21 ; CC1(C)c2ccccc2-c2ccc3c4ccccc4n(-c4nc(-c5ccccc5)nc(-c5cccc(-c6ccccc6)c5)n4)c3c21 ; CC1(C)c2ccccc2-c2ccc3c4ccccc4n(-c4cccc(-c5nc(-c6ccccc6)nc(-c6ccc(-c7ccccc7)cc6)n5)c4)c3c21 ; CC1(C)c2ccccc2-c2ccc3c4ccccc4n(-c4nc(-c5ccc(-c6ccccc6)cc5)c5ccccc5n4)c3c21]

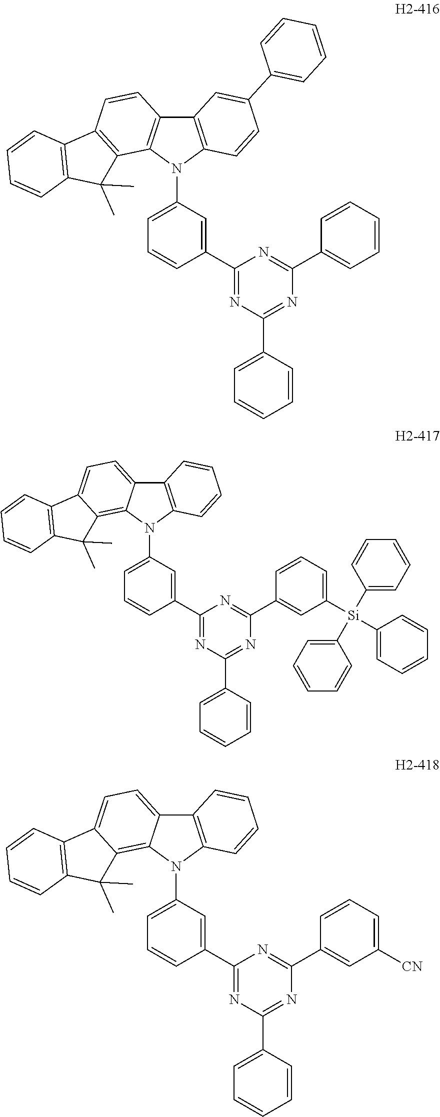

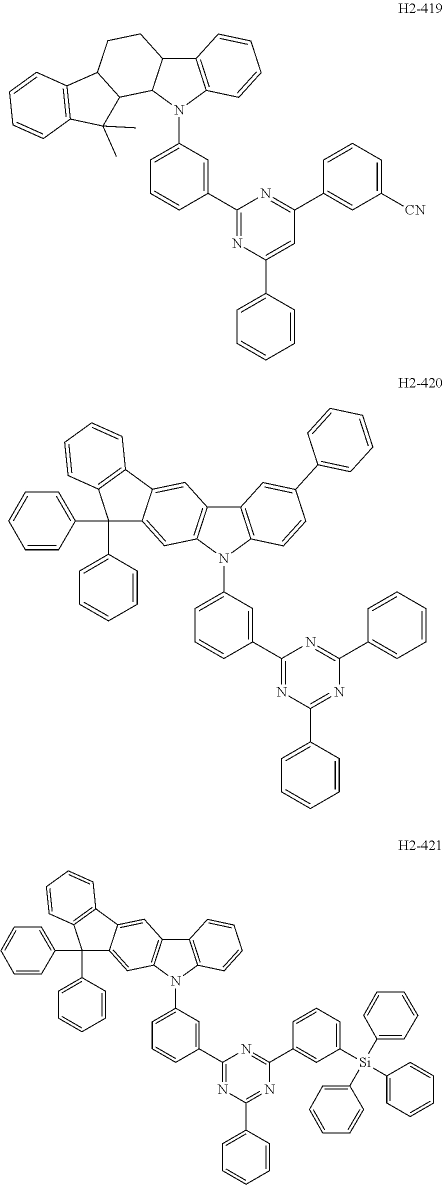 [SMILES: CC1(C)c2ccccc2C2CCC3c4ccccc4N(c4cccc(-c5nc(-c6ccccc6)cc(-c6cccc(C#N)c6)n5)c4)C3C21 ; c1ccc(-c2ccc3c(c2)c2cc4c(cc2n3-c2cccc(-c3nc(-c5ccccc5)nc(-c5ccccc5)n3)c2)C(c2ccccc2)(c2ccccc2)c2ccccc2-4)cc1 ; c1ccc(-c2nc(-c3cccc(-n4c5ccccc5c5cc6c(cc54)C(c4ccccc4)(c4ccccc4)c4ccccc4-6)c3)nc(-c3cccc([Si](c4ccccc4)(c4ccccc4)c4ccccc4)c3)n2)cc1]

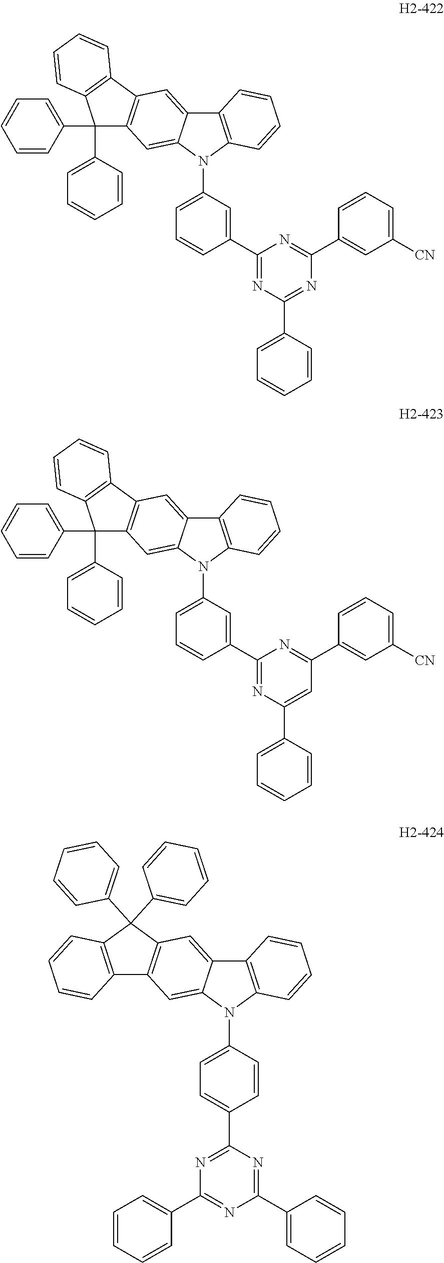 [SMILES: N#Cc1cccc(-c2nc(-c3ccccc3)nc(-c3cccc(-n4c5ccccc5c5cc6c(cc54)C(c4ccccc4)(c4ccccc4)c4ccccc4-6)c3)n2)c1 ; N#Cc1cccc(-c2cc(-c3ccccc3)nc(-c3cccc(-n4c5ccccc5c5cc6c(cc54)C(c4ccccc4)(c4ccccc4)c4ccccc4-6)c3)n2)c1 ; c1ccc(-c2nc(-c3ccccc3)nc(-c3ccc(-n4c5ccccc5c5cc6c(cc54)-c4ccccc4C6(c4ccccc4)c4ccccc4)cc3)n2)cc1]

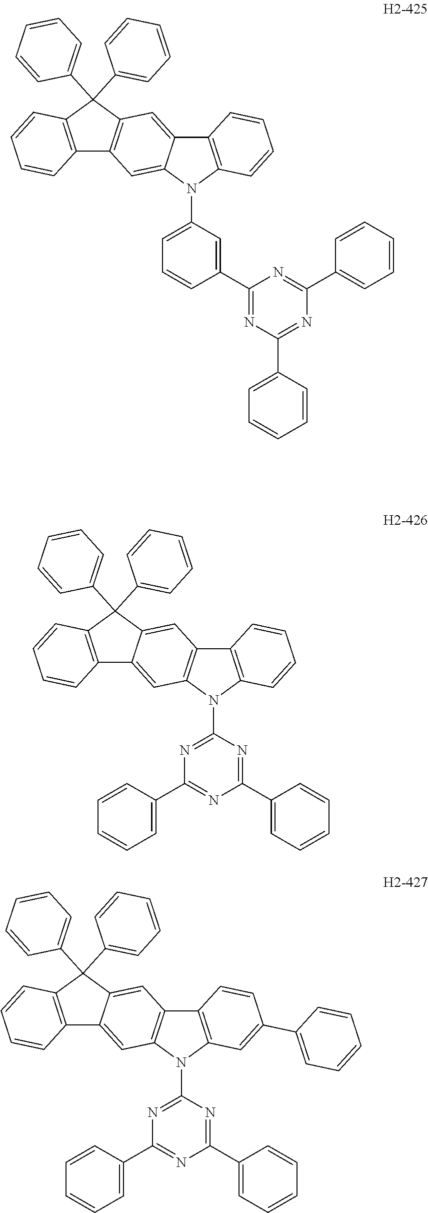

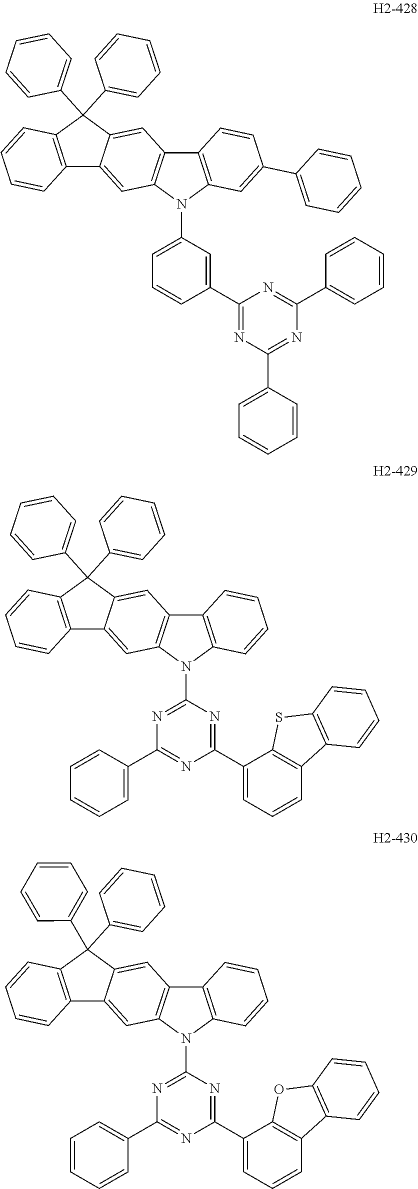 [SMILES: c1ccc(-c2ccc3c4cc5c(cc4n(-c4cccc(-c6nc(-c7ccccc7)nc(-c7ccccc7)n6)c4)c3c2)-c2ccccc2C5(c2ccccc2)c2ccccc2)cc1 ; c1ccc(-c2nc(-c3cccc4c3sc3ccccc34)nc(-n3c4ccccc4c4cc5c(cc43)-c3ccccc3C5(c3ccccc3)c3ccccc3)n2)cc1 ; c1ccc(-c2nc(-c3cccc4c3oc3ccccc34)nc(-n3c4ccccc4c4cc5c(cc43)-c3ccccc3C5(c3ccccc3)c3ccccc3)n2)cc1]

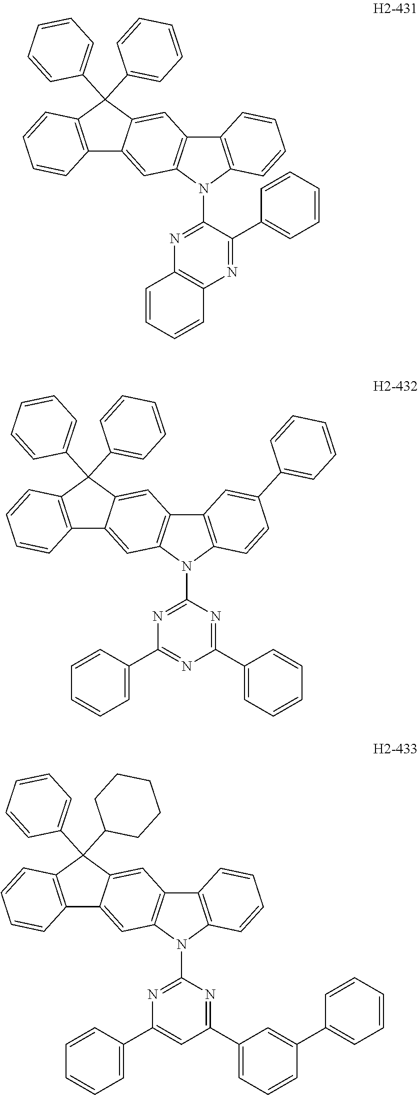 [SMILES: c1ccc(-c2nc3ccccc3nc2-n2c3ccccc3c3cc4c(cc32)-c2ccccc2C4(c2ccccc2)c2ccccc2)cc1 ; c1ccc(-c2ccc3c(c2)c2cc4c(cc2n3-c2nc(-c3ccccc3)nc(-c3ccccc3)n2)-c2ccccc2C4(c2ccccc2)c2ccccc2)cc1 ; c1ccc(-c2cccc(-c3cc(-c4ccccc4)nc(-n4c5ccccc5c5cc6c(cc54)-c4ccccc4C6(c4ccccc4)C4CCCCC4)n3)c2)cc1]

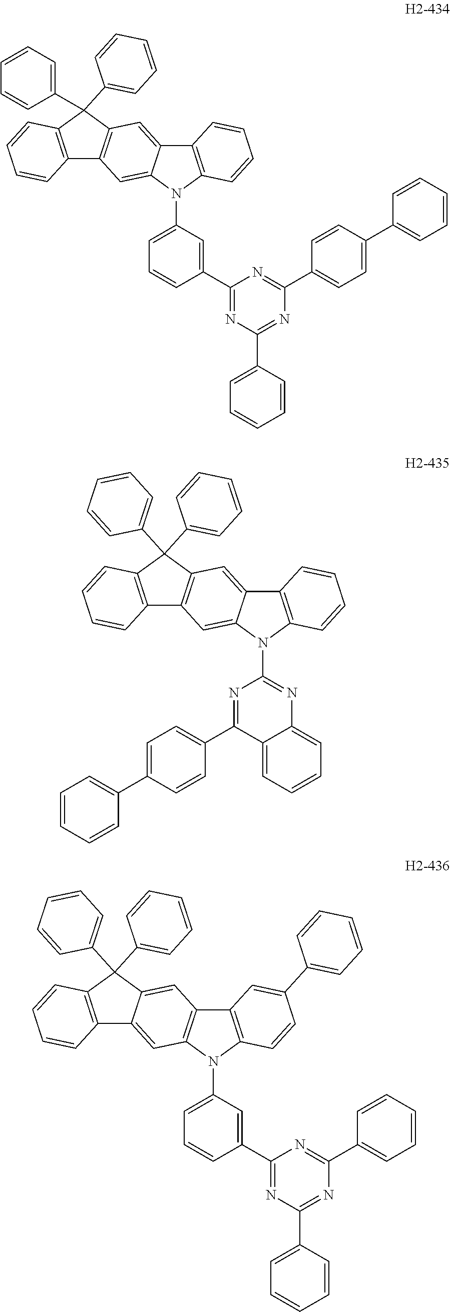 [SMILES: c1ccc(-c2ccc(-c3nc(-c4ccccc4)nc(-c4cccc(-n5c6ccccc6c6cc7c(cc65)-c5ccccc5C7(c5ccccc5)c5ccccc5)c4)n3)cc2)cc1 ; c1ccc(-c2ccc(-c3nc(-n4c5ccccc5c5cc6c(cc54)-c4ccccc4C6(c4ccccc4)c4ccccc4)nc4ccccc34)cc2)cc1 ; c1ccc(-c2ccc3c(c2)c2cc4c(cc2n3-c2cccc(-c3nc(-c5ccccc5)nc(-c5ccccc5)n3)c2)-c2ccccc2C4(c2ccccc2)c2ccccc2)cc1]

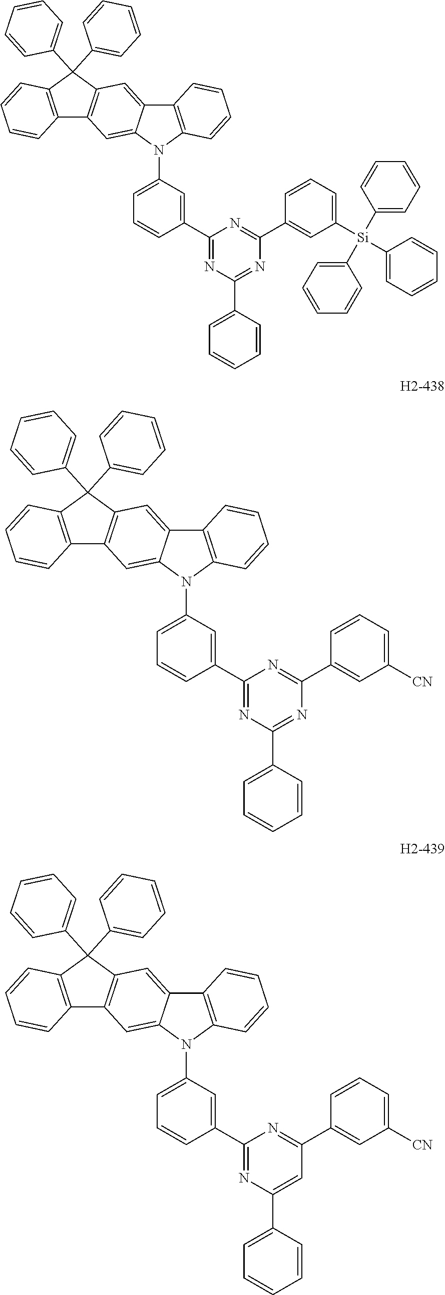 [SMILES: c1ccc(-c2nc(-c3cccc(-n4c5ccccc5c5cc6c(cc54)-c4ccccc4C6(c4ccccc4)c4ccccc4)c3)nc(-c3cccc([Si](c4ccccc4)(c4ccccc4)c4ccccc4)c3)n2)cc1 ; N#Cc1cccc(-c2nc(-c3ccccc3)nc(-c3cccc(-n4c5ccccc5c5cc6c(cc54)-c4ccccc4C6(c4ccccc4)c4ccccc4)c3)n2)c1 ; N#Cc1cccc(-c2cc(-c3ccccc3)nc(-c3cccc(-n4c5ccccc5c5cc6c(cc54)-c4ccccc4C6(c4ccccc4)c4ccccc4)c3)n2)c1]

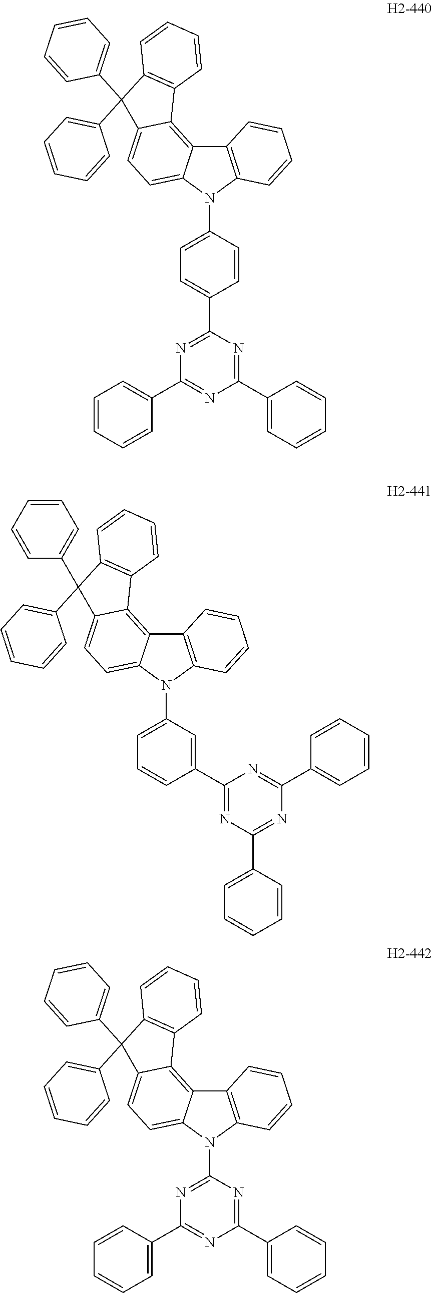 [SMILES: c1ccc(-c2nc(-c3ccccc3)nc(-c3ccc(-n4c5ccccc5c5c6c(ccc54)C(c4ccccc4)(c4ccccc4)c4ccccc4-6)cc3)n2)cc1 ; c1ccc(-c2nc(-c3ccccc3)nc(-c3cccc(-n4c5ccccc5c5c6c(ccc54)C(c4ccccc4)(c4ccccc4)c4ccccc4-6)c3)n2)cc1 ; c1ccc(-c2nc(-c3ccccc3)nc(-n3c4ccccc4c4c5c(ccc43)C(c3ccccc3)(c3ccccc3)c3ccccc3-5)n2)cc1]

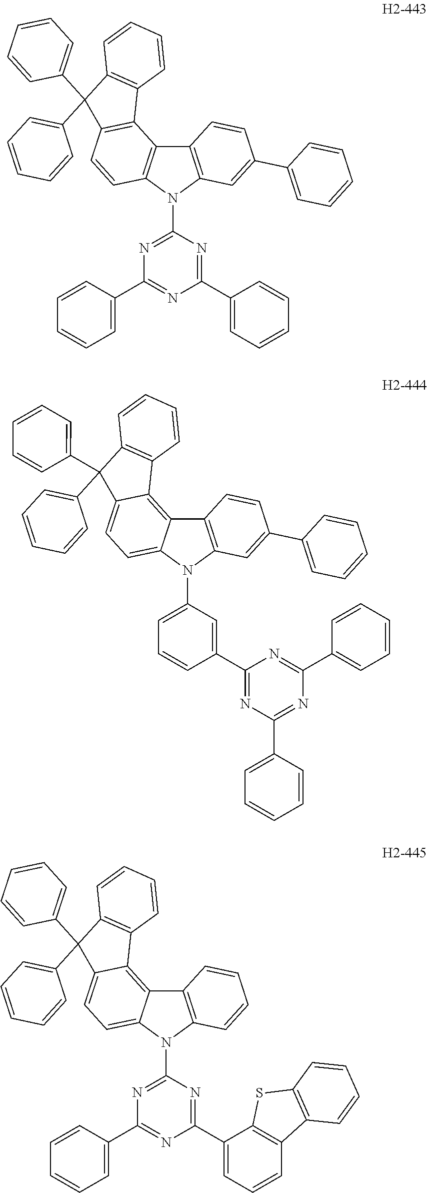 [SMILES: c1ccc(-c2ccc3c4c5c(ccc4n(-c4nc(-c6ccccc6)nc(-c6ccccc6)n4)c3c2)C(c2ccccc2)(c2ccccc2)c2ccccc2-5)cc1 ; c1ccc(-c2ccc3c4c5c(ccc4n(-c4cccc(-c6nc(-c7ccccc7)nc(-c7ccccc7)n6)c4)c3c2)C(c2ccccc2)(c2ccccc2)c2ccccc2-5)cc1 ; c1ccc(-c2nc(-c3cccc4c3sc3ccccc34)nc(-n3c4ccccc4c4c5c(ccc43)C(c3ccccc3)(c3ccccc3)c3ccccc3-5)n2)cc1]

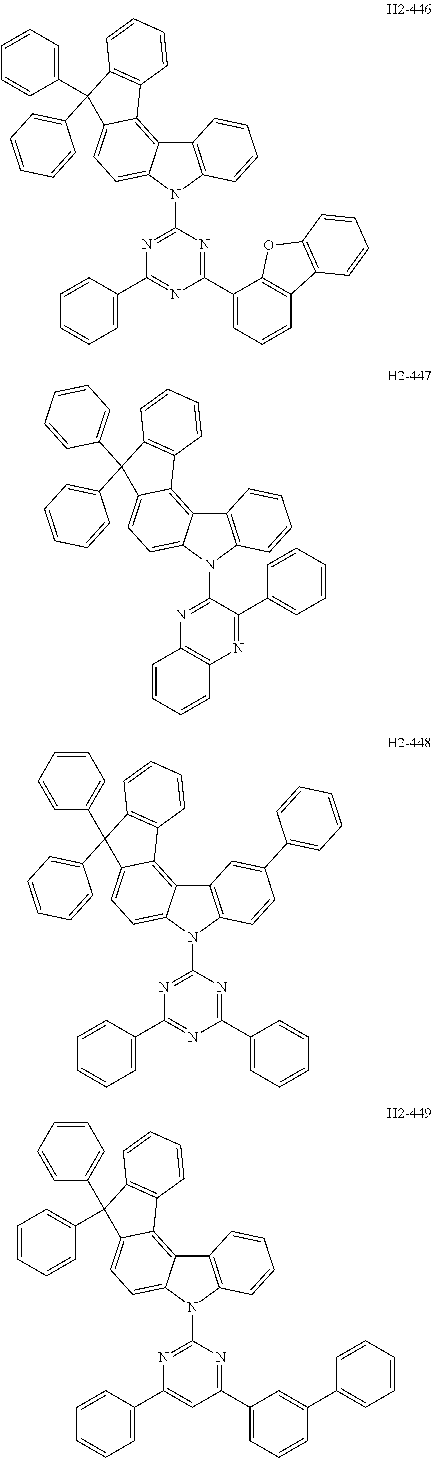

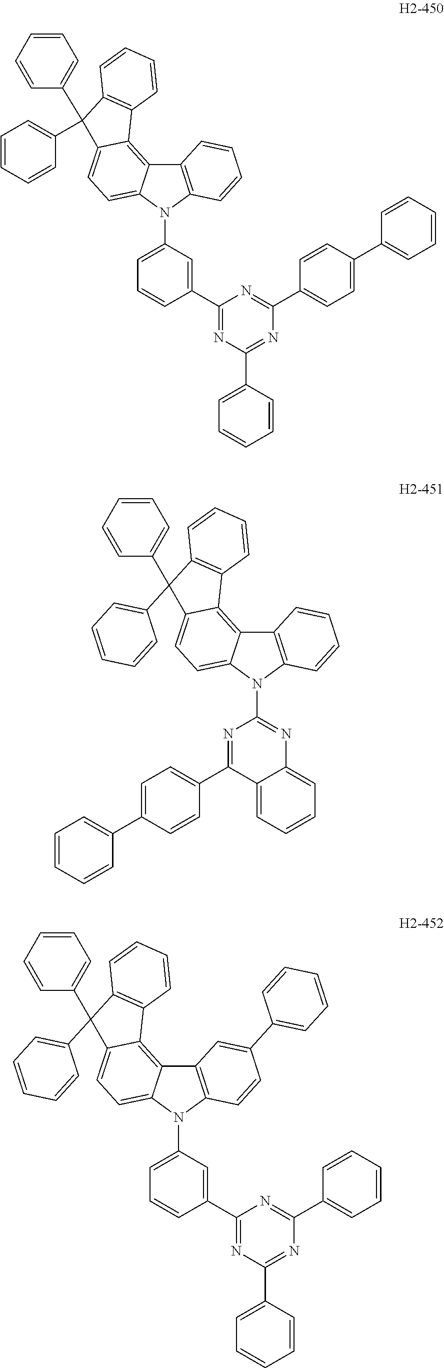

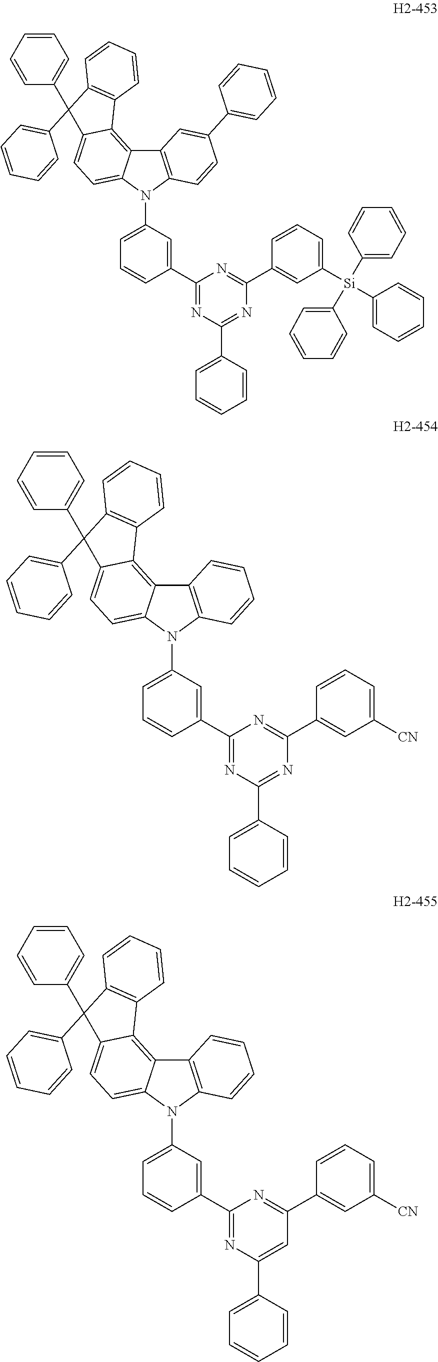

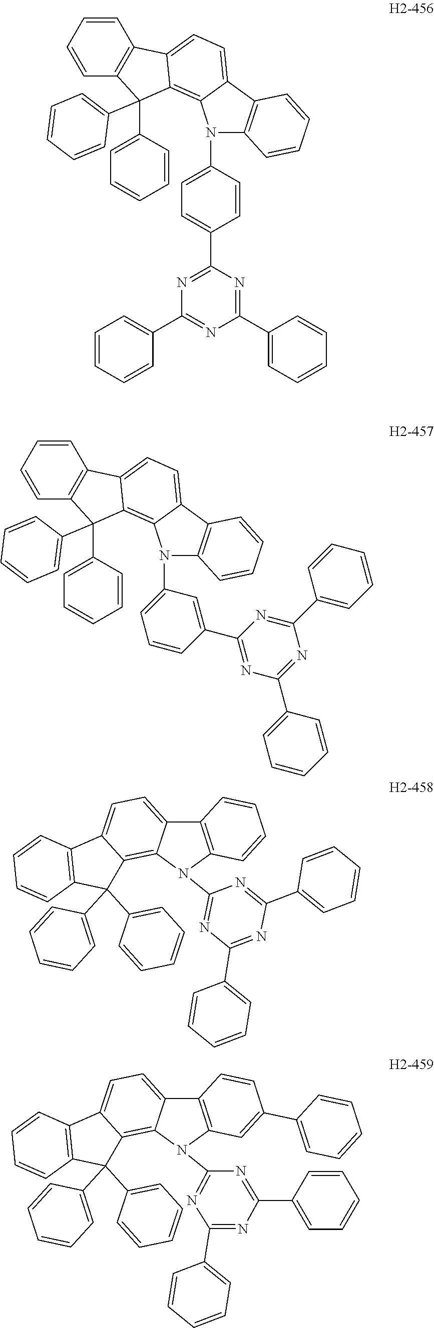

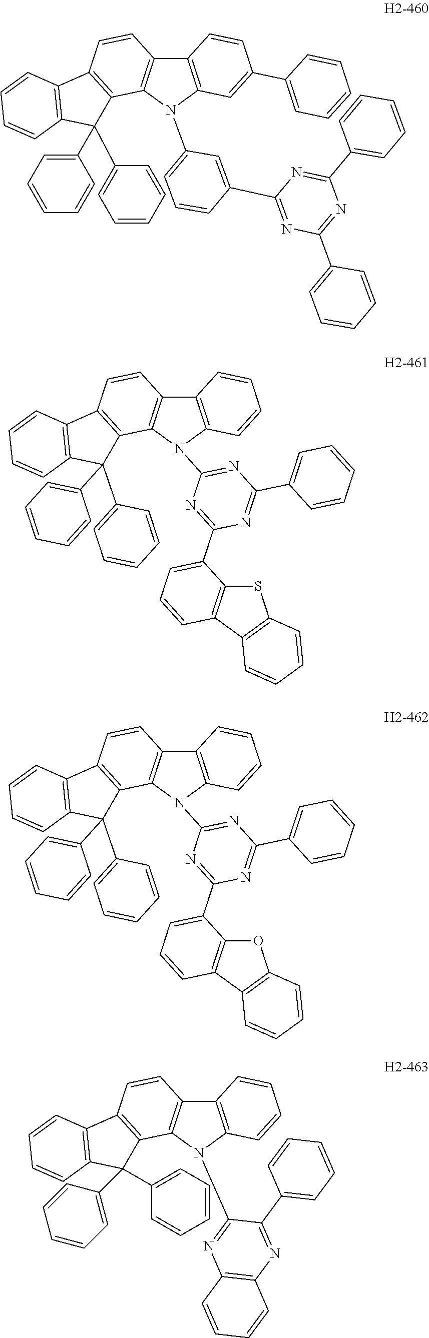

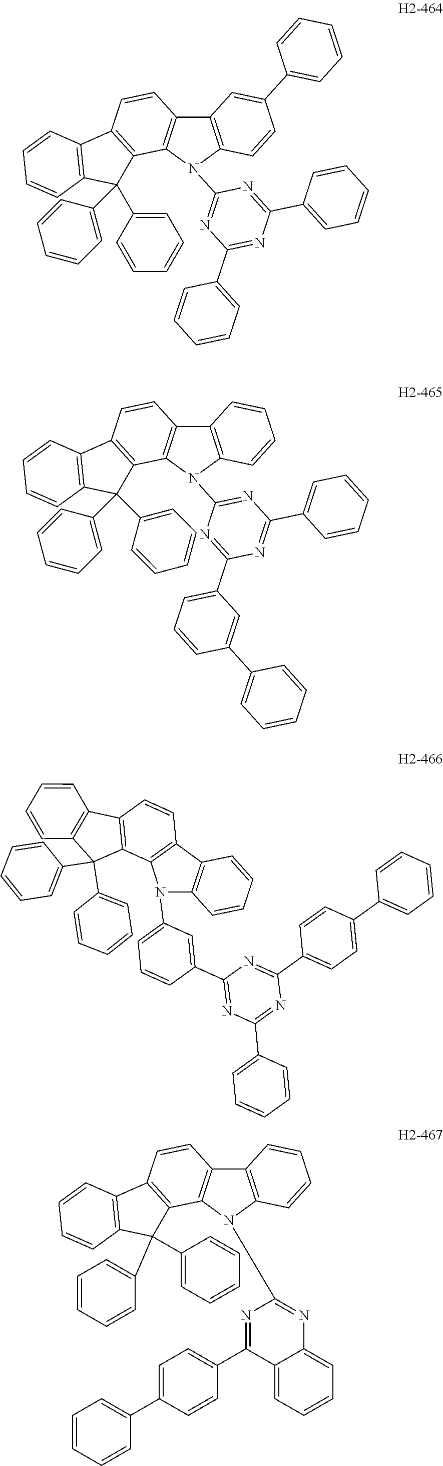

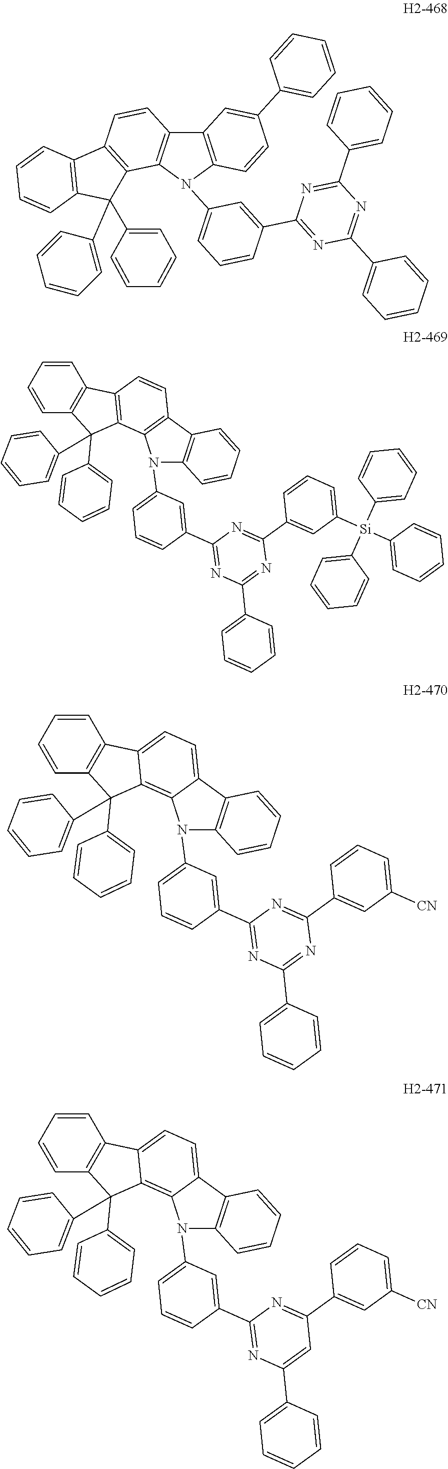 [SMILES: c1ccc(-c2ccc3c(c2)c2ccc4c(c2n3-c2cccc(-c3nc(-c5ccccc5)nc(-c5ccccc5)n3)c2)C(c2ccccc2)(c2ccccc2)c2ccccc2-4)cc1 ; c1ccc(-c2nc(-c3cccc(-n4c5ccccc5c5ccc6c(c54)C(c4ccccc4)(c4ccccc4)c4ccccc4-6)c3)nc(-c3cccc([Si](c4ccccc4)(c4ccccc4)c4ccccc4)c3)n2)cc1 ; N#Cc1cccc(-c2nc(-c3ccccc3)nc(-c3cccc(-n4c5ccccc5c5ccc6c(c54)C(c4ccccc4)(c4ccccc4)c4ccccc4-6)c3)n2)c1 ; N#Cc1cccc(-c2cc(-c3ccccc3)nc(-c3cccc(-n4c5ccccc5c5ccc6c(c54)C(c4ccccc4)(c4ccccc4)c4ccccc4-6)c3)n2)c1]

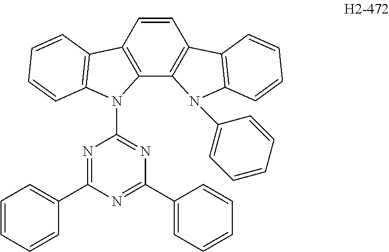 [SMILES: c1ccc(-c2nc(-c3ccccc3)nc(-n3c4ccccc4c4ccc5c6ccccc6n(-c6ccccc6)c5c43)n2)cc1]

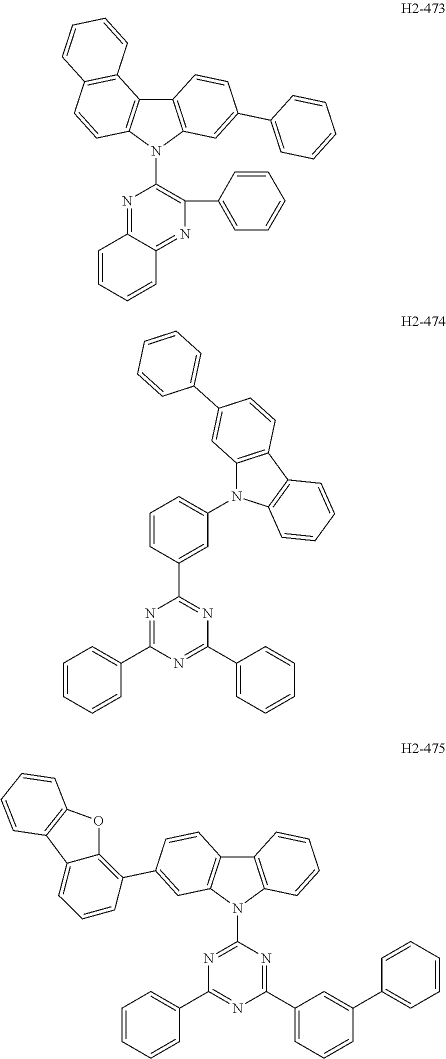

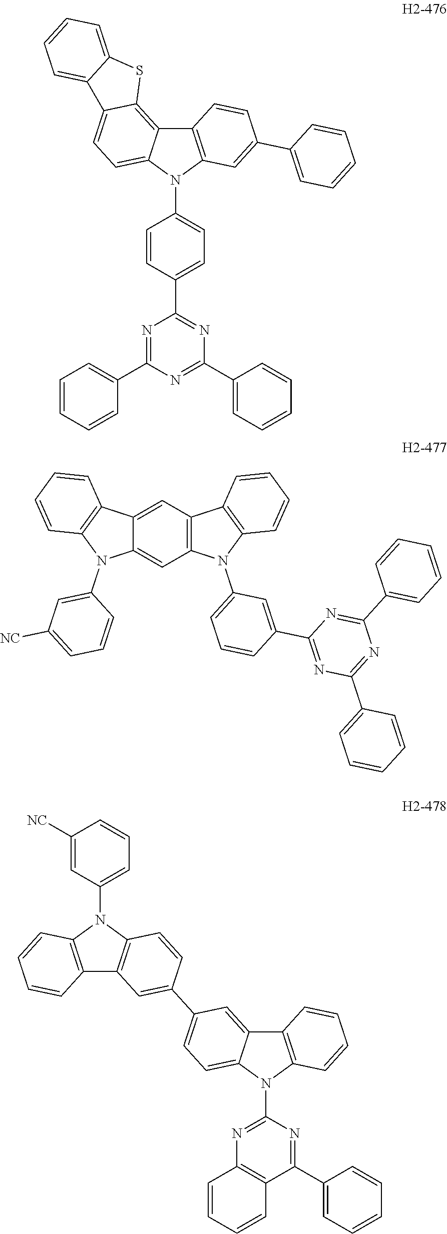

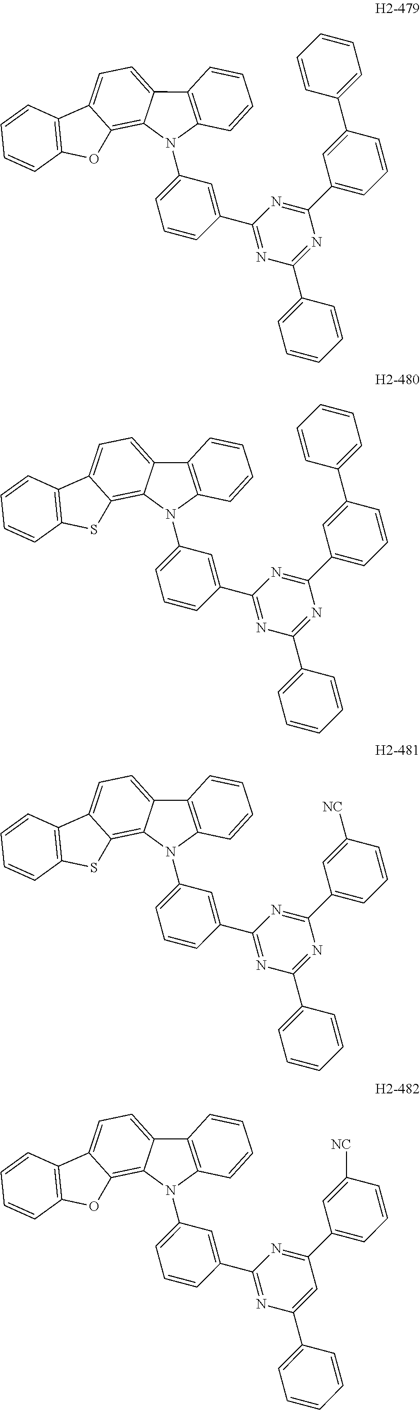

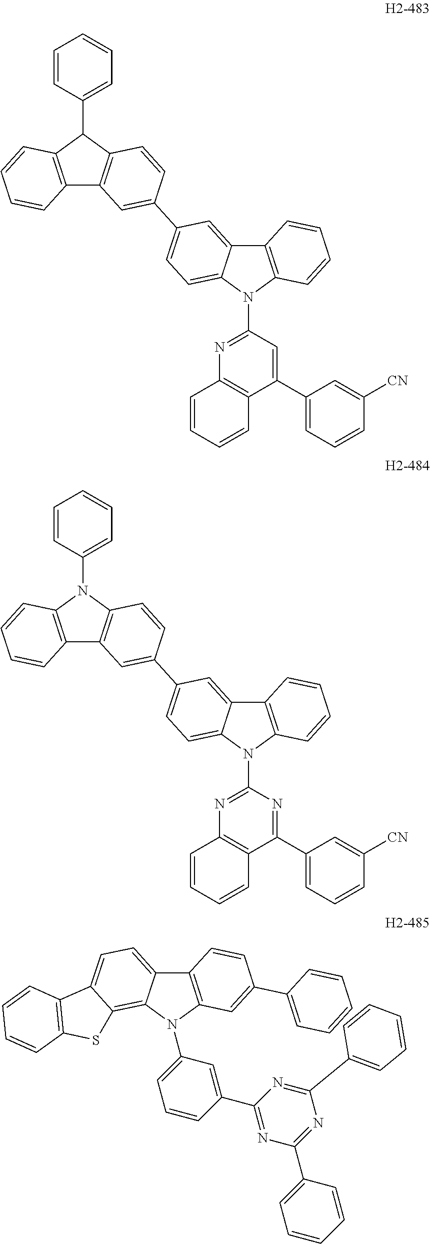

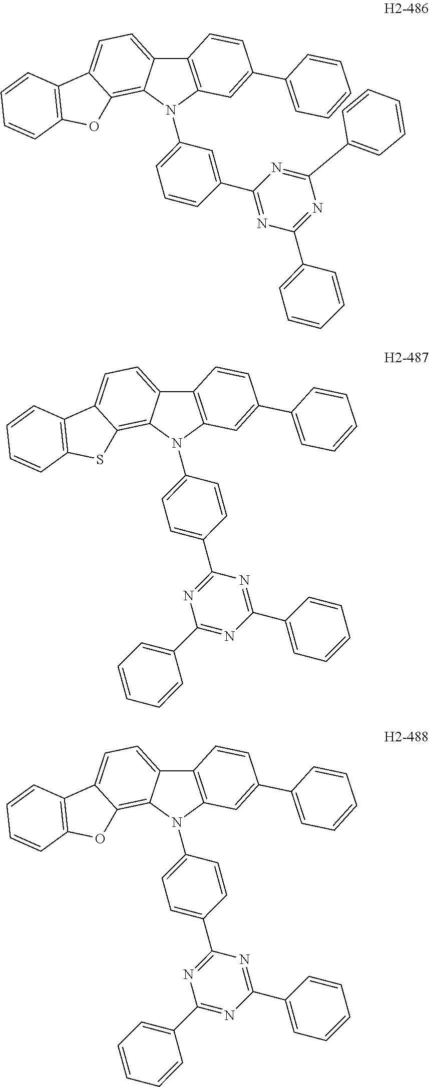

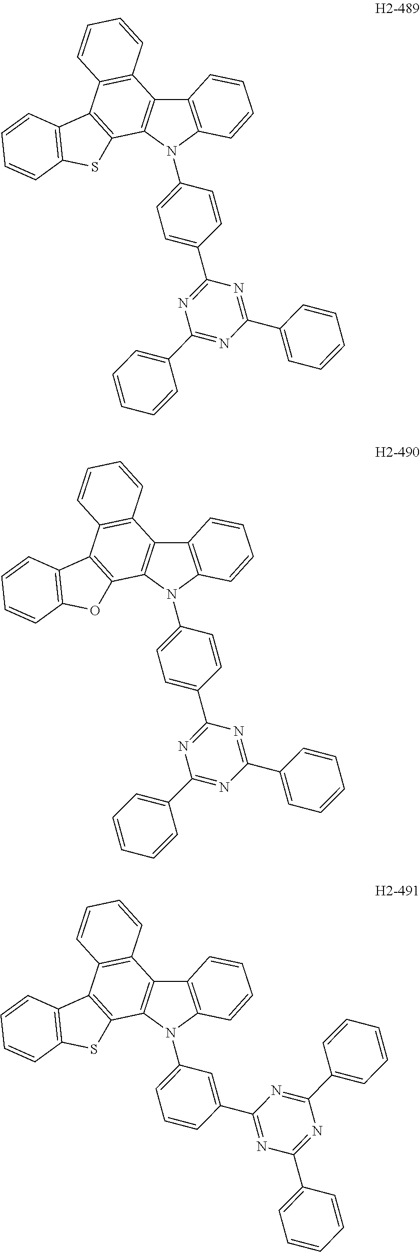 [SMILES: c1ccc(-c2nc(-c3ccccc3)nc(-c3ccc(-n4c5ccccc5c5c6ccccc6c6c7ccccc7sc6c54)cc3)n2)cc1 ; c1ccc(-c2nc(-c3ccccc3)nc(-c3ccc(-n4c5ccccc5c5c6ccccc6c6c7ccccc7oc6c54)cc3)n2)cc1 ; c1ccc(-c2nc(-c3ccccc3)nc(-c3cccc(-n4c5ccccc5c5c6ccccc6c6c7ccccc7sc6c54)c3)n2)cc1]

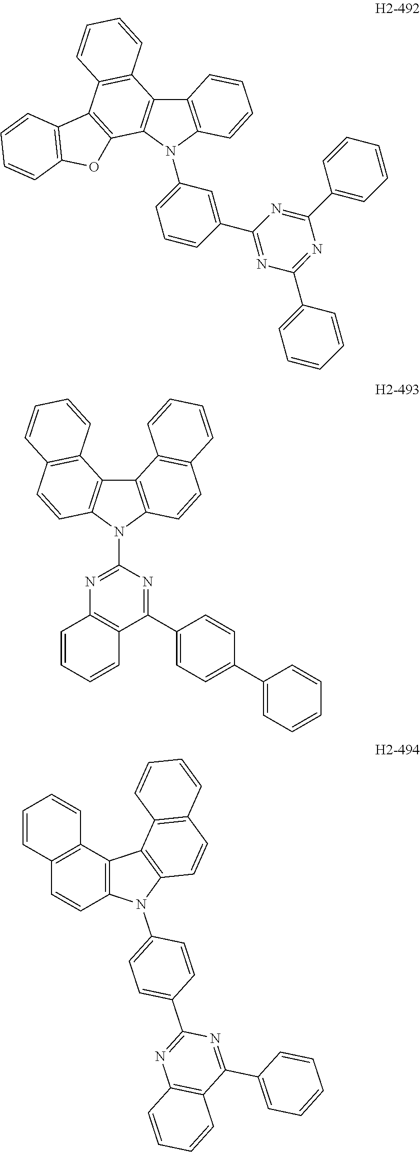 [SMILES: c1ccc(-c2nc(-c3ccccc3)nc(-c3cccc(-n4c5ccccc5c5c6ccccc6c6c7ccccc7oc6c54)c3)n2)cc1 ; c1ccc(-c2ccc(-c3nc(-n4c5ccc6ccccc6c5c5c6ccccc6ccc54)nc4ccccc34)cc2)cc1 ; c1ccc(-c2nc(-c3ccc(-n4c5ccc6ccccc6c5c5c6ccccc6ccc54)cc3)nc3ccccc23)cc1]

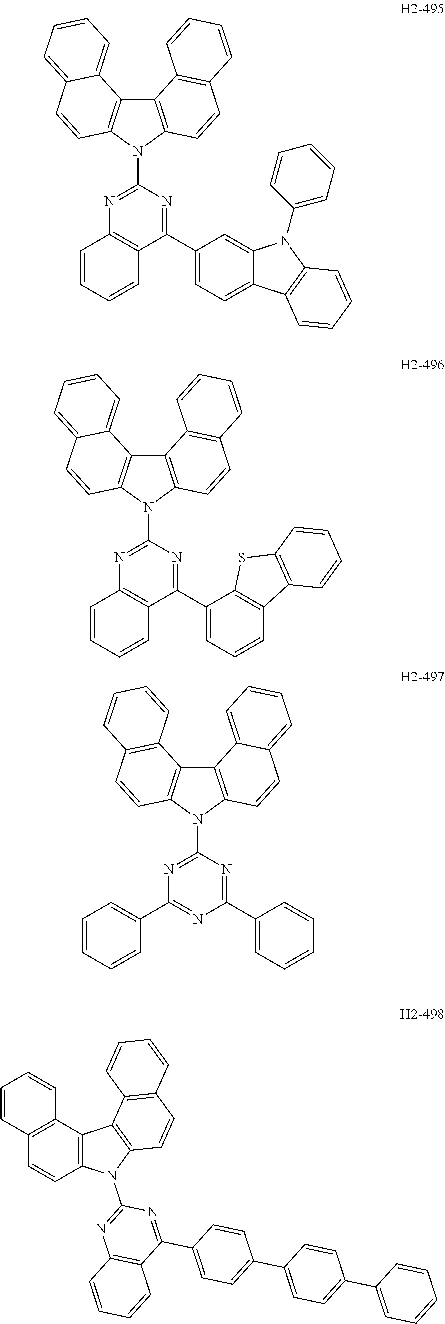

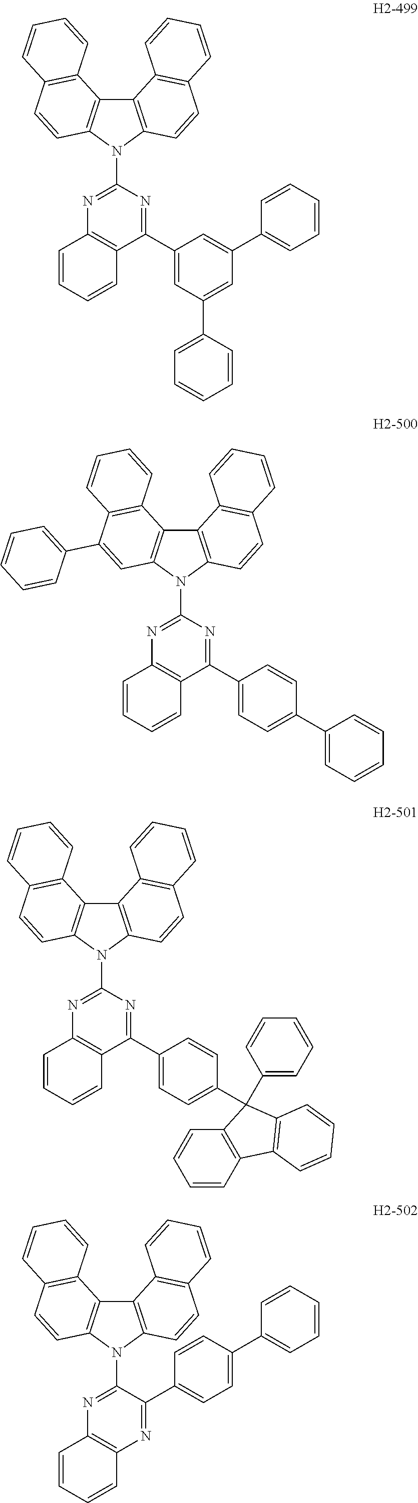

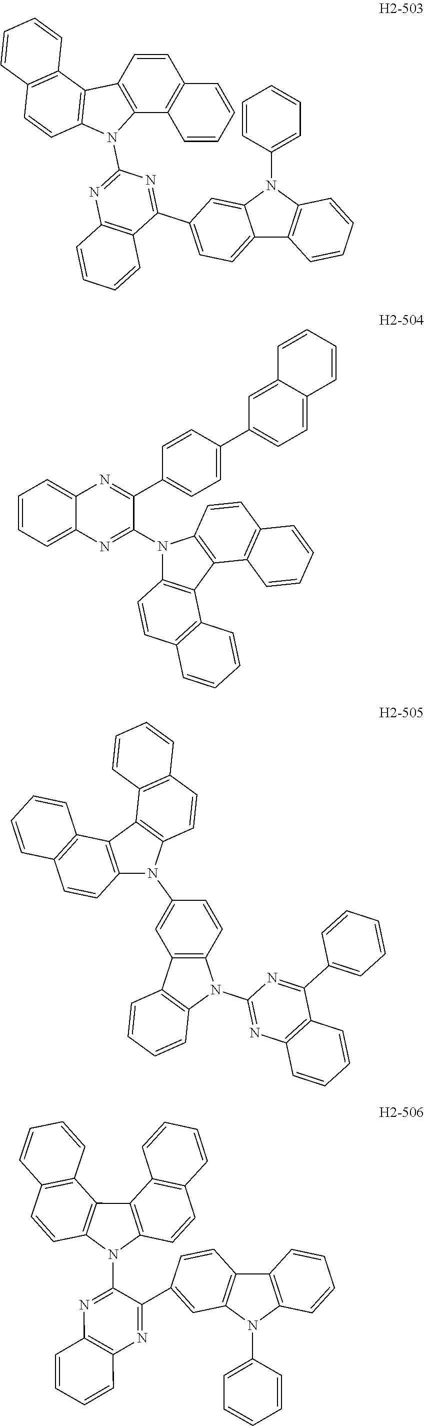 [SMILES: c1ccc(-n2c3ccccc3c3ccc(-c4nc(-n5c6ccc7ccccc7c6c6ccc7ccccc7c65)nc5ccccc45)cc32)cc1 ; c1ccc2cc(-c3ccc(-c4nc5ccccc5nc4-n4c5ccc6ccccc6c5c5c6ccccc6ccc54)cc3)ccc2c1 ; c1ccc(-c2nc(-n3c4ccccc4c4cc(-n5c6ccc7ccccc7c6c6c7ccccc7ccc65)ccc43)nc3ccccc23)cc1 ; c1ccc(-n2c3ccccc3c3ccc(-c4nc5ccccc5nc4-n4c5ccc6ccccc6c5c5c6ccccc6ccc54)cc32)cc1]

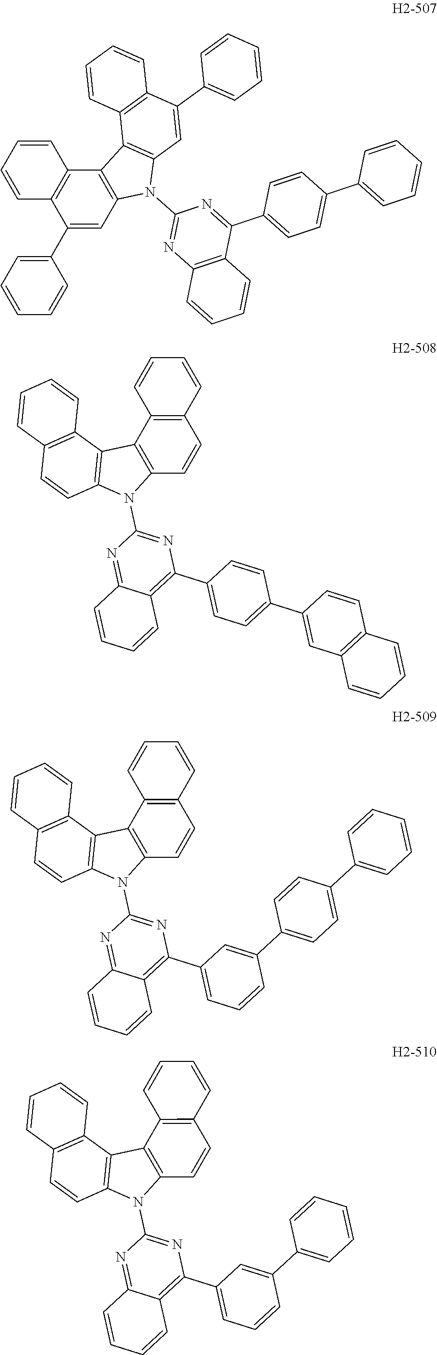

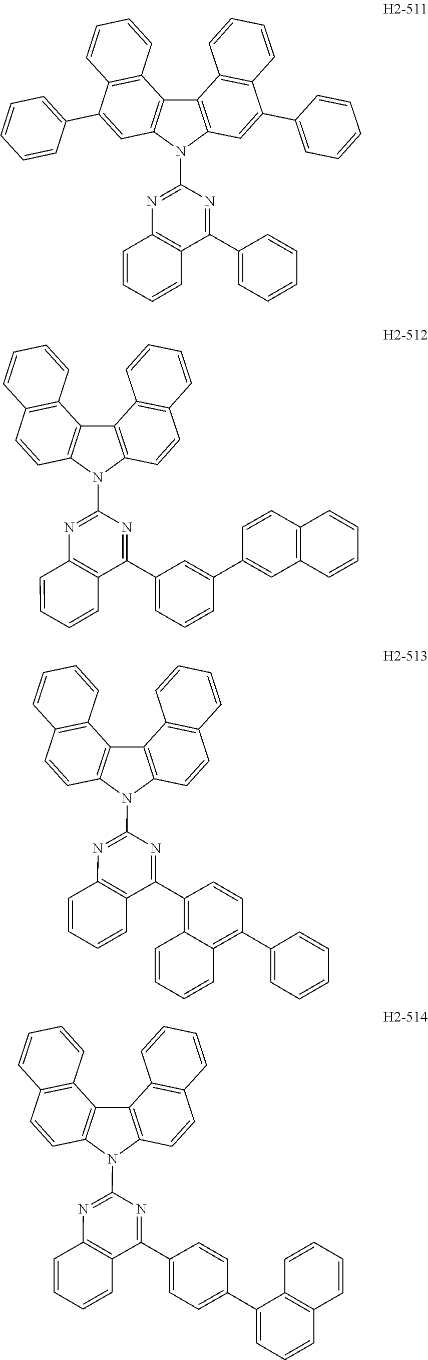

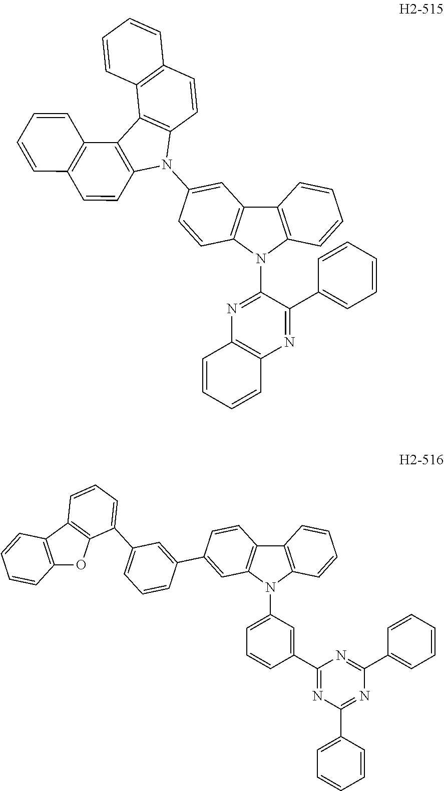 [SMILES: c1ccc(-c2nc3ccccc3nc2-n2c3ccccc3c3cc(-n4c5ccc6ccccc6c5c5c6ccccc6ccc54)ccc32)cc1 ; c1ccc(-c2nc(-c3ccccc3)nc(-c3cccc(-n4c5ccccc5c5ccc(-c6cccc(-c7cccc8c7oc7ccccc78)c6)cc54)c3)n2)cc1]

The organic electroluminescent device according to the present invention comprises an anode, a cathode, and at least one organic layer between the anode and the cathode. The organic layer comprises a light-emitting layer, and the light-emitting layer comprises a host and a phosphorescent dopant. The host material comprises plural host compounds, at least a first host compound of the plural host compounds is represented by formula 1, and a second host compound is represented by formula 2.

The light-emitting layer is a layer from which light is emitted, and can be a single layer or a multi-layer of which two or more layers are stacked. In the light-emitting layer, it is preferable that the doping concentration of the dopant compound based on the host compound is less than 20 wt %.

The organic layer comprises a light-emitting layer, and may further comprise at least one layer selected from the group consisting of a hole injection layer, a hole transport layer, an electron transport layer, an electron injection layer, an interlayer, a hole blocking layer, and an electron blocking layer.

According to the organic electroluminescent device of the present invention, the weight ratio of the first host material to the second host material is in the range of 1:99 to 99:1.

The dopant is preferably at least one phosphorescent dopant. The dopant materials applied to the organic electroluminescent device according to the present invention are not limited, but may be preferably selected from metallated complex compounds of iridium, osmium, copper, and platinum, more preferably selected from ortho-metallated complex compounds of iridium, osmium, copper and platinum, and even more preferably ortho-metallated iridium complex compounds.

The phosphorescent dopant is preferably selected from compounds represented by the following formulas 101 to 103.

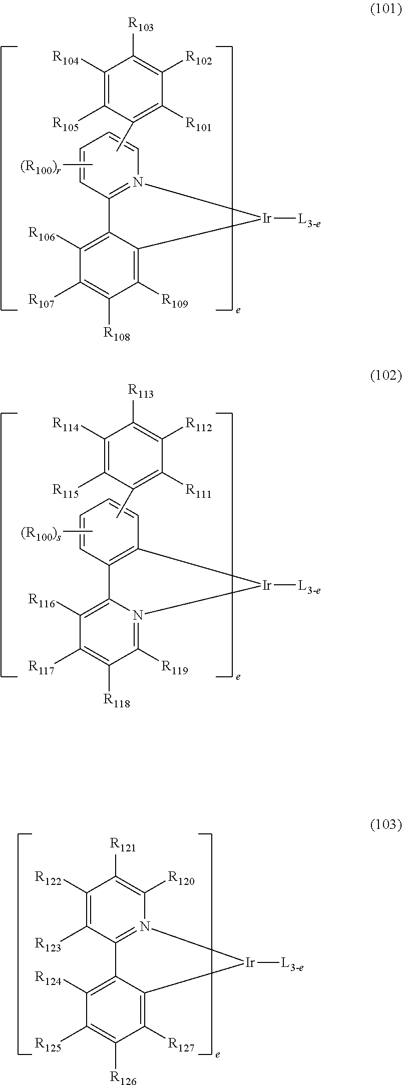

wherein L is selected from the following structures:

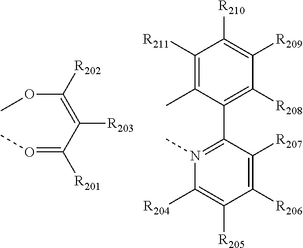

R₁₀₀ represents hydrogen, a substituted or unsubstituted (C1-C30)alkyl, or a substituted or unsubstituted (C3-C30)cycloalkyl;

R₁₀₁ to R₁₀₉, and R₁₁₁ to R₁₂₃ each independently represent hydrogen, deuterium, a halogen, a (C1-C30)alkyl unsubstituted or substituted with a halogen(s), a cyano, a substituted or unsubstituted (C1-C30)alkoxy, a substituted or unsubstituted (C6-C30)aryl, or a substituted or unsubstituted (C3-C30)cycloalkyl; adjacent substituents of R₁₀₆ to R₁₀₉ may be linked to each other to form a substituted or unsubstituted fused ring, e.g., fluorene unsubstituted or substituted with alkyl, dibenzothiophene unsubstituted or substituted with alkyl, or dibenzofuran unsubstituted or substituted with alkyl; and adjacent substituents of R₁₂₀ to R₁₂₃ may be linked to each other to form a substituted or unsubstituted fused ring, e.g., quinoline unsubstituted or substituted with alkyl or aryl;

R₁₂₄ to _{R127} each independently represent hydrogen, deuterium, a halogen, a substituted or unsubstituted (C1-C30)alkyl, or a substituted or unsubstituted (C6-C30)aryl; and adjacent substituents of R₁₂₄ to R₁₂₇ may be linked to each other to form a substituted or unsubstituted fused ring, e.g., fluorene unsubstituted or substituted with alkyl, dibenzothiophene unsubstituted or substituted with alkyl, or dibenzofuran unsubstituted or substituted with alkyl;

R₂₀₁ to R₂₁₁ each independently represent hydrogen, deuterium, a halogen, a (C1-C30)alkyl unsubstituted or substituted with a halogen(s), a substituted or unsubstituted (C3-C30)cycloalkyl, or a substituted or unsubstituted (C6-C30)aryl; and adjacent substituents of R₂₀₈ to R₂₁₁ may be linked to each other to form a substituted or unsubstituted fused ring, e.g., fluorene unsubstituted or substituted with alkyl, dibenzothiophene unsubstituted or substituted with alkyl, or dibenzofuran unsubstituted or substituted with alkyl;

r and s each independently represent an integer of 1 to 3; where r or s is an integer of 2 or more, each of R₁₀₀ may be the same or different; and

e represents an integer of 1 to 3.

Specifically, the phosphorescent dopant materials include the following:

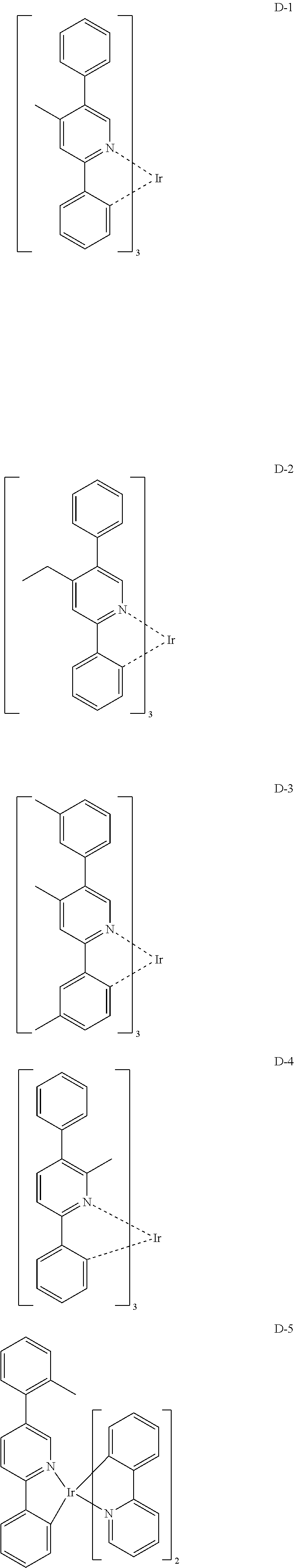

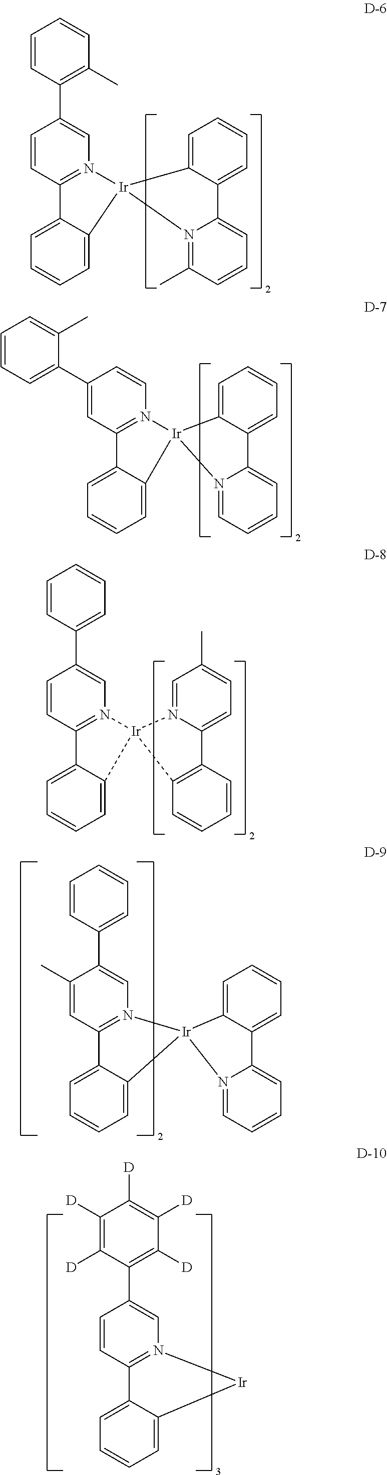

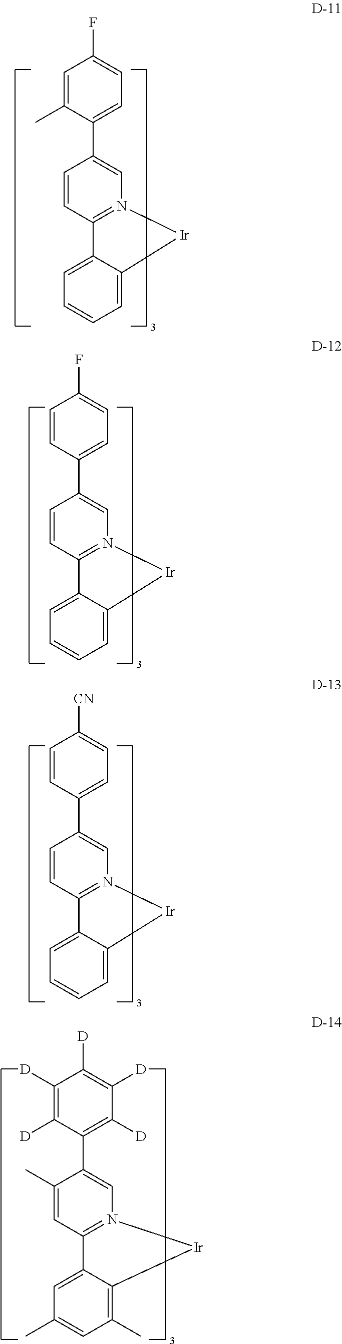

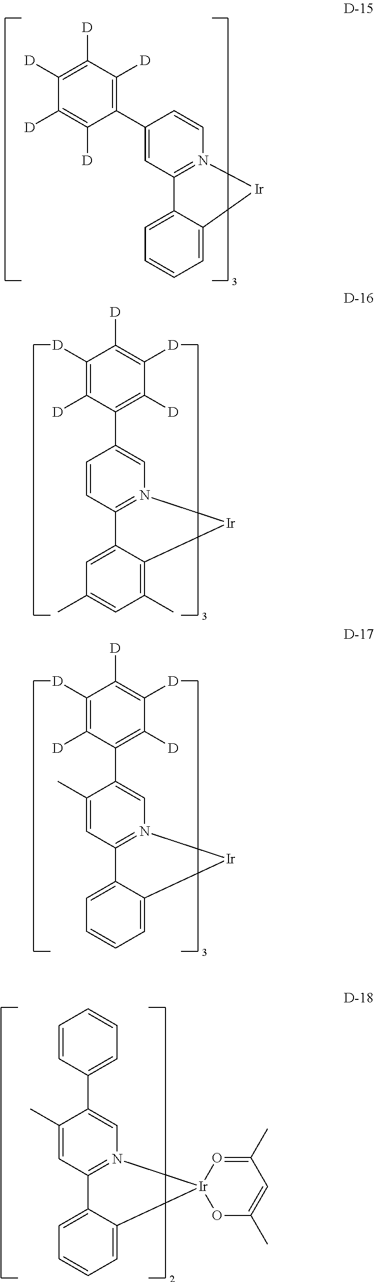

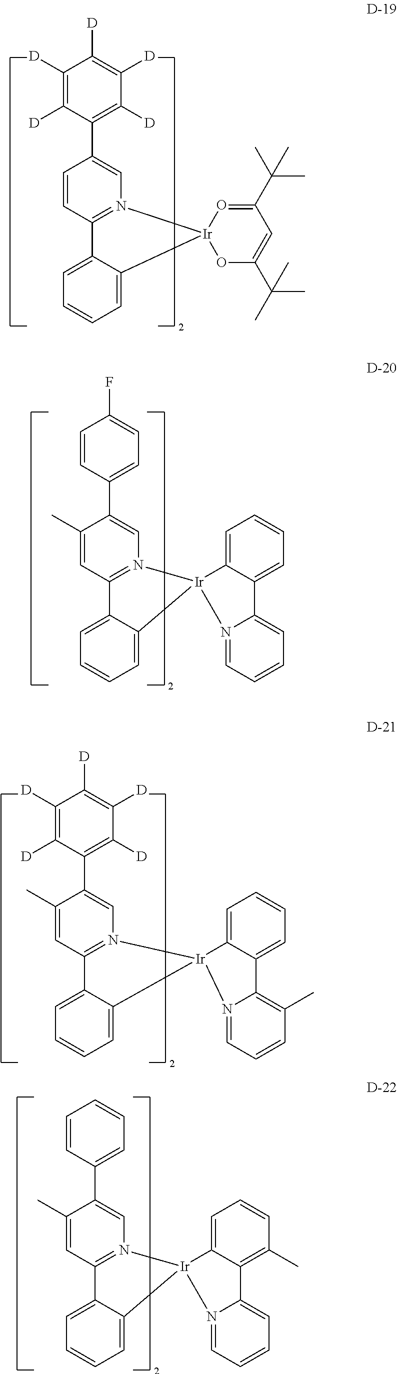

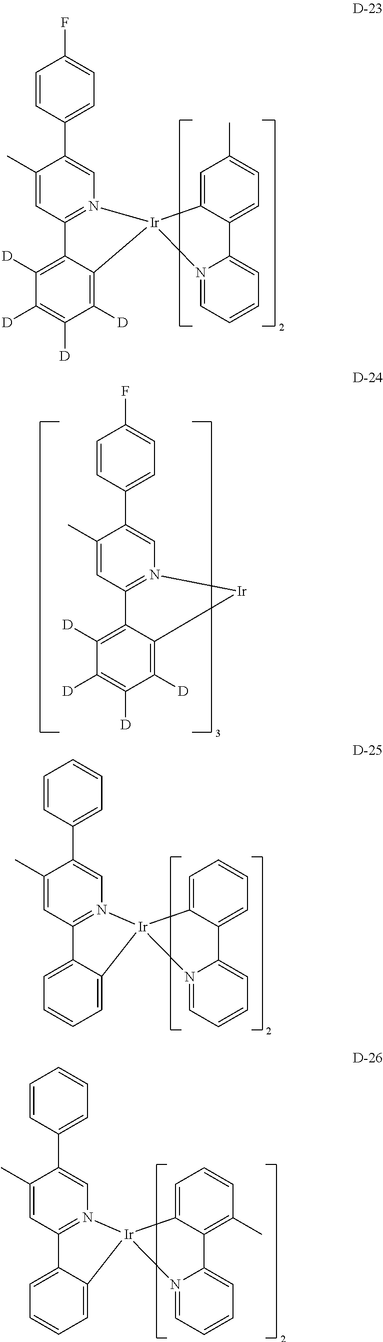

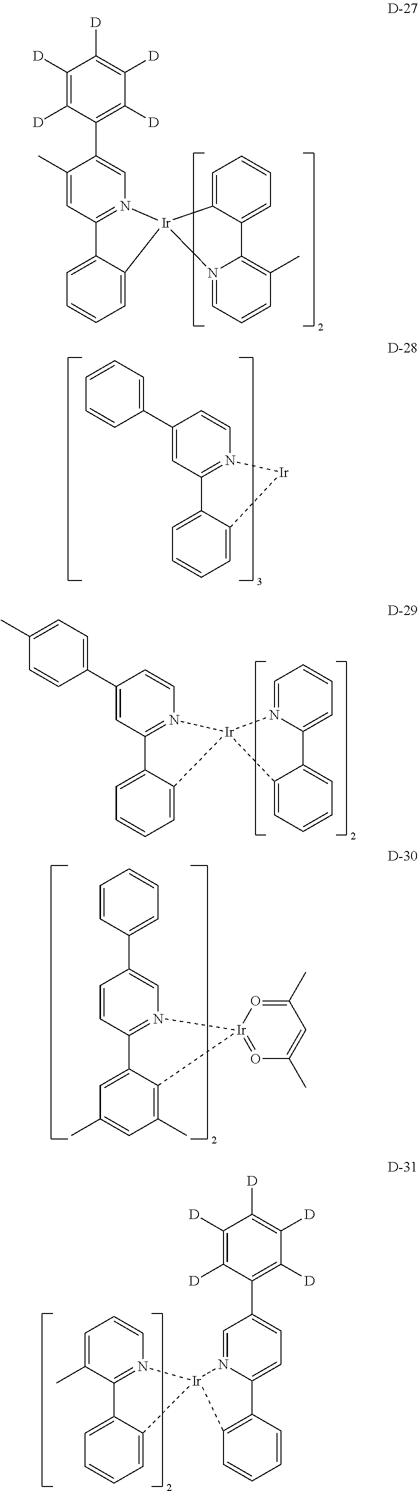

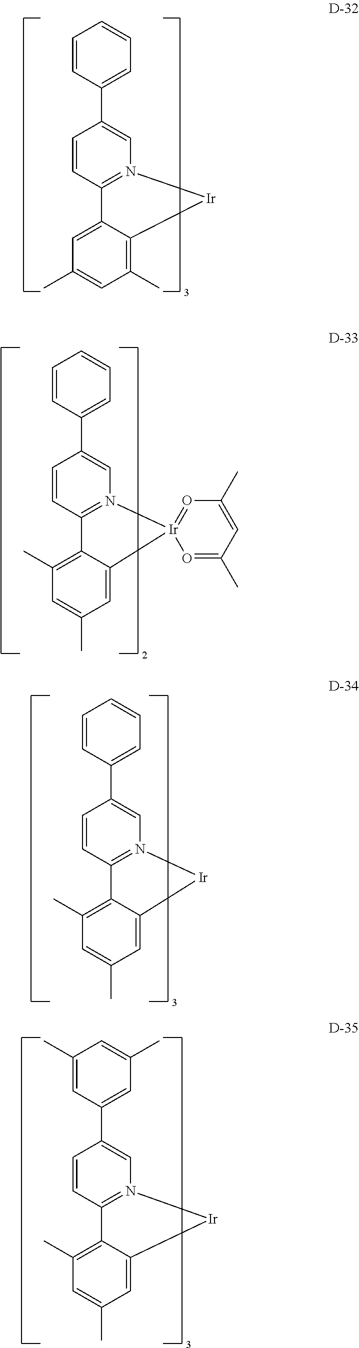

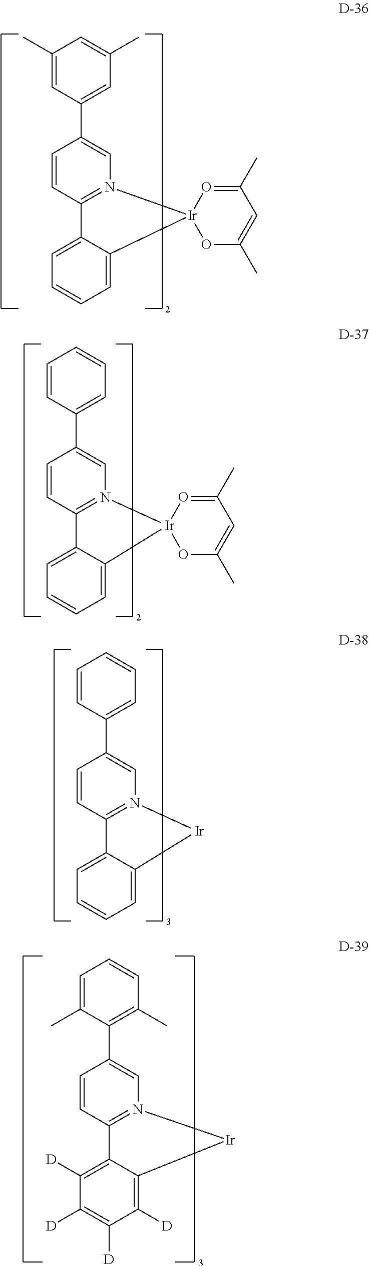

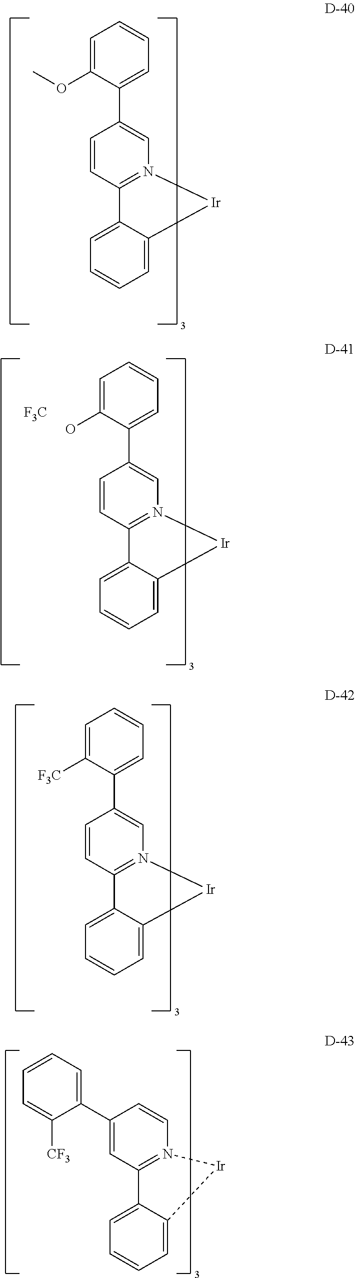

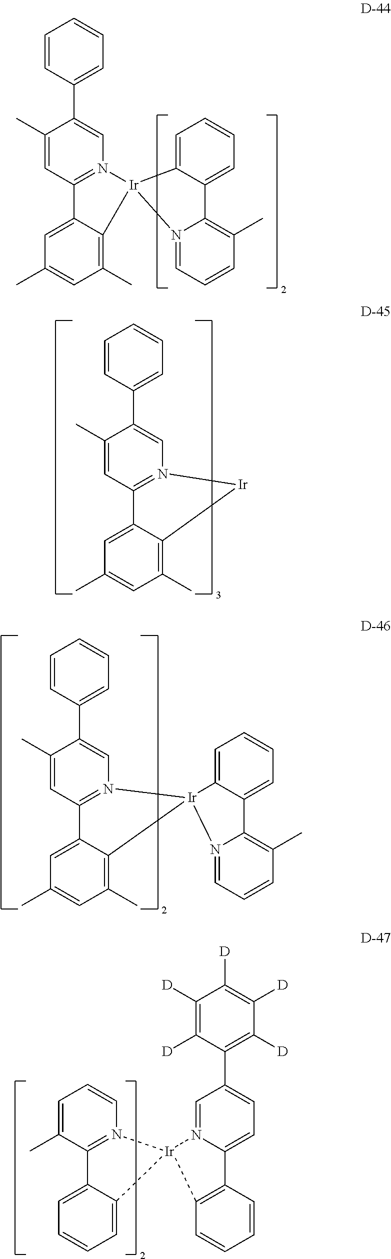

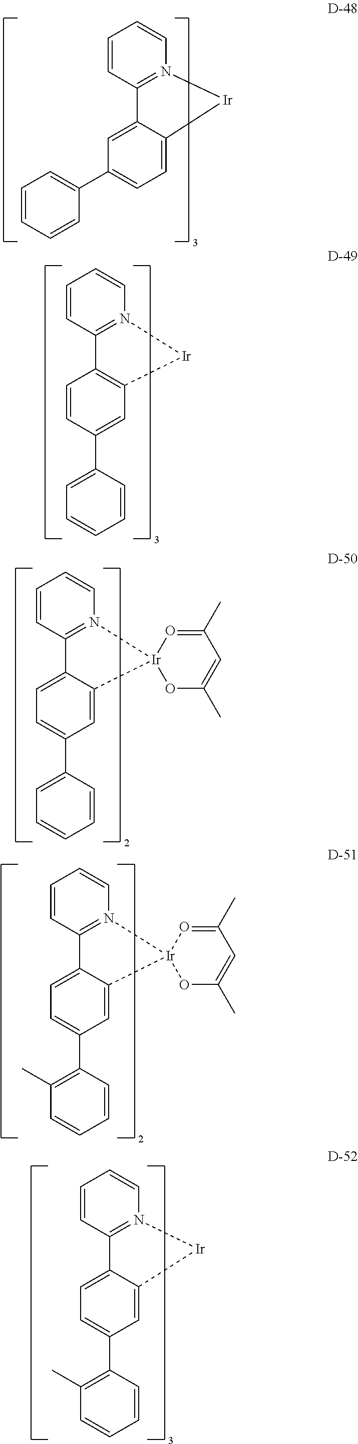

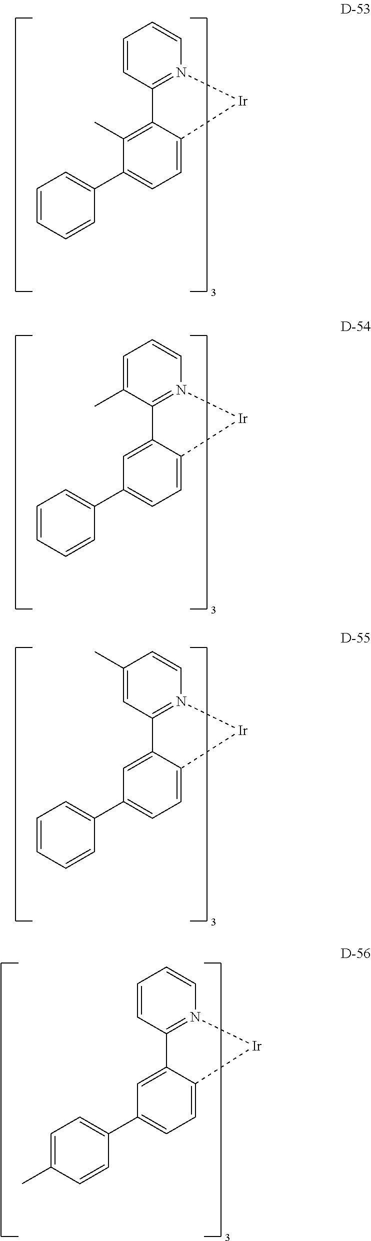

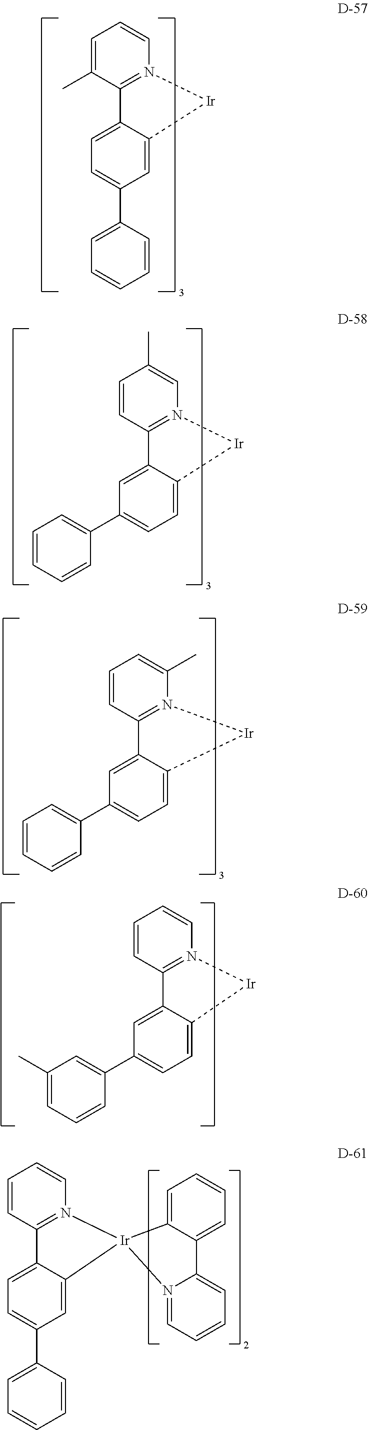

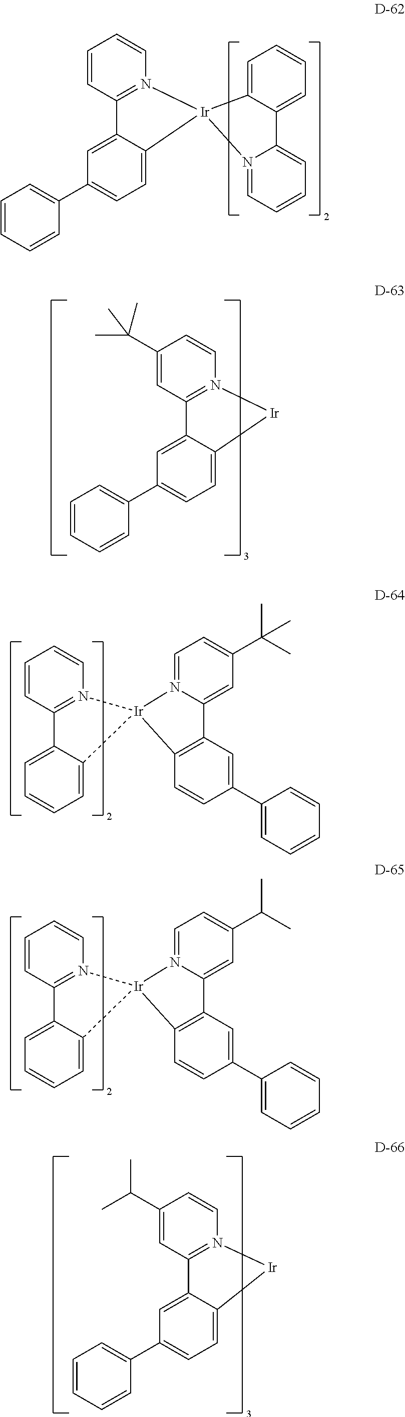

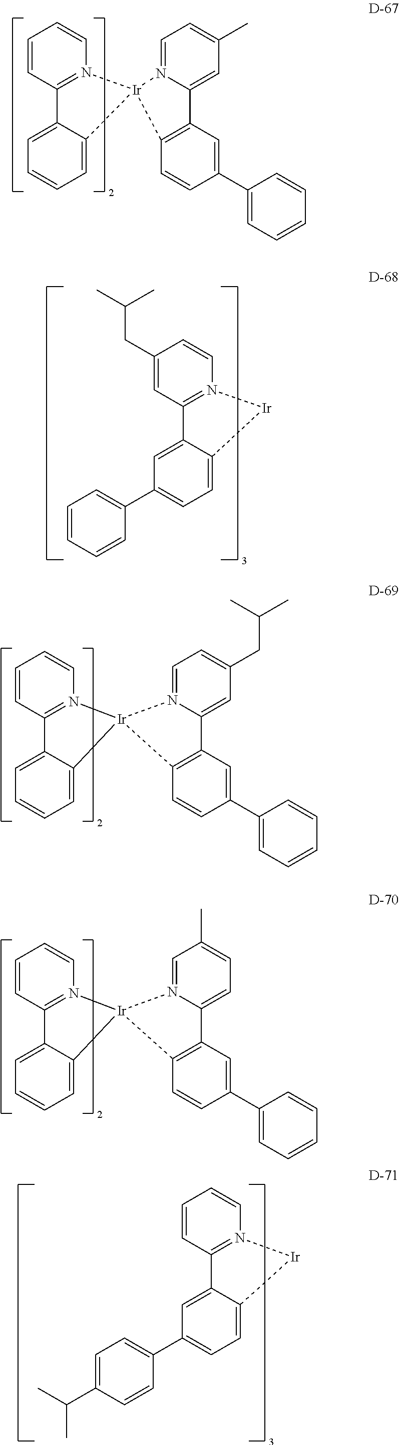

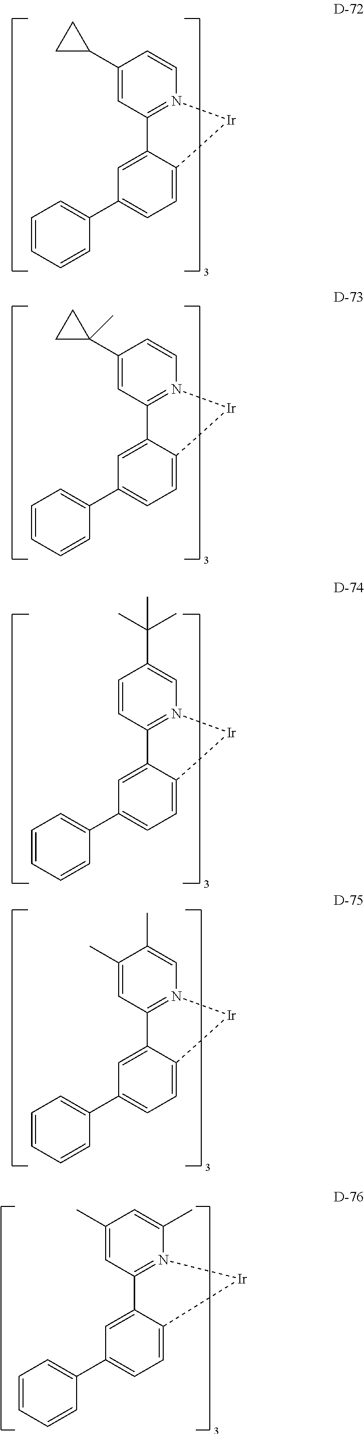

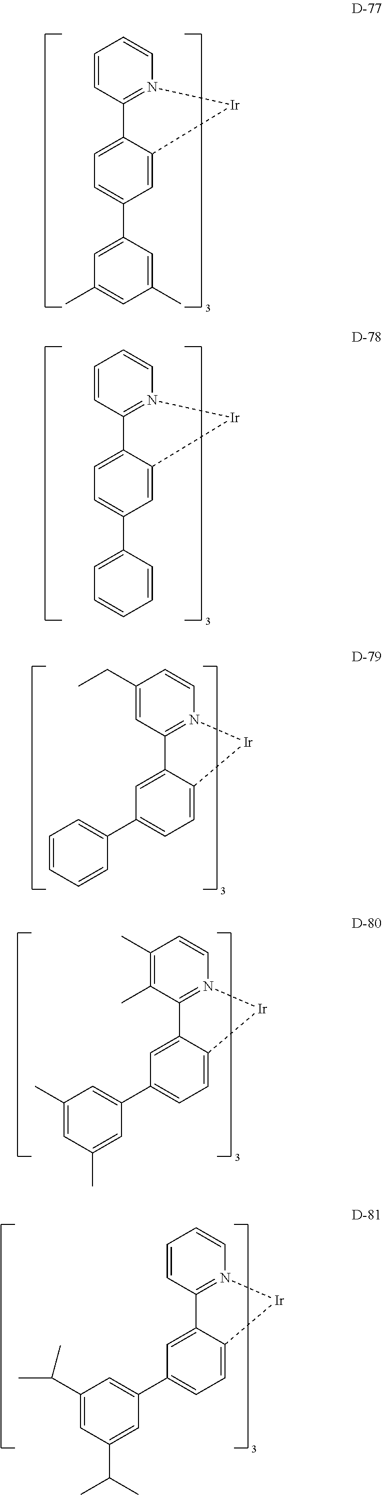

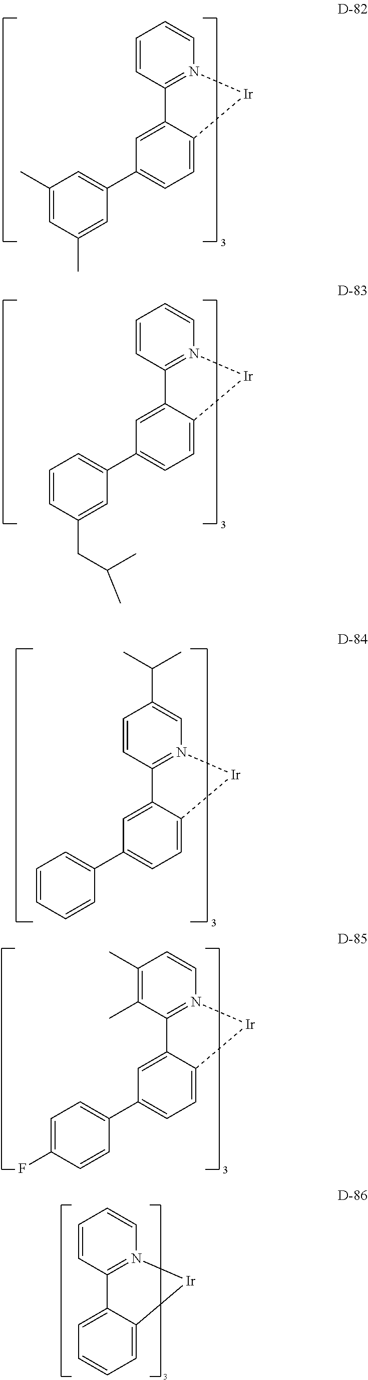

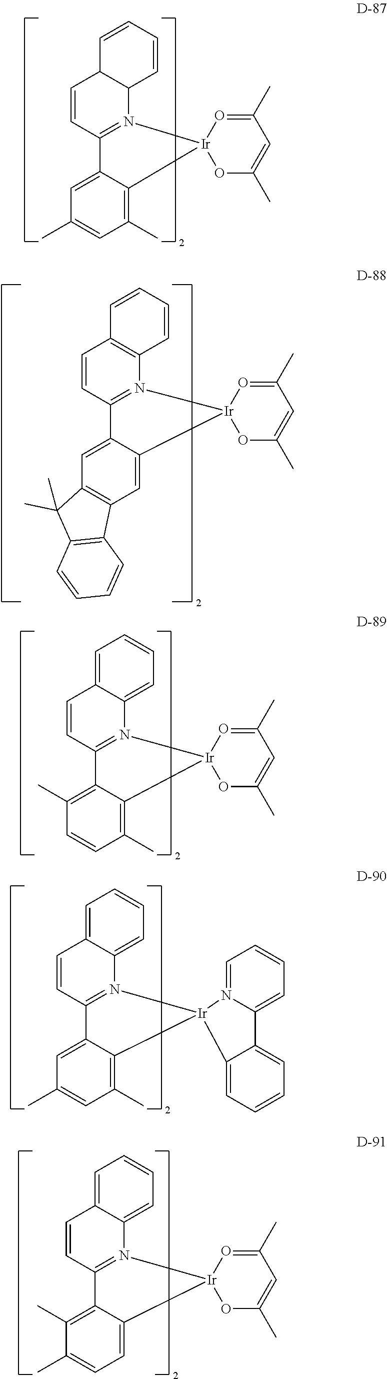

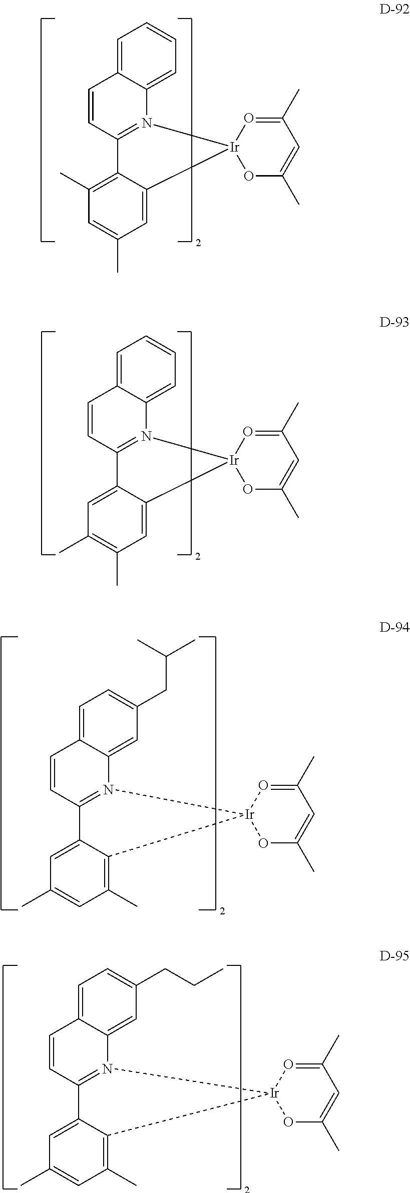

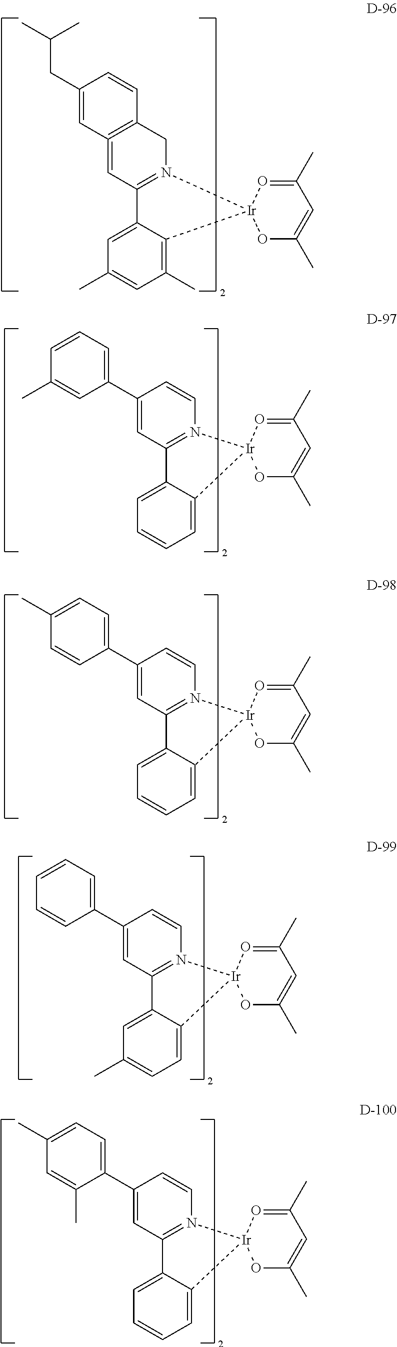

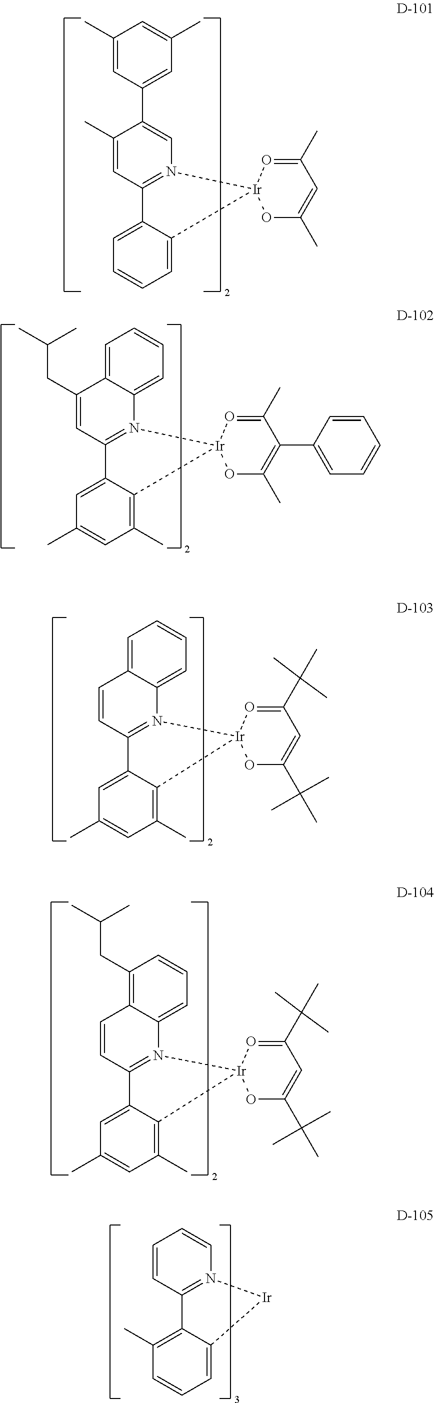

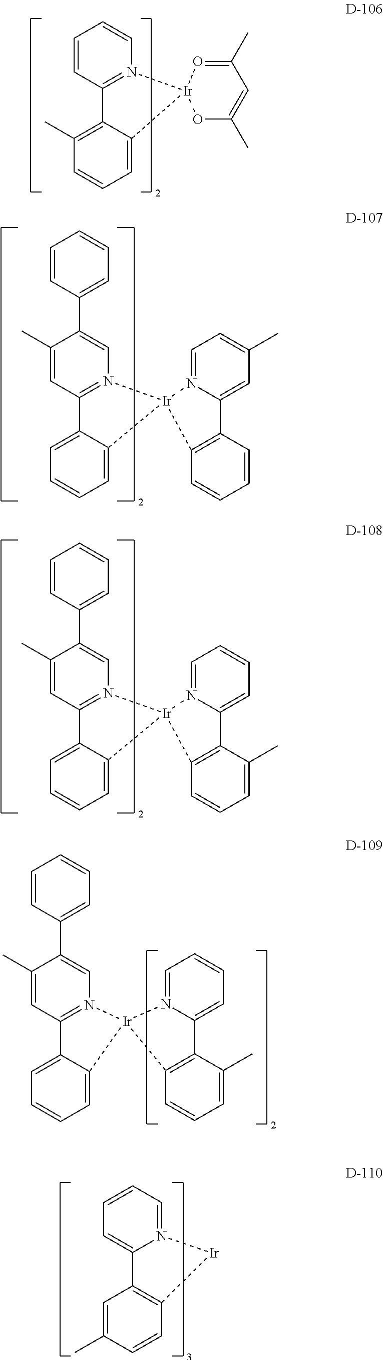

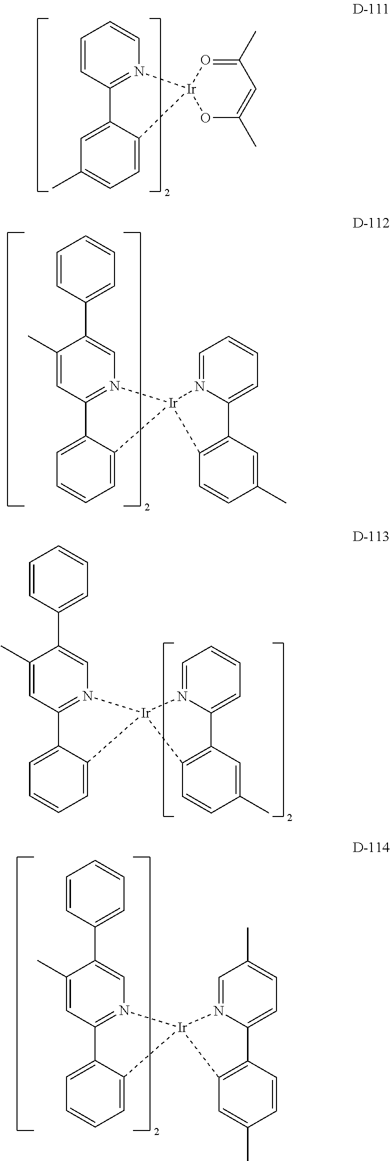

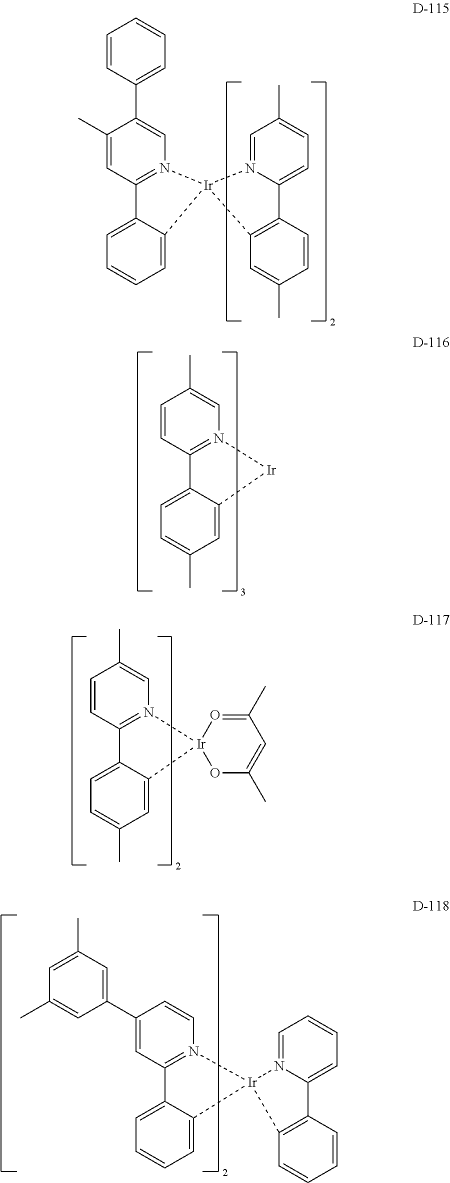

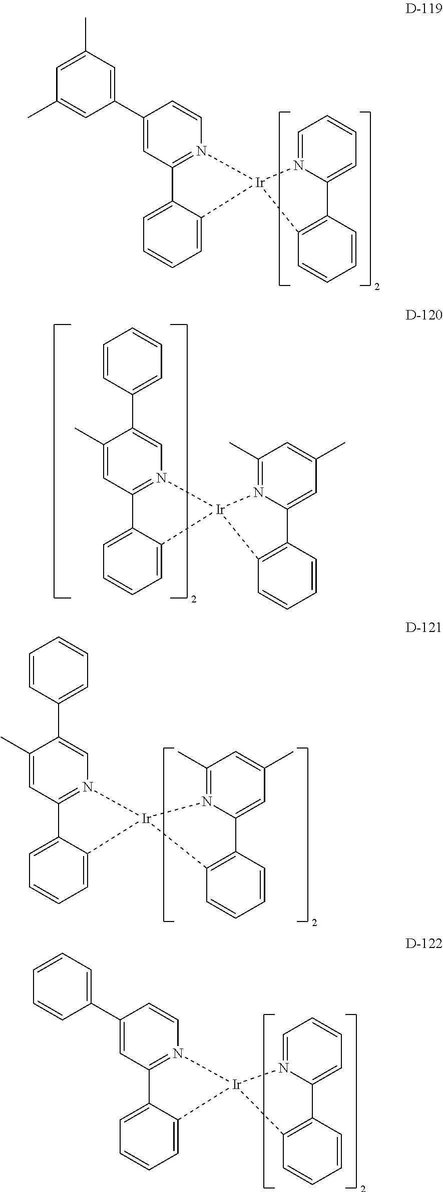

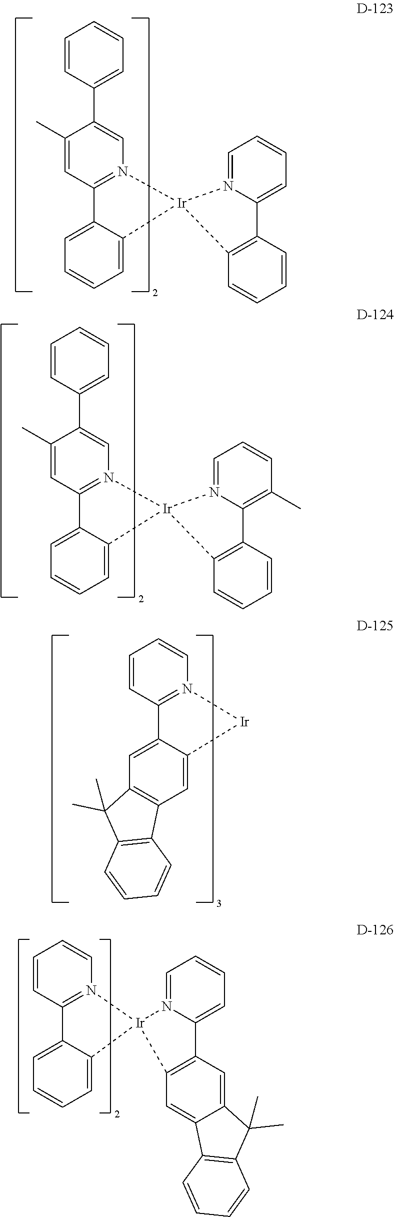

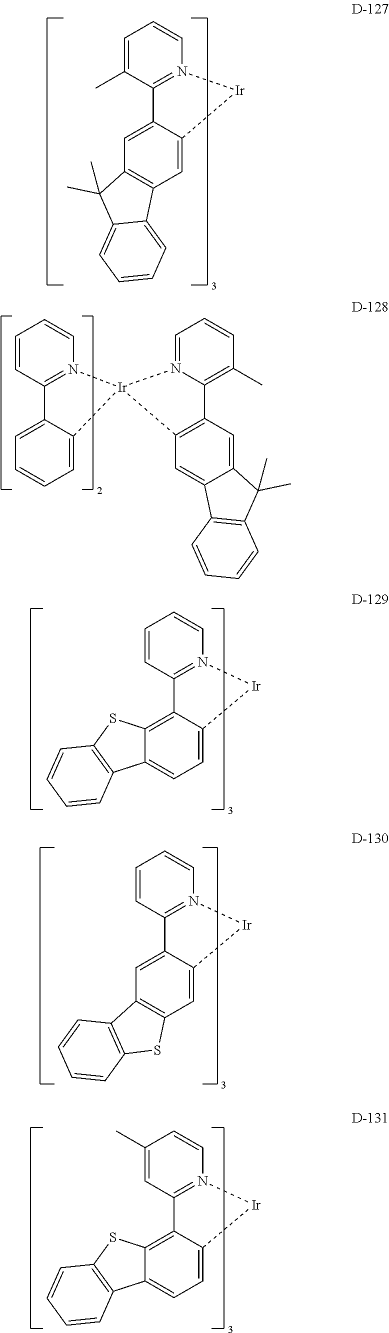

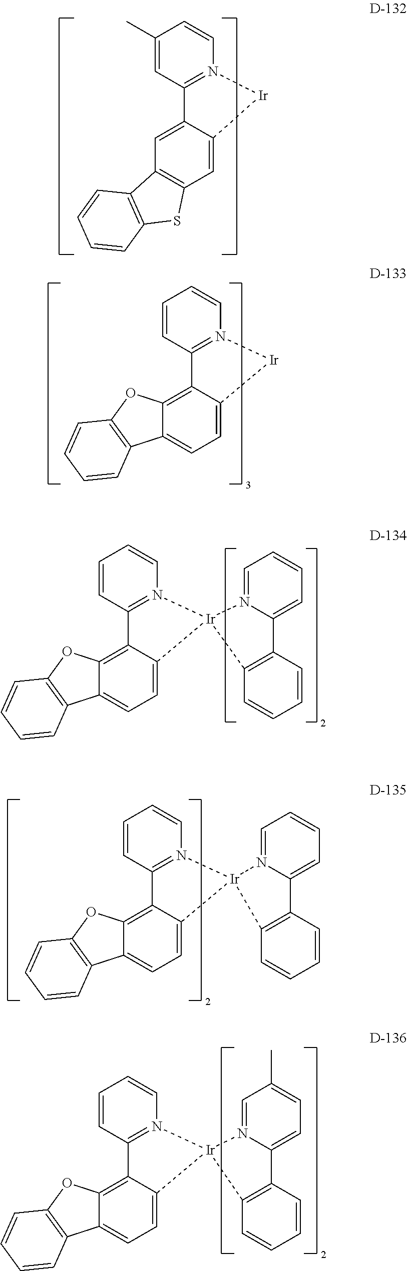

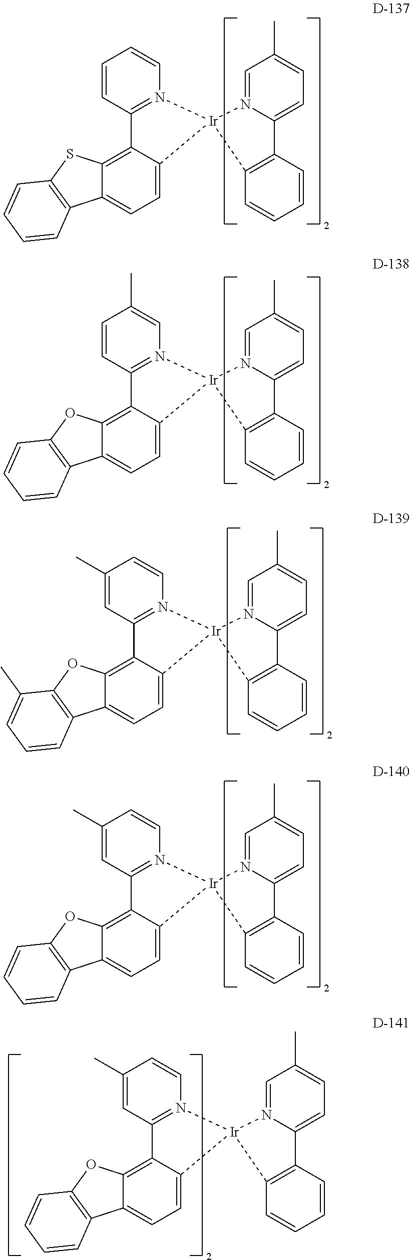

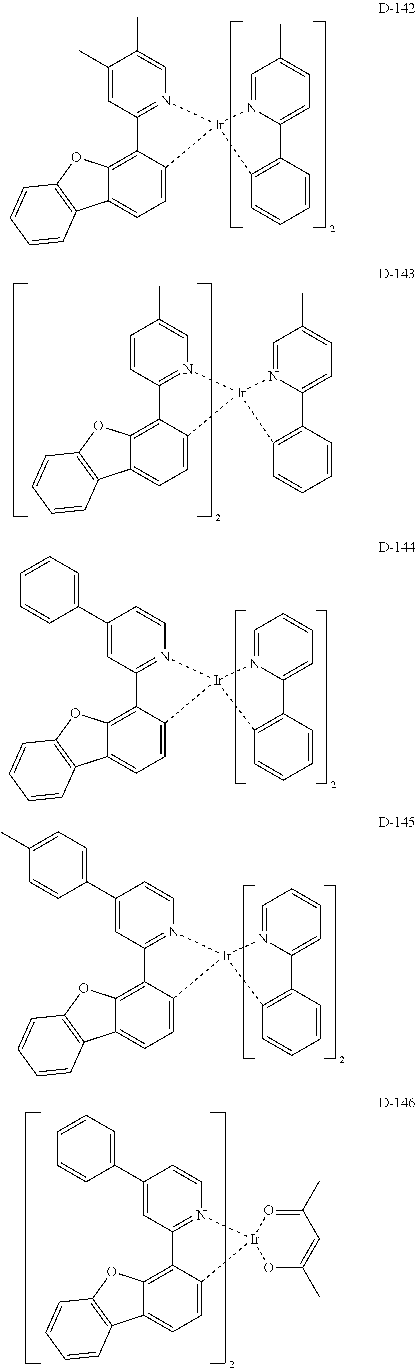

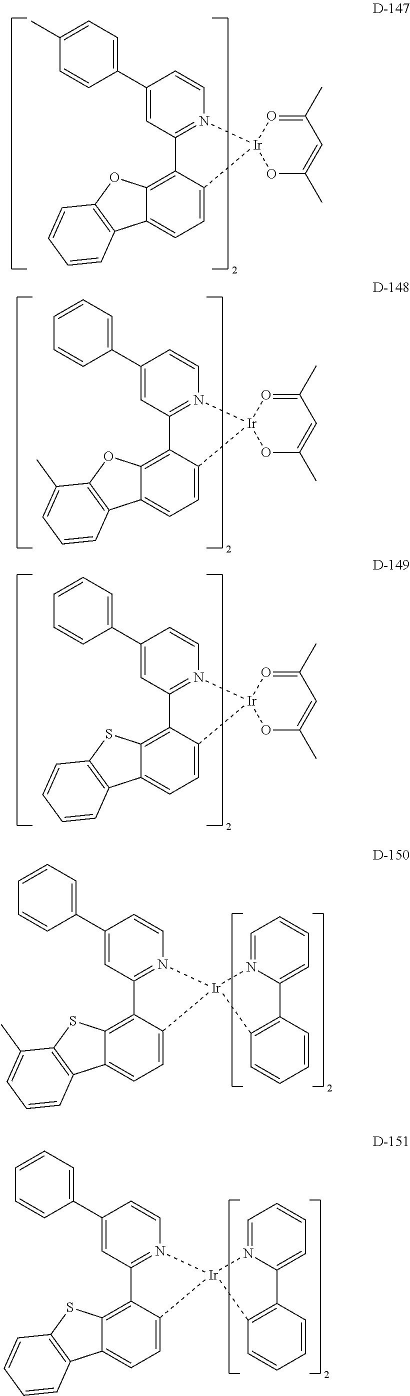

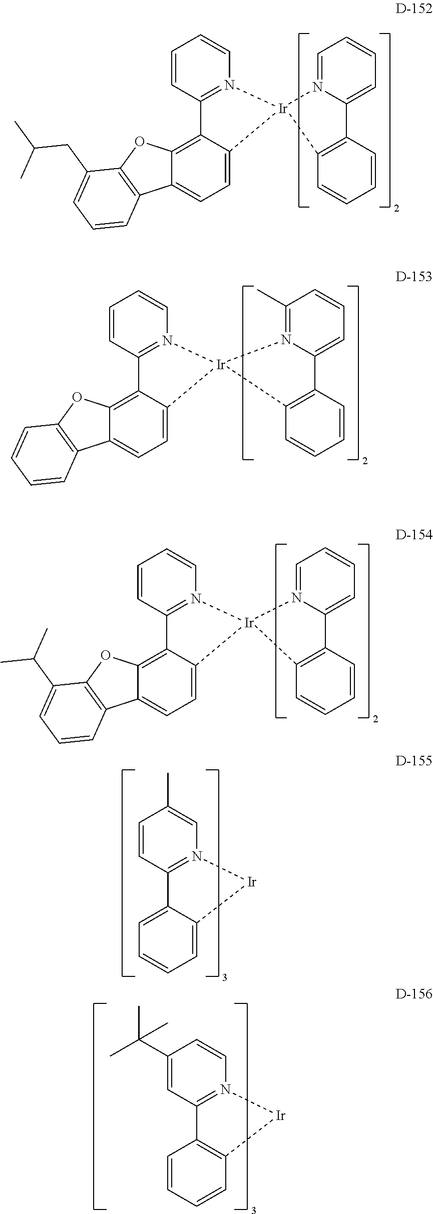

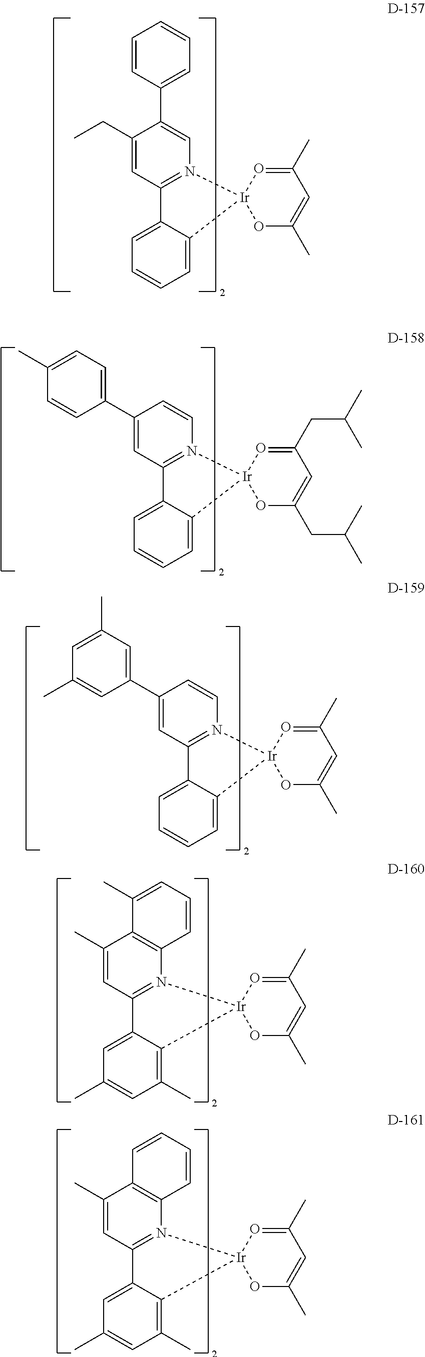

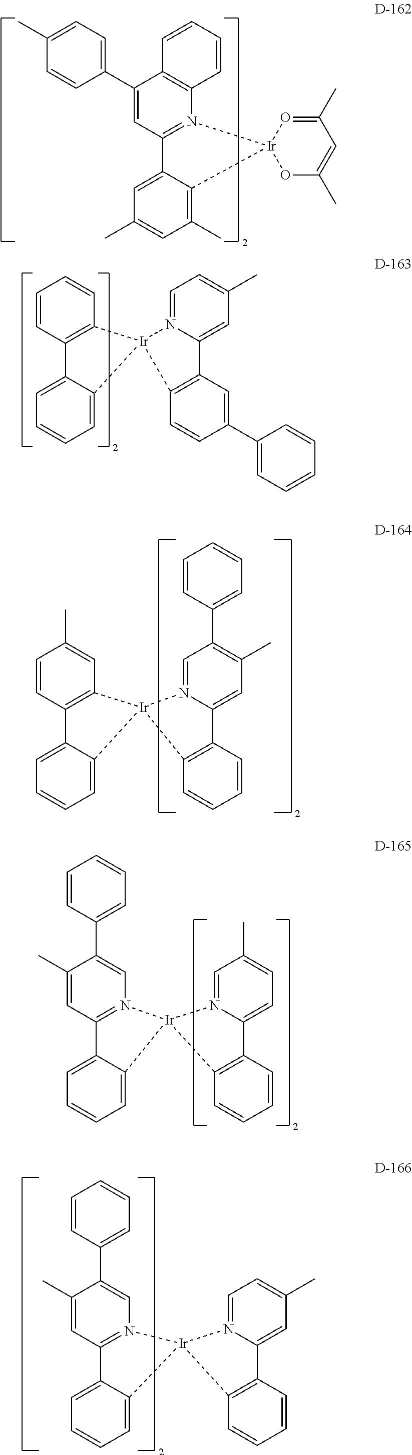

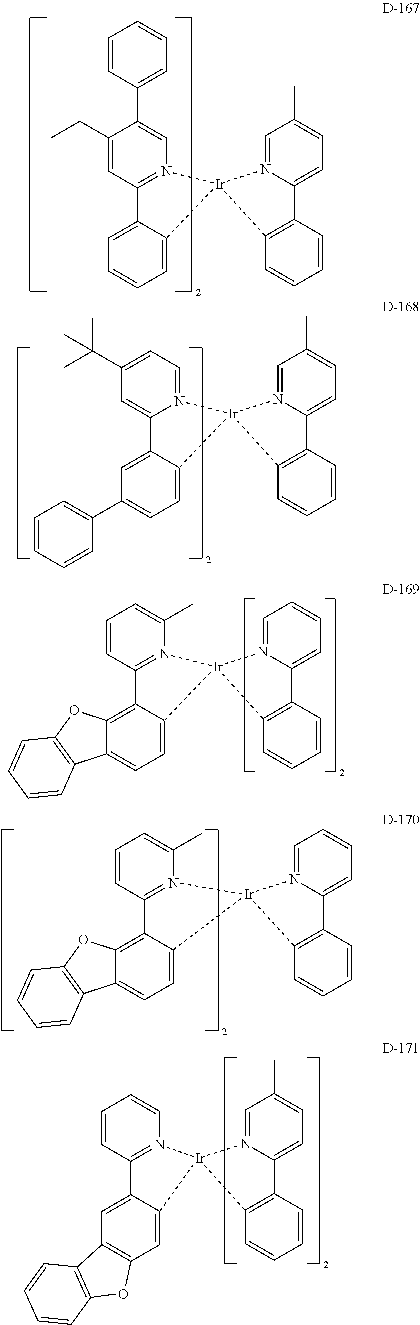

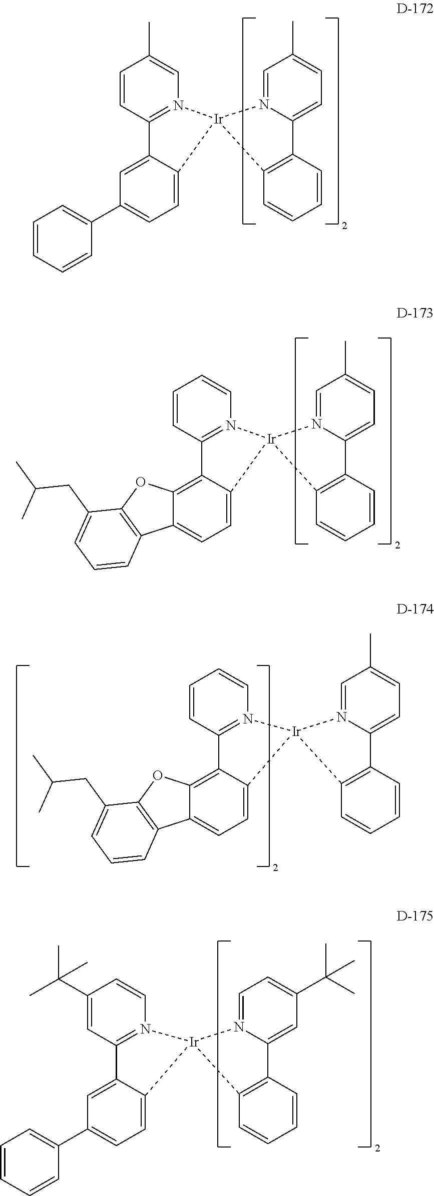

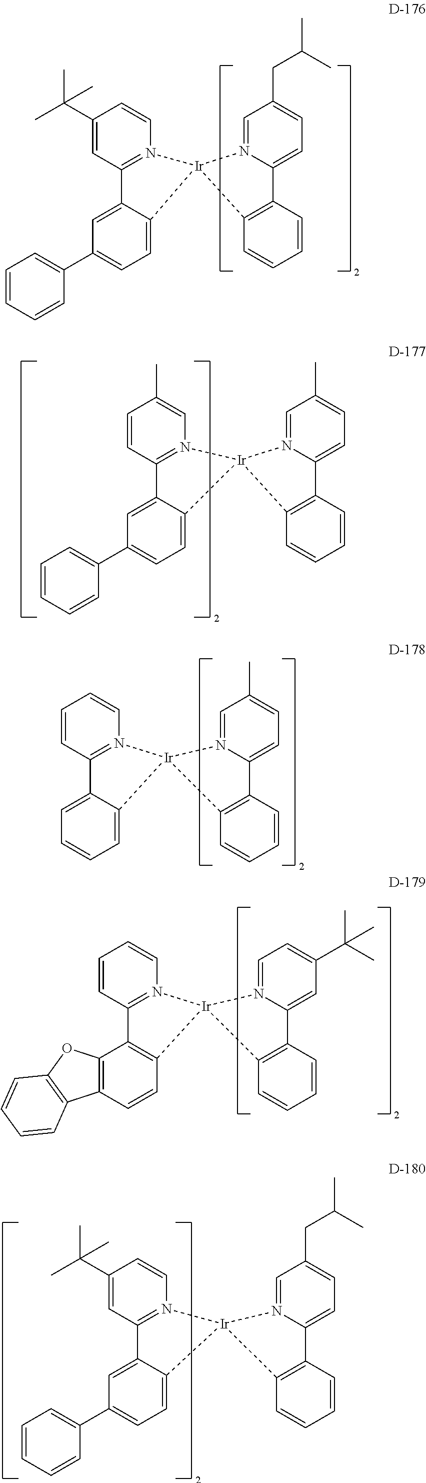

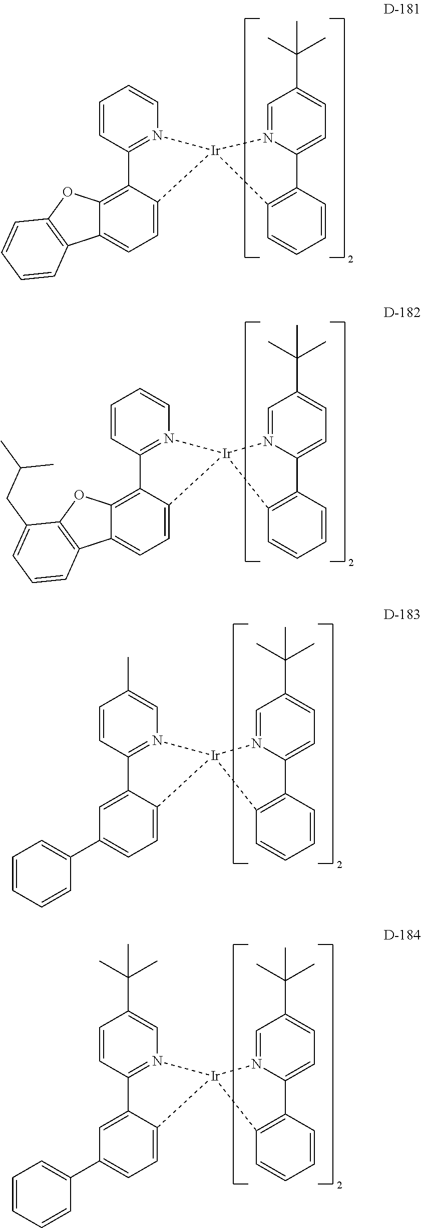

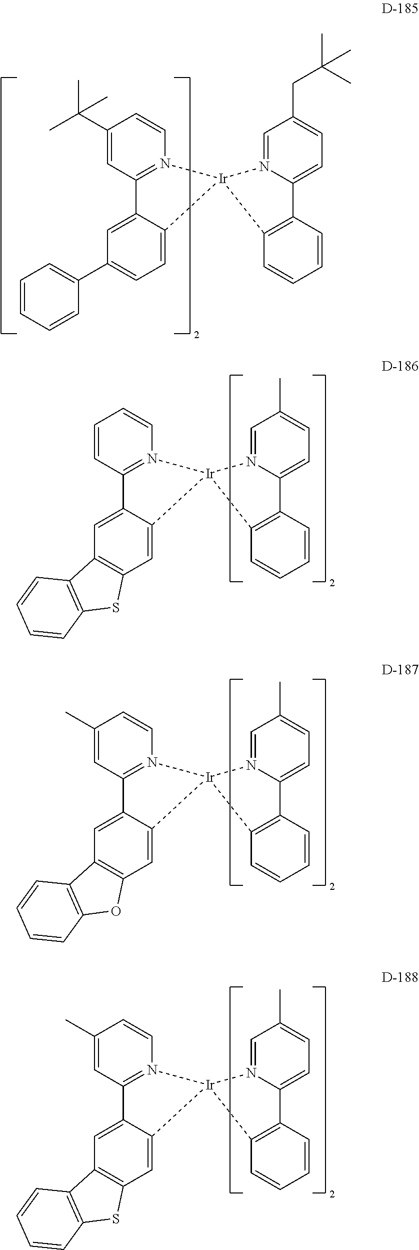

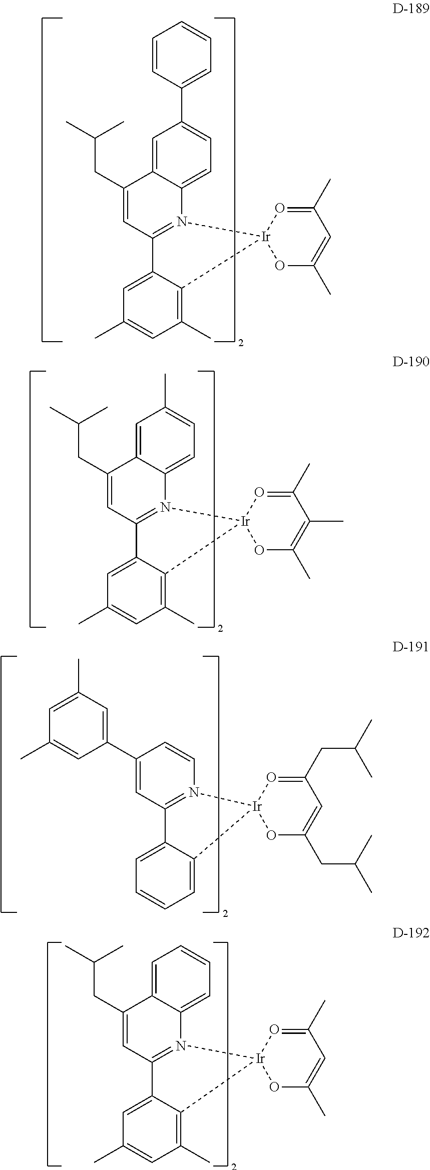

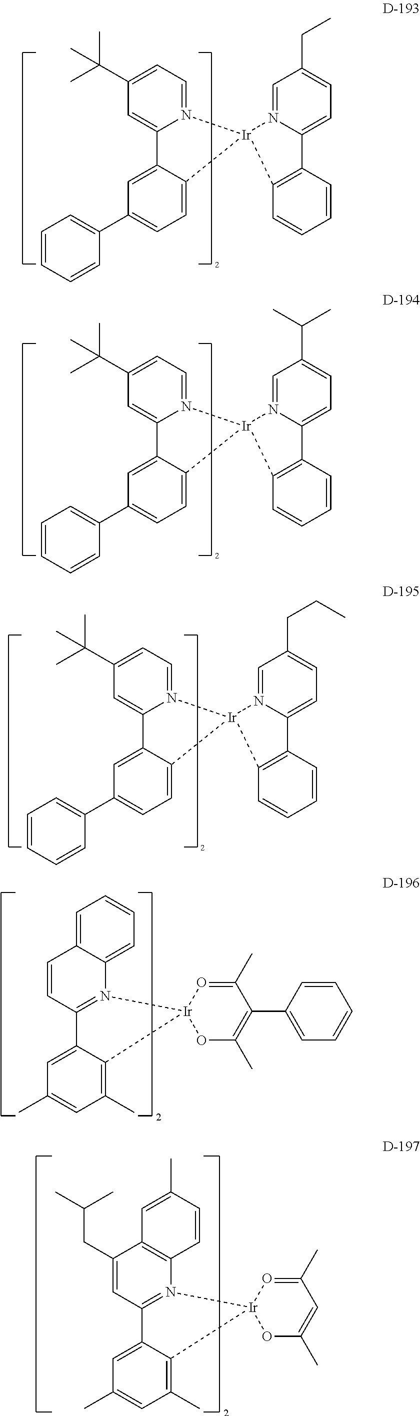

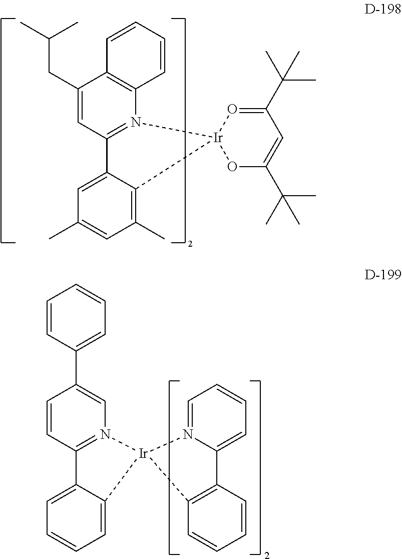

The organic electroluminescent device according to the present invention may further comprise at least one compound selected from the group consisting of arylamine-based compounds and styrylarylamine-based compounds in the organic layer.

In addition, in the organic electroluminescent device according to the present invention, the organic layer may further comprise at least one metal selected from the group consisting of metals of Group 1, metals of Group 2, transition metals of the 4^{th} period, transition metals of the 5^{th} period, lanthanides and organic metals of d-transition elements of the Periodic Table, or at least one complex compound comprising said metal.

According to the present invention, at least one layer (hereinafter, “a surface layer”) is preferably placed on an inner surface(s) of one or both electrodes; selected from a chalcogenide layer, a metal halide layer and a metal oxide layer. Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, said chalcogenide includes SiOₓ(1≦X≦2, AlOₓ(1≦X≦1.5), SiON, SiAlON, etc.; said metal halide includes LiF, MgF₂, CaF₂, a rare earth metal fluoride, etc.; and said metal oxide includes Cs₂O, Li₂O, MgO, SrO, BaO, CaO, etc.

Between the anode and the light-emitting layer, a hole injection layer, a hole transport layer, an electron blocking layer, or a combination thereof can be used. Multi-layers can be used for the hole injection layer in order to lower the hole injection barrier (or hole injection voltage) from the anode to the hole transport layer or the electron blocking layer. Two compounds can be simultaneously used in each layer. The hole transport layer and the electron blocking layer can also be formed of multi-layers.

Between the light-emitting layer and the cathode, a layer selected from an electron buffer layer, a hole blocking layer, an electron transport layer, or an electron injection layer, or formed by a combination thereof can be used. Multi-layers can be used for the electron buffer layer in order to control the injection of the electrons and enhance the interfacial characteristics between the light-emitting layer and the electron injection layer. Two compounds can be simultaneously used in each layer. The hole blocking layer and the electron transport layer can also be formed of multi-layers, and each layer can comprise two or more compounds.

In the organic electroluminescent device according to the present invention, a mixed region of an electron transport compound and a reductive dopant, or a mixed region of a hole transport compound and an oxidative dopant is preferably placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge-generating layer to prepare an electroluminescent device having two or more electroluminescent layers and emitting white light.

In order to form each layer of the organic electroluminescent device of the present invention, dry film-forming methods such as vacuum evaporation, sputtering, plasma and ion plating methods, or wet film-forming methods such as ink jet printing, nozzle printing, slot coating, spin coating, dip coating, and flow coating methods can be used. The first and second host compounds of the present invention may be co-evaporated or mixture-evaporated.

When using a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

Herein, a co-evaporation indicates a process for two or more materials to be deposited as a mixture, by introducing each of the two or more materials into respective crucible cells, and applying an electric current to the cells for each of the materials to be evaporated. Herein, a mixture-evaporation indicates a process for two or more materials to be deposited as a mixture, by mixing the two or more materials in one crucible cell before the deposition, and applying an electric current to the cell for the mixture to be evaporated.

By using the organic electroluminescent device of the present invention, a display system or a lighting system can be produced.

Hereinafter, the luminescent properties of the device comprising the host compound of the present invention will be explained in detail with reference to the following examples.

### DEVICE EXAMPLE 1

### Preparation of an OLED Device Comprising the First Host Compound and the Second Host Compound of the Present Invention

An OLED device was produced using the organic electroluminescent compound according to the present invention. A transparent electrode indium tin oxide (ITO) thin film (10 f2/sq) on a glass substrate for an organic light-emitting diode (OLED) device (Geomatec) was subjected to an ultrasonic washing with acetone, ethanol, and distilled water, sequentially, and then was stored in isopropanol. The ITO substrate was then mounted on a substrate holder of a vacuum vapor depositing apparatus. HI-1 was introduced into a cell of said vacuum vapor depositing apparatus, and then the pressure in the chamber of said apparatus was controlled to 10⁻⁶ torr. Thereafter, an electric current was applied to the cell to evaporate the above introduced material, thereby forming a first hole injection layer having a thickness of 80 nm on the ITO substrate. Next, HI-2 was introduced into another cell of said vacuum vapor depositing apparatus, and was evaporated by applying an electric current to the cell, thereby forming a second hole injection layer having a thickness of 5 nm on the first hole injection layer. HT-1 was then introduced into another cell of said vacuum vapor depositing apparatus, and was evaporated by applying an electric current to the cell, thereby forming a first hole transport layer having a thickness of 10 nm on the second hole injection layer. HT-3 was then introduced into another cell of said vacuum vapor depositing apparatus, and was evaporated by applying an electric current to the cell, thereby forming a second hole transport layer having a thickness of 60 nm on the first hole transport layer. Compound H-44 was introduced into one cell of said vacuum vapor depositing apparatus as a first host, compound H2-132 was introduced into another cell as a second host, and compound D-96 was introduced into another cell as a dopant. The two host materials were evaporated at the same rate in an amount of 50 wt %, respectively, while the dopant was evaporated at a different rate from the host materials, so that the dopant was deposited in a doping amount of 4 wt % based on the total amount of the host and dopant to coevaporate and form a light-emitting layer having a thickness of 30 nm on the second hole transport layer. ET-1 and EI-1 were then introduced into two cells of the vacuum vapor depositing apparatus, respectively, and evaporated at 1:1 rate to form an electron transport layer having a thickness of 30 nm on the light-emitting layer. After depositing EI-1 as an electron injection layer having a thickness of 2 nm on the electron transport layer, an Al cathode having a thickness of 80 nm was deposited by another vacuum vapor deposition apparatus. Thus, an OLED device was produced.

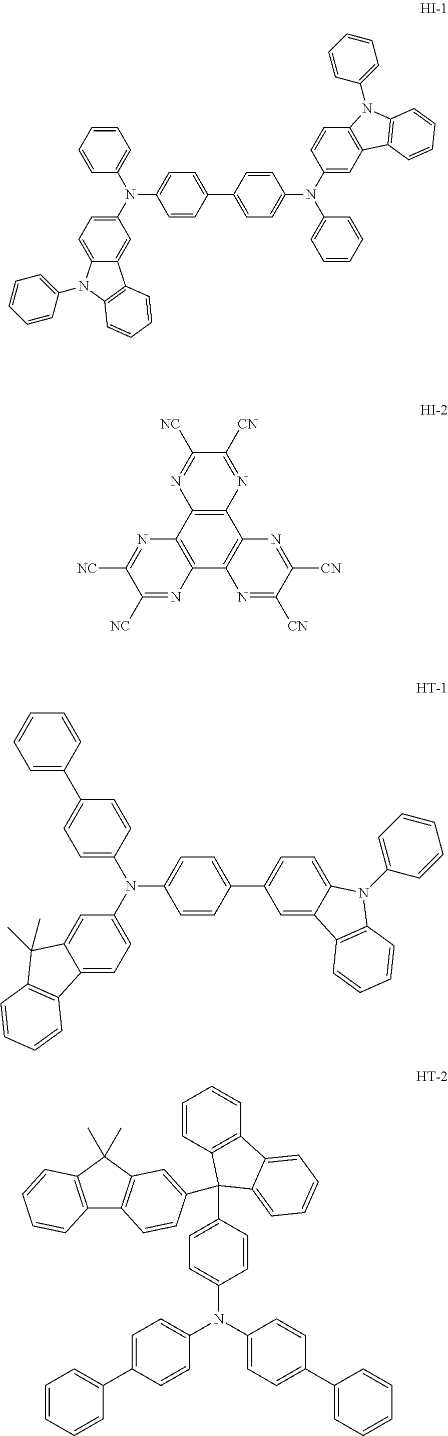 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(C3(c4ccc(N(c5ccc(-c6ccccc6)cc5)c5ccc(-c6ccccc6)cc5)cc4)c4ccccc4-c4ccccc43)cc21]

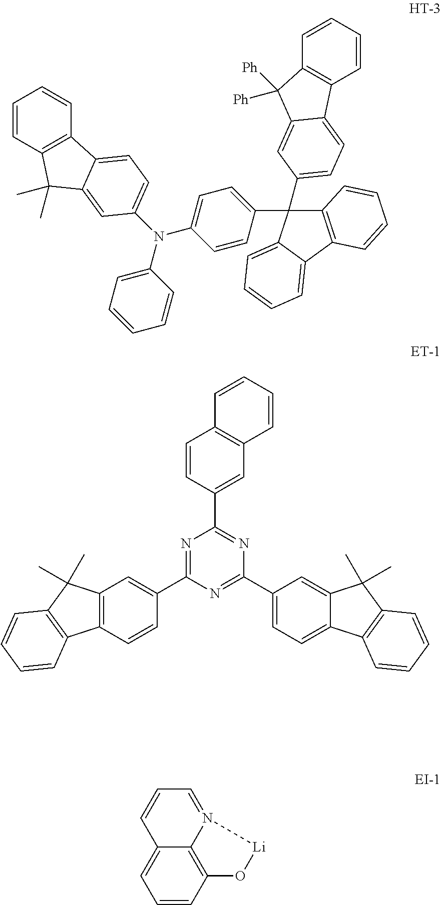

The produced OLED device showed a red emission having a luminance of 5000 cd/m² and a current efficiency of 27.1 cd/A at 4.9 V. The time period for the luminance to decrease to 95% at 5,000 nit was 44 hours or longer.

### DEVICE EXAMPLE 2

### Preparation of an OLED Device Comprising the First Host Compound and the Second Host Compound of the Present Invention

An OLED device was produced in the same manner as in Device Example 1, except for using compound H2-156 as the second host of the light-emitting material.

The produced OLED device showed a red emission having a luminance of 5000 cd/m² and a current efficiency of 23.4 cd/A at 5.4 V. The time period for the luminance to decrease to 95% at 5,000 nit was 147 hours or longer.

### DEVICE EXAMPLE 3

### Preparation of an OLED Device Comprising the First Host Compound and the Second Host Compound of the Present Invention

An OLED device was produced in the same manner as in Device Example 1, except for using HT-2 instead of HT-3 as the second hole transport layer, and using compound H2-16 as the second host of the light-emitting material.

The produced OLED device showed a red emission having a luminance of 5000 cd/m² and a current efficiency of 27.7 cd/A at 5 V. The time period for the luminance to decrease to 95% at 5,000 nit was 26 hours or longer.

### DEVICE EXAMPLE 4

### Preparation of an OLED Device Comprising the First Host Compound and the Second Host Compound of the Present Invention

An OLED device was produced in the same manner as in Device Example 3, except for using compound H2-516 as the second host of the light-emitting material.

The produced OLED device showed a red emission having a luminance of 5000 cd/m² and a current efficiency of 26.1 cd/A at 4.7 V. The time period for the luminance to decrease to 95% at 5,000 nit was 29 hours or longer.

### DEVICE EXAMPLE 5

### Preparation of an OLED Device Comprising the First Host Compound and the Second Host Compound of the Present Invention

An OLED device was produced in the same manner as in Device Example 3, except for using compound H2-21 as the second host of the light-emitting material.

The produced OLED device showed a red emission having a luminance of 5000 cd/m² and a current efficiency of 26.7 cd/A at 5.7 V. The time period for the luminance to decrease to 95% at 5,000 nit was 167 hours or longer.

### DEVICE EXAMPLE 6

### Preparation of an OLED Device Comprising the First Host Compound and the Second Host Compound of the Present Invention

An OLED device was produced in the same manner as in Device Example 3, except for using compound H2-41 as the second host of the light-emitting material. The produced OLED device showed a red emission having a luminance of 5000 cd/m² and a current efficiency of 27.0 cd/A at 4.4 V. The time period for the luminance to decrease to 95% at 5,000 nit was 64 hours or longer.

### DEVICE EXAMPLE 7

### Preparation of an OLED Device Comprising the First Host Compound and the Second Host Compound of the Present Invention

An OLED device was produced in the same manner as in Device Example 3, except for using compound H2-495 as the second host of the light-emitting material.

The produced OLED device showed a red emission having a luminance of 5000 cd/m² and a current efficiency of 25.1 cd/A at 5.5 V. The time period for the luminance to decrease to 95% at 5,000 nit was 106 hours or longer.

### DEVICE EXAMPLE 8

### Preparation of an OLED Device Comprising the First Host Compound and the Second Host Compound of the Present Invention

An OLED device was produced in the same manner as in Device Example 3, except for using compound H2-154 as the second host of the light-emitting material.

The produced OLED device showed a red emission having a luminance of 5000 cd/m² and a current efficiency of 25.8 cd/A at 5.8 V. The time period for the luminance to decrease to 95% at 5,000 nit was 106 hours or longer.

### COMPARATIVE EXAMPLE

### Preparation of an OLED Device Comprising only the First Host Compound of the Present Invention as a Host

An OLED device was produced in the same manner as in Device Examples 3, except for using only compound H-44 as the first host, and not using a second host of the light-emitting material.

The produced OLED device showed a red emission having a luminance of 5000 cd/m² and a current efficiency of 20 cd/A at 5.7 V. The time period for the luminance to decrease to 95% at 5,000 nit was 10 hours or longer.

When the plural hosts according to the present invention are used, effects of higher luminous efficiency/power efficiency and much more improved driving lifespan are provided compared to the conventional devices. Specifically, a high efficiency is maintained at high luminance, which is an advantageous characteristic in recent trends requiring UHD.

1. An organic electroluminescent device comprising at least one light-emitting layer between an anode and a cathode, wherein the light-emitting layer comprises a host and a phosphorescent dopant, the host comprises plural host compounds, at least a first host compound of the plural host compounds is a metal complex derivative represented by the following formula 1, and a second host compound is represented by the following formula 2. 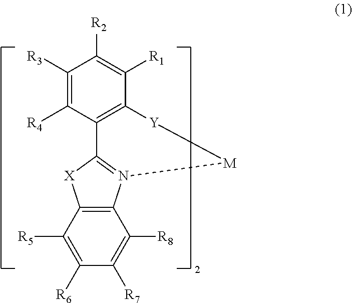
wherein
M represents a divalent metal;
Y represents O or S;
X represents NR₉, O, or S;
R₁ to R₉ each independently represent hydrogen, a halogen, a cyano, a substituted or unsubstituted (C1-C60)alkyl, a substituted or unsubstituted (C3-C60)cycloalkyl, a substituted or unsubstituted (C6-C60)aryl, a substituted or unsubstituted 4- to 60-membered heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted mono- or di- (C6-C30)arylamino; or are linked to each other to form a mono- or polycyclic, (C3-C30) alicyclic or aromatic ring, whose carbon atom(s) may be replaced with at least one hetero atom selected from nitrogen, oxygen, and sulfur; 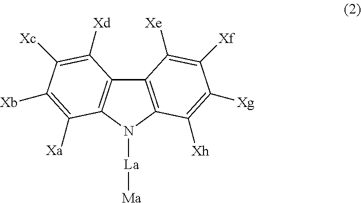
wherein
Ma represents a substituted or unsubstituted 5- to 30-membered nitrogen-containing heteroaryl;
La represents a single bond, a substituted or unsubstituted (C6-C30)arylene, or a substituted or unsubstituted 3- to 30-membered heteroarylene;
Xa to Xh each independently represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted 3- to 30-membered heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted mono- or di- (C6-C30)arylamino; or are linked to each other to form a substituted or unsubstituted mono- or polycyclic, (C3-C30) alicyclic or aromatic ring, whose carbon atom(s) may be replaced with at least one hetero atom selected from nitrogen, oxygen, and sulfur; and
the heteroaryl(ene) contains at least one hetero atom selected from B, N, O, S, Si, and P.

2. The organic electroluminescent device according to claim 1, wherein formula 1 is represented by one of the following formulas 3 to 8: 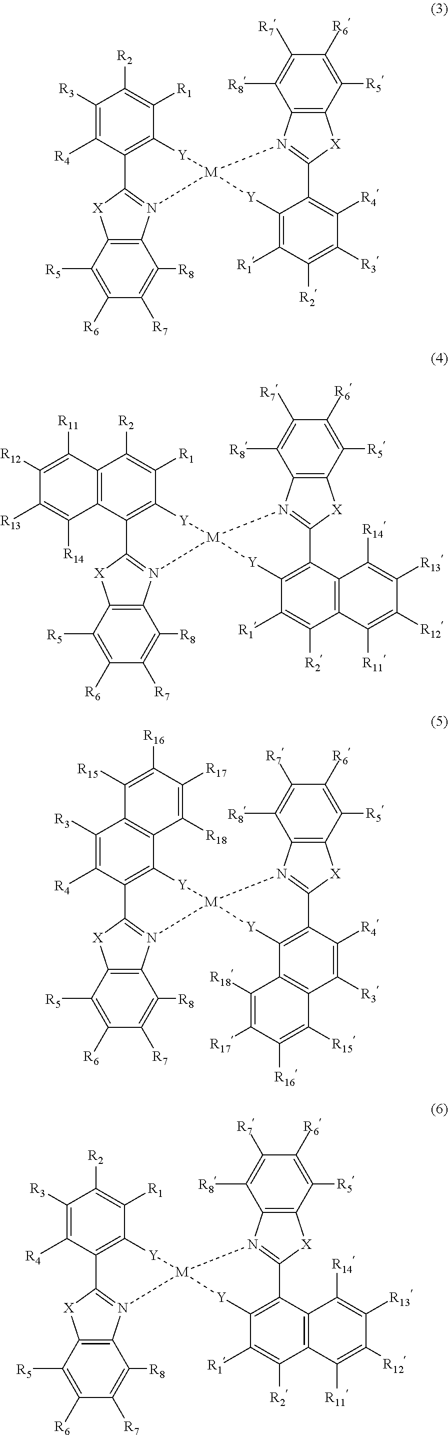 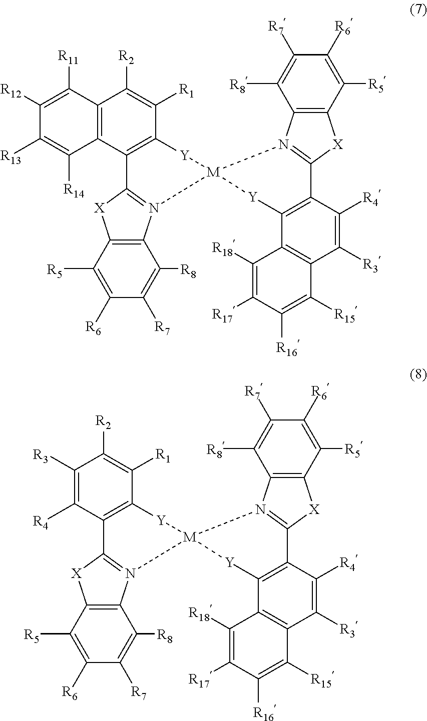
wherein
M, Y, X, and R₁ to R₈ are as defined in claim 1, and
R₁₁ to R₁₈, R₁′ to R₈′, and R₁₁′ to R₁₈^{′} each independently represent hydrogen, a halogen, a cyano, a substituted or unsubstituted (C1-C60)alkyl, a substituted or unsubstituted (C3-C60)cycloalkyl, a substituted or unsubstituted (C6-C60)aryl, a substituted or unsubstituted 4- to 60-membered heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted mono- or di- (C6-C30)arylamino; or are linked to each other to form a mono- or polycyclic, (C3-C30) alicyclic or aromatic ring, whose carbon atom(s) may be replaced with at least one hetero atom selected from nitrogen, oxygen, and sulfur.

3. The organic electroluminescent device according to claim 1, wherein in formula 1,
M represents Be or Zn, Y represents O, X represents NR₉, O, or S, R₁ to R₉ each independently represent hydrogen, a halogen, a cyano, a substituted or unsubstituted (C1-C30)alkyl, a substituted or unsubstituted (C6-C30)aryl, a substituted or unsubstituted tri(C1-C30)alkylsilyl, a substituted or unsubstituted tri(C6-C30)arylsilyl, or a substituted or unsubstituted di(C6-C30)arylamino; or are linked to each other to form a mono- or polycyclic, (C3-C30) alicyclic or aromatic ring.

4. The organic electroluminescent device according to claim 1, wherein in formula 2,
La is represented by a single bond, a carbazolylene, or one of the following formulas 9 to 21: 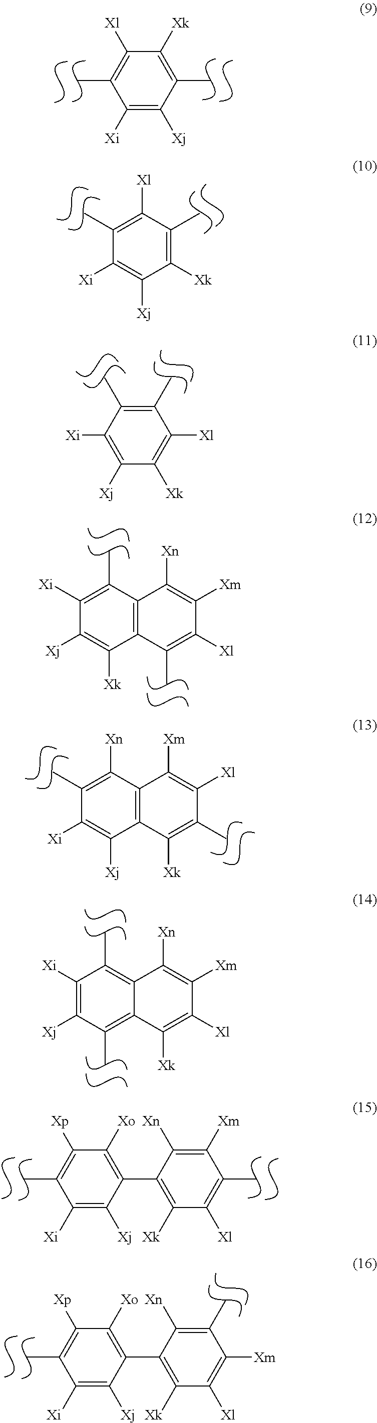 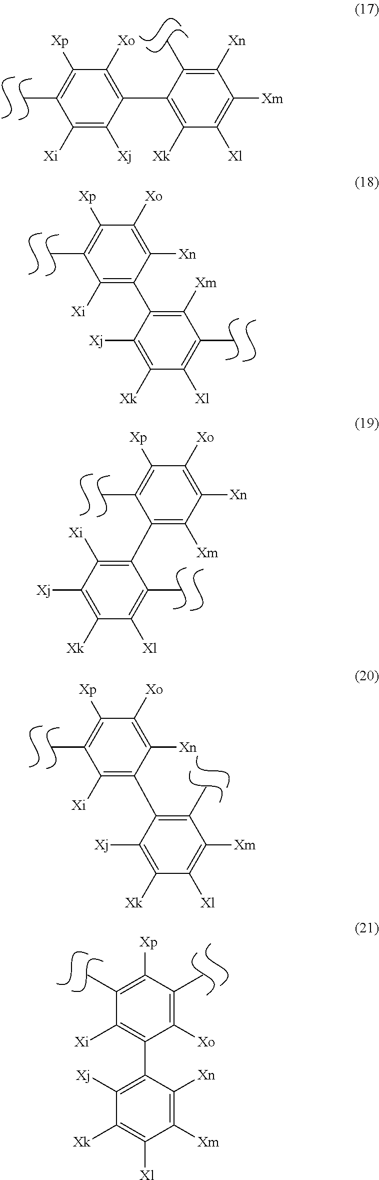
wherein
Xi to Xp each independently represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted 3- to 30-membered heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted mono- or di- (C6-C30)arylamino; or are linked to each other to form a substituted or unsubstituted, mono- or polycyclic, (C3-C30) alicyclic or aromatic ring, whose carbon atom(s) may be replaced with at least one hetero atom selected from nitrogen, oxygen, and sulfur.

5. The organic electroluminescent device according to claim 1, wherein in formula 2,
Ma represents a monocyclic ring-type heteroaryl selected from a group consisting of a substituted or unsubstituted pyrrolyl, a substituted or unsubstituted imidazolyl, a substituted or unsubstituted pyrazolyl, a substituted or unsubstituted triazinyl, a substituted or unsubstituted tetrazinyl, a substituted or unsubstituted triazolyl, a substituted or unsubstituted tetrazolyl, a substituted or unsubstituted pyridyl, a substituted or unsubstituted pyrazinyl, a substituted or unsubstituted pyrimidinyl, and a substituted or unsubstituted pyridazinyl, or a fused ring-type heteroaryl selected from a group consisting of a substituted or unsubstituted benzimidazolyl, a substituted or unsubstituted isoindolyl, a substituted or unsubstituted indolyl, a substituted or unsubstituted indazolyl, a substituted or unsubstituted benzothiadiazolyl, a substituted or unsubstituted quinolyl, a substituted or unsubstituted isoquinolyl, a substituted or unsubstituted cinnolinyl, a substituted or unsubstituted quinazolinyl, a substituted or unsubstituted naphthyridinyl, a substituted or unsubstituted quinoxalinyl, a substituted or unsubstituted carbazolyl, and a substituted or unsubstituted phenanthridinyl.

6. The organic electroluminescent device according to claim 1, wherein in formula 2,
Xa to Xh each independently represent hydrogen, a cyano, a substituted or unsubstituted (C6-C15)aryl, a substituted or unsubstituted 10- to 20-membered heteroaryl, or a substituted or unsubstituted tri(C6-C10)arylsilyl; or are linked to each other to form a substituted or unsubstituted mono- or polycyclic, (C6-C20) aromatic ring.

7. The organic electroluminescent device according to claim 1, wherein the compound represented by formula 1 is selected from the group consisting of:

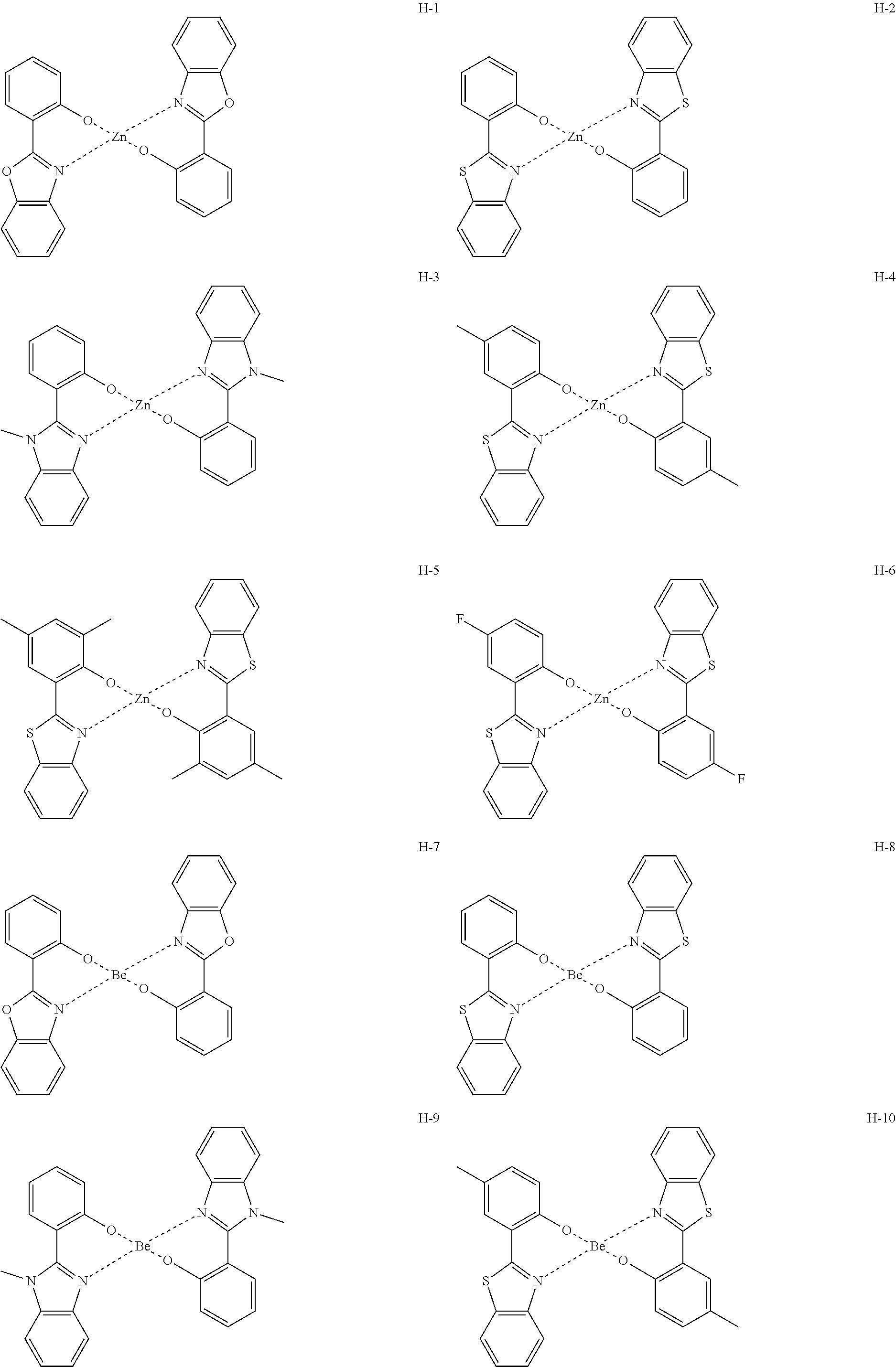

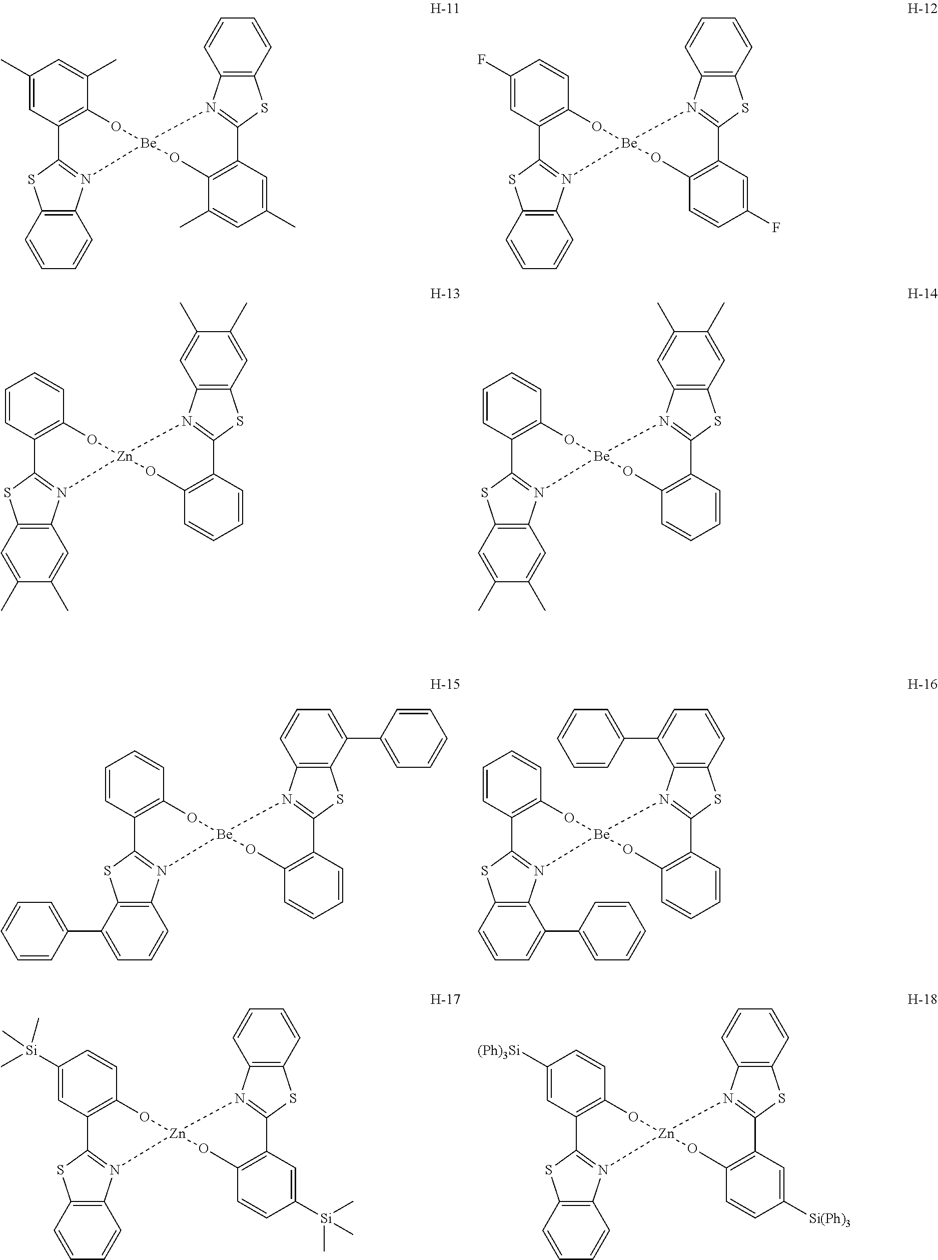

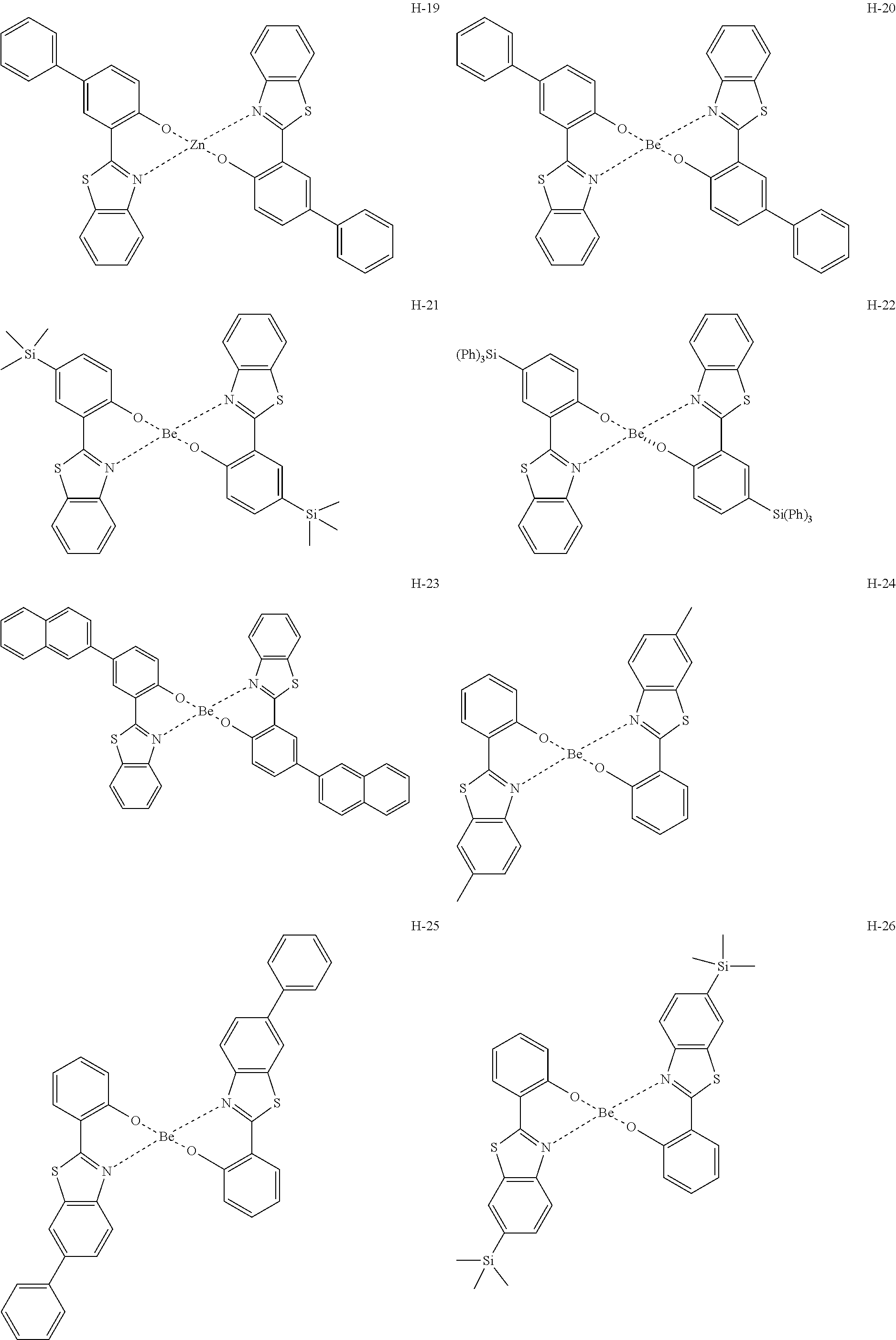

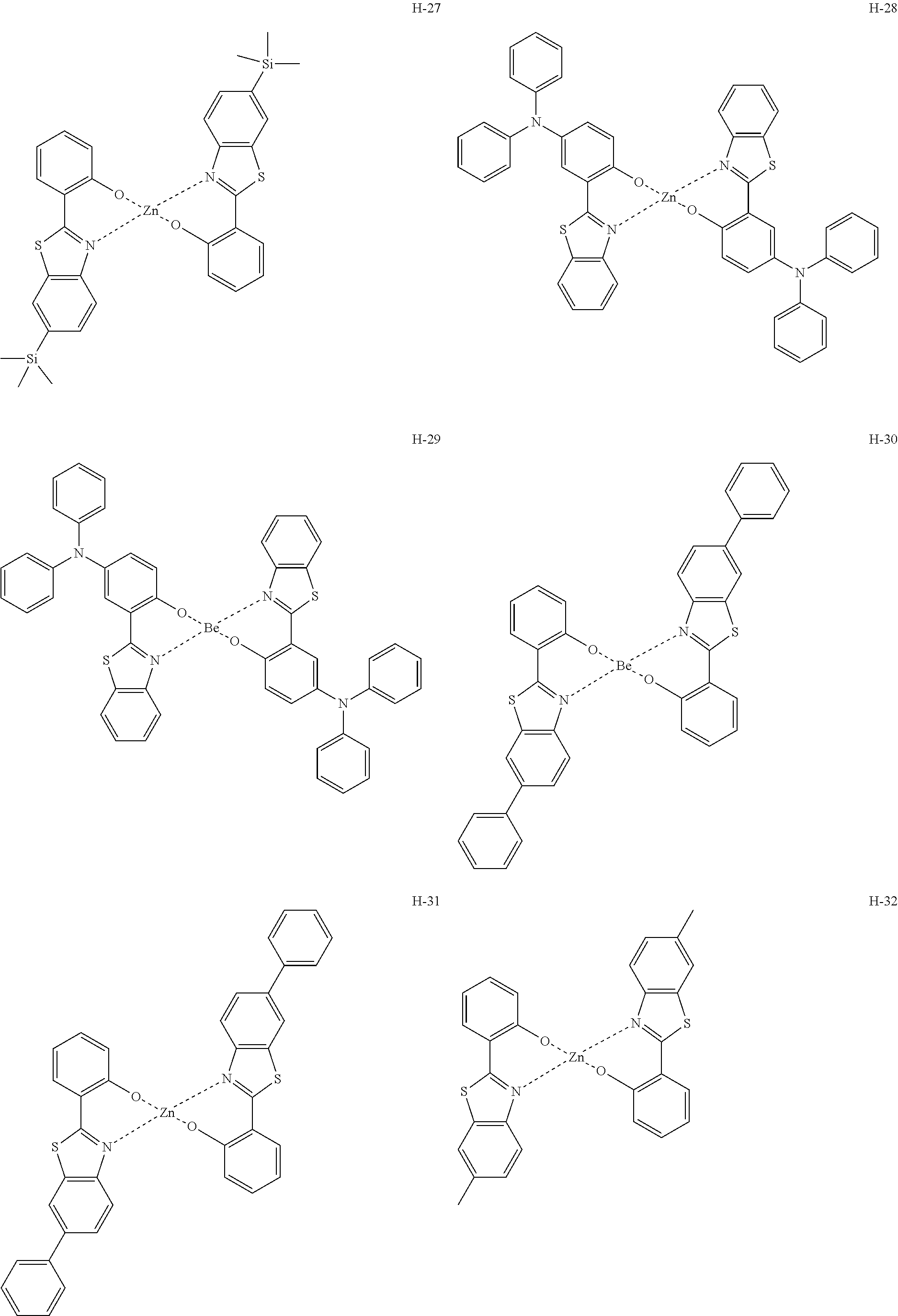

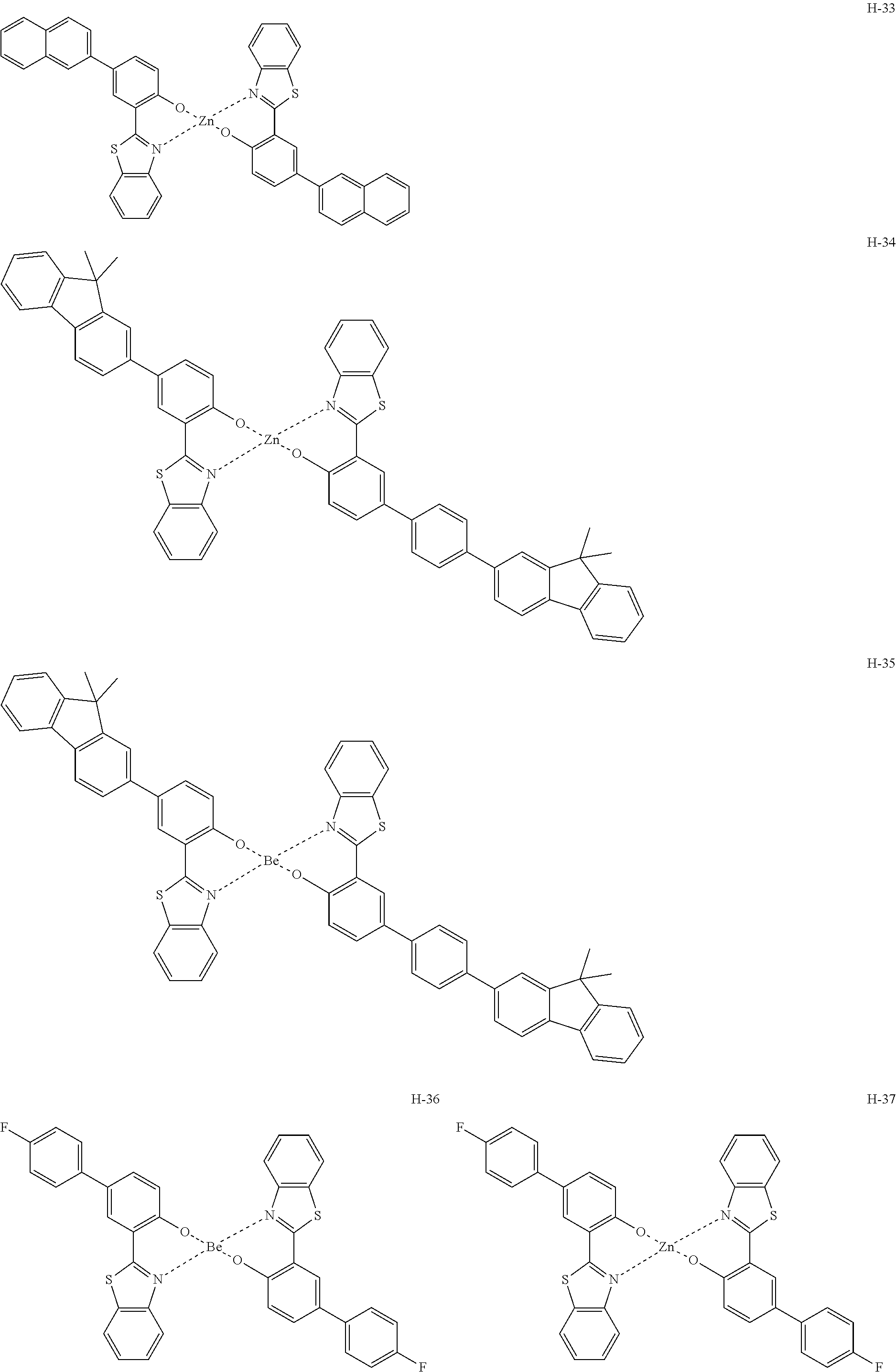

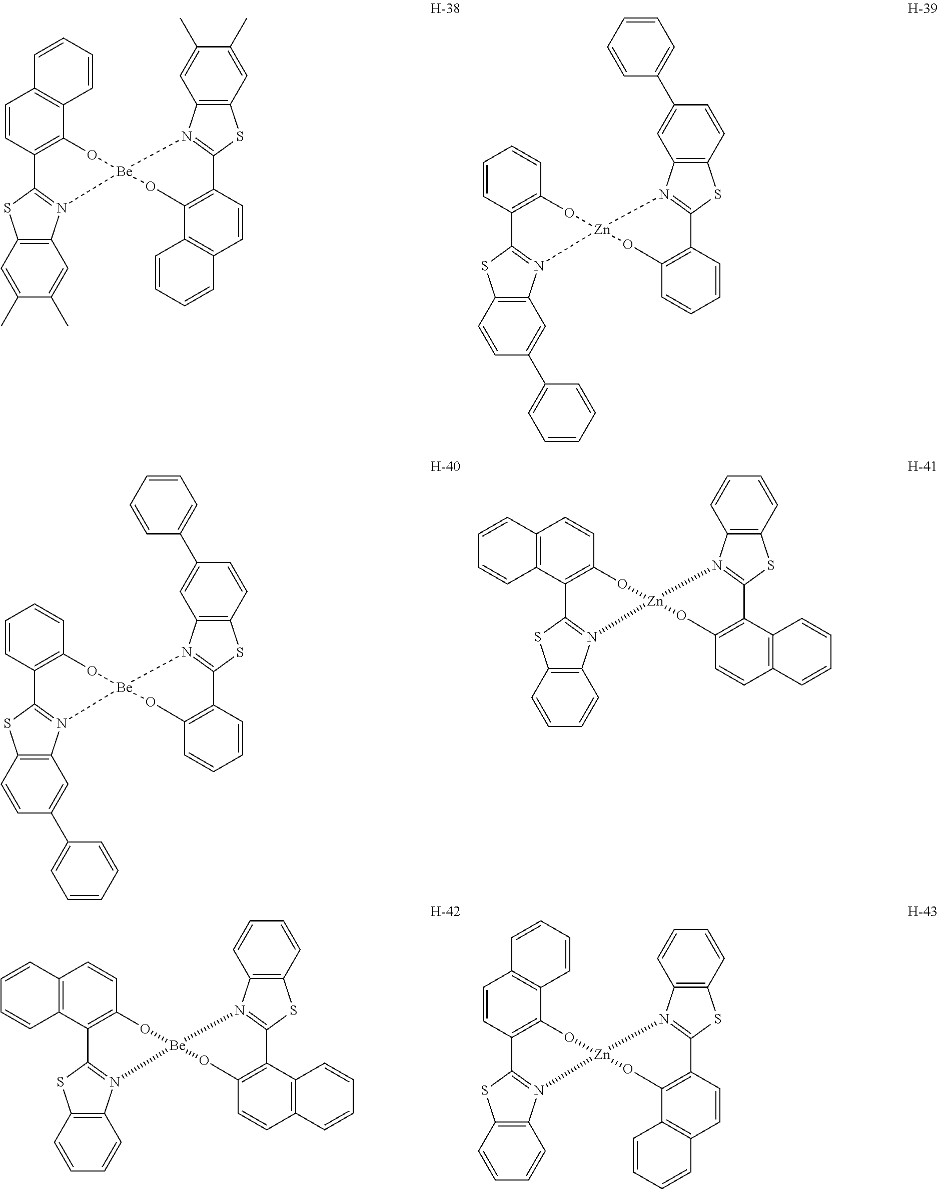

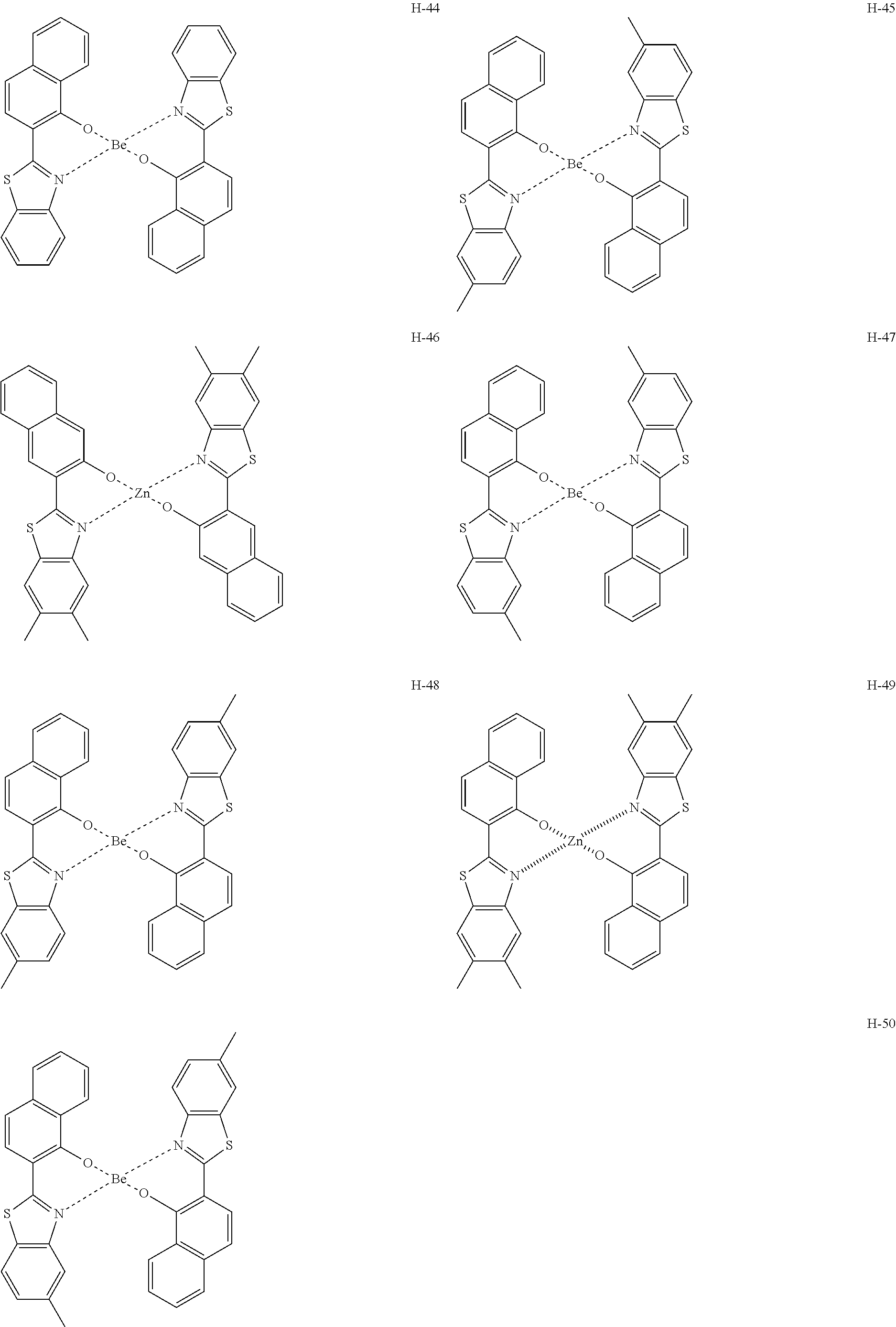

8. The organic electroluminescent device according to claim 1, wherein the compound represented by formula 2 is selected from the group consisting of: 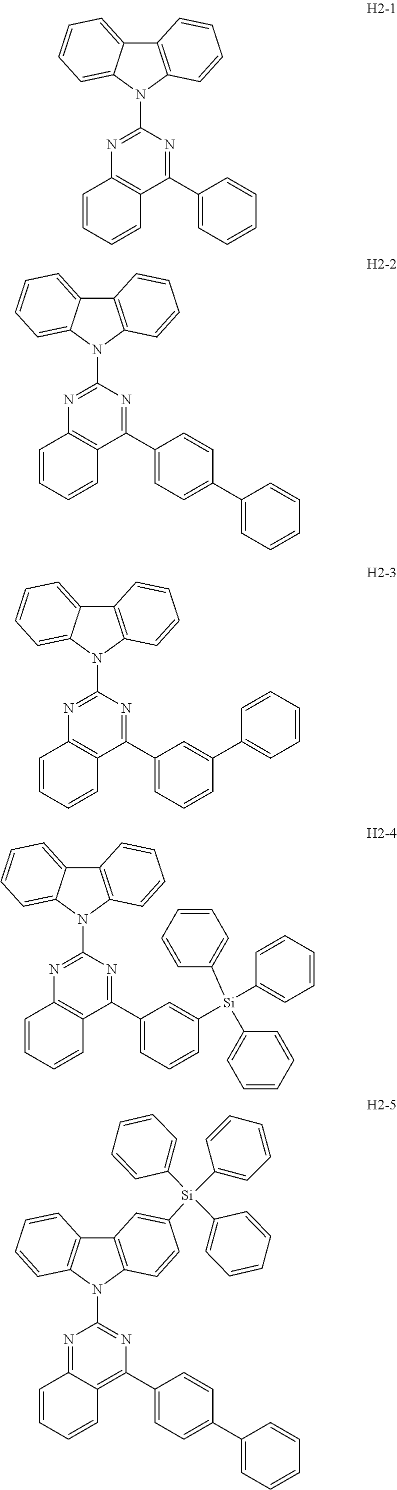 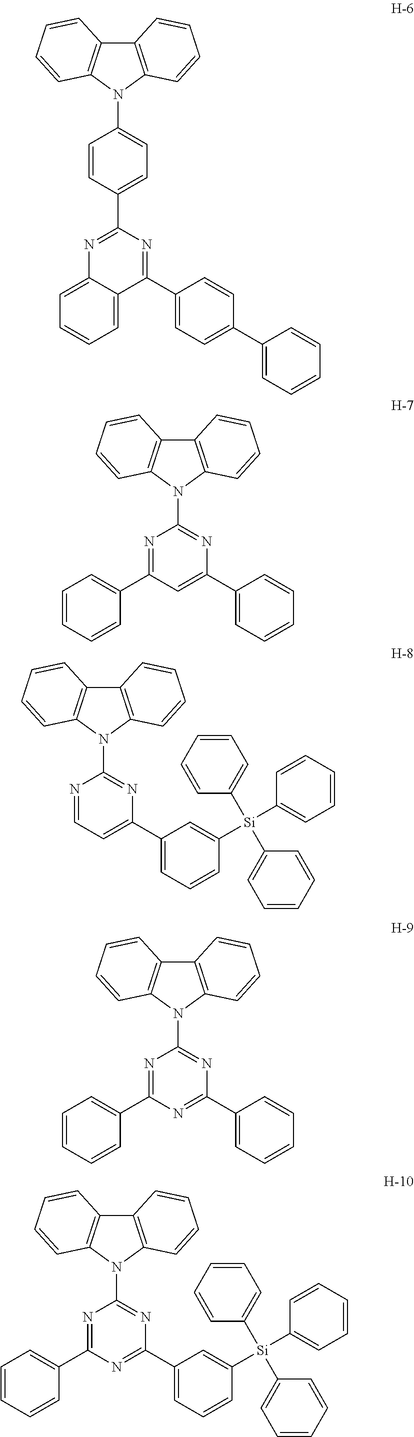 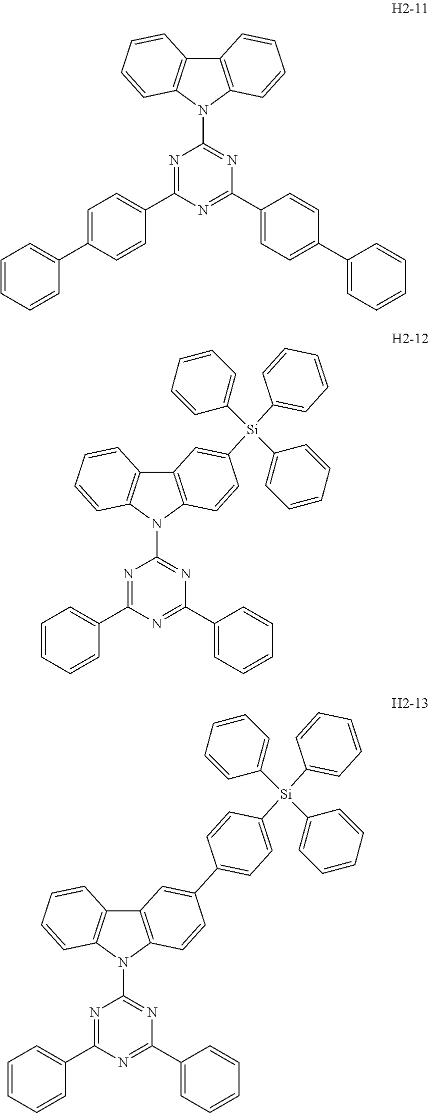 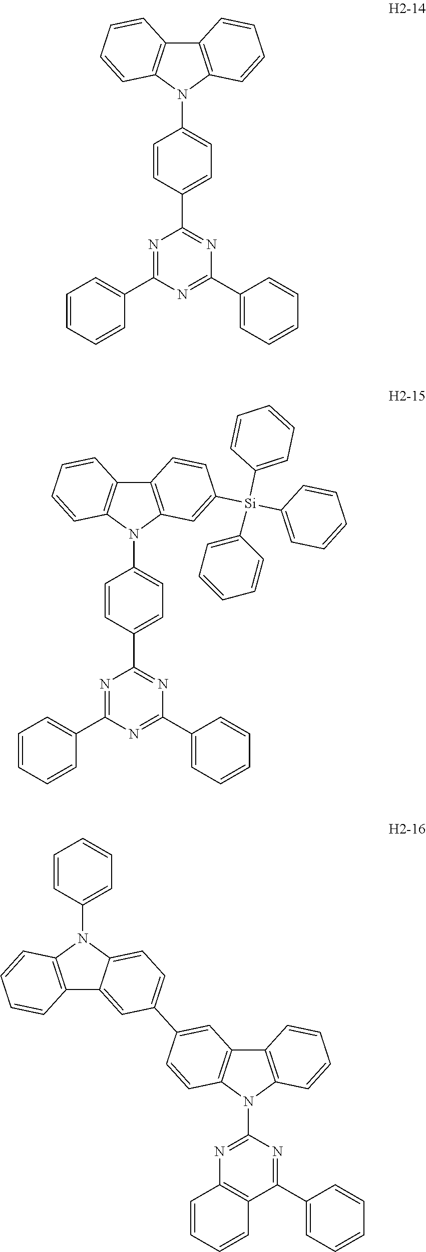 [SMILES: c1ccc(-c2nc(-c3ccccc3)nc(-c3ccc(-n4c5ccccc5c5ccccc54)cc3)n2)cc1 ; c1ccc(-c2nc(-c3ccccc3)nc(-c3ccc(-n4c5ccccc5c5ccc([Si](c6ccccc6)(c6ccccc6)c6ccccc6)cc54)cc3)n2)cc1 ; c1ccc(-c2nc(-n3c4ccccc4c4cc(-c5ccc6c(c5)c5ccccc5n6-c5ccccc5)ccc43)nc3ccccc23)cc1] 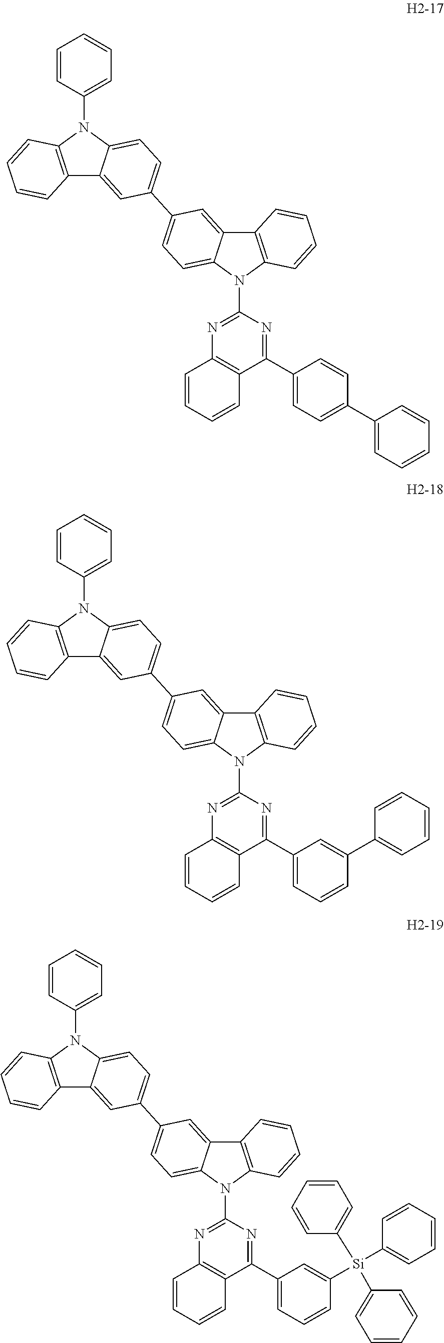 [SMILES: c1ccc(-c2ccc(-c3nc(-n4c5ccccc5c5cc(-c6ccc7c(c6)c6ccccc6n7-c6ccccc6)ccc54)nc4ccccc34)cc2)cc1 ; c1ccc(-c2cccc(-c3nc(-n4c5ccccc5c5cc(-c6ccc7c(c6)c6ccccc6n7-c6ccccc6)ccc54)nc4ccccc34)c2)cc1 ; c1ccc(-n2c3ccccc3c3cc(-c4ccc5c(c4)c4ccccc4n5-c4nc(-c5cccc([Si](c6ccccc6)(c6ccccc6)c6ccccc6)c5)c5ccccc5n4)ccc32)cc1] 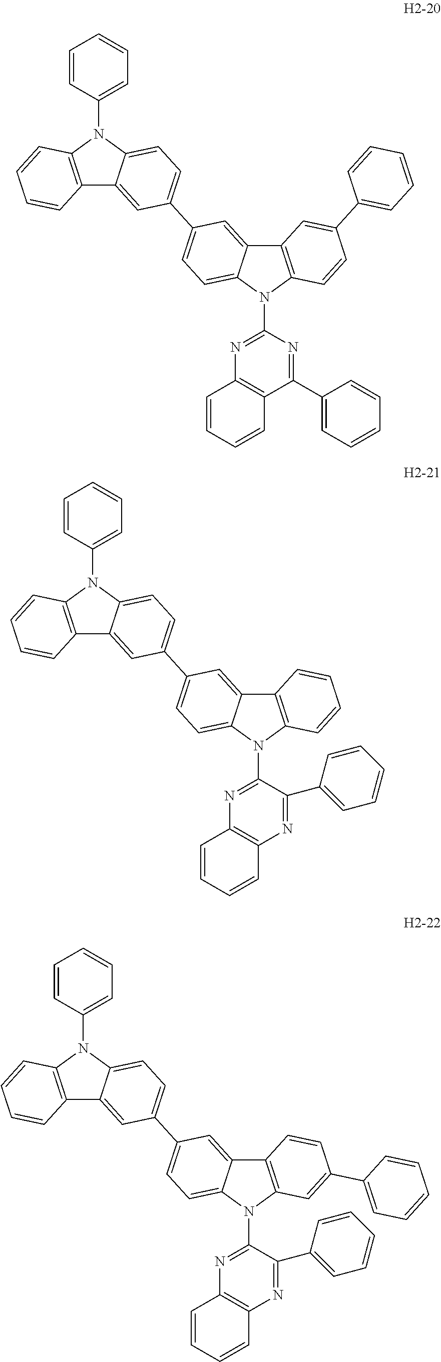 [SMILES: c1ccc(-c2ccc3c(c2)c2cc(-c4ccc5c(c4)c4ccccc4n5-c4ccccc4)ccc2n3-c2nc(-c3ccccc3)c3ccccc3n2)cc1 ; c1ccc(-c2nc3ccccc3nc2-n2c3ccccc3c3cc(-c4ccc5c(c4)c4ccccc4n5-c4ccccc4)ccc32)cc1 ; c1ccc(-c2ccc3c4cc(-c5ccc6c(c5)c5ccccc5n6-c5ccccc5)ccc4n(-c4nc5ccccc5nc4-c4ccccc4)c3c2)cc1] 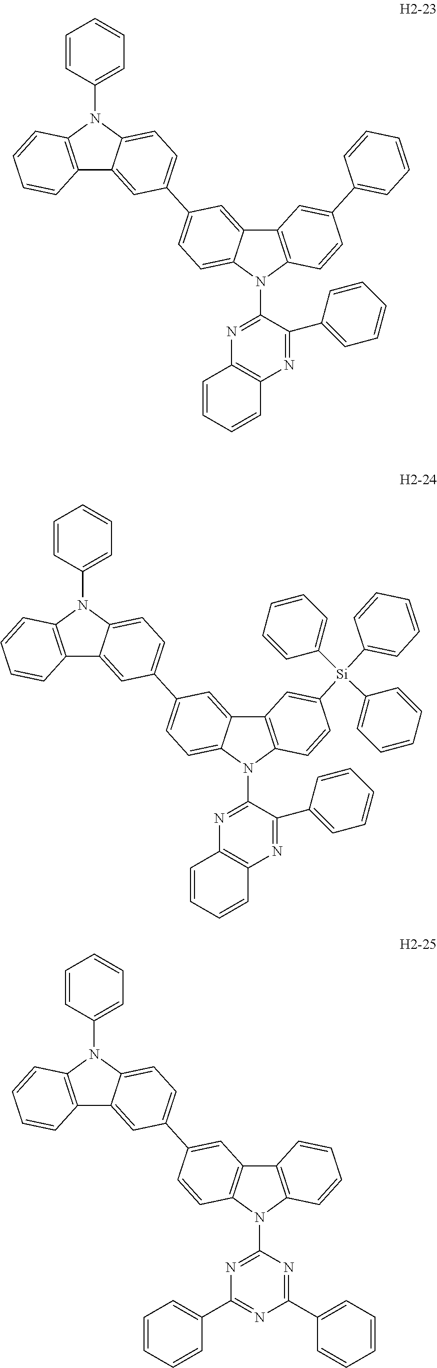 [SMILES: c1ccc(-c2ccc3c(c2)c2cc(-c4ccc5c(c4)c4ccccc4n5-c4ccccc4)ccc2n3-c2nc3ccccc3nc2-c2ccccc2)cc1 ; c1ccc(-c2nc3ccccc3nc2-n2c3ccc(-c4ccc5c(c4)c4ccccc4n5-c4ccccc4)cc3c3cc([Si](c4ccccc4)(c4ccccc4)c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1] 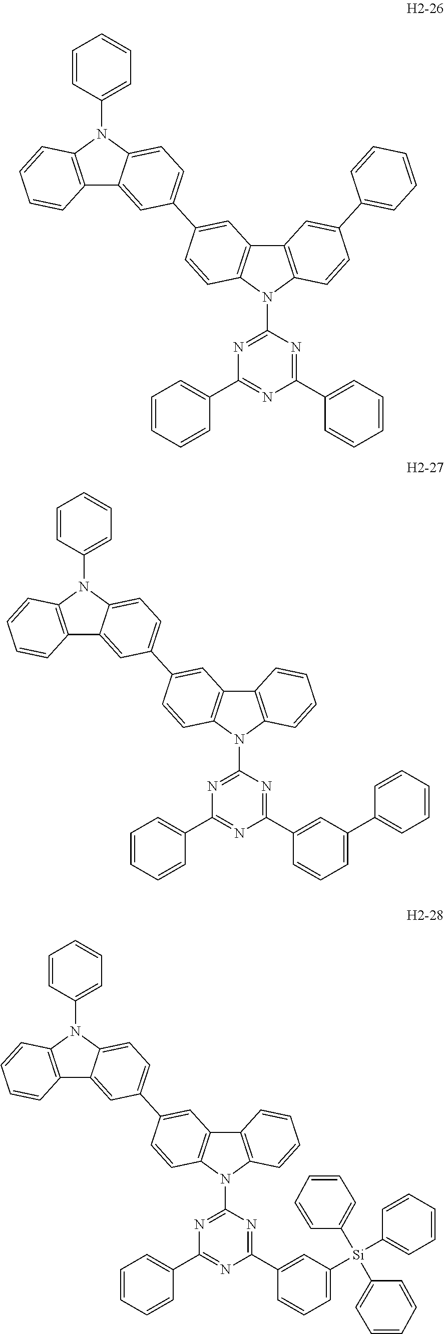 [SMILES: c1ccc(-c2ccc3c(c2)c2cc(-c4ccc5c(c4)c4ccccc4n5-c4ccccc4)ccc2n3-c2nc(-c3ccccc3)nc(-c3ccccc3)n2)cc1 ; c1ccc(-c2cccc(-c3nc(-c4ccccc4)nc(-n4c5ccccc5c5cc(-c6ccc7c(c6)c6ccccc6n7-c6ccccc6)ccc54)n3)c2)cc1 ; c1ccc(-c2nc(-c3cccc([Si](c4ccccc4)(c4ccccc4)c4ccccc4)c3)nc(-n3c4ccccc4c4cc(-c5ccc6c(c5)c5ccccc5n6-c5ccccc5)ccc43)n2)cc1] 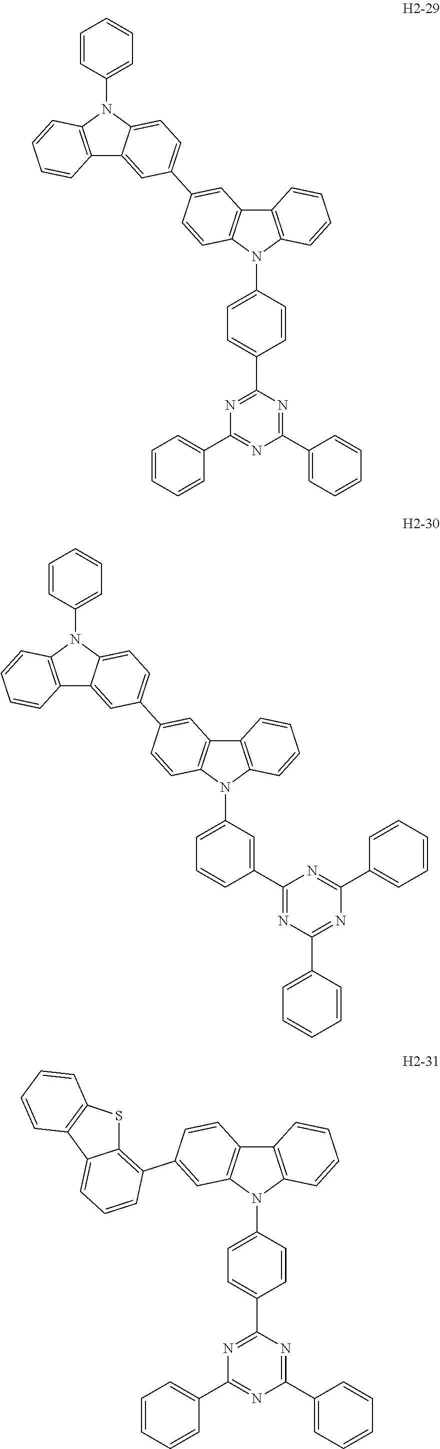 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2nc(-c3ccccc3)nc(-c3ccc(-n4c5ccccc5c5ccc(-c6cccc7c6sc6ccccc67)cc54)cc3)n2)cc1] 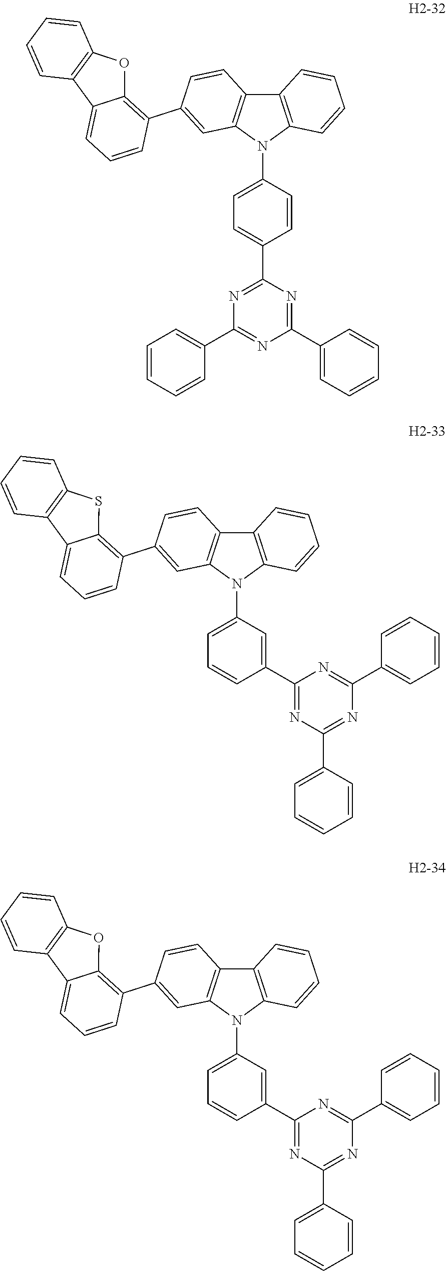 [SMILES: c1ccc(-c2nc(-c3ccccc3)nc(-c3ccc(-n4c5ccccc5c5ccc(-c6cccc7c6oc6ccccc67)cc54)cc3)n2)cc1 ; c1ccc(-c2nc(-c3ccccc3)nc(-c3cccc(-n4c5ccccc5c5ccc(-c6cccc7c6sc6ccccc67)cc54)c3)n2)cc1 ; c1ccc(-c2nc(-c3ccccc3)nc(-c3cccc(-n4c5ccccc5c5ccc(-c6cccc7c6oc6ccccc67)cc54)c3)n2)cc1] 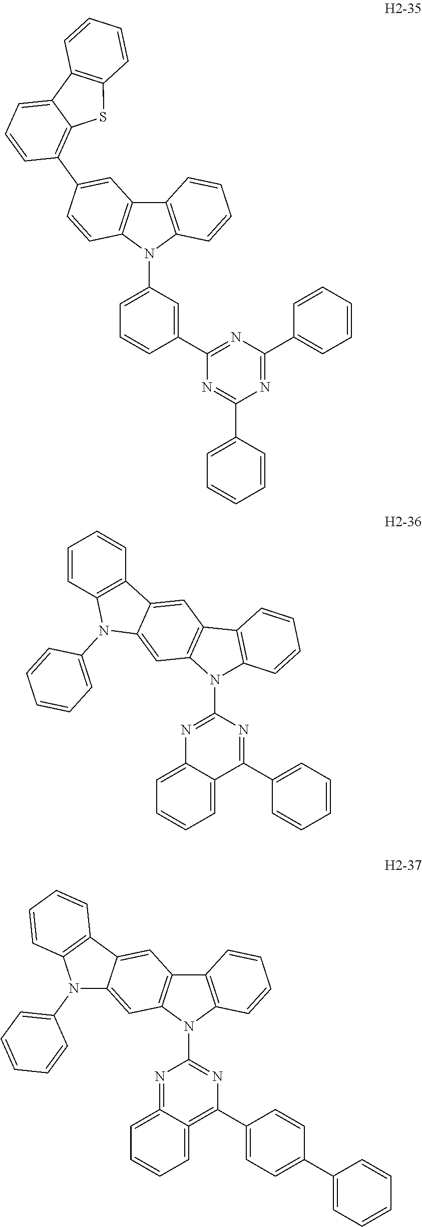 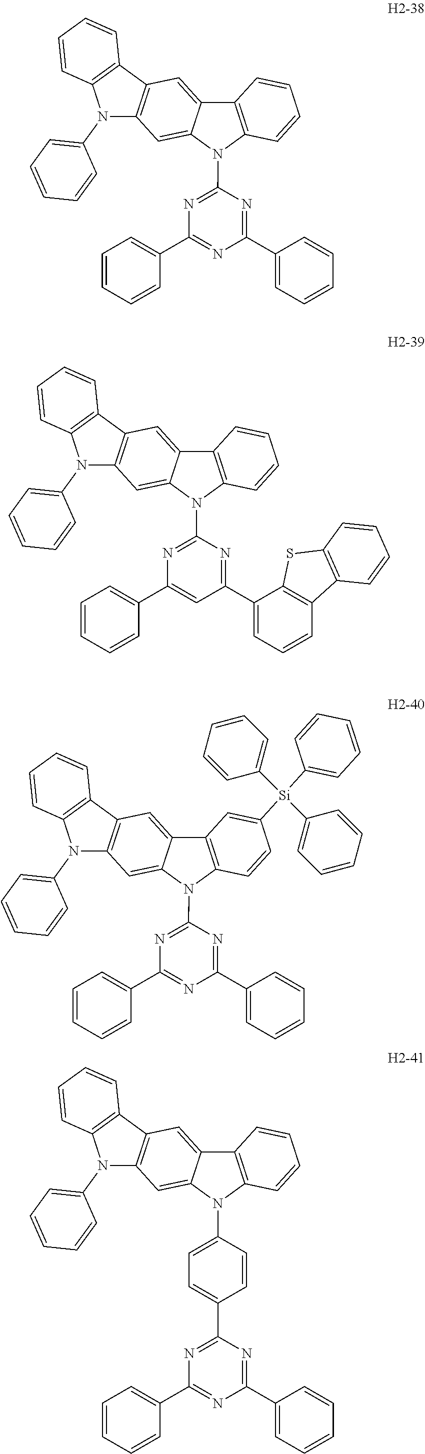 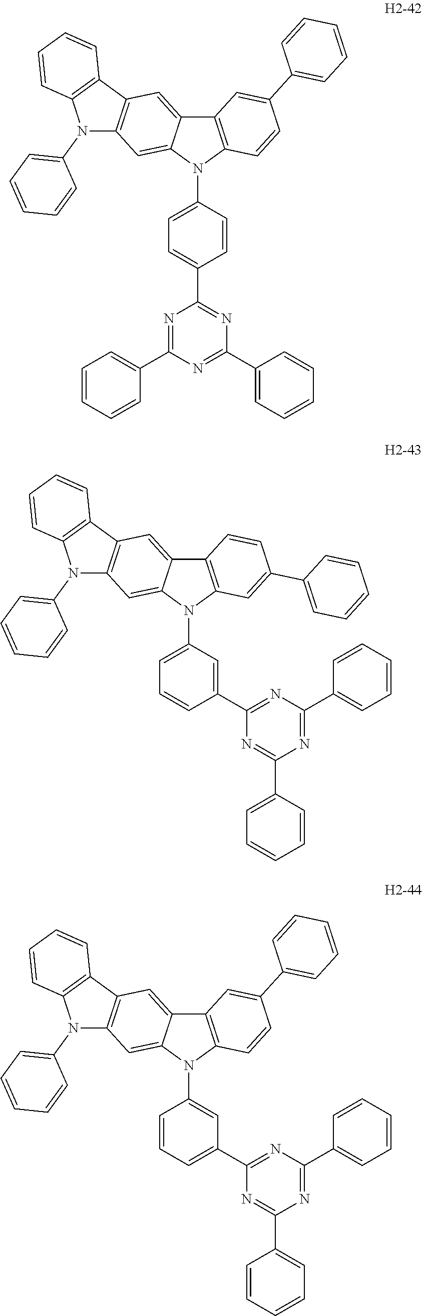 [SMILES: c1ccc(-c2ccc3c(c2)c2cc4c5ccccc5n(-c5ccccc5)c4cc2n3-c2ccc(-c3nc(-c4ccccc4)nc(-c4ccccc4)n3)cc2)cc1 ; c1ccc(-c2ccc3c4cc5c6ccccc6n(-c6ccccc6)c5cc4n(-c4cccc(-c5nc(-c6ccccc6)nc(-c6ccccc6)n5)c4)c3c2)cc1 ; c1ccc(-c2ccc3c(c2)c2cc4c5ccccc5n(-c5ccccc5)c4cc2n3-c2cccc(-c3nc(-c4ccccc4)nc(-c4ccccc4)n3)c2)cc1] 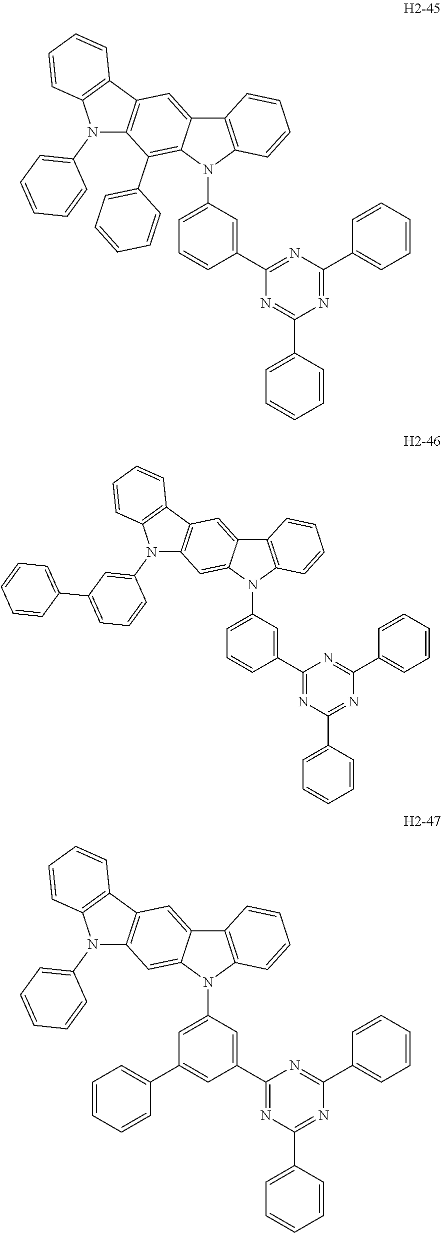 [SMILES: c1ccc(-c2nc(-c3ccccc3)nc(-c3cccc(-n4c5ccccc5c5cc6c7ccccc7n(-c7ccccc7)c6c(-c6ccccc6)c54)c3)n2)cc1 ; c1ccc(-c2cccc(-n3c4ccccc4c4cc5c6ccccc6n(-c6cccc(-c7nc(-c8ccccc8)nc(-c8ccccc8)n7)c6)c5cc43)c2)cc1 ; c1ccc(-c2cc(-c3nc(-c4ccccc4)nc(-c4ccccc4)n3)cc(-n3c4ccccc4c4cc5c6ccccc6n(-c6ccccc6)c5cc43)c2)cc1] 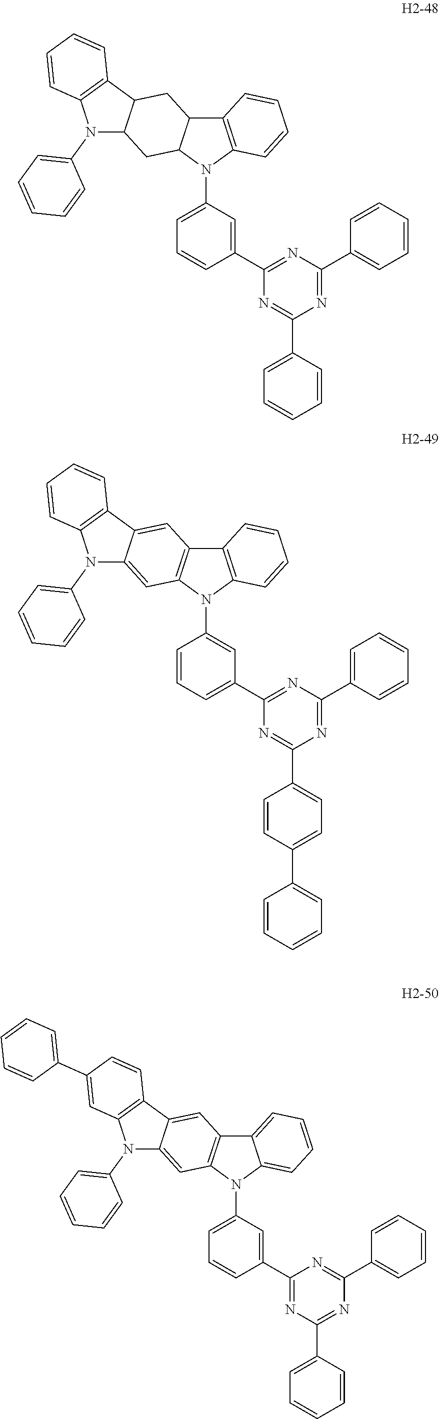 [SMILES: c1ccc(-c2nc(-c3ccccc3)nc(-c3cccc(N4c5ccccc5C5CC6c7ccccc7N(c7ccccc7)C6CC54)c3)n2)cc1 ; c1ccc(-c2ccc(-c3nc(-c4ccccc4)nc(-c4cccc(-n5c6ccccc6c6cc7c8ccccc8n(-c8ccccc8)c7cc65)c4)n3)cc2)cc1 ; c1ccc(-c2ccc3c4cc5c6ccccc6n(-c6cccc(-c7nc(-c8ccccc8)nc(-c8ccccc8)n7)c6)c5cc4n(-c4ccccc4)c3c2)cc1] 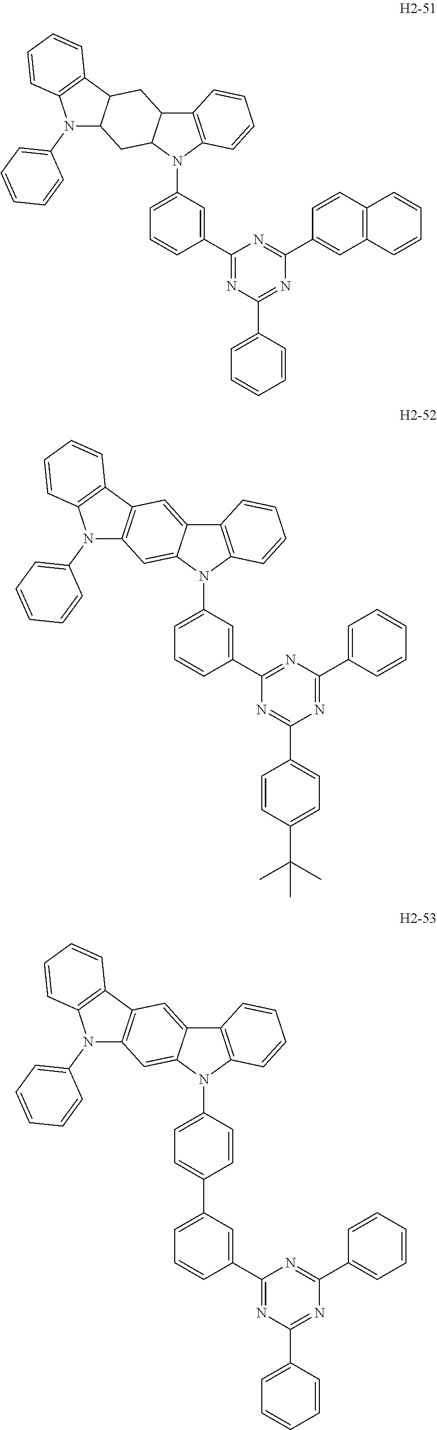 [SMILES: c1ccc(-c2nc(-c3cccc(N4c5ccccc5C5CC6c7ccccc7N(c7ccccc7)C6CC54)c3)nc(-c3ccc4ccccc4c3)n2)cc1 ; CC(C)(C)c1ccc(-c2nc(-c3ccccc3)nc(-c3cccc(-n4c5ccccc5c5cc6c7ccccc7n(-c7ccccc7)c6cc54)c3)n2)cc1 ; c1ccc(-c2nc(-c3ccccc3)nc(-c3cccc(-c4ccc(-n5c6ccccc6c6cc7c8ccccc8n(-c8ccccc8)c7cc65)cc4)c3)n2)cc1] 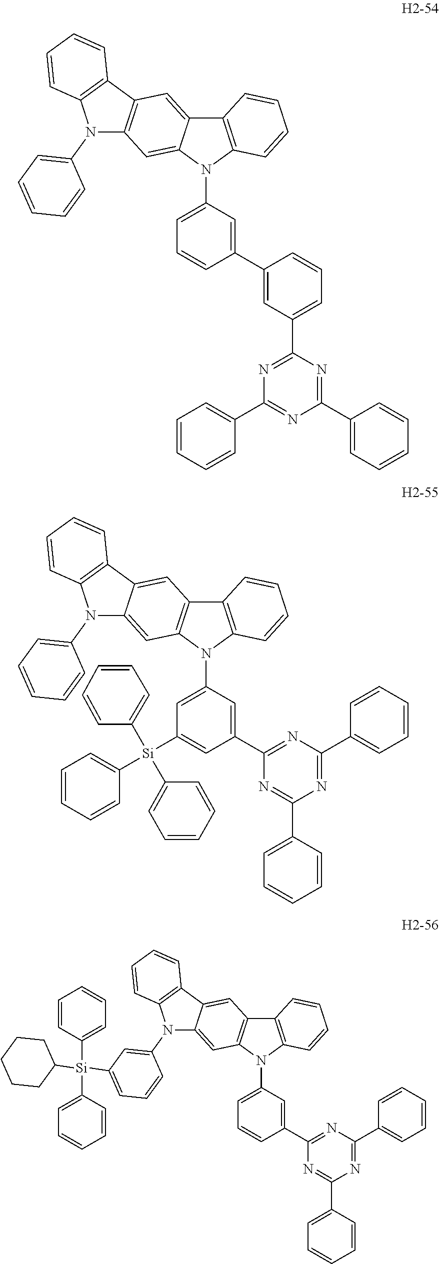 [SMILES: c1ccc(-c2nc(-c3ccccc3)nc(-c3cccc(-c4cccc(-n5c6ccccc6c6cc7c8ccccc8n(-c8ccccc8)c7cc65)c4)c3)n2)cc1 ; c1ccc(-c2nc(-c3ccccc3)nc(-c3cc(-n4c5ccccc5c5cc6c7ccccc7n(-c7ccccc7)c6cc54)cc([Si](c4ccccc4)(c4ccccc4)c4ccccc4)c3)n2)cc1 ; c1ccc(-c2nc(-c3ccccc3)nc(-c3cccc(-n4c5ccccc5c5cc6c7ccccc7n(-c7cccc([Si](c8ccccc8)(c8ccccc8)C8CCCCC8)c7)c6cc54)c3)n2)cc1] 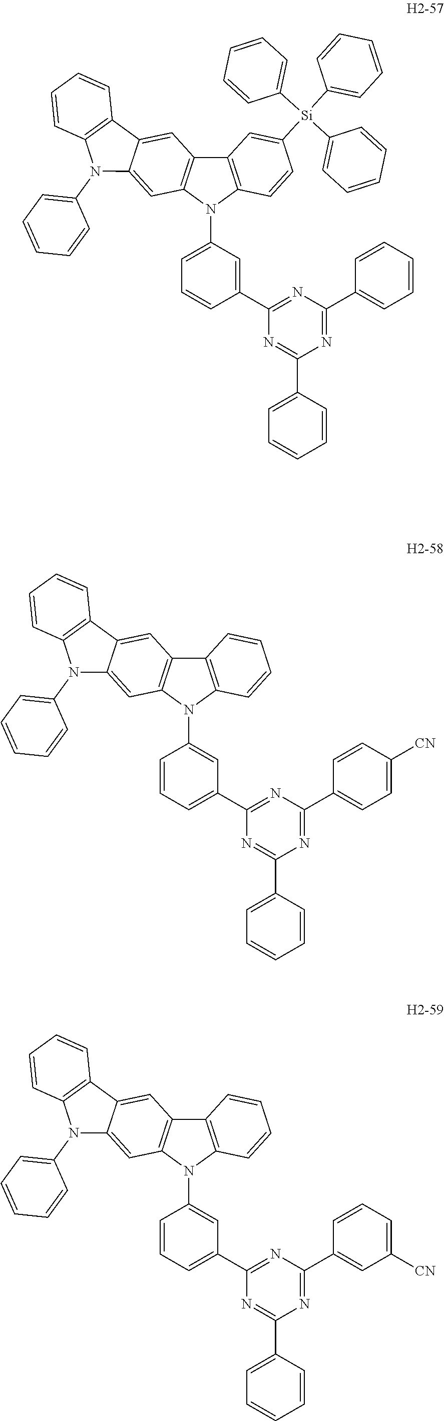 [SMILES: c1ccc(-c2nc(-c3ccccc3)nc(-c3cccc(-n4c5ccc([Si](c6ccccc6)(c6ccccc6)c6ccccc6)cc5c5cc6c7ccccc7n(-c7ccccc7)c6cc54)c3)n2)cc1 ; N#Cc1ccc(-c2nc(-c3ccccc3)nc(-c3cccc(-n4c5ccccc5c5cc6c7ccccc7n(-c7ccccc7)c6cc54)c3)n2)cc1 ; N#Cc1cccc(-c2nc(-c3ccccc3)nc(-c3cccc(-n4c5ccccc5c5cc6c7ccccc7n(-c7ccccc7)c6cc54)c3)n2)c1] 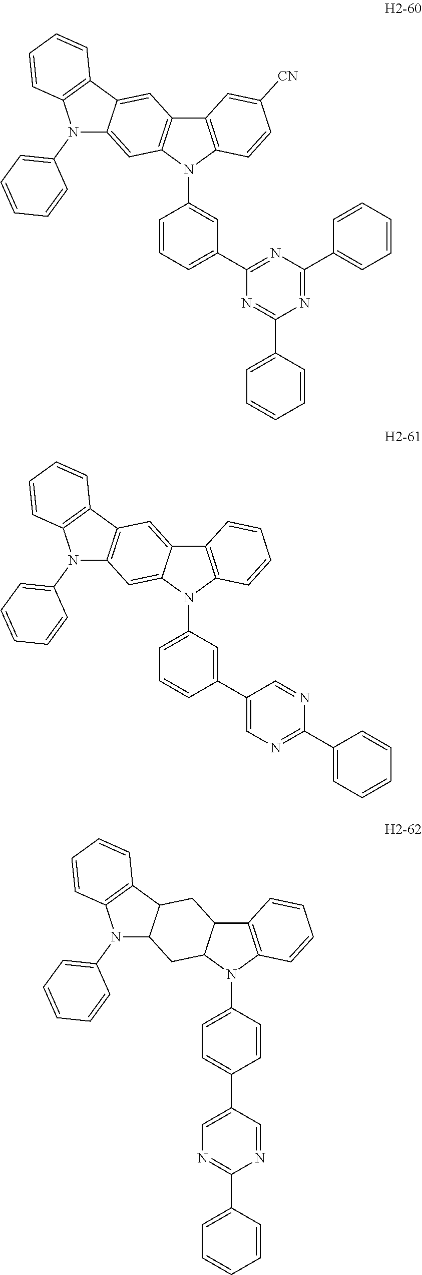 [SMILES: N#Cc1ccc2c(c1)c1cc3c4ccccc4n(-c4ccccc4)c3cc1n2-c1cccc(-c2nc(-c3ccccc3)nc(-c3ccccc3)n2)c1 ; c1ccc(-c2ncc(-c3cccc(-n4c5ccccc5c5cc6c7ccccc7n(-c7ccccc7)c6cc54)c3)cn2)cc1 ; c1ccc(-c2ncc(-c3ccc(N4c5ccccc5C5CC6c7ccccc7N(c7ccccc7)C6CC54)cc3)cn2)cc1] 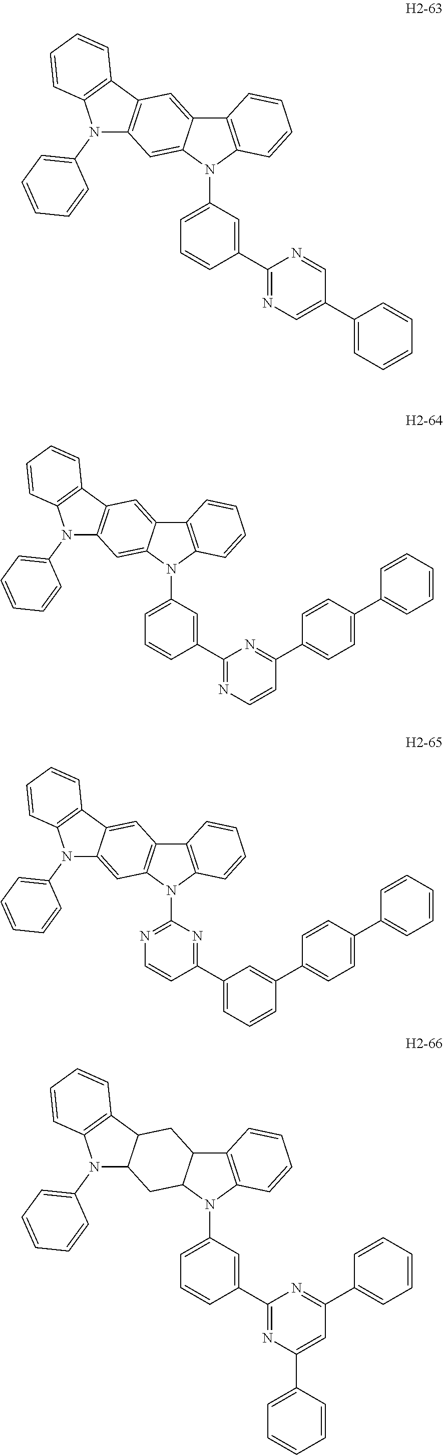 [SMILES: c1ccc(-c2cnc(-c3cccc(-n4c5ccccc5c5cc6c7ccccc7n(-c7ccccc7)c6cc54)c3)nc2)cc1 ; c1ccc(-c2ccc(-c3ccnc(-c4cccc(-n5c6ccccc6c6cc7c8ccccc8n(-c8ccccc8)c7cc65)c4)n3)cc2)cc1 ; c1ccc(-c2ccc(-c3cccc(-c4ccnc(-n5c6ccccc6c6cc7c8ccccc8n(-c8ccccc8)c7cc65)n4)c3)cc2)cc1 ; c1ccc(-c2cc(-c3ccccc3)nc(-c3cccc(N4c5ccccc5C5CC6c7ccccc7N(c7ccccc7)C6CC54)c3)n2)cc1] 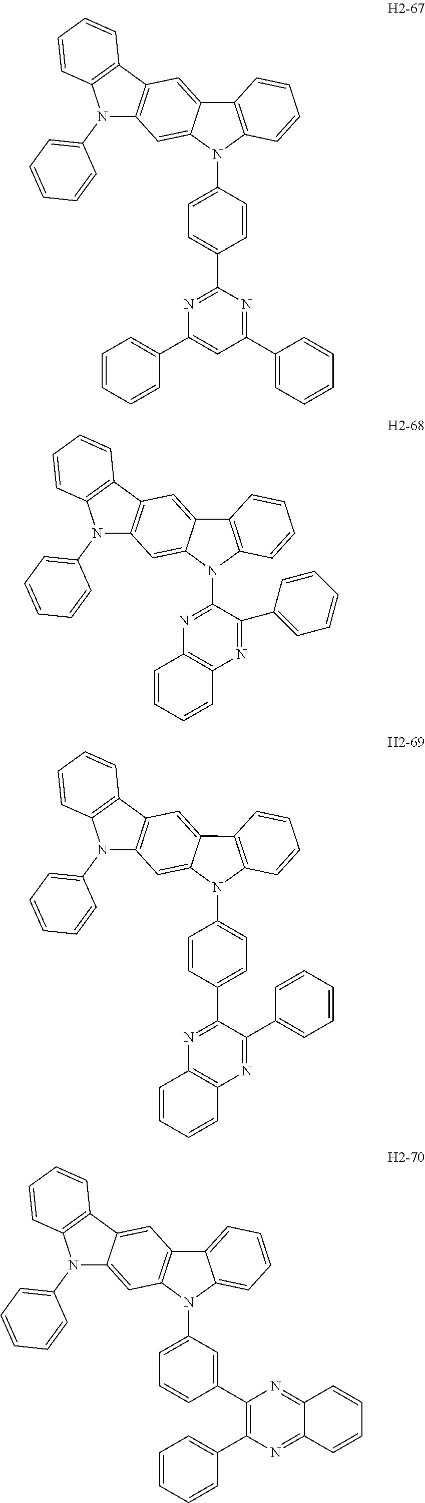 [SMILES: c1ccc(-c2cc(-c3ccccc3)nc(-c3ccc(-n4c5ccccc5c5cc6c7ccccc7n(-c7ccccc7)c6cc54)cc3)n2)cc1 ; c1ccc(-c2nc3ccccc3nc2-n2c3ccccc3c3cc4c5ccccc5n(-c5ccccc5)c4cc32)cc1 ; c1ccc(-c2nc3ccccc3nc2-c2ccc(-n3c4ccccc4c4cc5c6ccccc6n(-c6ccccc6)c5cc43)cc2)cc1 ; c1ccc(-c2nc3ccccc3nc2-c2cccc(-n3c4ccccc4c4cc5c6ccccc6n(-c6ccccc6)c5cc43)c2)cc1] 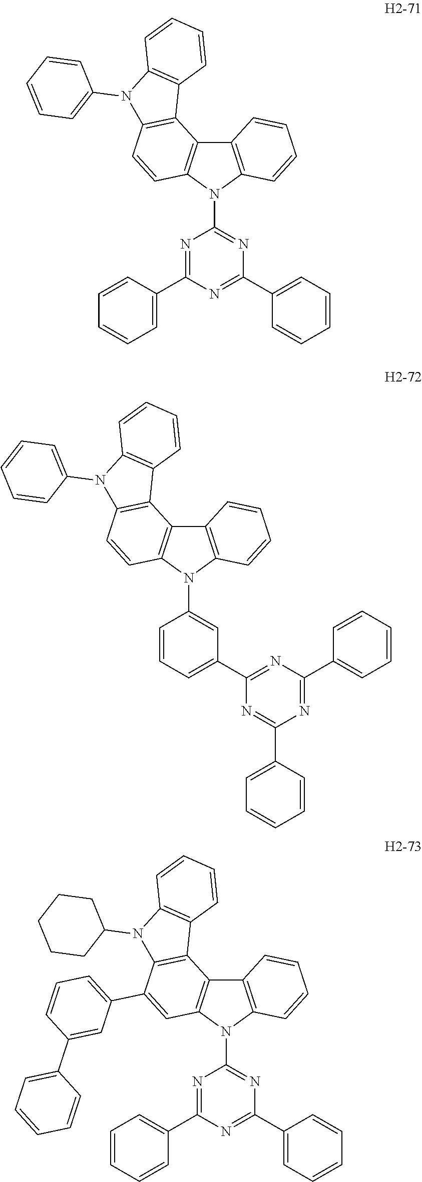 [SMILES: c1ccc(-c2nc(-c3ccccc3)nc(-n3c4ccccc4c4c5c6ccccc6n(-c6ccccc6)c5ccc43)n2)cc1 ; c1ccc(-c2nc(-c3ccccc3)nc(-c3cccc(-n4c5ccccc5c5c6c7ccccc7n(-c7ccccc7)c6ccc54)c3)n2)cc1 ; c1ccc(-c2cccc(-c3cc4c(c5ccccc5n4-c4nc(-c5ccccc5)nc(-c5ccccc5)n4)c4c5ccccc5n(C5CCCCC5)c34)c2)cc1] 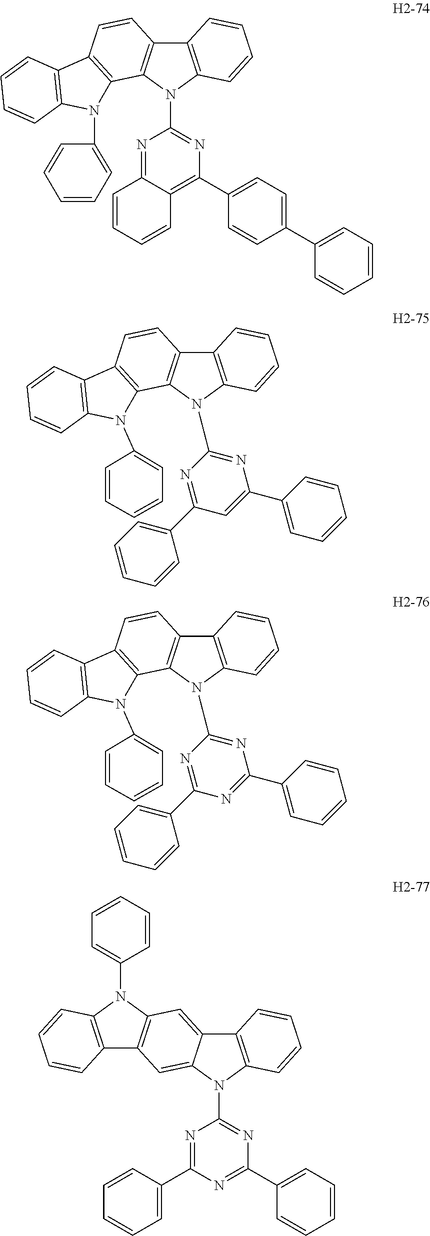 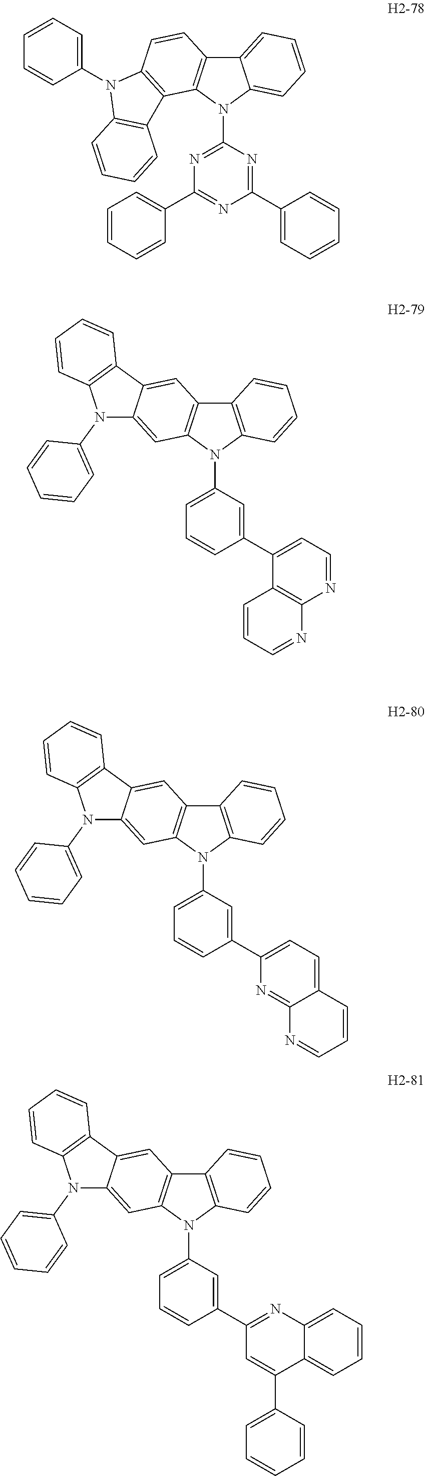 [SMILES: c1ccc(-c2nc(-c3ccccc3)nc(-n3c4ccccc4c4ccc5c(c6ccccc6n5-c5ccccc5)c43)n2)cc1 ; c1ccc(-n2c3ccccc3c3cc4c5ccccc5n(-c5cccc(-c6ccnc7ncccc67)c5)c4cc32)cc1 ; c1ccc(-n2c3ccccc3c3cc4c5ccccc5n(-c5cccc(-c6ccc7cccnc7n6)c5)c4cc32)cc1 ; c1ccc(-c2cc(-c3cccc(-n4c5ccccc5c5cc6c7ccccc7n(-c7ccccc7)c6cc54)c3)nc3ccccc23)cc1] 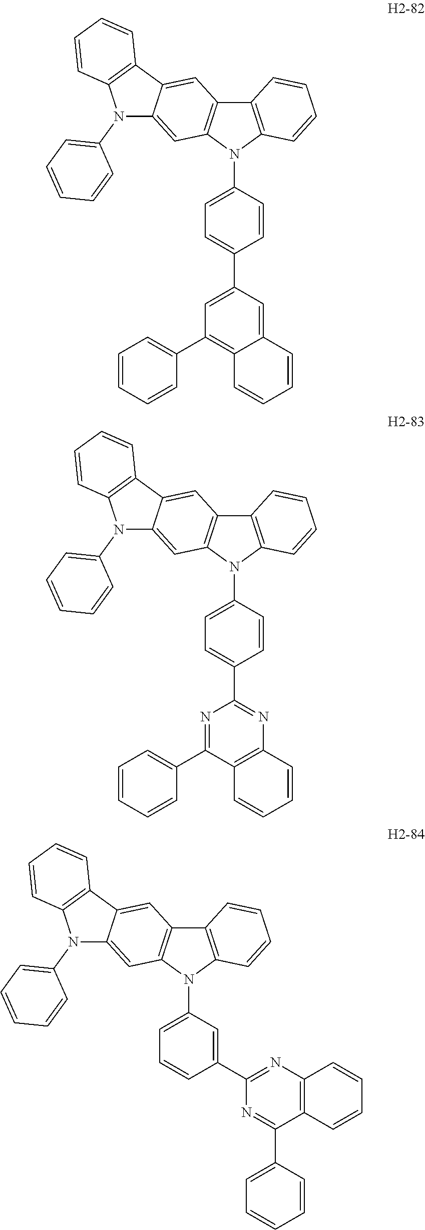 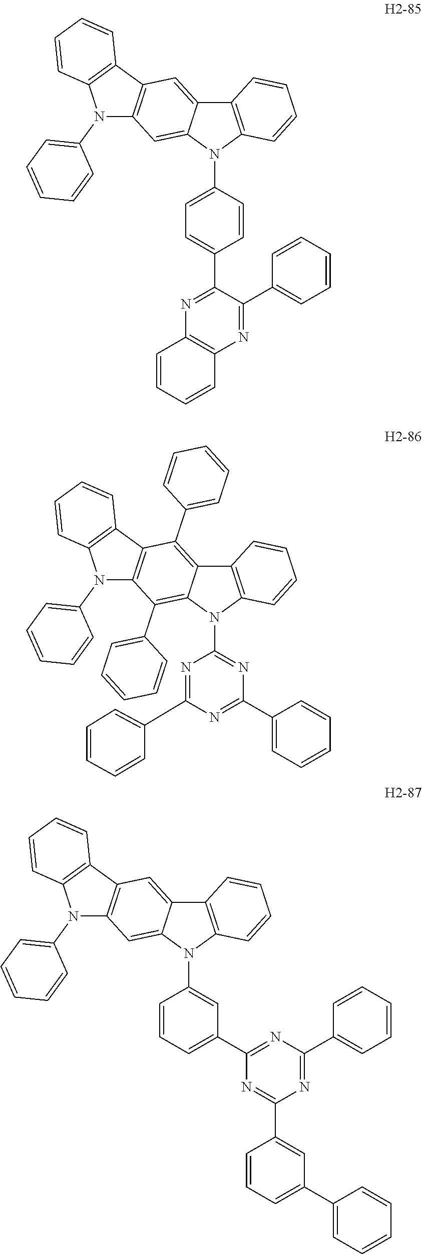 [SMILES: c1ccc(-c2nc3ccccc3nc2-c2ccc(-n3c4ccccc4c4cc5c6ccccc6n(-c6ccccc6)c5cc43)cc2)cc1 ; c1ccc(-c2nc(-c3ccccc3)nc(-n3c4ccccc4c4c(-c5ccccc5)c5c6ccccc6n(-c6ccccc6)c5c(-c5ccccc5)c43)n2)cc1 ; c1ccc(-c2cccc(-c3nc(-c4ccccc4)nc(-c4cccc(-n5c6ccccc6c6cc7c8ccccc8n(-c8ccccc8)c7cc65)c4)n3)c2)cc1] 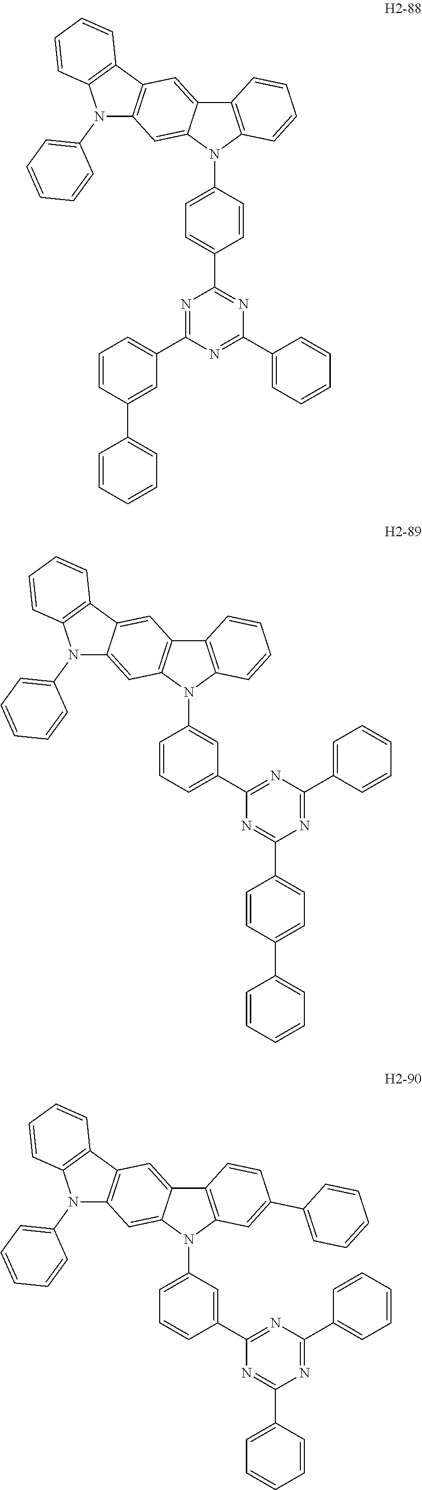 [SMILES: c1ccc(-c2cccc(-c3nc(-c4ccccc4)nc(-c4ccc(-n5c6ccccc6c6cc7c8ccccc8n(-c8ccccc8)c7cc65)cc4)n3)c2)cc1 ; c1ccc(-c2ccc(-c3nc(-c4ccccc4)nc(-c4cccc(-n5c6ccccc6c6cc7c8ccccc8n(-c8ccccc8)c7cc65)c4)n3)cc2)cc1 ; c1ccc(-c2ccc3c4cc5c6ccccc6n(-c6ccccc6)c5cc4n(-c4cccc(-c5nc(-c6ccccc6)nc(-c6ccccc6)n5)c4)c3c2)cc1] 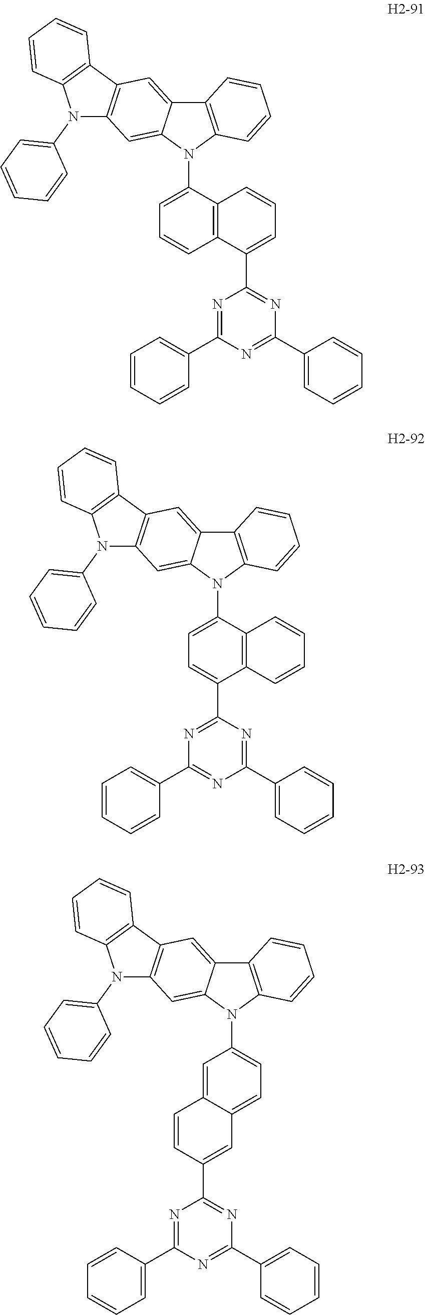 [SMILES: c1ccc(-c2nc(-c3ccccc3)nc(-c3cccc4c(-n5c6ccccc6c6cc7c8ccccc8n(-c8ccccc8)c7cc65)cccc34)n2)cc1 ; c1ccc(-c2nc(-c3ccccc3)nc(-c3ccc(-n4c5ccccc5c5cc6c7ccccc7n(-c7ccccc7)c6cc54)c4ccccc34)n2)cc1 ; c1ccc(-c2nc(-c3ccccc3)nc(-c3ccc4cc(-n5c6ccccc6c6cc7c8ccccc8n(-c8ccccc8)c7cc65)ccc4c3)n2)cc1] 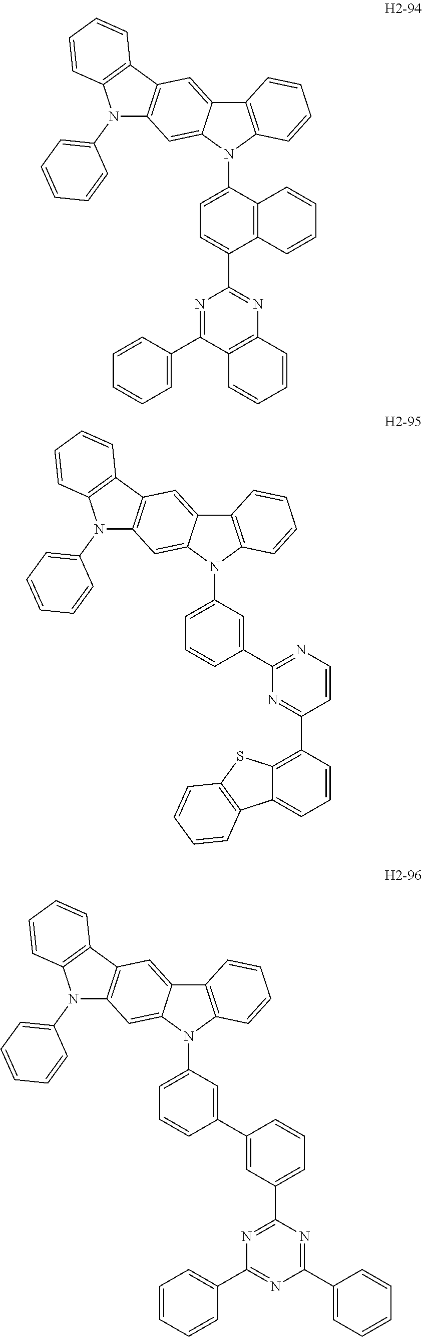 [SMILES: c1ccc(-c2nc(-c3ccc(-n4c5ccccc5c5cc6c7ccccc7n(-c7ccccc7)c6cc54)c4ccccc34)nc3ccccc23)cc1 ; c1ccc(-n2c3ccccc3c3cc4c5ccccc5n(-c5cccc(-c6nccc(-c7cccc8c7sc7ccccc78)n6)c5)c4cc32)cc1 ; c1ccc(-c2nc(-c3ccccc3)nc(-c3cccc(-c4cccc(-n5c6ccccc6c6cc7c8ccccc8n(-c8ccccc8)c7cc65)c4)c3)n2)cc1] 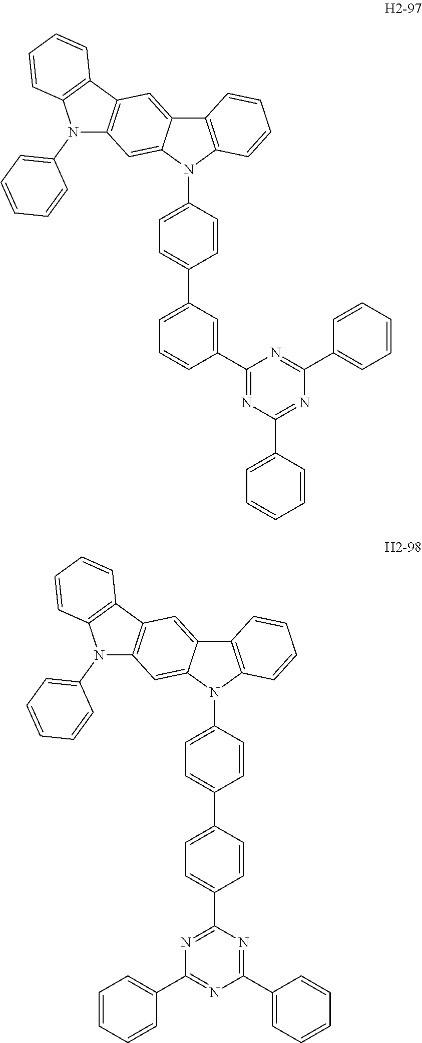 [SMILES: c1ccc(-c2nc(-c3ccccc3)nc(-c3cccc(-c4ccc(-n5c6ccccc6c6cc7c8ccccc8n(-c8ccccc8)c7cc65)cc4)c3)n2)cc1 ; c1ccc(-c2nc(-c3ccccc3)nc(-c3ccc(-c4ccc(-n5c6ccccc6c6cc7c8ccccc8n(-c8ccccc8)c7cc65)cc4)cc3)n2)cc1] 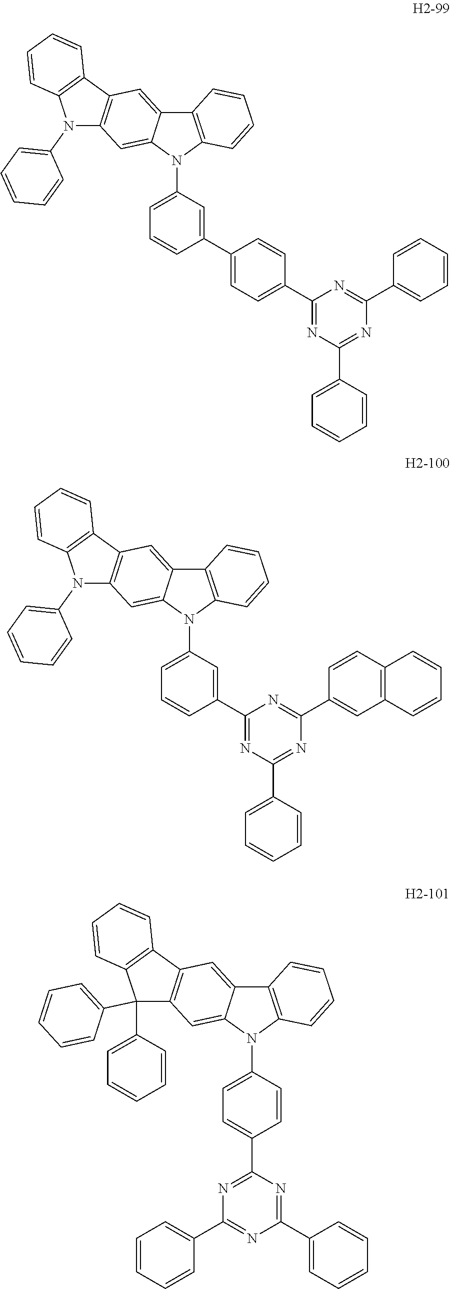 [SMILES: c1ccc(-c2nc(-c3ccccc3)nc(-c3ccc(-c4cccc(-n5c6ccccc6c6cc7c8ccccc8n(-c8ccccc8)c7cc65)c4)cc3)n2)cc1 ; c1ccc(-c2nc(-c3cccc(-n4c5ccccc5c5cc6c7ccccc7n(-c7ccccc7)c6cc54)c3)nc(-c3ccc4ccccc4c3)n2)cc1 ; c1ccc(-c2nc(-c3ccccc3)nc(-c3ccc(-n4c5ccccc5c5cc6c(cc54)C(c4ccccc4)(c4ccccc4)c4ccccc4-6)cc3)n2)cc1] 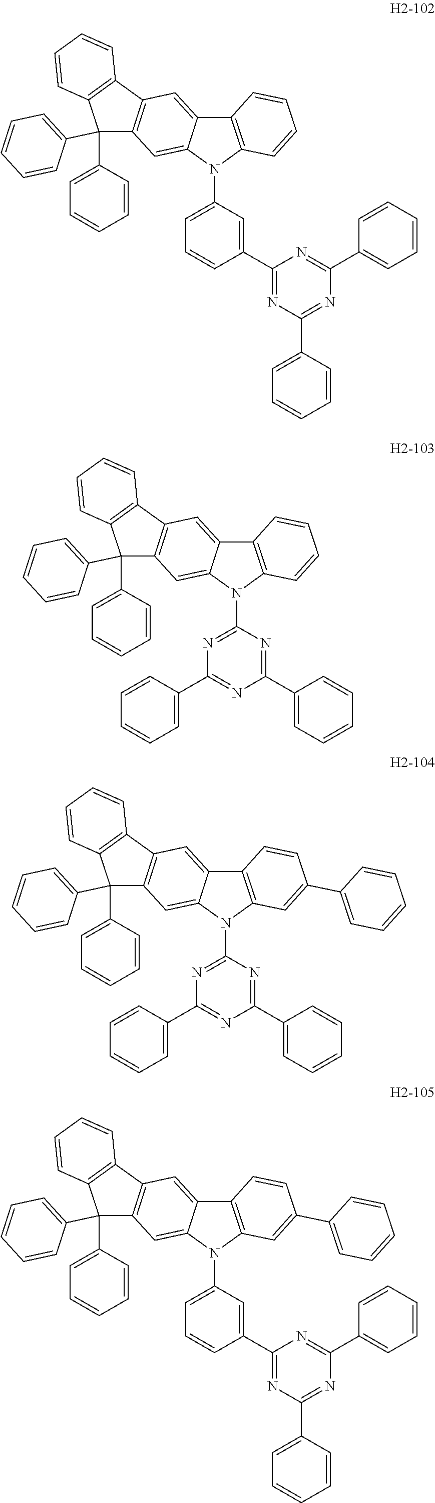 [SMILES: c1ccc(-c2nc(-c3ccccc3)nc(-c3cccc(-n4c5ccccc5c5cc6c(cc54)C(c4ccccc4)(c4ccccc4)c4ccccc4-6)c3)n2)cc1 ; c1ccc(-c2nc(-c3ccccc3)nc(-n3c4ccccc4c4cc5c(cc43)C(c3ccccc3)(c3ccccc3)c3ccccc3-5)n2)cc1 ; c1ccc(-c2ccc3c4cc5c(cc4n(-c4nc(-c6ccccc6)nc(-c6ccccc6)n4)c3c2)C(c2ccccc2)(c2ccccc2)c2ccccc2-5)cc1 ; c1ccc(-c2ccc3c4cc5c(cc4n(-c4cccc(-c6nc(-c7ccccc7)nc(-c7ccccc7)n6)c4)c3c2)C(c2ccccc2)(c2ccccc2)c2ccccc2-5)cc1] 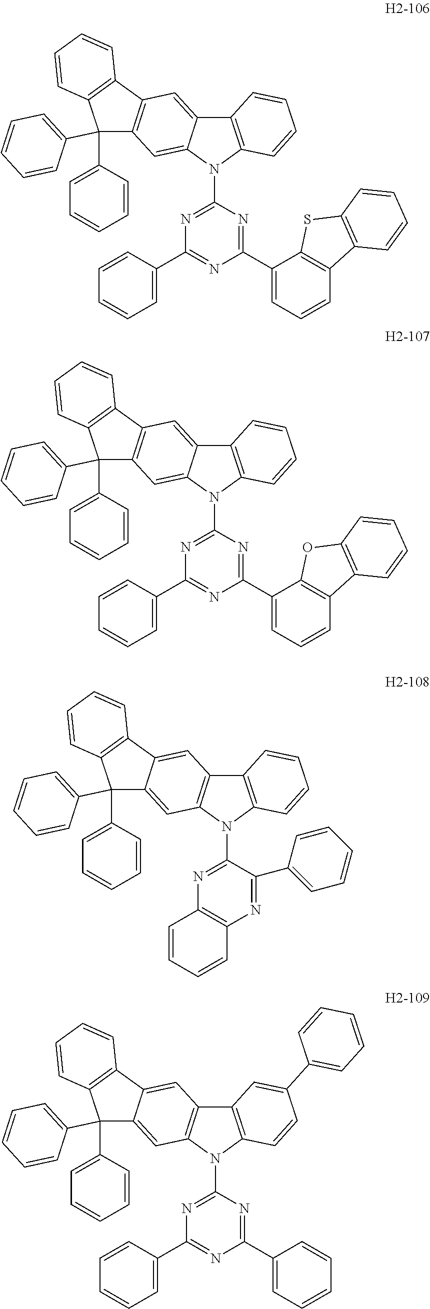 [SMILES: c1ccc(-c2nc(-c3cccc4c3sc3ccccc34)nc(-n3c4ccccc4c4cc5c(cc43)C(c3ccccc3)(c3ccccc3)c3ccccc3-5)n2)cc1 ; c1ccc(-c2nc(-c3cccc4c3oc3ccccc34)nc(-n3c4ccccc4c4cc5c(cc43)C(c3ccccc3)(c3ccccc3)c3ccccc3-5)n2)cc1 ; c1ccc(-c2nc3ccccc3nc2-n2c3ccccc3c3cc4c(cc32)C(c2ccccc2)(c2ccccc2)c2ccccc2-4)cc1 ; c1ccc(-c2ccc3c(c2)c2cc4c(cc2n3-c2nc(-c3ccccc3)nc(-c3ccccc3)n2)C(c2ccccc2)(c2ccccc2)c2ccccc2-4)cc1] 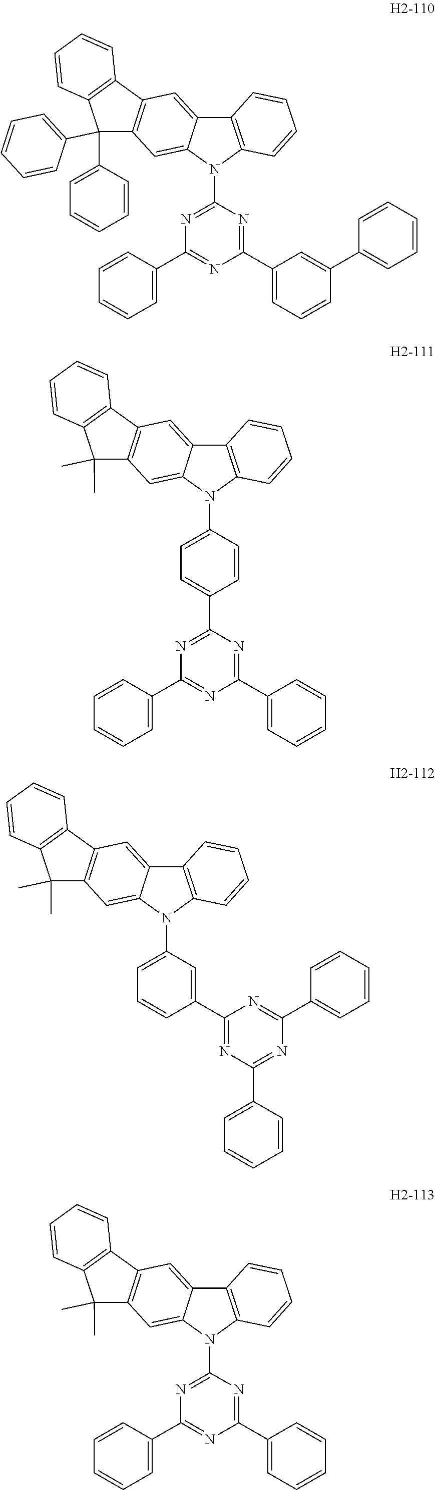 [SMILES: c1ccc(-c2cccc(-c3nc(-c4ccccc4)nc(-n4c5ccccc5c5cc6c(cc54)C(c4ccccc4)(c4ccccc4)c4ccccc4-6)n3)c2)cc1 ; CC1(C)c2ccccc2-c2cc3c4ccccc4n(-c4ccc(-c5nc(-c6ccccc6)nc(-c6ccccc6)n5)cc4)c3cc21 ; CC1(C)c2ccccc2-c2cc3c4ccccc4n(-c4cccc(-c5nc(-c6ccccc6)nc(-c6ccccc6)n5)c4)c3cc21 ; CC1(C)c2ccccc2-c2cc3c4ccccc4n(-c4nc(-c5ccccc5)nc(-c5ccccc5)n4)c3cc21] 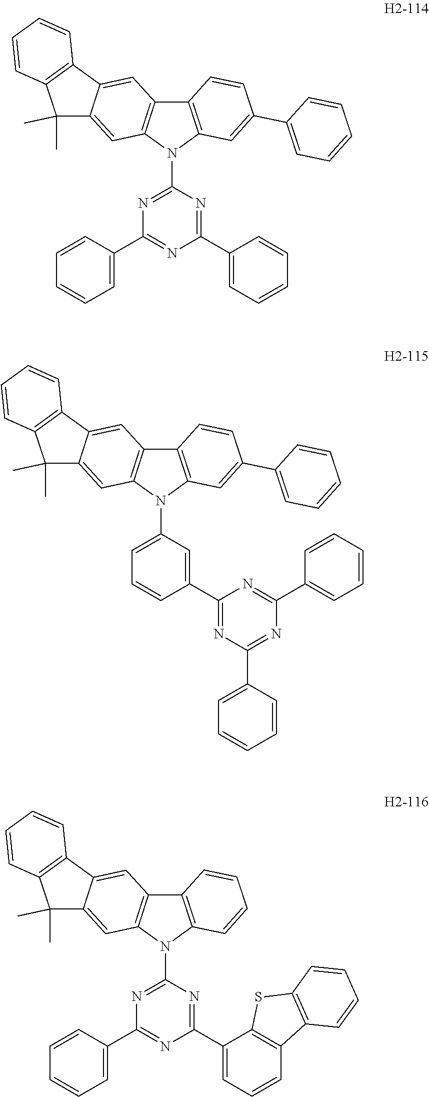 [SMILES: CC1(C)c2ccccc2-c2cc3c4ccc(-c5ccccc5)cc4n(-c4nc(-c5ccccc5)nc(-c5ccccc5)n4)c3cc21 ; CC1(C)c2ccccc2-c2cc3c4ccc(-c5ccccc5)cc4n(-c4cccc(-c5nc(-c6ccccc6)nc(-c6ccccc6)n5)c4)c3cc21 ; CC1(C)c2ccccc2-c2cc3c4ccccc4n(-c4nc(-c5ccccc5)nc(-c5cccc6c5sc5ccccc56)n4)c3cc21] 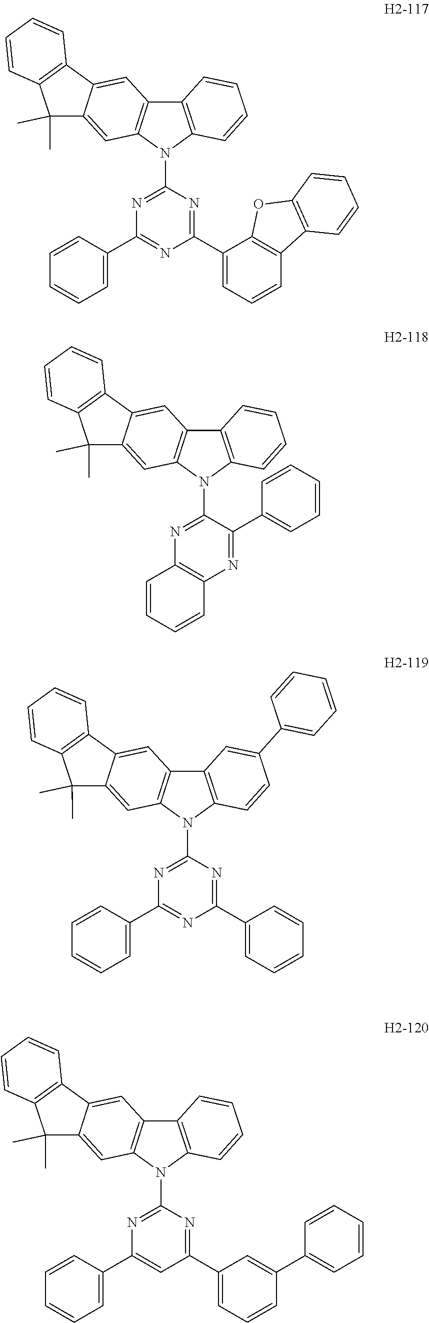 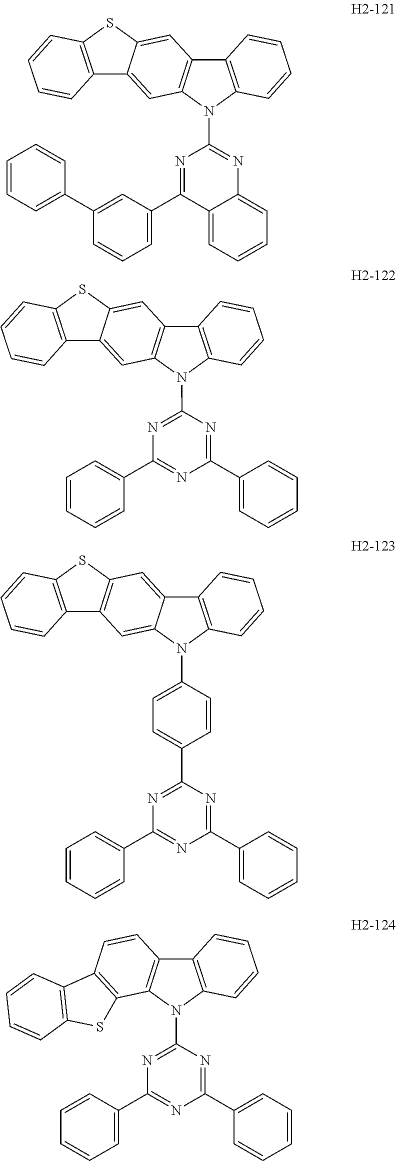 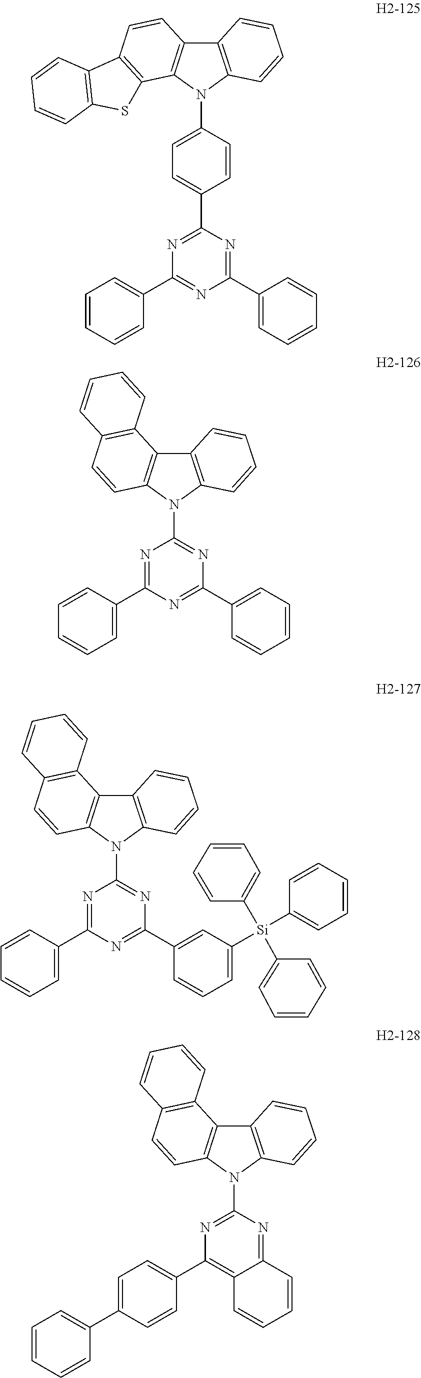 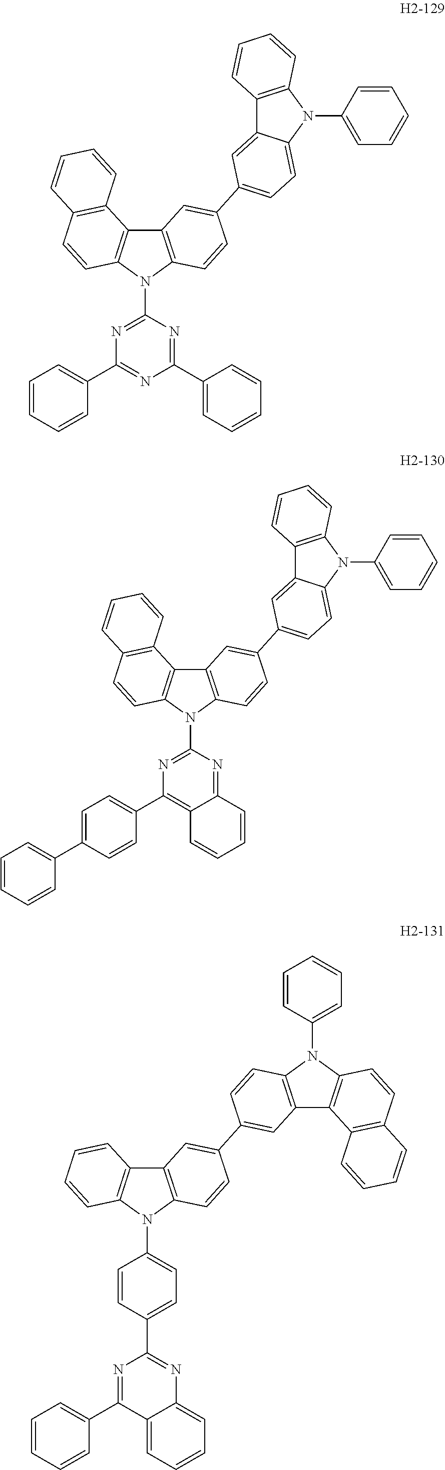 [SMILES: c1ccc(-c2nc(-c3ccccc3)nc(-n3c4ccc(-c5ccc6c(c5)c5ccccc5n6-c5ccccc5)cc4c4c5ccccc5ccc43)n2)cc1 ; c1ccc(-c2ccc(-c3nc(-n4c5ccc(-c6ccc7c(c6)c6ccccc6n7-c6ccccc6)cc5c5c6ccccc6ccc54)nc4ccccc34)cc2)cc1 ; c1ccc(-c2nc(-c3ccc(-n4c5ccccc5c5cc(-c6ccc7c(c6)c6c8ccccc8ccc6n7-c6ccccc6)ccc54)cc3)nc3ccccc23)cc1] 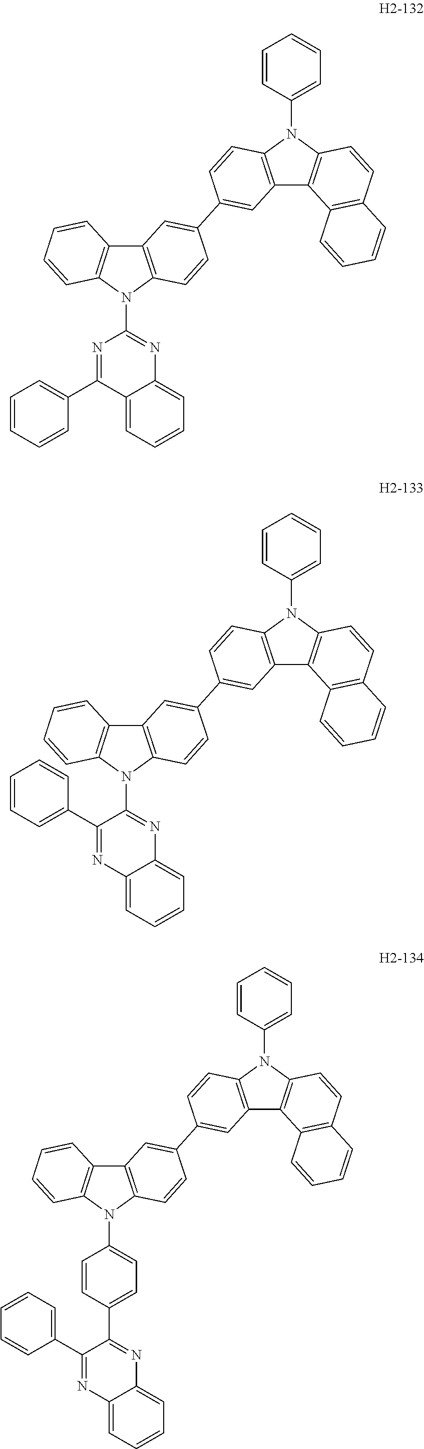 [SMILES: c1ccc(-c2nc(-n3c4ccccc4c4cc(-c5ccc6c(c5)c5c7ccccc7ccc5n6-c5ccccc5)ccc43)nc3ccccc23)cc1 ; c1ccc(-c2nc3ccccc3nc2-n2c3ccccc3c3cc(-c4ccc5c(c4)c4c6ccccc6ccc4n5-c4ccccc4)ccc32)cc1 ; c1ccc(-c2nc3ccccc3nc2-c2ccc(-n3c4ccccc4c4cc(-c5ccc6c(c5)c5c7ccccc7ccc5n6-c5ccccc5)ccc43)cc2)cc1] 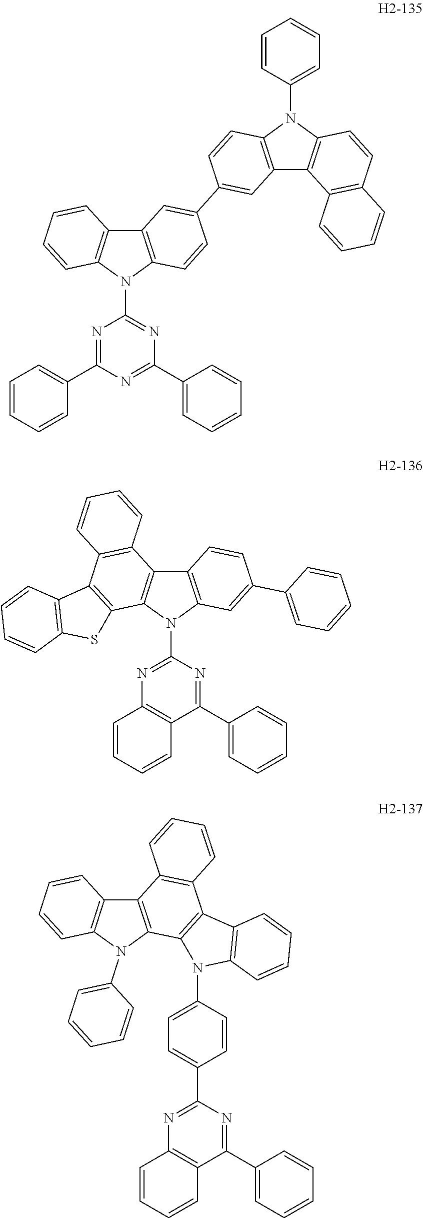 [SMILES: c1ccc(-c2nc(-c3ccccc3)nc(-n3c4ccccc4c4cc(-c5ccc6c(c5)c5c7ccccc7ccc5n6-c5ccccc5)ccc43)n2)cc1 ; c1ccc(-c2ccc3c4c5ccccc5c5c6ccccc6sc5c4n(-c4nc(-c5ccccc5)c5ccccc5n4)c3c2)cc1 ; c1ccc(-c2nc(-c3ccc(-n4c5ccccc5c5c6ccccc6c6c7ccccc7n(-c7ccccc7)c6c54)cc3)nc3ccccc23)cc1] 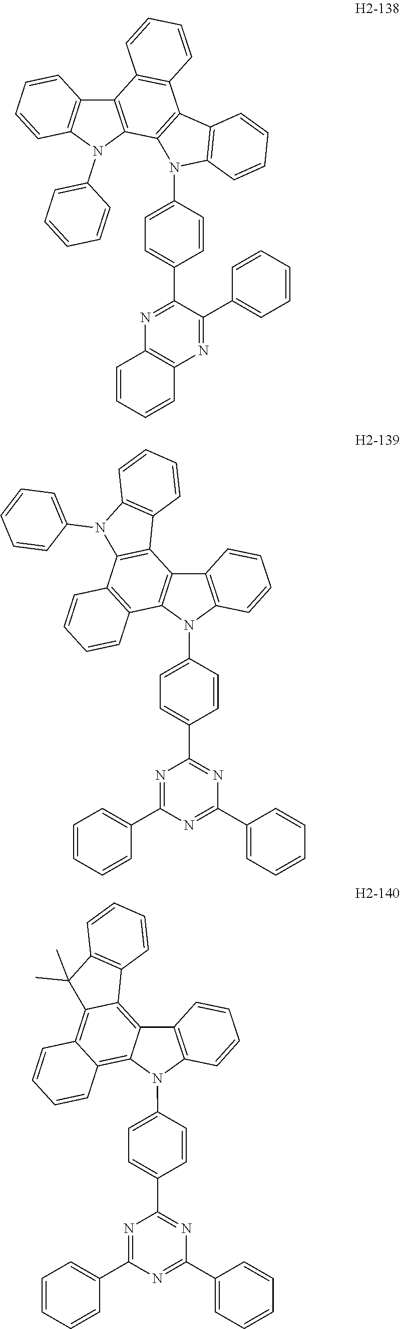 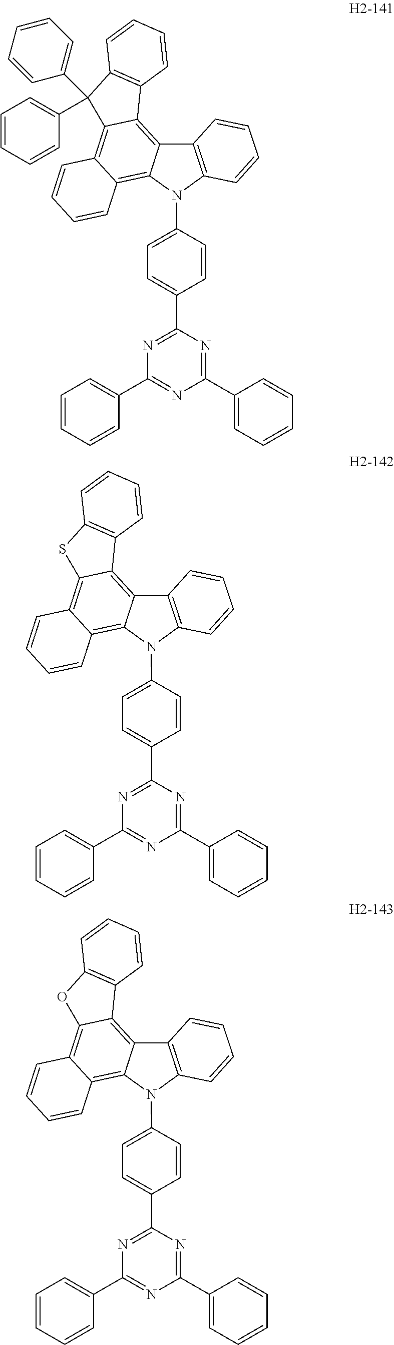 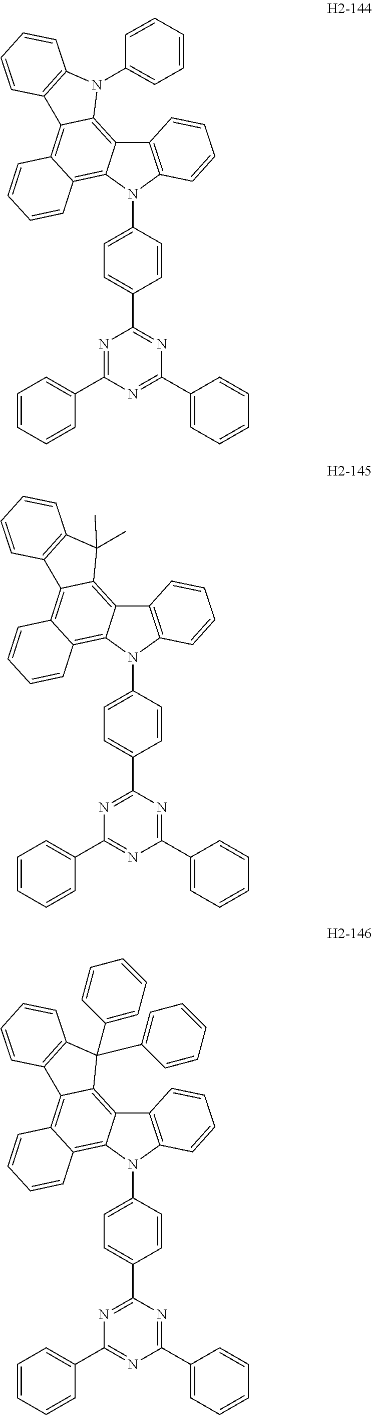 [SMILES: c1ccc(-c2nc(-c3ccccc3)nc(-c3ccc(-n4c5ccccc5c5c4c4ccccc4c4c6ccccc6n(-c6ccccc6)c45)cc3)n2)cc1 ; CC1(C)c2ccccc2-c2c1c1c3ccccc3n(-c3ccc(-c4nc(-c5ccccc5)nc(-c5ccccc5)n4)cc3)c1c1ccccc21 ; c1ccc(-c2nc(-c3ccccc3)nc(-c3ccc(-n4c5ccccc5c5c6c(c7ccccc7c54)-c4ccccc4C6(c4ccccc4)c4ccccc4)cc3)n2)cc1] 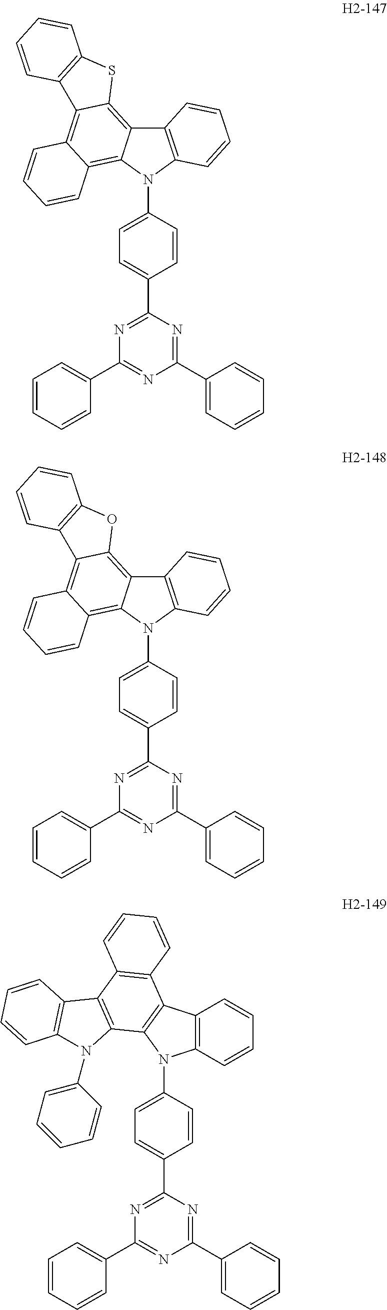 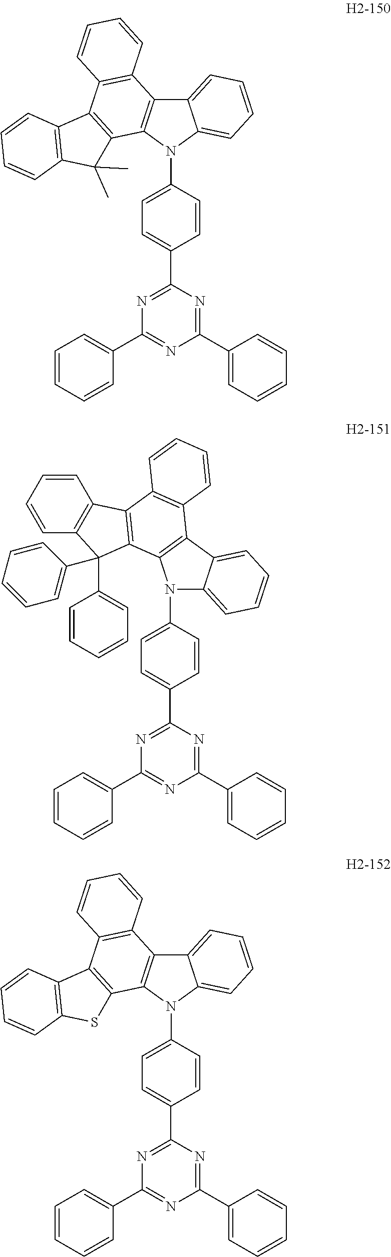 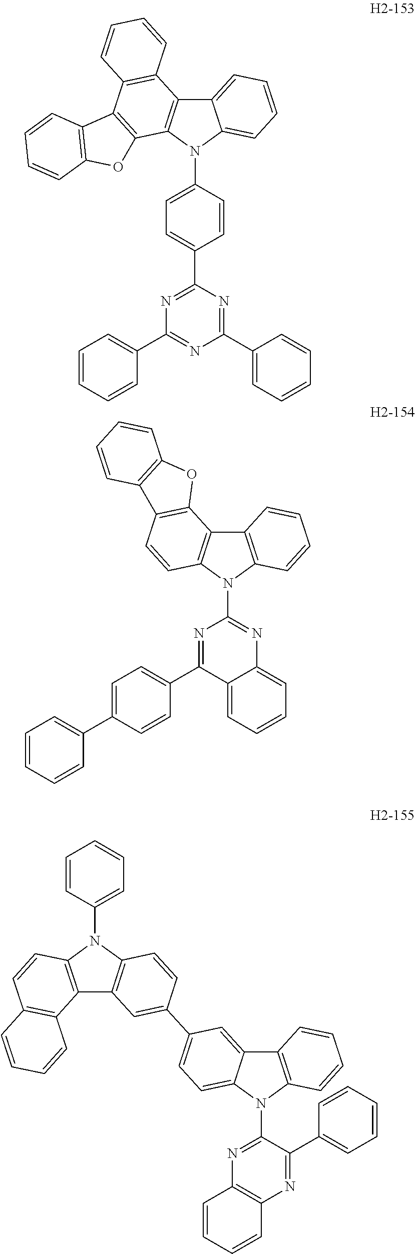 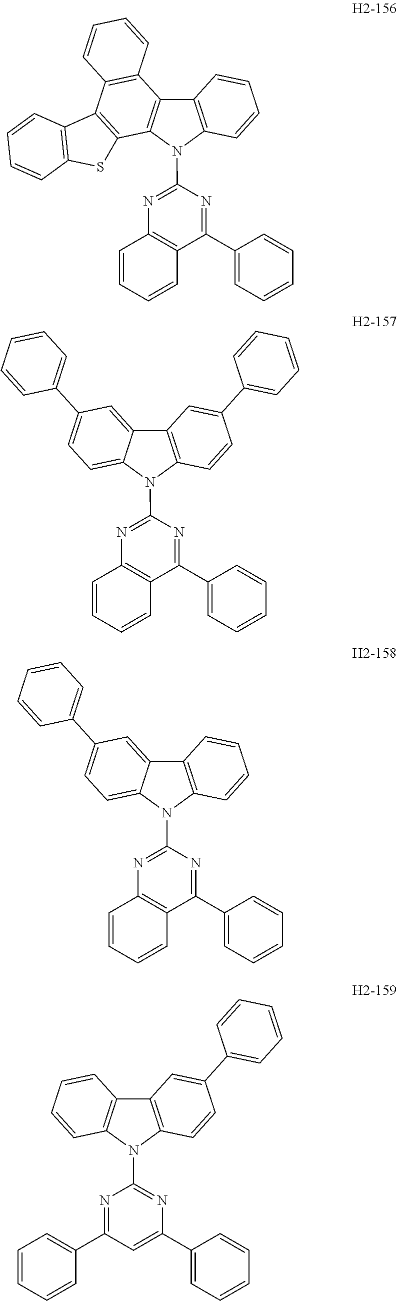 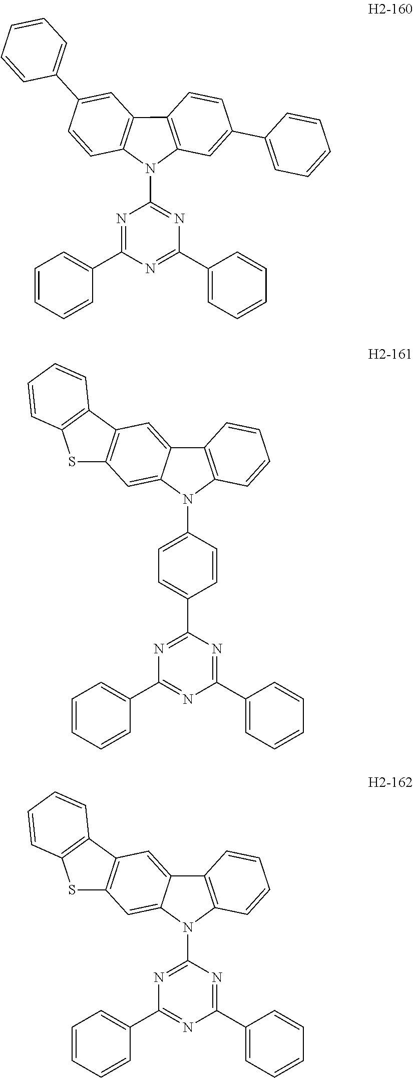 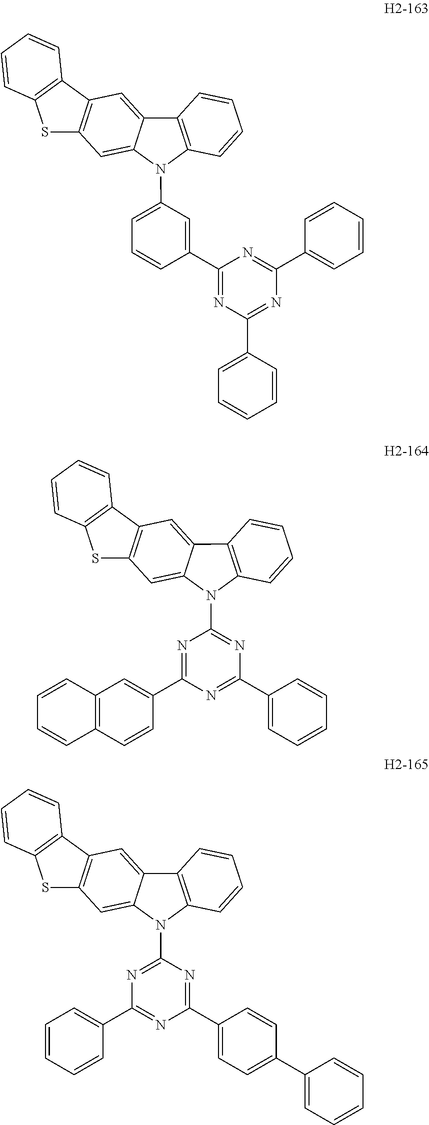 [SMILES: c1ccc(-c2nc(-c3ccccc3)nc(-c3cccc(-n4c5ccccc5c5cc6c(cc54)sc4ccccc46)c3)n2)cc1 ; c1ccc(-c2nc(-c3ccc4ccccc4c3)nc(-n3c4ccccc4c4cc5c(cc43)sc3ccccc35)n2)cc1 ; c1ccc(-c2ccc(-c3nc(-c4ccccc4)nc(-n4c5ccccc5c5cc6c(cc54)sc4ccccc46)n3)cc2)cc1] 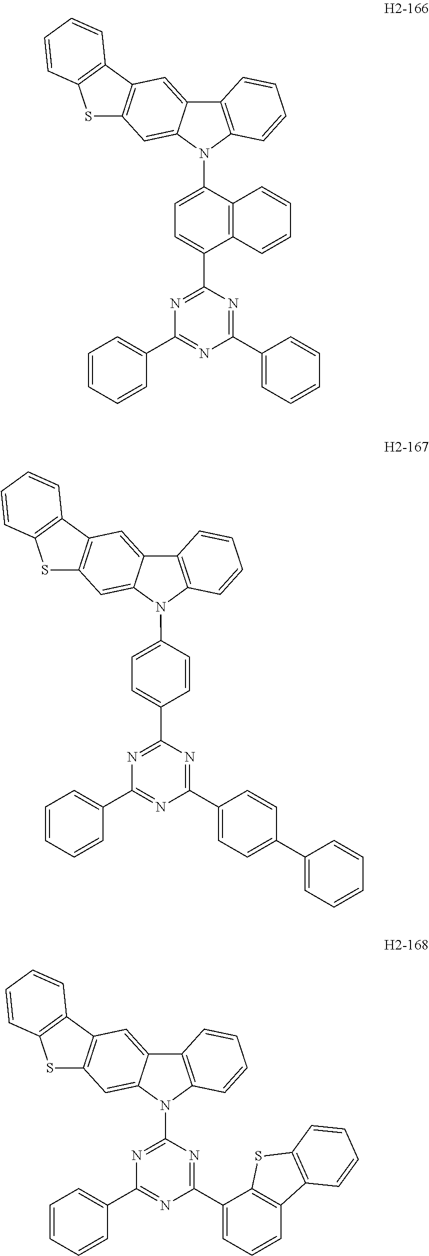 [SMILES: c1ccc(-c2nc(-c3ccccc3)nc(-c3ccc(-n4c5ccccc5c5cc6c(cc54)sc4ccccc46)c4ccccc34)n2)cc1 ; c1ccc(-c2ccc(-c3nc(-c4ccccc4)nc(-c4ccc(-n5c6ccccc6c6cc7c(cc65)sc5ccccc57)cc4)n3)cc2)cc1 ; c1ccc(-c2nc(-c3cccc4c3sc3ccccc34)nc(-n3c4ccccc4c4cc5c(cc43)sc3ccccc35)n2)cc1] 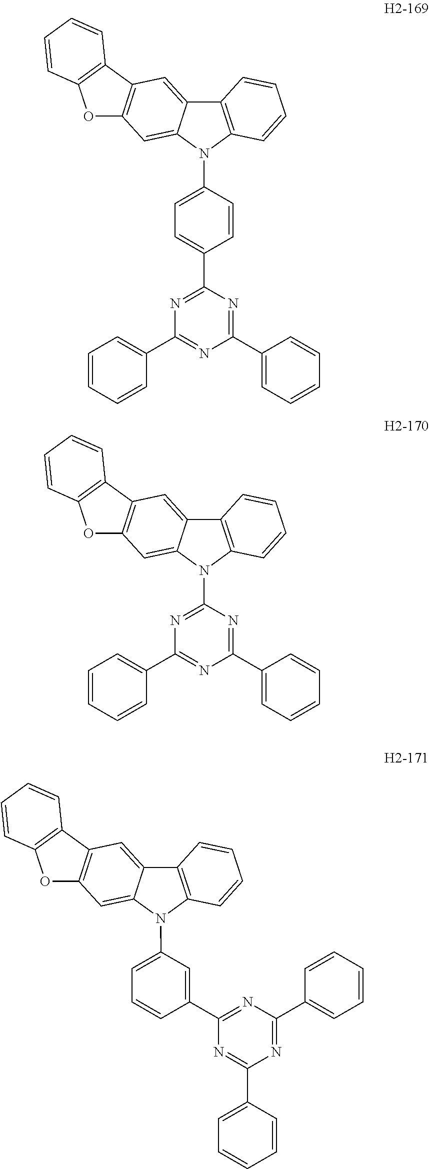 [SMILES: c1ccc(-c2nc(-c3ccccc3)nc(-c3ccc(-n4c5ccccc5c5cc6c(cc54)oc4ccccc46)cc3)n2)cc1 ; c1ccc(-c2nc(-c3ccccc3)nc(-n3c4ccccc4c4cc5c(cc43)oc3ccccc35)n2)cc1 ; c1ccc(-c2nc(-c3ccccc3)nc(-c3cccc(-n4c5ccccc5c5cc6c(cc54)oc4ccccc46)c3)n2)cc1] 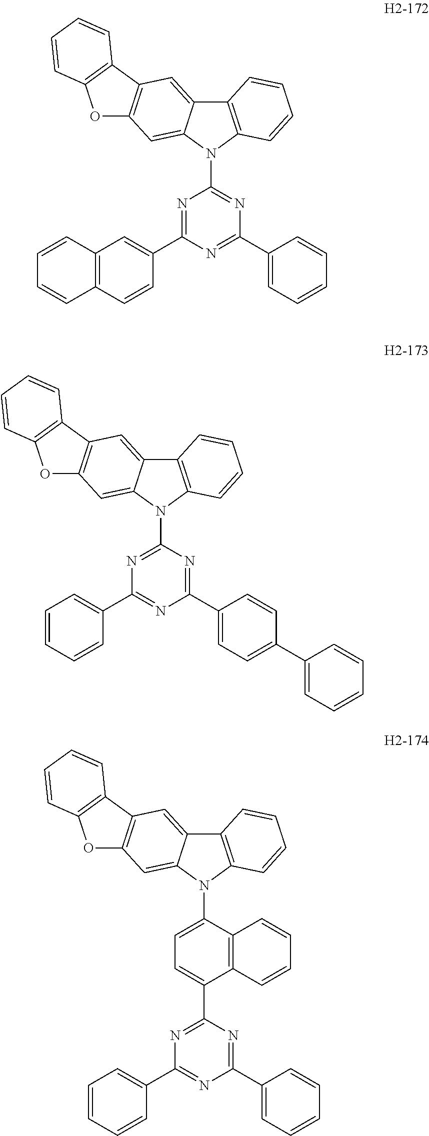 [SMILES: c1ccc(-c2nc(-c3ccc4ccccc4c3)nc(-n3c4ccccc4c4cc5c(cc43)oc3ccccc35)n2)cc1 ; c1ccc(-c2ccc(-c3nc(-c4ccccc4)nc(-n4c5ccccc5c5cc6c(cc54)oc4ccccc46)n3)cc2)cc1 ; c1ccc(-c2nc(-c3ccccc3)nc(-c3ccc(-n4c5ccccc5c5cc6c(cc54)oc4ccccc46)c4ccccc34)n2)cc1] 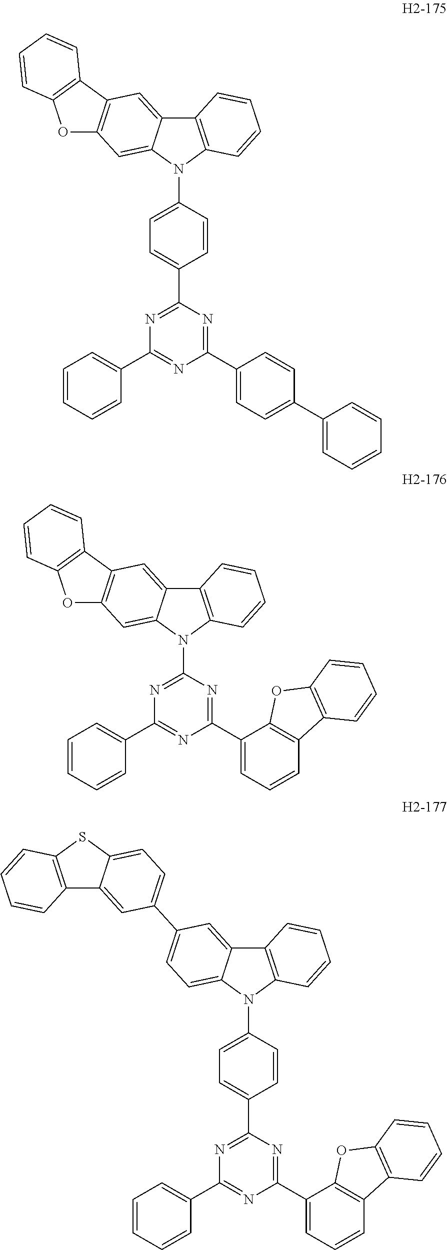 [SMILES: c1ccc(-c2ccc(-c3nc(-c4ccccc4)nc(-c4ccc(-n5c6ccccc6c6cc7c(cc65)oc5ccccc57)cc4)n3)cc2)cc1 ; c1ccc(-c2nc(-c3cccc4c3oc3ccccc34)nc(-n3c4ccccc4c4cc5c(cc43)oc3ccccc35)n2)cc1 ; c1ccc(-c2nc(-c3ccc(-n4c5ccccc5c5cc(-c6ccc7sc8ccccc8c7c6)ccc54)cc3)nc(-c3cccc4c3oc3ccccc34)n2)cc1] 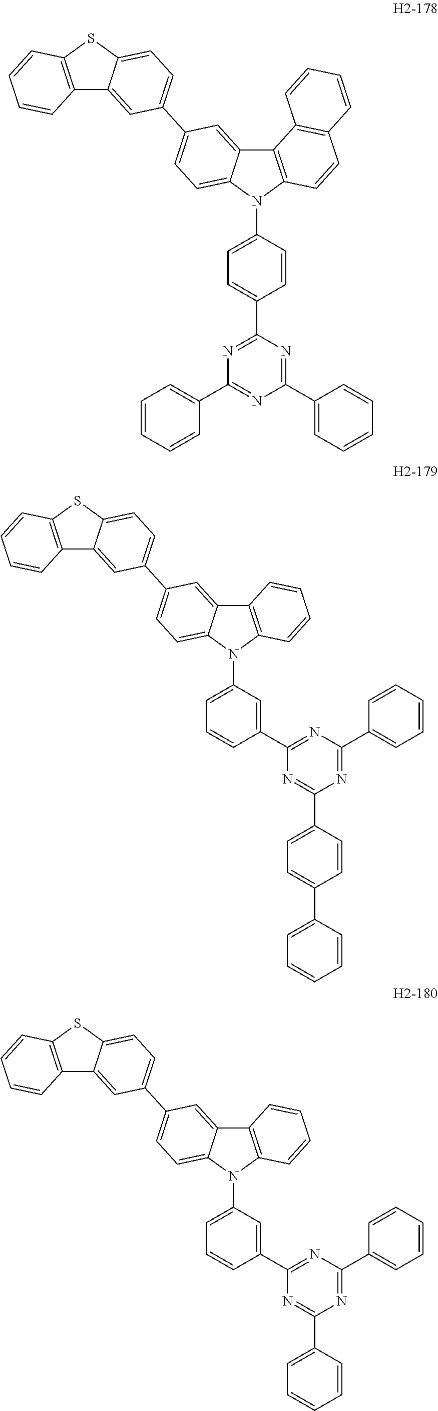 [SMILES: c1ccc(-c2nc(-c3ccccc3)nc(-c3ccc(-n4c5ccc(-c6ccc7sc8ccccc8c7c6)cc5c5c6ccccc6ccc54)cc3)n2)cc1 ; c1ccc(-c2ccc(-c3nc(-c4ccccc4)nc(-c4cccc(-n5c6ccccc6c6cc(-c7ccc8sc9ccccc9c8c7)ccc65)c4)n3)cc2)cc1 ; c1ccc(-c2nc(-c3ccccc3)nc(-c3cccc(-n4c5ccccc5c5cc(-c6ccc7sc8ccccc8c7c6)ccc54)c3)n2)cc1] 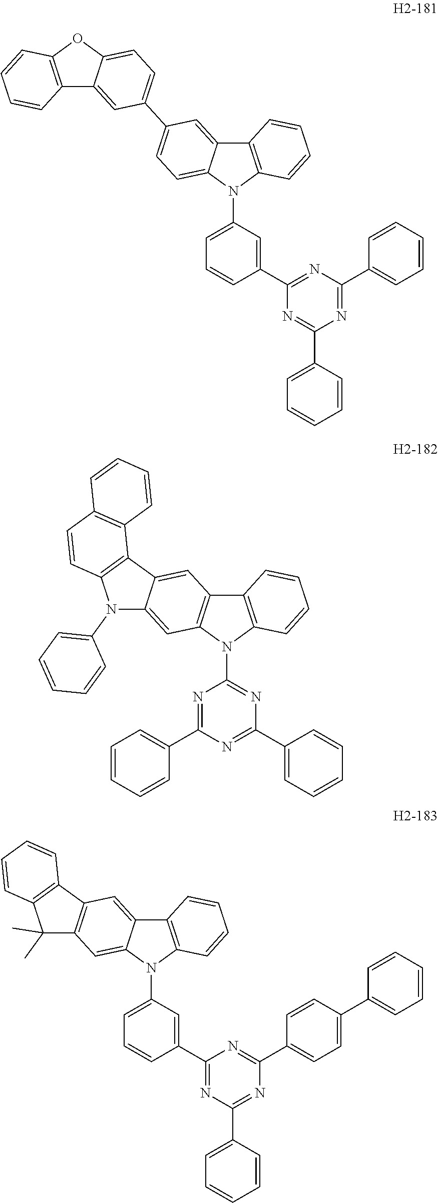 [SMILES: c1ccc(-c2nc(-c3ccccc3)nc(-c3cccc(-n4c5ccccc5c5cc(-c6ccc7oc8ccccc8c7c6)ccc54)c3)n2)cc1 ; c1ccc(-c2nc(-c3ccccc3)nc(-n3c4ccccc4c4cc5c6c7ccccc7ccc6n(-c6ccccc6)c5cc43)n2)cc1 ; CC1(C)c2ccccc2-c2cc3c4ccccc4n(-c4cccc(-c5nc(-c6ccccc6)nc(-c6ccc(-c7ccccc7)cc6)n5)c4)c3cc21] 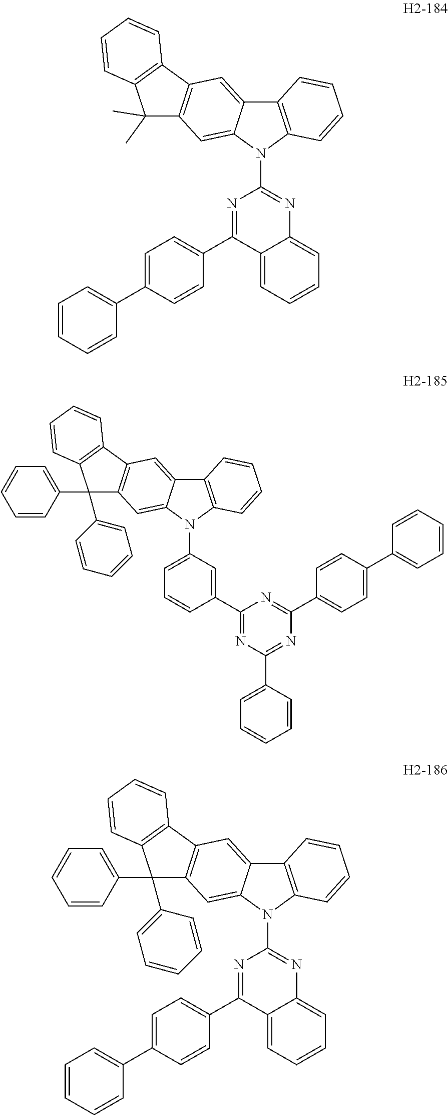 [SMILES: CC1(C)c2ccccc2-c2cc3c4ccccc4n(-c4nc(-c5ccc(-c6ccccc6)cc5)c5ccccc5n4)c3cc21 ; c1ccc(-c2ccc(-c3nc(-c4ccccc4)nc(-c4cccc(-n5c6ccccc6c6cc7c(cc65)C(c5ccccc5)(c5ccccc5)c5ccccc5-7)c4)n3)cc2)cc1 ; c1ccc(-c2ccc(-c3nc(-n4c5ccccc5c5cc6c(cc54)C(c4ccccc4)(c4ccccc4)c4ccccc4-6)nc4ccccc34)cc2)cc1] 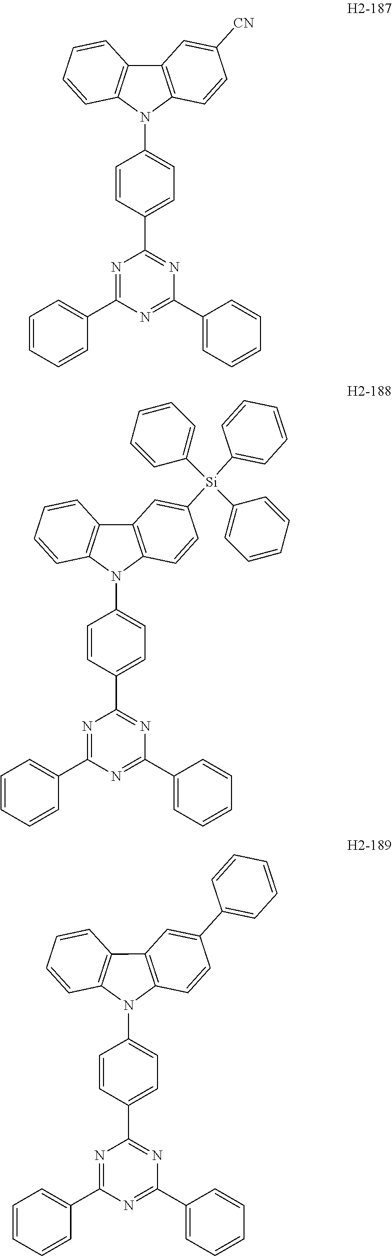 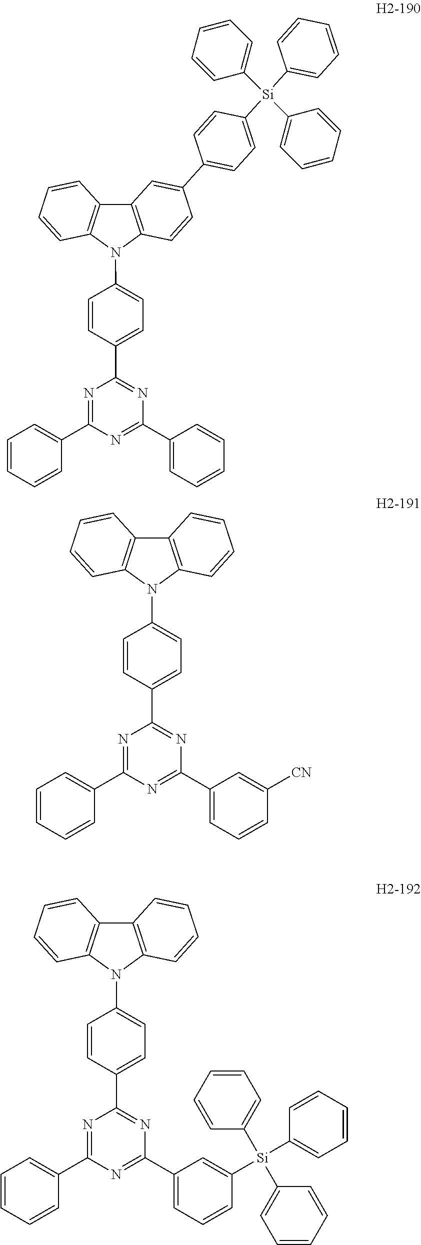 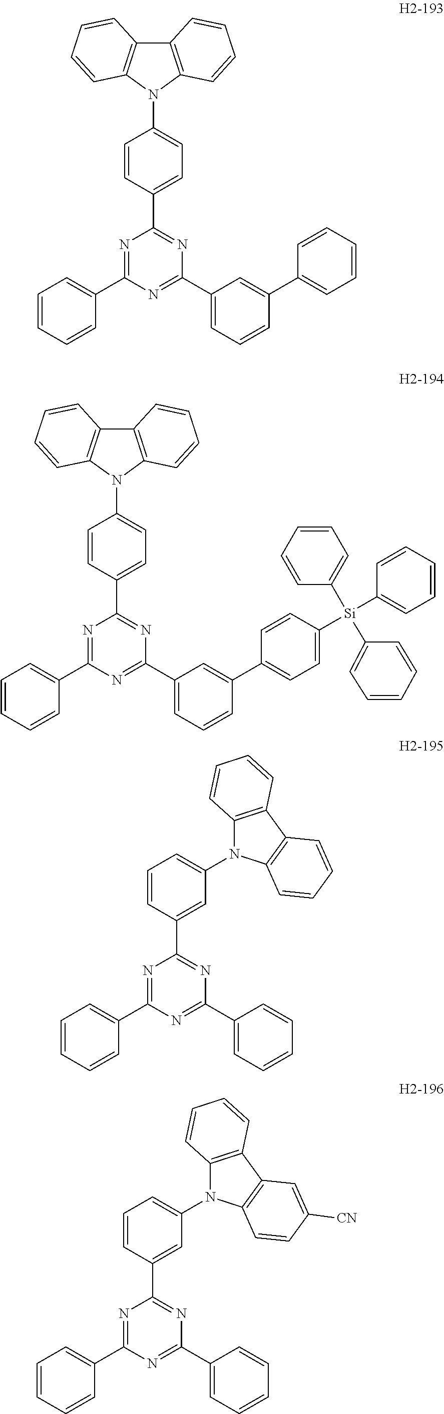 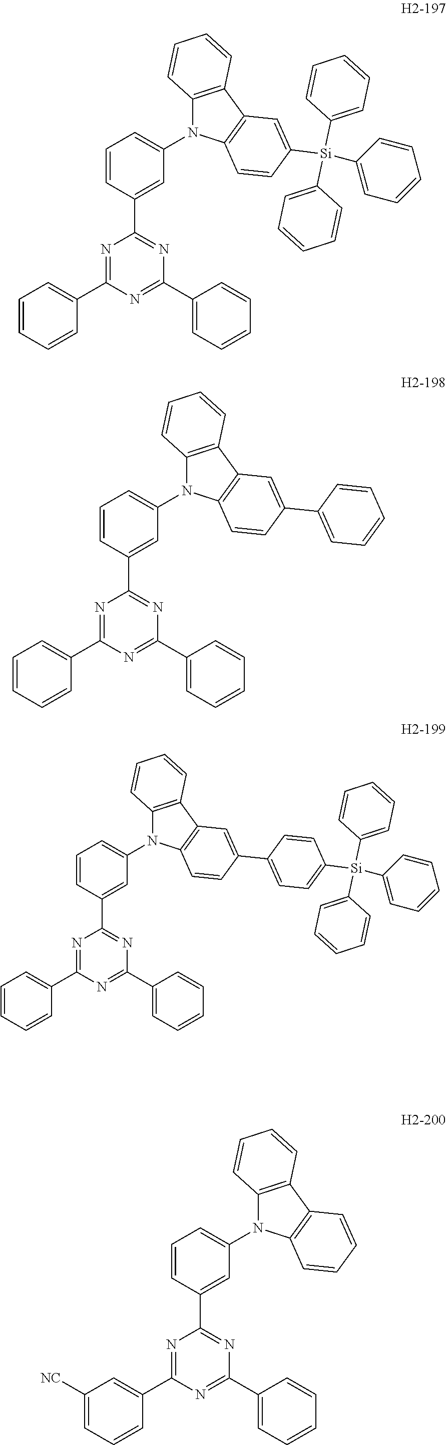 [SMILES: c1ccc(-c2nc(-c3ccccc3)nc(-c3cccc(-n4c5ccccc5c5cc([Si](c6ccccc6)(c6ccccc6)c6ccccc6)ccc54)c3)n2)cc1 ; c1ccc(-c2ccc3c(c2)c2ccccc2n3-c2cccc(-c3nc(-c4ccccc4)nc(-c4ccccc4)n3)c2)cc1 ; c1ccc(-c2nc(-c3ccccc3)nc(-c3cccc(-n4c5ccccc5c5cc(-c6ccc([Si](c7ccccc7)(c7ccccc7)c7ccccc7)cc6)ccc54)c3)n2)cc1 ; N#Cc1cccc(-c2nc(-c3ccccc3)nc(-c3cccc(-n4c5ccccc5c5ccccc54)c3)n2)c1] 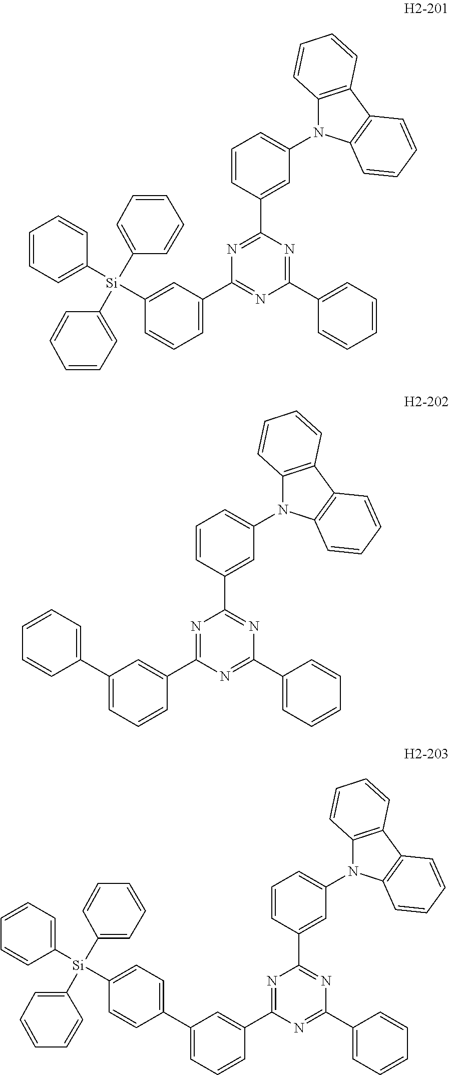 [SMILES: c1ccc(-c2nc(-c3cccc(-n4c5ccccc5c5ccccc54)c3)nc(-c3cccc([Si](c4ccccc4)(c4ccccc4)c4ccccc4)c3)n2)cc1 ; c1ccc(-c2cccc(-c3nc(-c4ccccc4)nc(-c4cccc(-n5c6ccccc6c6ccccc65)c4)n3)c2)cc1 ; c1ccc(-c2nc(-c3cccc(-c4ccc([Si](c5ccccc5)(c5ccccc5)c5ccccc5)cc4)c3)nc(-c3cccc(-n4c5ccccc5c5ccccc54)c3)n2)cc1] 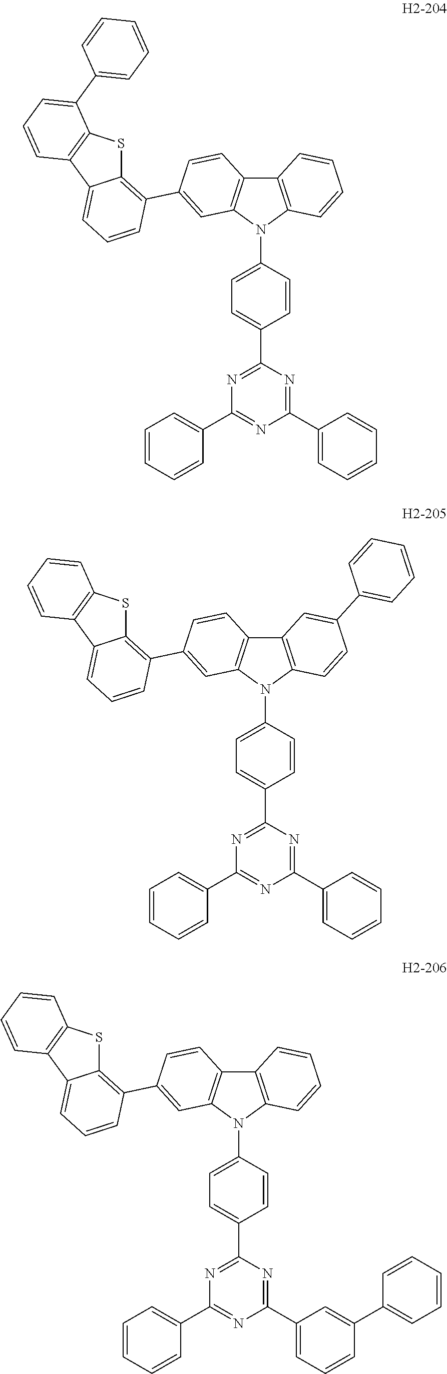 [SMILES: c1ccc(-c2nc(-c3ccccc3)nc(-c3ccc(-n4c5ccccc5c5ccc(-c6cccc7c6sc6c(-c8ccccc8)cccc67)cc54)cc3)n2)cc1 ; c1ccc(-c2ccc3c(c2)c2ccc(-c4cccc5c4sc4ccccc45)cc2n3-c2ccc(-c3nc(-c4ccccc4)nc(-c4ccccc4)n3)cc2)cc1 ; c1ccc(-c2cccc(-c3nc(-c4ccccc4)nc(-c4ccc(-n5c6ccccc6c6ccc(-c7cccc8c7sc7ccccc78)cc65)cc4)n3)c2)cc1] 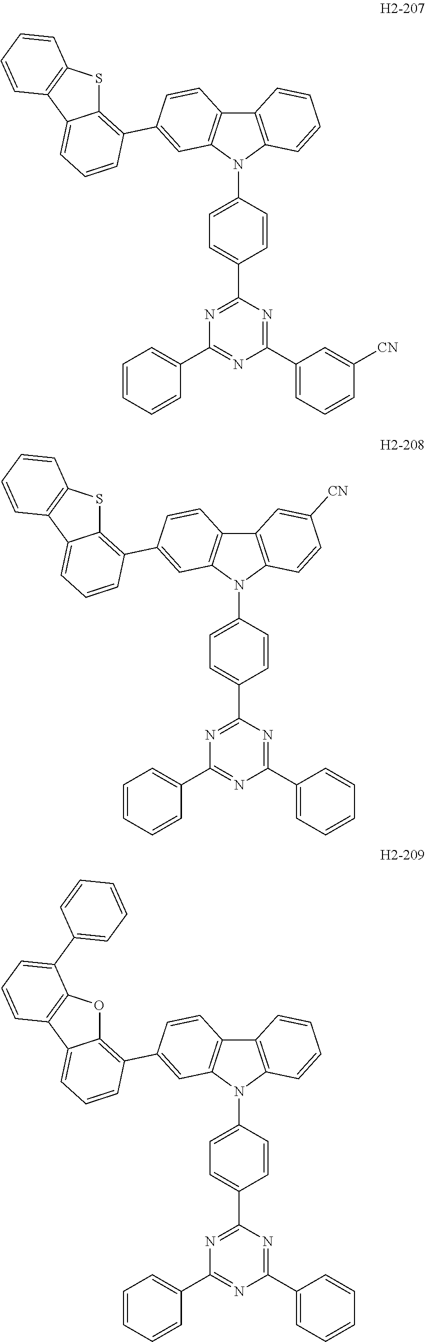 [SMILES: N#Cc1cccc(-c2nc(-c3ccccc3)nc(-c3ccc(-n4c5ccccc5c5ccc(-c6cccc7c6sc6ccccc67)cc54)cc3)n2)c1 ; N#Cc1ccc2c(c1)c1ccc(-c3cccc4c3sc3ccccc34)cc1n2-c1ccc(-c2nc(-c3ccccc3)nc(-c3ccccc3)n2)cc1 ; c1ccc(-c2nc(-c3ccccc3)nc(-c3ccc(-n4c5ccccc5c5ccc(-c6cccc7c6oc6c(-c8ccccc8)cccc67)cc54)cc3)n2)cc1] 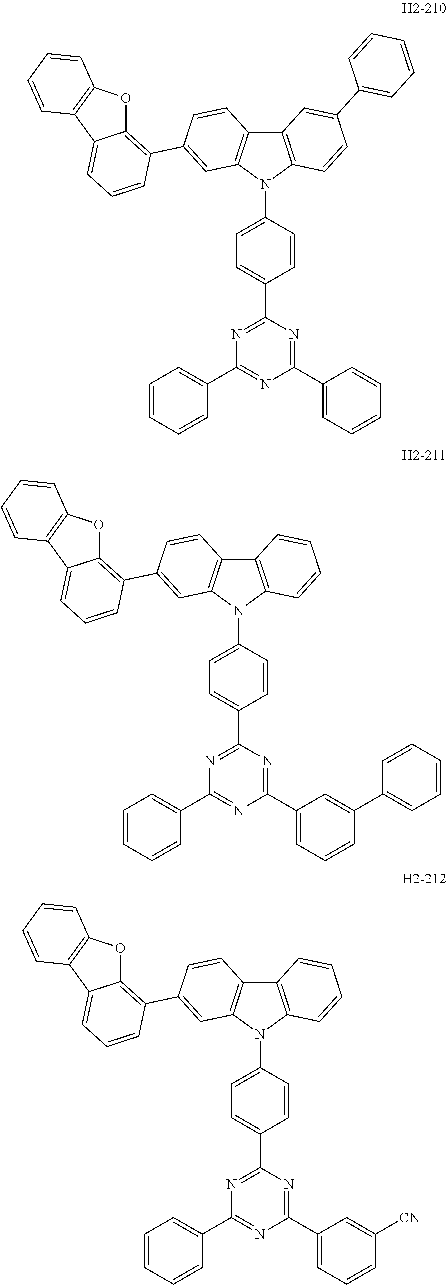 [SMILES: c1ccc(-c2ccc3c(c2)c2ccc(-c4cccc5c4oc4ccccc45)cc2n3-c2ccc(-c3nc(-c4ccccc4)nc(-c4ccccc4)n3)cc2)cc1 ; c1ccc(-c2cccc(-c3nc(-c4ccccc4)nc(-c4ccc(-n5c6ccccc6c6ccc(-c7cccc8c7oc7ccccc78)cc65)cc4)n3)c2)cc1 ; N#Cc1cccc(-c2nc(-c3ccccc3)nc(-c3ccc(-n4c5ccccc5c5ccc(-c6cccc7c6oc6ccccc67)cc54)cc3)n2)c1] 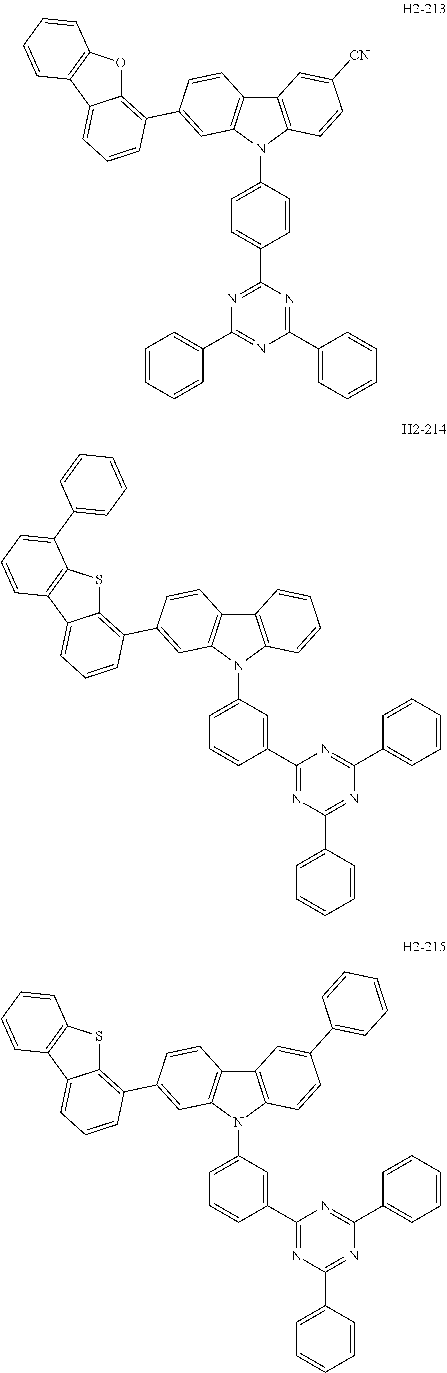 [SMILES: N#Cc1ccc2c(c1)c1ccc(-c3cccc4c3oc3ccccc34)cc1n2-c1ccc(-c2nc(-c3ccccc3)nc(-c3ccccc3)n2)cc1 ; c1ccc(-c2nc(-c3ccccc3)nc(-c3cccc(-n4c5ccccc5c5ccc(-c6cccc7c6sc6c(-c8ccccc8)cccc67)cc54)c3)n2)cc1 ; c1ccc(-c2ccc3c(c2)c2ccc(-c4cccc5c4sc4ccccc45)cc2n3-c2cccc(-c3nc(-c4ccccc4)nc(-c4ccccc4)n3)c2)cc1] 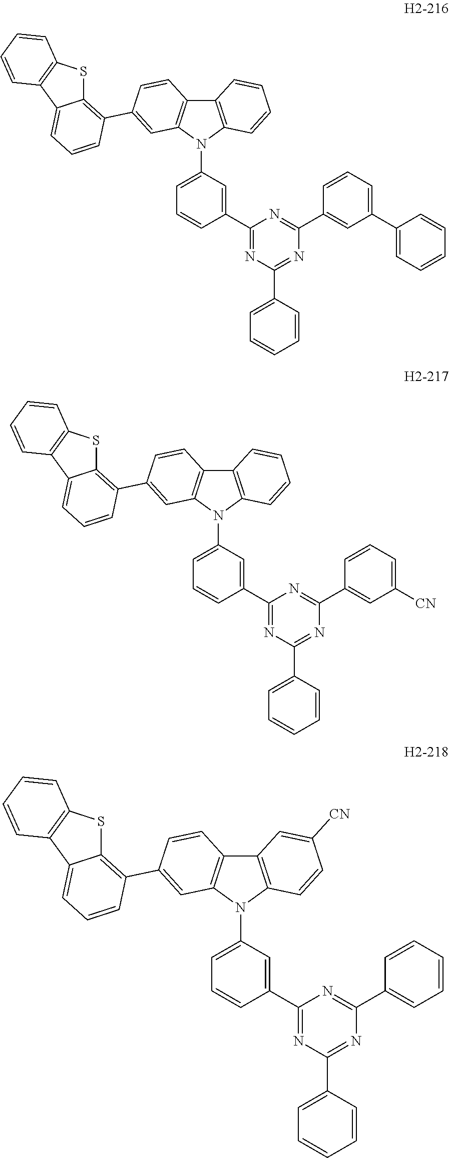 [SMILES: c1ccc(-c2cccc(-c3nc(-c4ccccc4)nc(-c4cccc(-n5c6ccccc6c6ccc(-c7cccc8c7sc7ccccc78)cc65)c4)n3)c2)cc1 ; N#Cc1cccc(-c2nc(-c3ccccc3)nc(-c3cccc(-n4c5ccccc5c5ccc(-c6cccc7c6sc6ccccc67)cc54)c3)n2)c1 ; N#Cc1ccc2c(c1)c1ccc(-c3cccc4c3sc3ccccc34)cc1n2-c1cccc(-c2nc(-c3ccccc3)nc(-c3ccccc3)n2)c1] 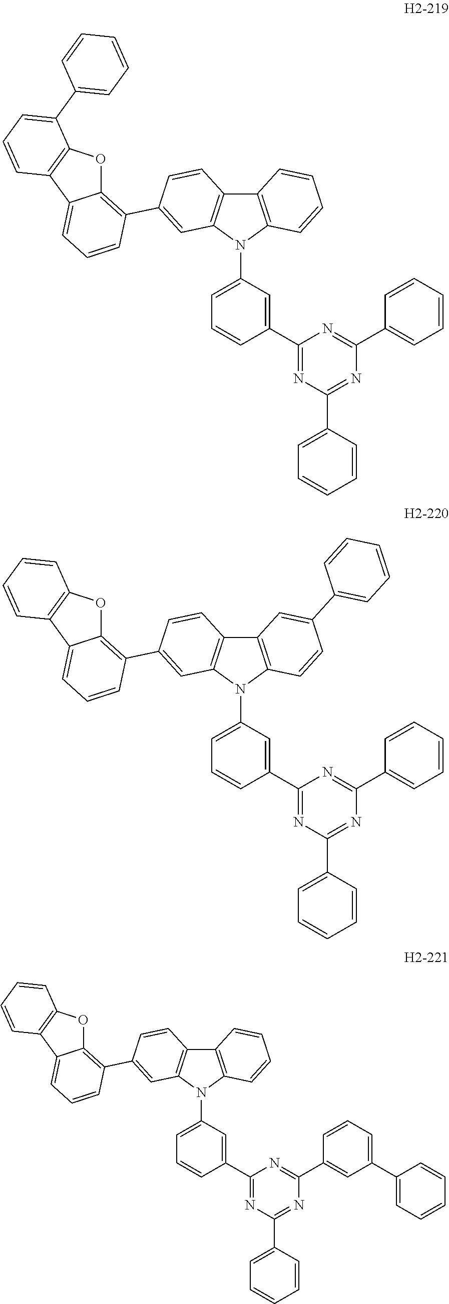 [SMILES: c1ccc(-c2nc(-c3ccccc3)nc(-c3cccc(-n4c5ccccc5c5ccc(-c6cccc7c6oc6c(-c8ccccc8)cccc67)cc54)c3)n2)cc1 ; c1ccc(-c2ccc3c(c2)c2ccc(-c4cccc5c4oc4ccccc45)cc2n3-c2cccc(-c3nc(-c4ccccc4)nc(-c4ccccc4)n3)c2)cc1 ; c1ccc(-c2cccc(-c3nc(-c4ccccc4)nc(-c4cccc(-n5c6ccccc6c6ccc(-c7cccc8c7oc7ccccc78)cc65)c4)n3)c2)cc1] 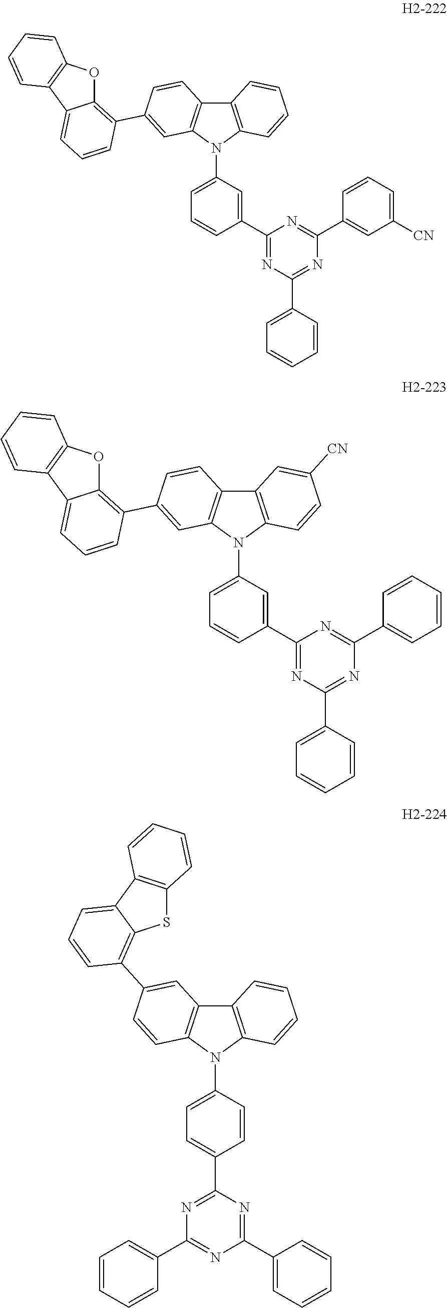 [SMILES: N#Cc1cccc(-c2nc(-c3ccccc3)nc(-c3cccc(-n4c5ccccc5c5ccc(-c6cccc7c6oc6ccccc67)cc54)c3)n2)c1 ; N#Cc1ccc2c(c1)c1ccc(-c3cccc4c3oc3ccccc34)cc1n2-c1cccc(-c2nc(-c3ccccc3)nc(-c3ccccc3)n2)c1 ; c1ccc(-c2nc(-c3ccccc3)nc(-c3ccc(-n4c5ccccc5c5cc(-c6cccc7c6sc6ccccc67)ccc54)cc3)n2)cc1] 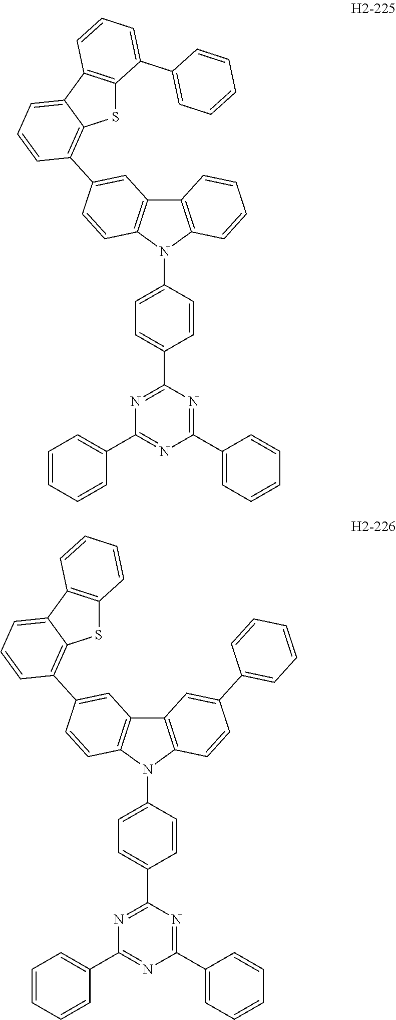 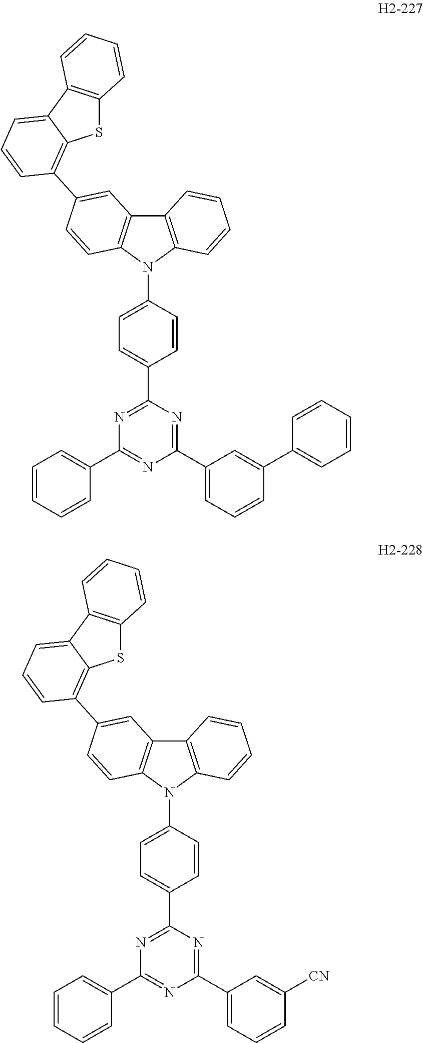 [SMILES: c1ccc(-c2cccc(-c3nc(-c4ccccc4)nc(-c4ccc(-n5c6ccccc6c6cc(-c7cccc8c7sc7ccccc78)ccc65)cc4)n3)c2)cc1 ; N#Cc1cccc(-c2nc(-c3ccccc3)nc(-c3ccc(-n4c5ccccc5c5cc(-c6cccc7c6sc6ccccc67)ccc54)cc3)n2)c1] 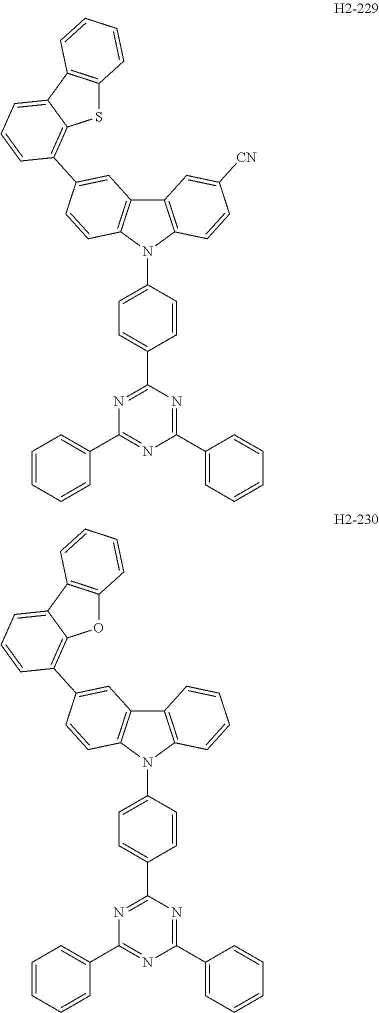 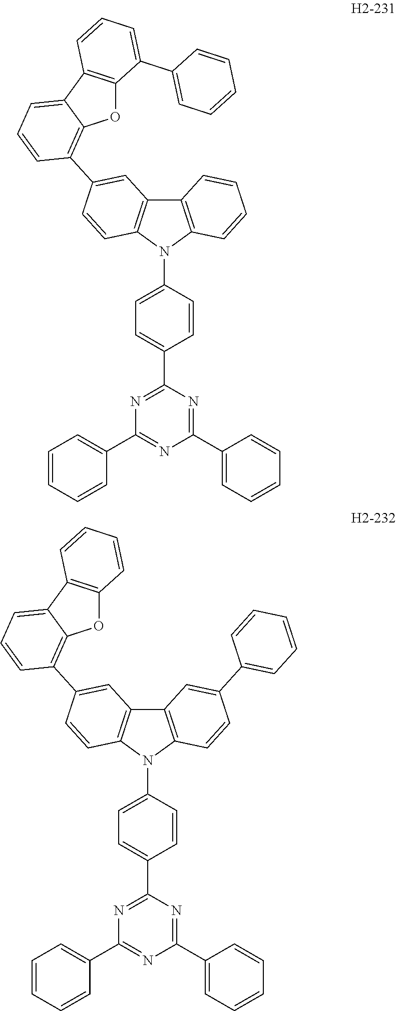 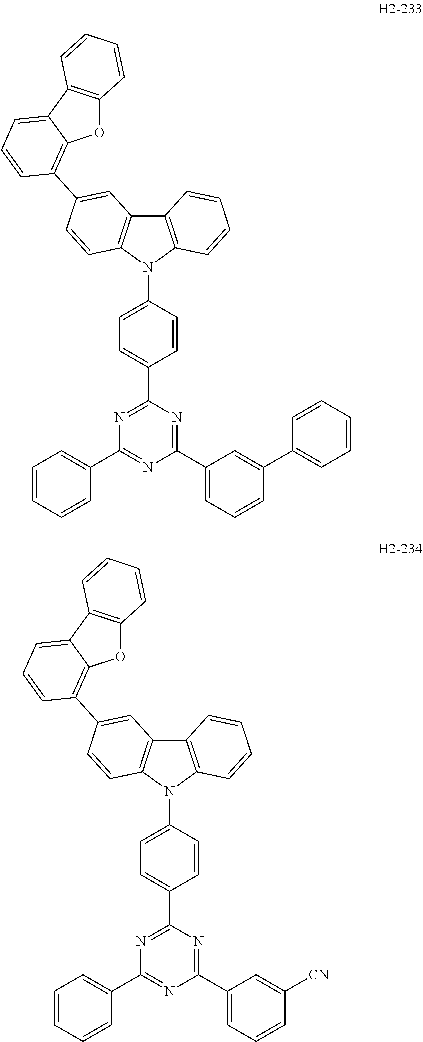 [SMILES: c1ccc(-c2cccc(-c3nc(-c4ccccc4)nc(-c4ccc(-n5c6ccccc6c6cc(-c7cccc8c7oc7ccccc78)ccc65)cc4)n3)c2)cc1 ; N#Cc1cccc(-c2nc(-c3ccccc3)nc(-c3ccc(-n4c5ccccc5c5cc(-c6cccc7c6oc6ccccc67)ccc54)cc3)n2)c1] 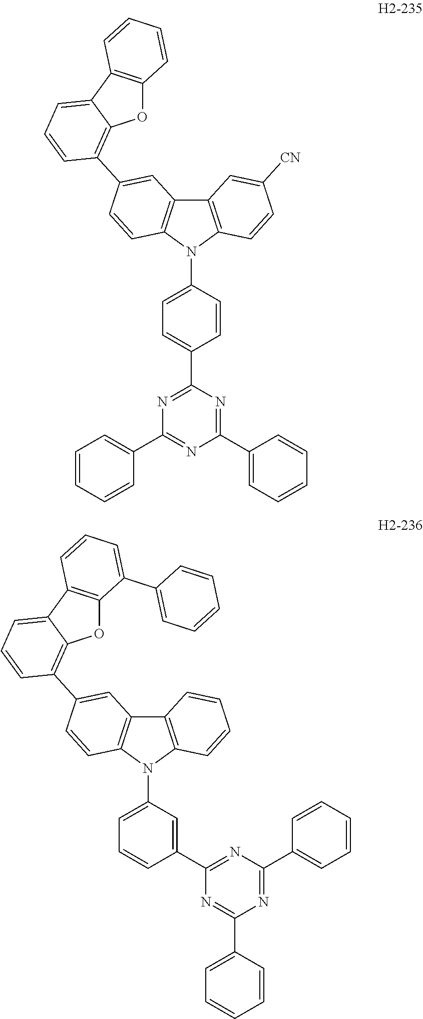 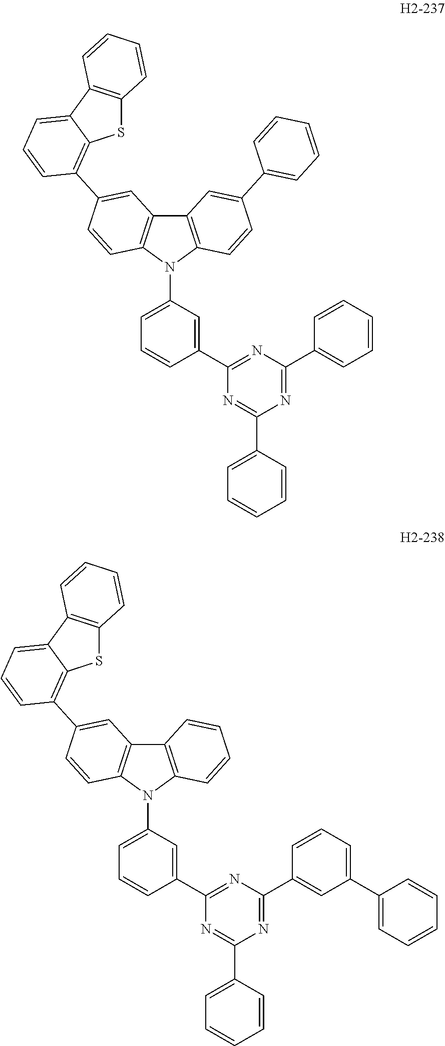 [SMILES: c1ccc(-c2ccc3c(c2)c2cc(-c4cccc5c4sc4ccccc45)ccc2n3-c2cccc(-c3nc(-c4ccccc4)nc(-c4ccccc4)n3)c2)cc1 ; c1ccc(-c2cccc(-c3nc(-c4ccccc4)nc(-c4cccc(-n5c6ccccc6c6cc(-c7cccc8c7sc7ccccc78)ccc65)c4)n3)c2)cc1] 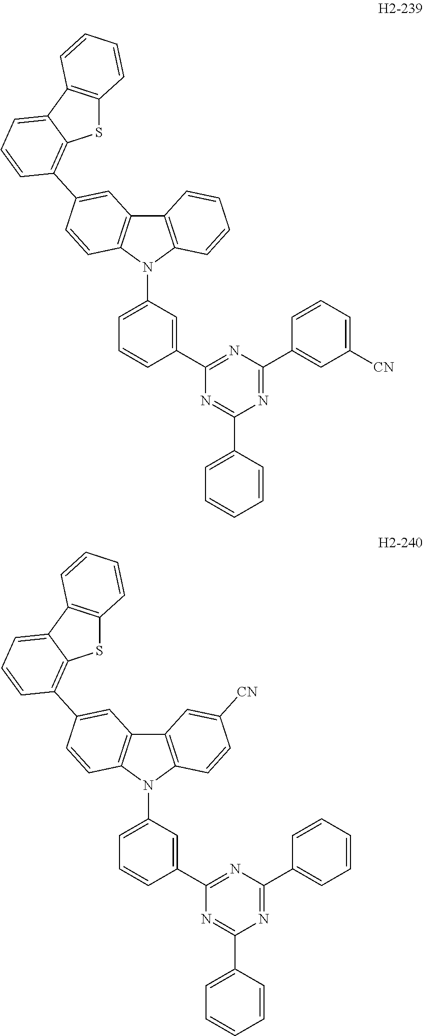 [SMILES: N#Cc1cccc(-c2nc(-c3ccccc3)nc(-c3cccc(-n4c5ccccc5c5cc(-c6cccc7c6sc6ccccc67)ccc54)c3)n2)c1 ; N#Cc1ccc2c(c1)c1cc(-c3cccc4c3sc3ccccc34)ccc1n2-c1cccc(-c2nc(-c3ccccc3)nc(-c3ccccc3)n2)c1] 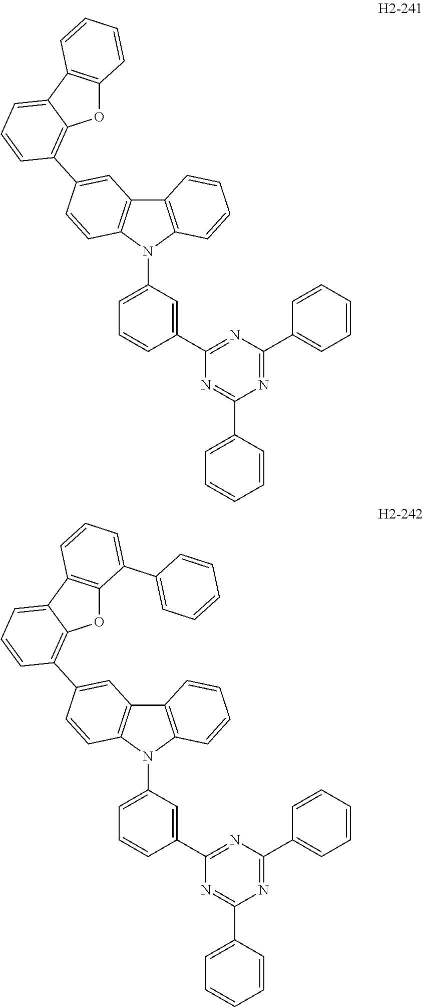 [SMILES: c1ccc(-c2nc(-c3ccccc3)nc(-c3cccc(-n4c5ccccc5c5cc(-c6cccc7c6oc6ccccc67)ccc54)c3)n2)cc1 ; c1ccc(-c2nc(-c3ccccc3)nc(-c3cccc(-n4c5ccccc5c5cc(-c6cccc7c6oc6c(-c8ccccc8)cccc67)ccc54)c3)n2)cc1] 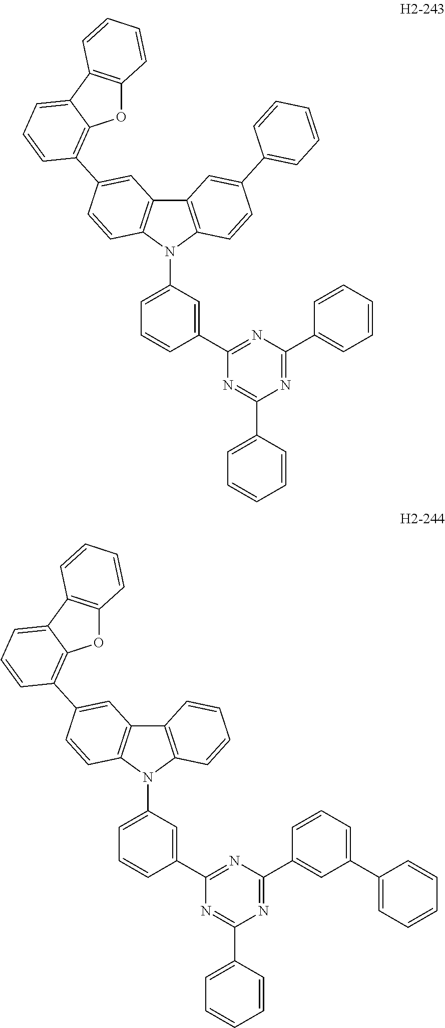 [SMILES: c1ccc(-c2ccc3c(c2)c2cc(-c4cccc5c4oc4ccccc45)ccc2n3-c2cccc(-c3nc(-c4ccccc4)nc(-c4ccccc4)n3)c2)cc1 ; c1ccc(-c2cccc(-c3nc(-c4ccccc4)nc(-c4cccc(-n5c6ccccc6c6cc(-c7cccc8c7oc7ccccc78)ccc65)c4)n3)c2)cc1] 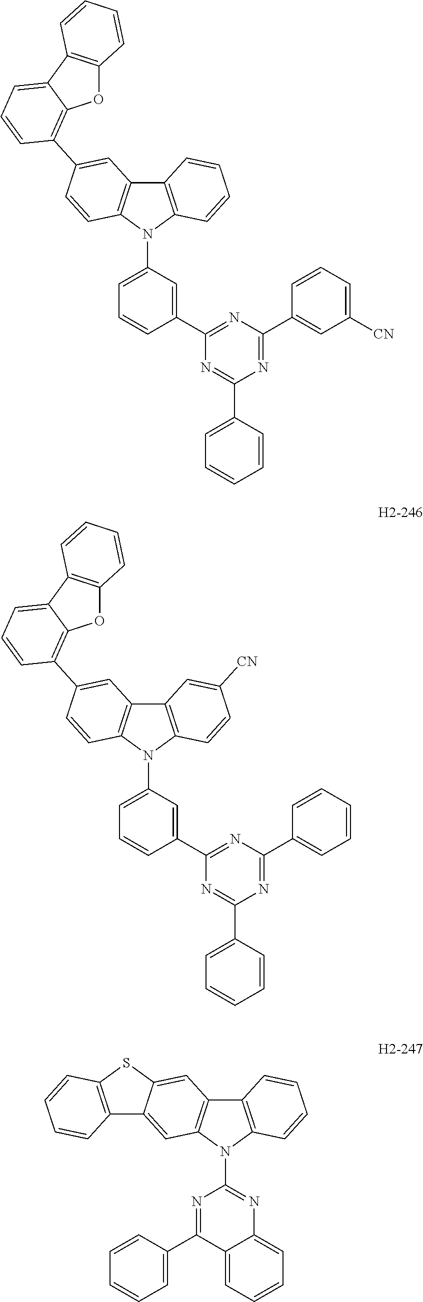 [SMILES: N#Cc1cccc(-c2nc(-c3ccccc3)nc(-c3cccc(-n4c5ccccc5c5cc(-c6cccc7c6oc6ccccc67)ccc54)c3)n2)c1 ; N#Cc1ccc2c(c1)c1cc(-c3cccc4c3oc3ccccc34)ccc1n2-c1cccc(-c2nc(-c3ccccc3)nc(-c3ccccc3)n2)c1 ; c1ccc(-c2nc(-n3c4ccccc4c4cc5sc6ccccc6c5cc43)nc3ccccc23)cc1] 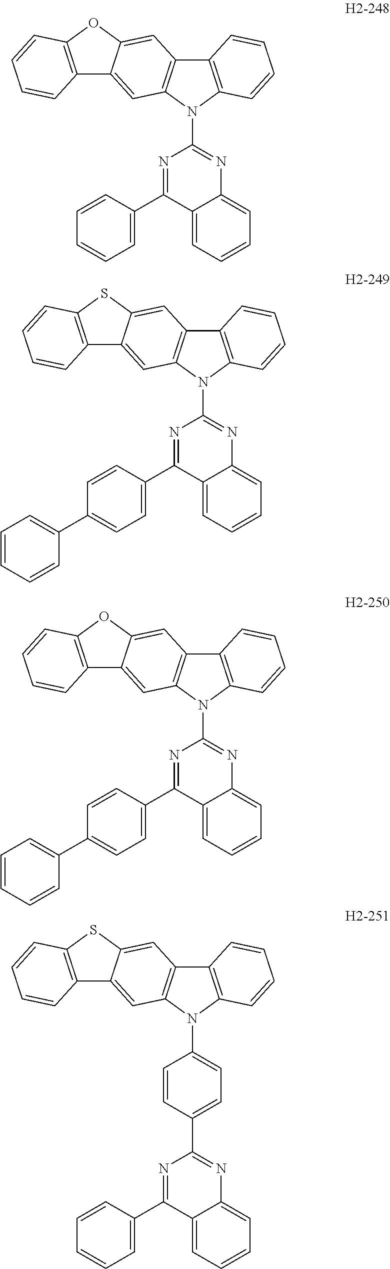 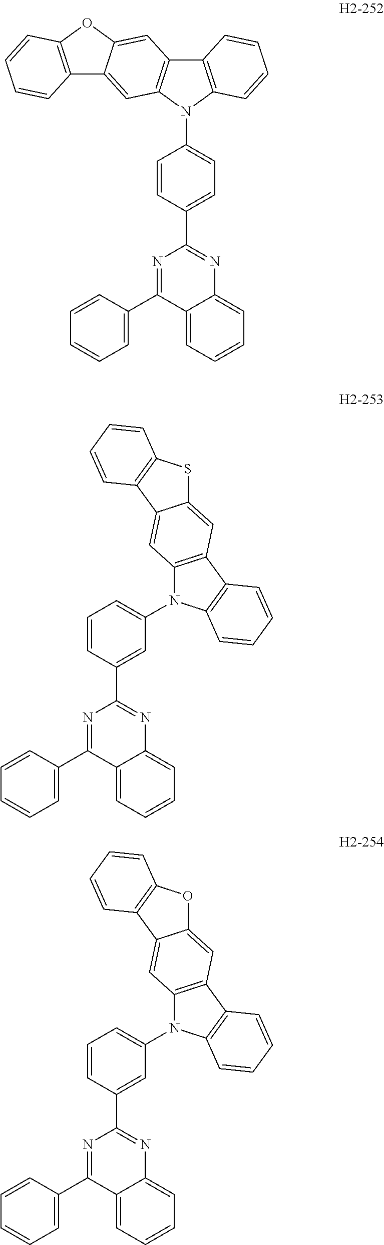 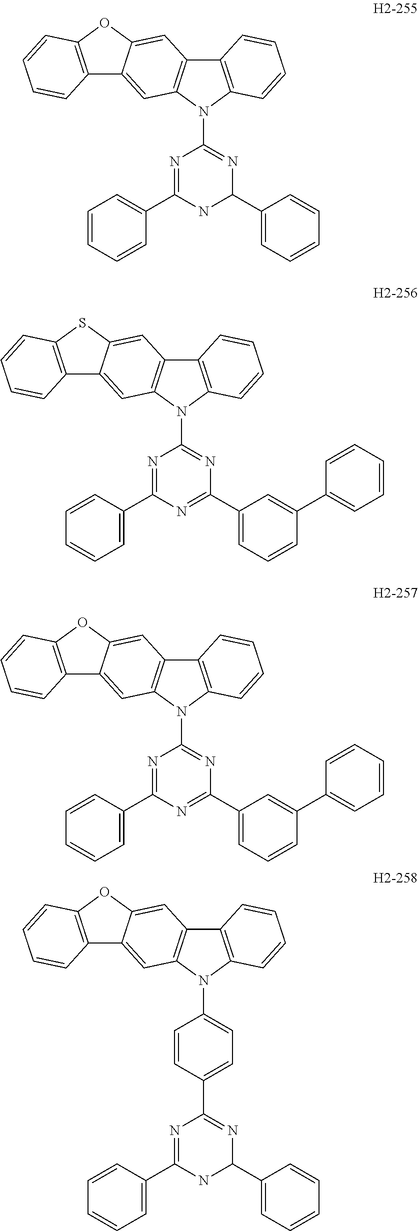 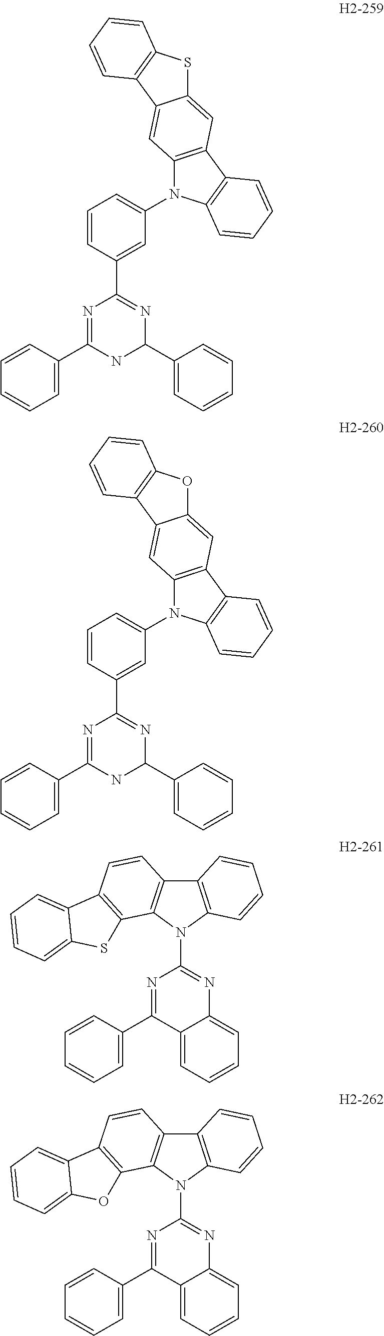 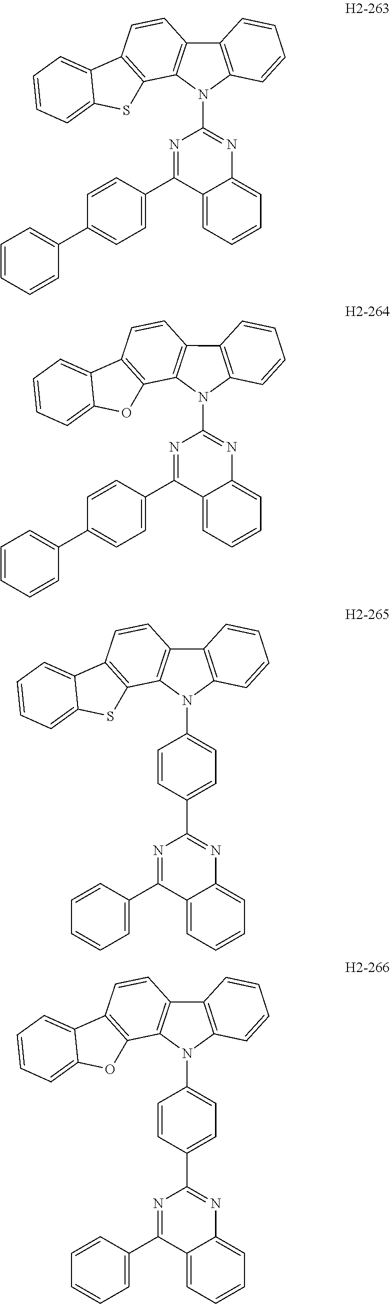 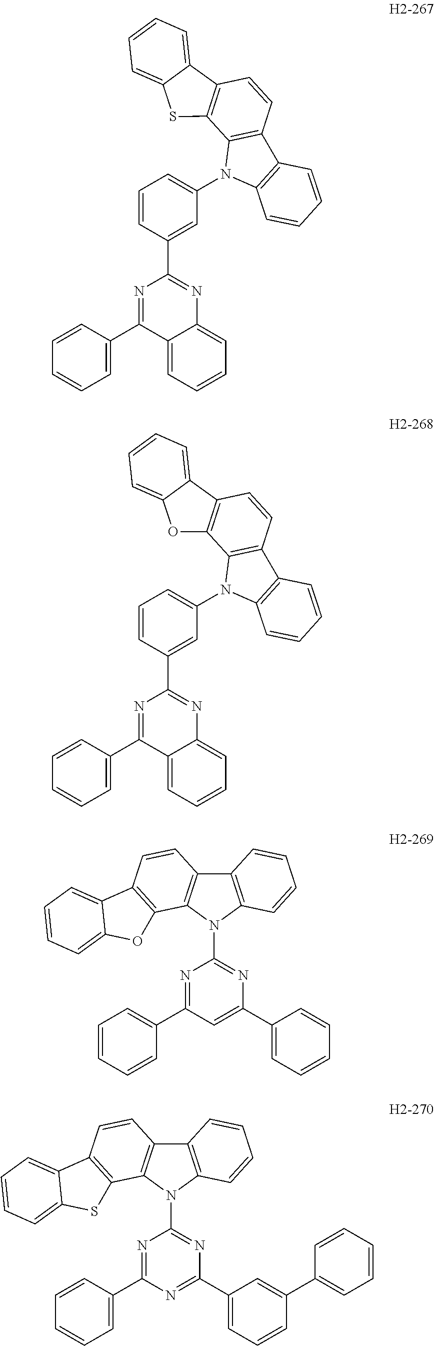 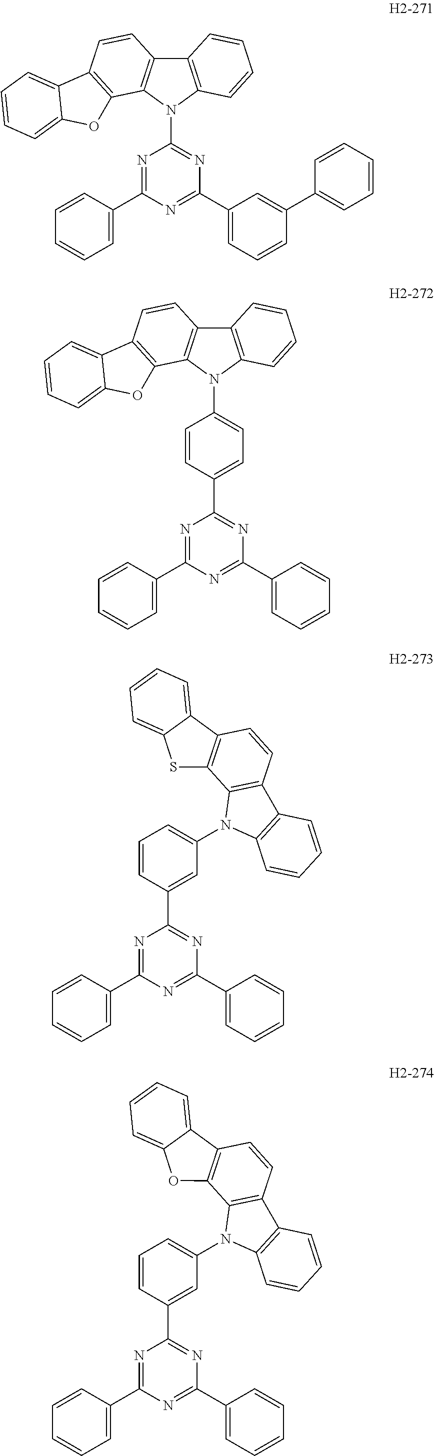 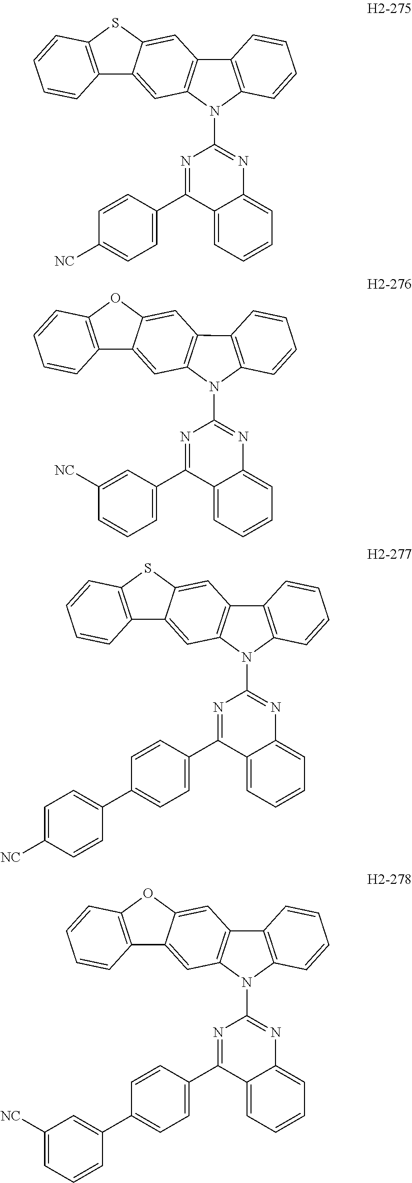 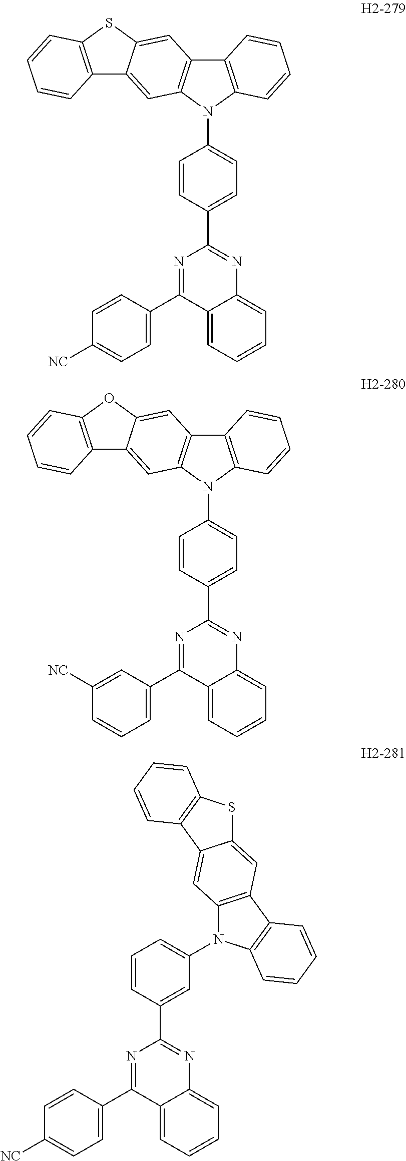 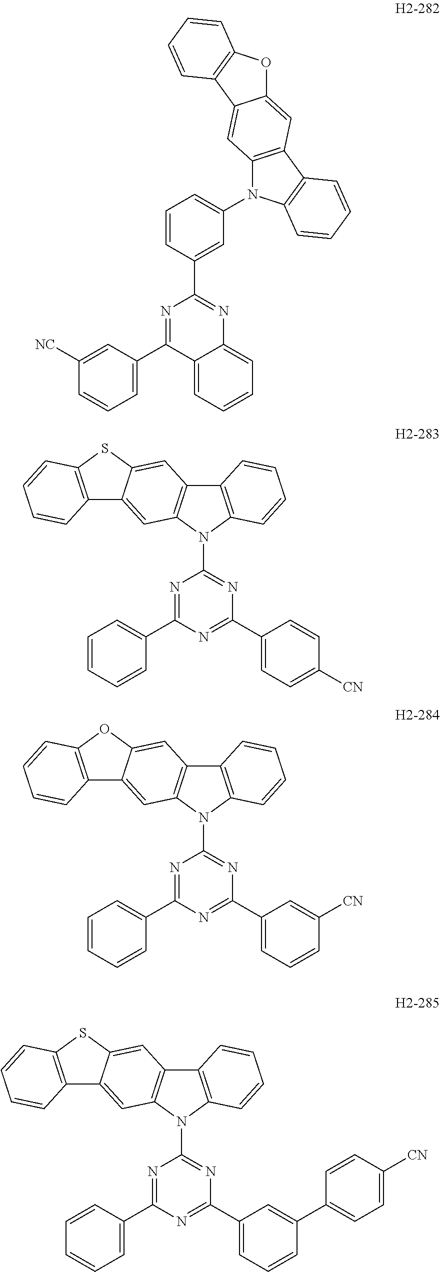 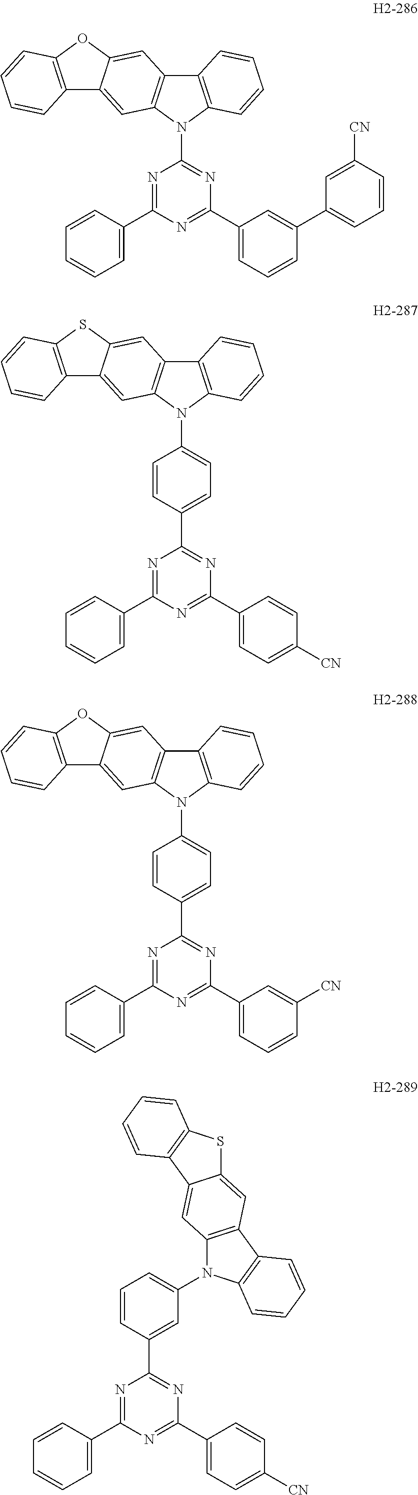 [SMILES: N#Cc1cccc(-c2cccc(-c3nc(-c4ccccc4)nc(-n4c5ccccc5c5cc6oc7ccccc7c6cc54)n3)c2)c1 ; N#Cc1ccc(-c2nc(-c3ccccc3)nc(-c3ccc(-n4c5ccccc5c5cc6sc7ccccc7c6cc54)cc3)n2)cc1 ; N#Cc1cccc(-c2nc(-c3ccccc3)nc(-c3ccc(-n4c5ccccc5c5cc6oc7ccccc7c6cc54)cc3)n2)c1 ; N#Cc1ccc(-c2nc(-c3ccccc3)nc(-c3cccc(-n4c5ccccc5c5cc6sc7ccccc7c6cc54)c3)n2)cc1] 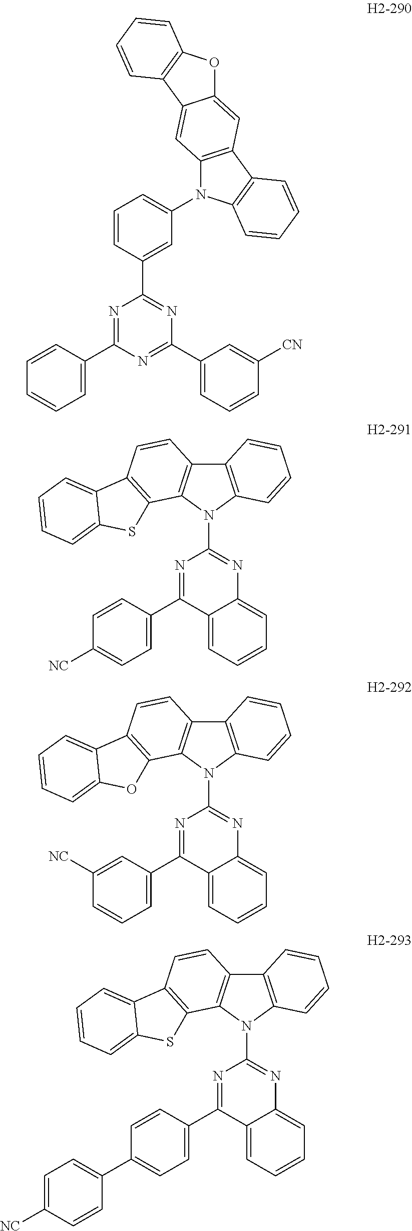 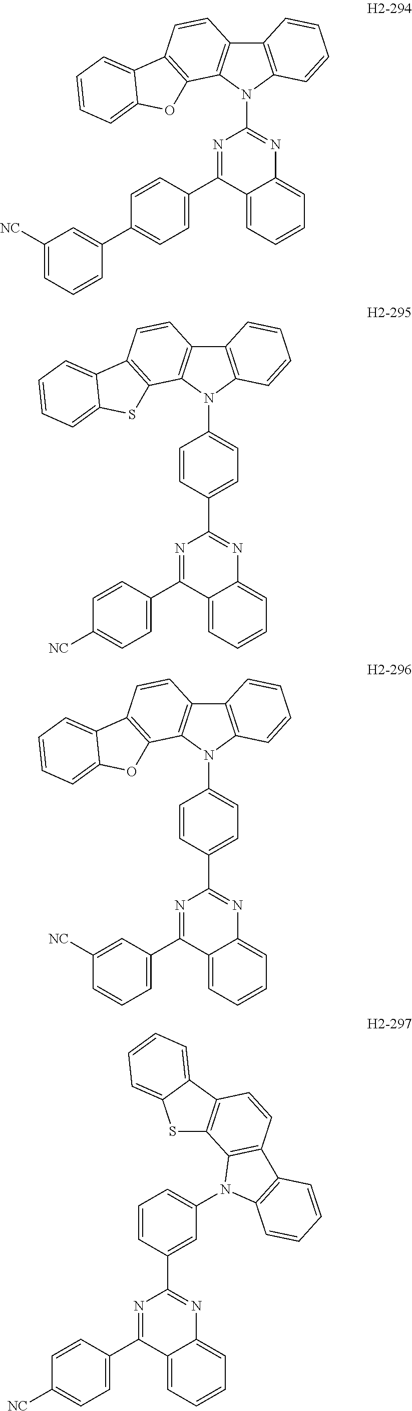 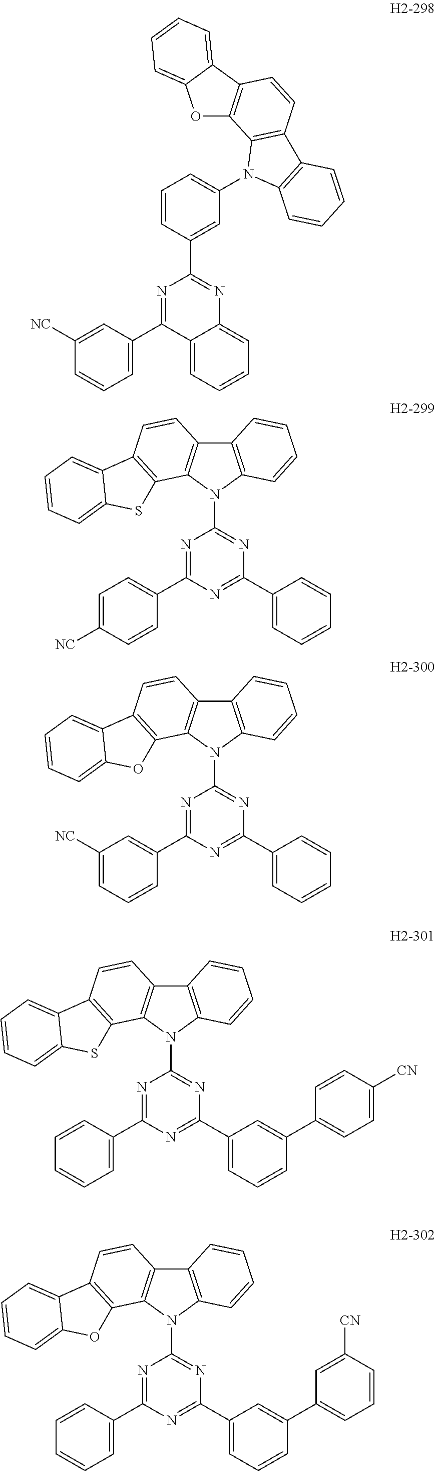 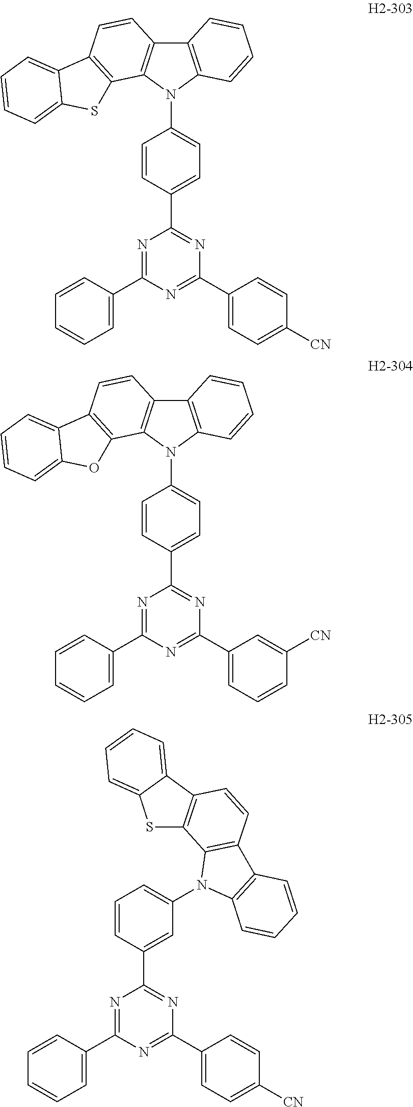 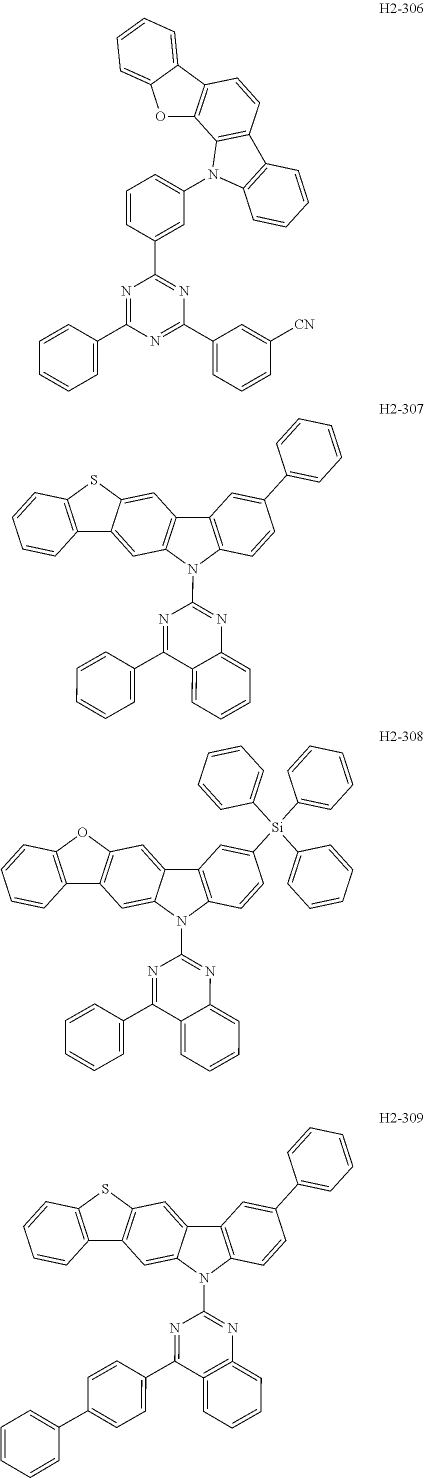 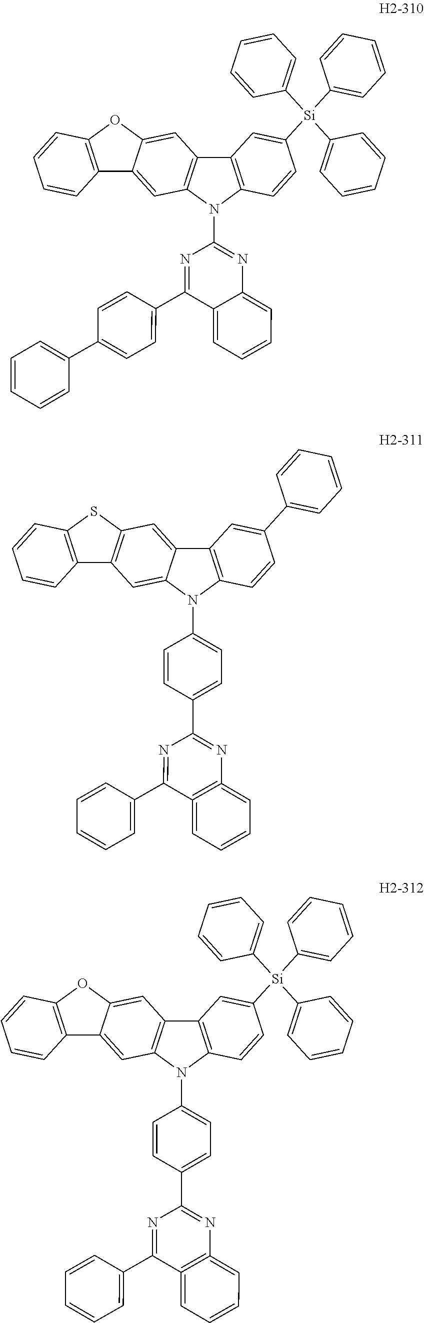 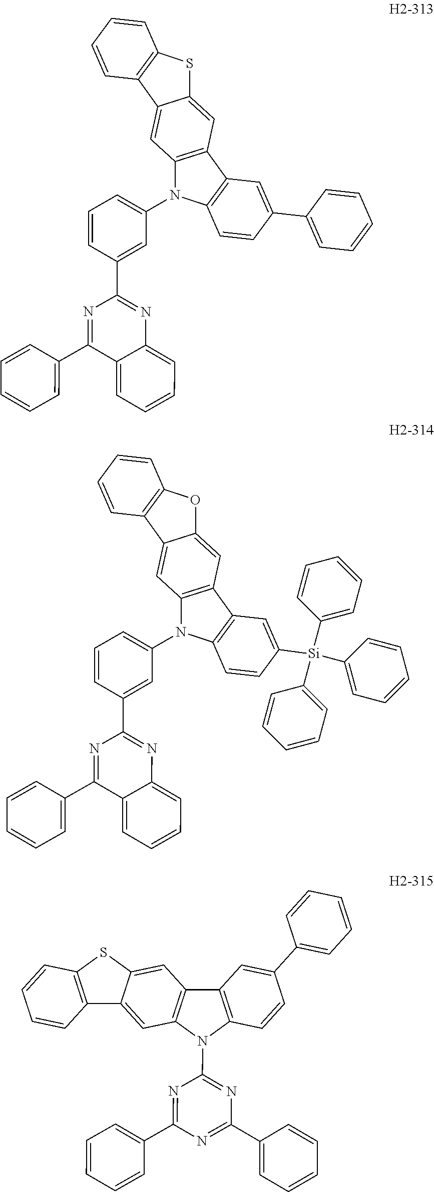 [SMILES: c1ccc(-c2ccc3c(c2)c2cc4sc5ccccc5c4cc2n3-c2cccc(-c3nc(-c4ccccc4)c4ccccc4n3)c2)cc1 ; c1ccc(-c2nc(-c3cccc(-n4c5ccc([Si](c6ccccc6)(c6ccccc6)c6ccccc6)cc5c5cc6oc7ccccc7c6cc54)c3)nc3ccccc23)cc1 ; c1ccc(-c2ccc3c(c2)c2cc4sc5ccccc5c4cc2n3-c2nc(-c3ccccc3)nc(-c3ccccc3)n2)cc1] 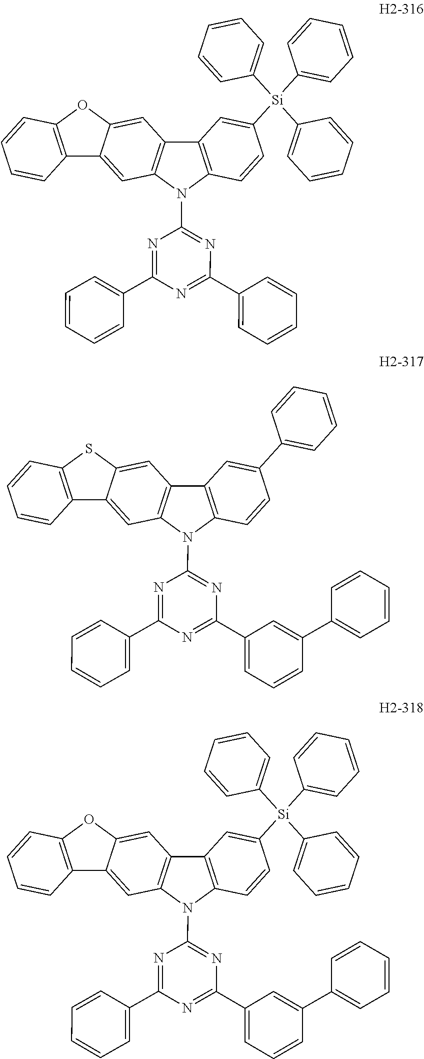 [SMILES: c1ccc(-c2nc(-c3ccccc3)nc(-n3c4ccc([Si](c5ccccc5)(c5ccccc5)c5ccccc5)cc4c4cc5oc6ccccc6c5cc43)n2)cc1 ; c1ccc(-c2cccc(-c3nc(-c4ccccc4)nc(-n4c5ccc(-c6ccccc6)cc5c5cc6sc7ccccc7c6cc54)n3)c2)cc1 ; c1ccc(-c2cccc(-c3nc(-c4ccccc4)nc(-n4c5ccc([Si](c6ccccc6)(c6ccccc6)c6ccccc6)cc5c5cc6oc7ccccc7c6cc54)n3)c2)cc1] 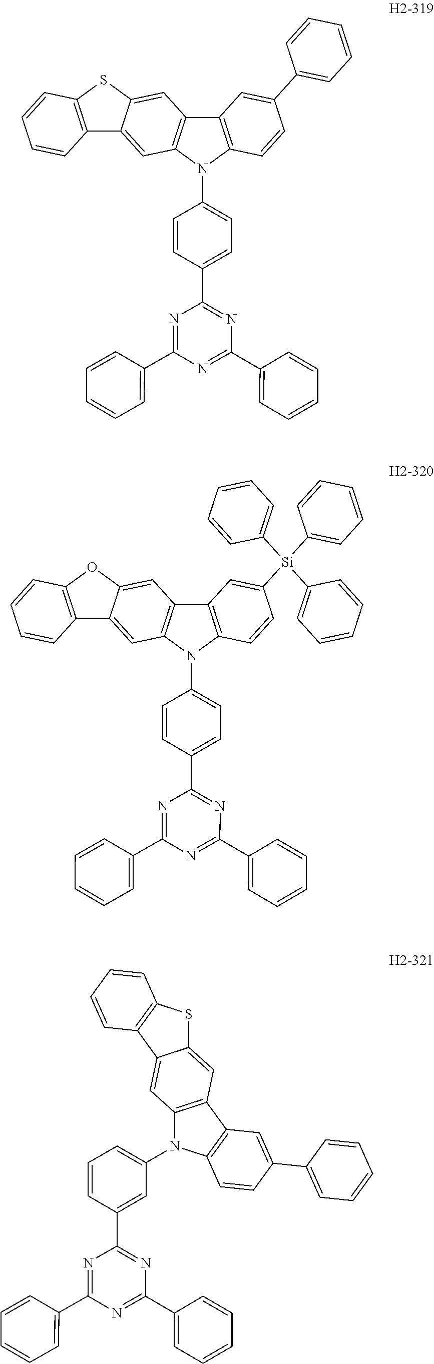 [SMILES: c1ccc(-c2ccc3c(c2)c2cc4sc5ccccc5c4cc2n3-c2ccc(-c3nc(-c4ccccc4)nc(-c4ccccc4)n3)cc2)cc1 ; c1ccc(-c2nc(-c3ccccc3)nc(-c3ccc(-n4c5ccc([Si](c6ccccc6)(c6ccccc6)c6ccccc6)cc5c5cc6oc7ccccc7c6cc54)cc3)n2)cc1 ; c1ccc(-c2ccc3c(c2)c2cc4sc5ccccc5c4cc2n3-c2cccc(-c3nc(-c4ccccc4)nc(-c4ccccc4)n3)c2)cc1] 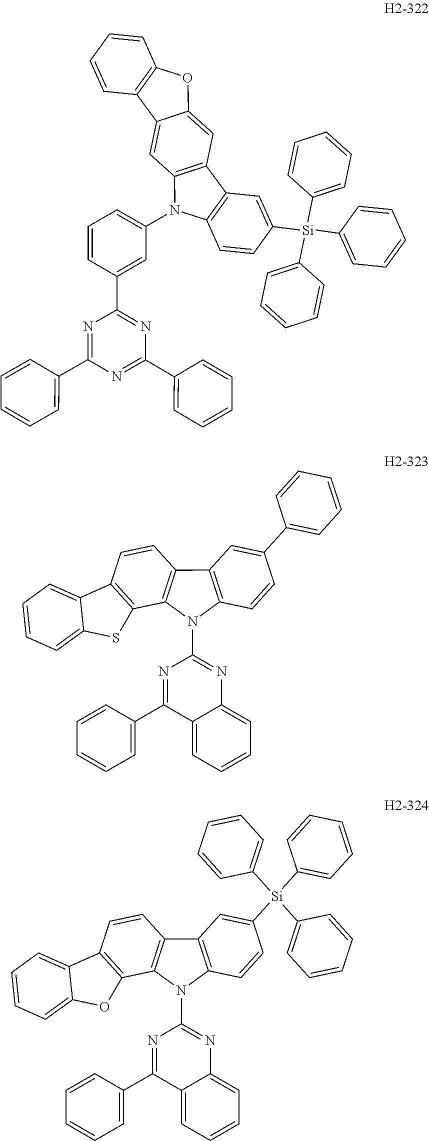 [SMILES: c1ccc(-c2nc(-c3ccccc3)nc(-c3cccc(-n4c5ccc([Si](c6ccccc6)(c6ccccc6)c6ccccc6)cc5c5cc6oc7ccccc7c6cc54)c3)n2)cc1 ; c1ccc(-c2ccc3c(c2)c2ccc4c5ccccc5sc4c2n3-c2nc(-c3ccccc3)c3ccccc3n2)cc1 ; c1ccc(-c2nc(-n3c4ccc([Si](c5ccccc5)(c5ccccc5)c5ccccc5)cc4c4ccc5c6ccccc6oc5c43)nc3ccccc23)cc1] 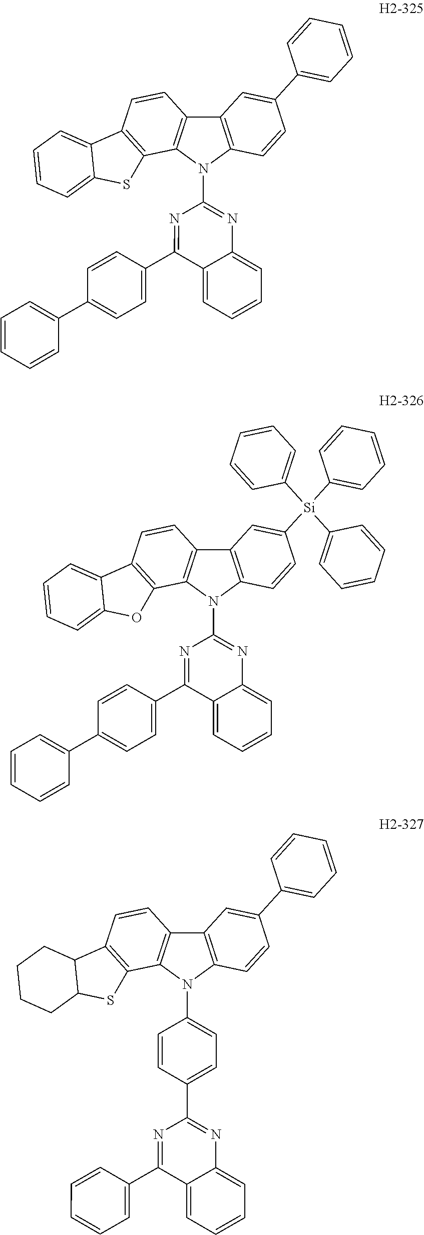 [SMILES: c1ccc(-c2ccc(-c3nc(-n4c5ccc(-c6ccccc6)cc5c5ccc6c7ccccc7sc6c54)nc4ccccc34)cc2)cc1 ; c1ccc(-c2ccc(-c3nc(-n4c5ccc([Si](c6ccccc6)(c6ccccc6)c6ccccc6)cc5c5ccc6c7ccccc7oc6c54)nc4ccccc34)cc2)cc1 ; c1ccc(-c2ccc3c(c2)c2ccc4c(c2n3-c2ccc(-c3nc(-c5ccccc5)c5ccccc5n3)cc2)SC2CCCCC42)cc1] 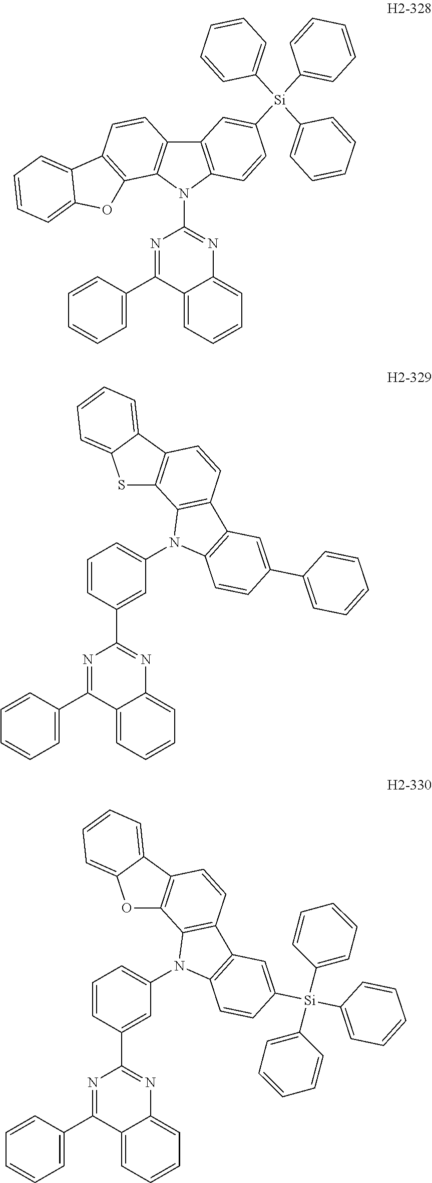 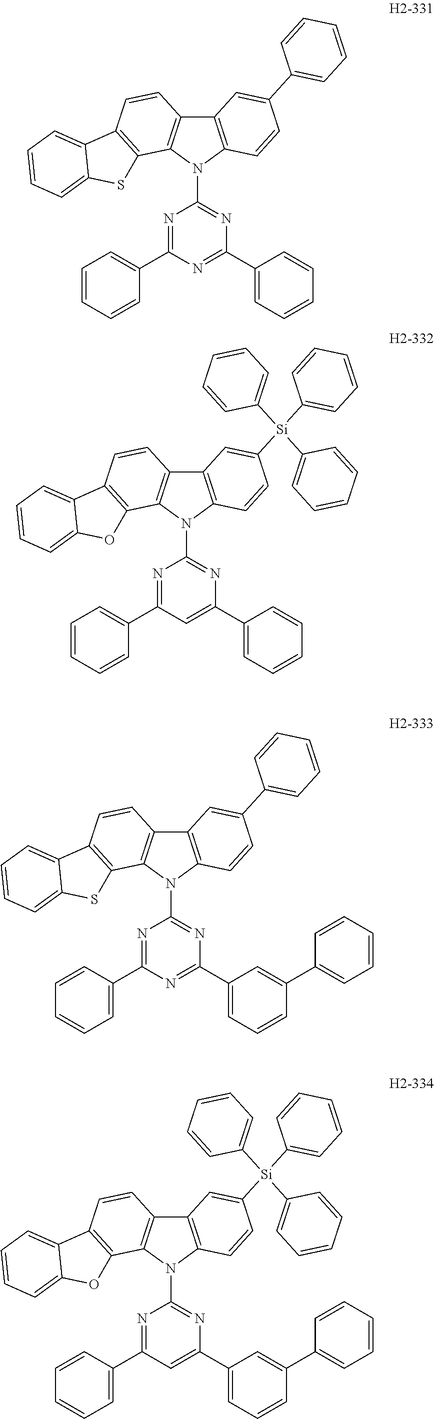 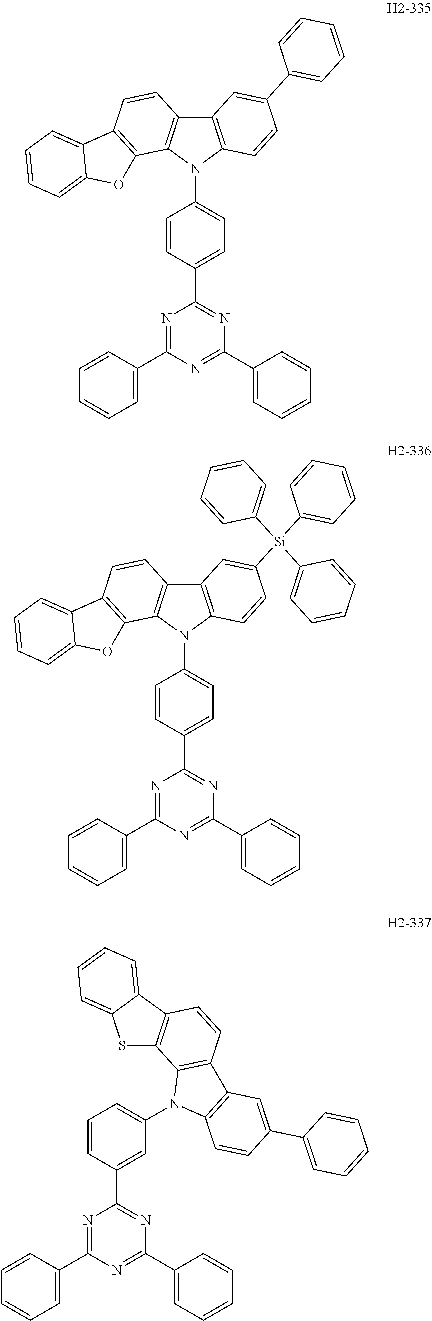 [SMILES: c1ccc(-c2ccc3c(c2)c2ccc4c5ccccc5oc4c2n3-c2ccc(-c3nc(-c4ccccc4)nc(-c4ccccc4)n3)cc2)cc1 ; c1ccc(-c2nc(-c3ccccc3)nc(-c3ccc(-n4c5ccc([Si](c6ccccc6)(c6ccccc6)c6ccccc6)cc5c5ccc6c7ccccc7oc6c54)cc3)n2)cc1 ; c1ccc(-c2ccc3c(c2)c2ccc4c5ccccc5sc4c2n3-c2cccc(-c3nc(-c4ccccc4)nc(-c4ccccc4)n3)c2)cc1] 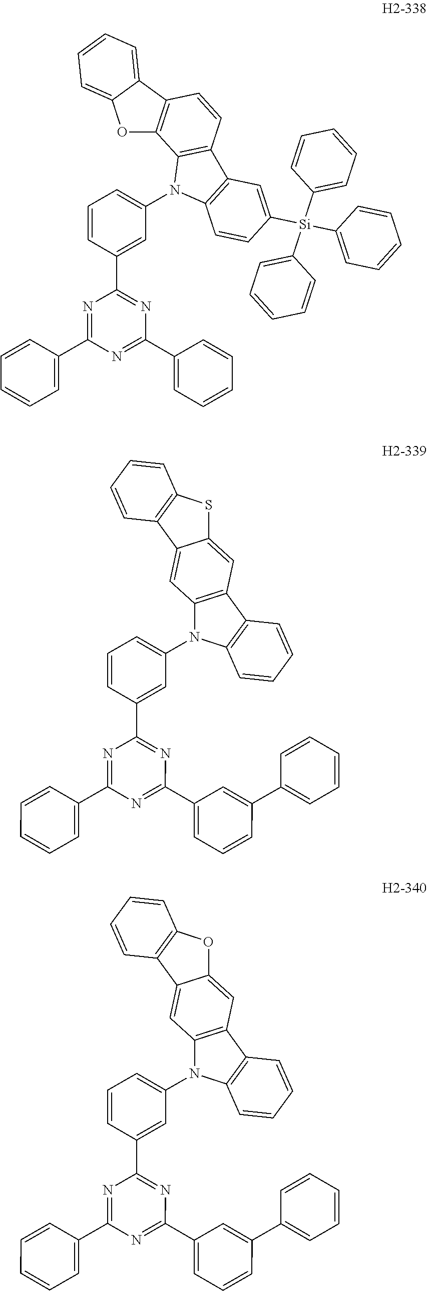 [SMILES: c1ccc(-c2nc(-c3ccccc3)nc(-c3cccc(-n4c5ccc([Si](c6ccccc6)(c6ccccc6)c6ccccc6)cc5c5ccc6c7ccccc7oc6c54)c3)n2)cc1 ; c1ccc(-c2cccc(-c3nc(-c4ccccc4)nc(-c4cccc(-n5c6ccccc6c6cc7sc8ccccc8c7cc65)c4)n3)c2)cc1 ; c1ccc(-c2cccc(-c3nc(-c4ccccc4)nc(-c4cccc(-n5c6ccccc6c6cc7oc8ccccc8c7cc65)c4)n3)c2)cc1] 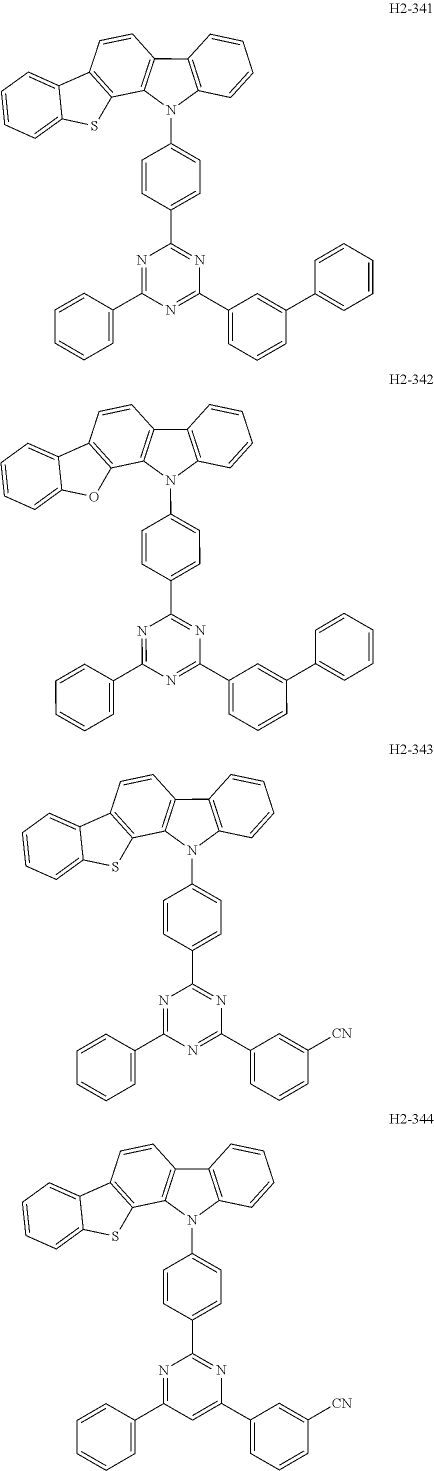 [SMILES: c1ccc(-c2cccc(-c3nc(-c4ccccc4)nc(-c4ccc(-n5c6ccccc6c6ccc7c8ccccc8sc7c65)cc4)n3)c2)cc1 ; c1ccc(-c2cccc(-c3nc(-c4ccccc4)nc(-c4ccc(-n5c6ccccc6c6ccc7c8ccccc8oc7c65)cc4)n3)c2)cc1 ; N#Cc1cccc(-c2nc(-c3ccccc3)nc(-c3ccc(-n4c5ccccc5c5ccc6c7ccccc7sc6c54)cc3)n2)c1 ; N#Cc1cccc(-c2cc(-c3ccccc3)nc(-c3ccc(-n4c5ccccc5c5ccc6c7ccccc7sc6c54)cc3)n2)c1] 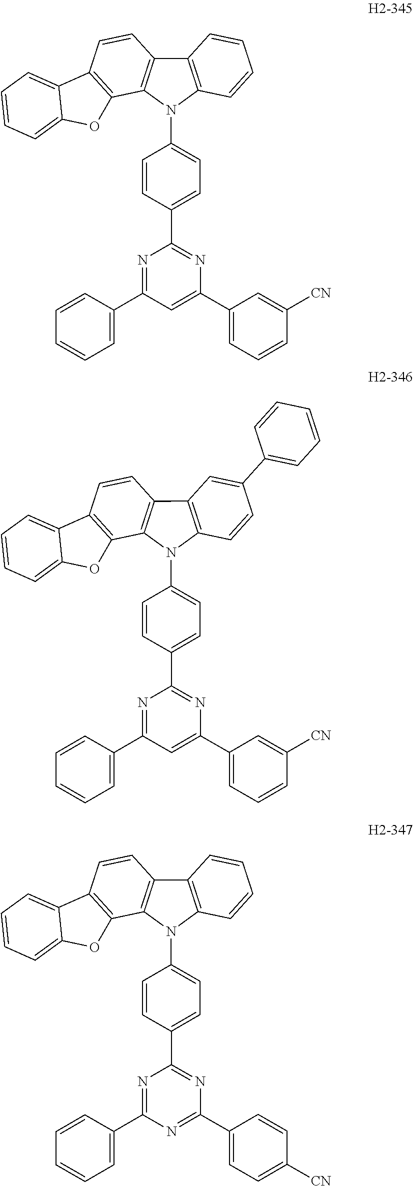 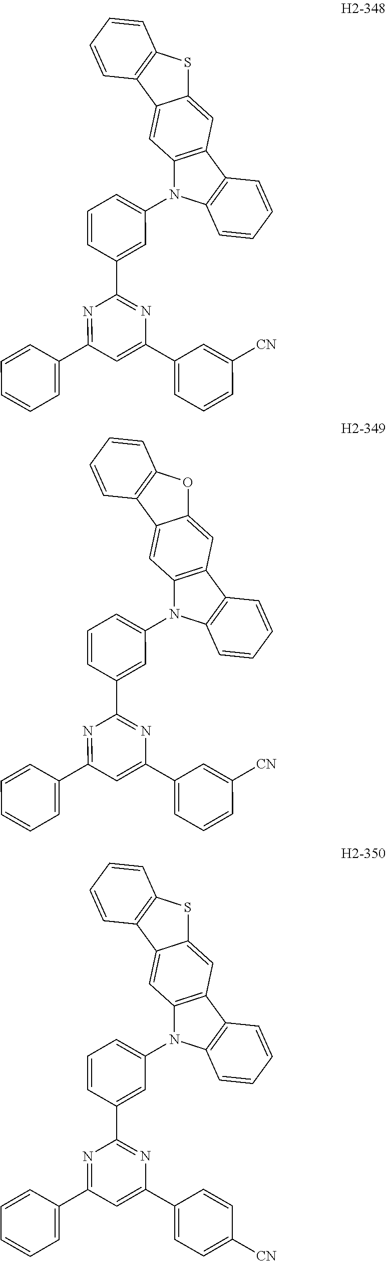 [SMILES: N#Cc1cccc(-c2cc(-c3ccccc3)nc(-c3cccc(-n4c5ccccc5c5cc6sc7ccccc7c6cc54)c3)n2)c1 ; N#Cc1cccc(-c2cc(-c3ccccc3)nc(-c3cccc(-n4c5ccccc5c5cc6oc7ccccc7c6cc54)c3)n2)c1 ; N#Cc1ccc(-c2cc(-c3ccccc3)nc(-c3cccc(-n4c5ccccc5c5cc6sc7ccccc7c6cc54)c3)n2)cc1] 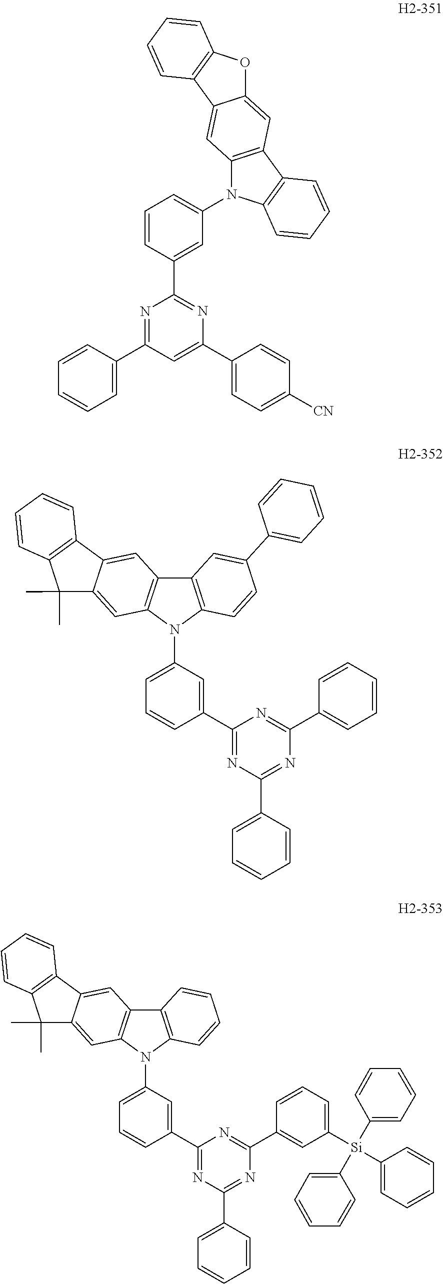 [SMILES: N#Cc1ccc(-c2cc(-c3ccccc3)nc(-c3cccc(-n4c5ccccc5c5cc6oc7ccccc7c6cc54)c3)n2)cc1 ; CC1(C)c2ccccc2-c2cc3c4cc(-c5ccccc5)ccc4n(-c4cccc(-c5nc(-c6ccccc6)nc(-c6ccccc6)n5)c4)c3cc21 ; CC1(C)c2ccccc2-c2cc3c4ccccc4n(-c4cccc(-c5nc(-c6ccccc6)nc(-c6cccc([Si](c7ccccc7)(c7ccccc7)c7ccccc7)c6)n5)c4)c3cc21] 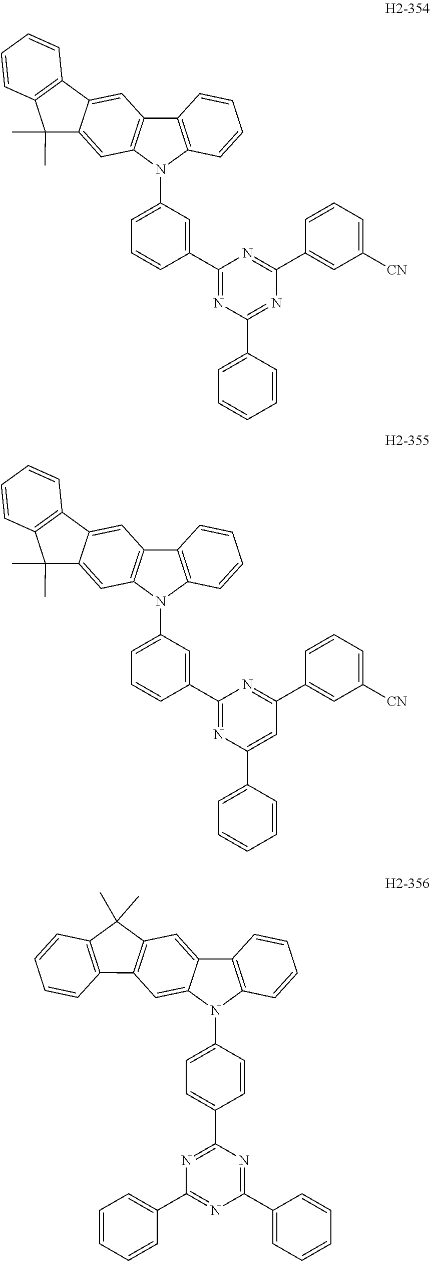 [SMILES: CC1(C)c2ccccc2-c2cc3c4ccccc4n(-c4cccc(-c5nc(-c6ccccc6)nc(-c6cccc(C#N)c6)n5)c4)c3cc21 ; CC1(C)c2ccccc2-c2cc3c4ccccc4n(-c4cccc(-c5nc(-c6ccccc6)cc(-c6cccc(C#N)c6)n5)c4)c3cc21 ; CC1(C)c2ccccc2-c2cc3c(cc21)c1ccccc1n3-c1ccc(-c2nc(-c3ccccc3)nc(-c3ccccc3)n2)cc1] 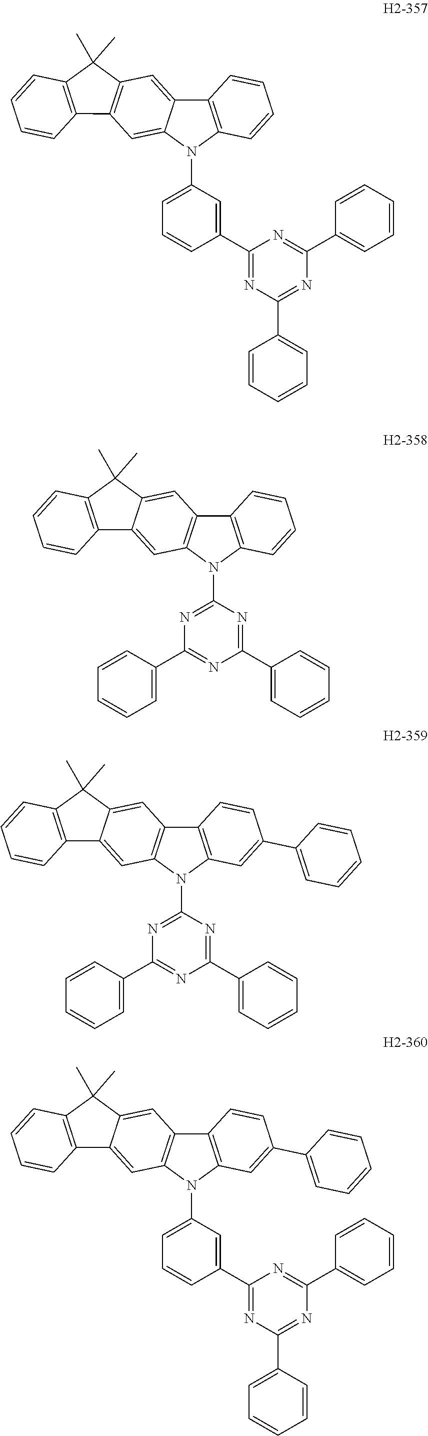 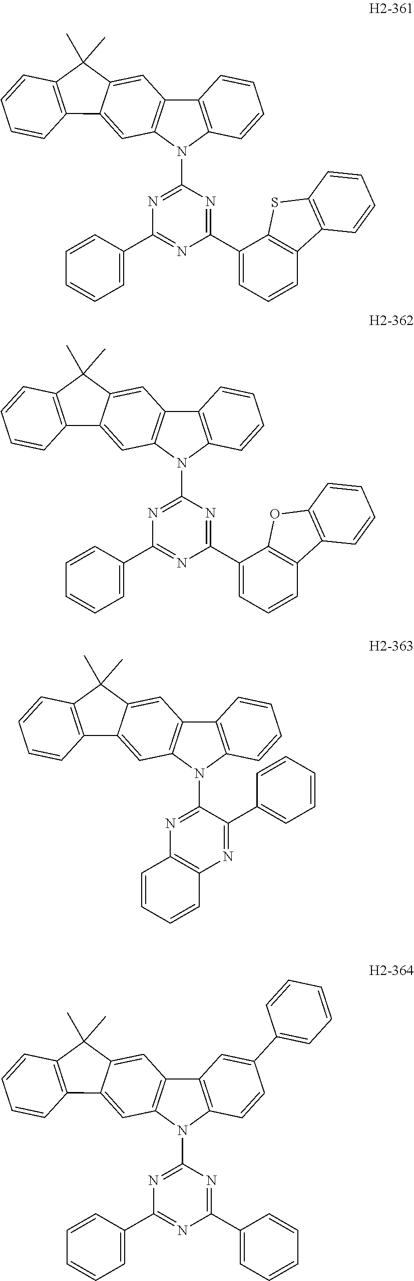 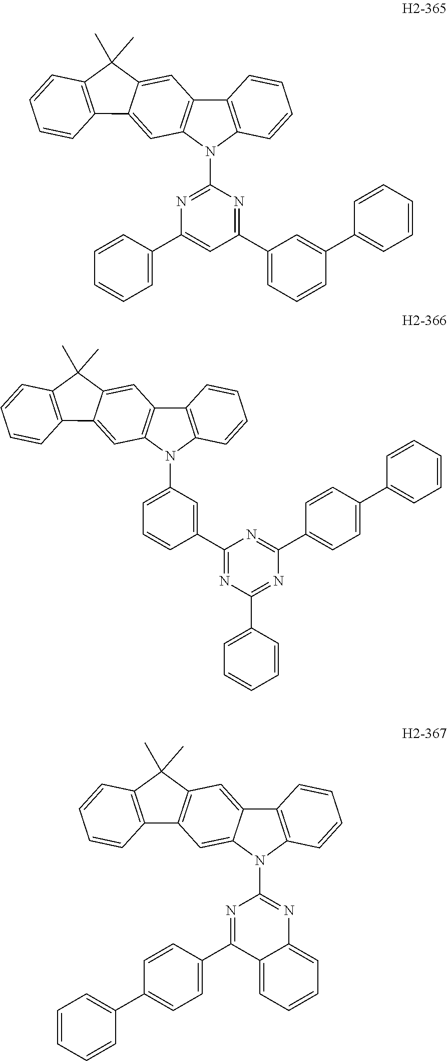 [SMILES: CC1(C)c2ccccc2-c2cc3c(cc21)c1ccccc1n3-c1nc(-c2ccccc2)cc(-c2cccc(-c3ccccc3)c2)n1 ; CC1(C)c2ccccc2-c2cc3c(cc21)c1ccccc1n3-c1cccc(-c2nc(-c3ccccc3)nc(-c3ccc(-c4ccccc4)cc3)n2)c1 ; CC1(C)c2ccccc2-c2cc3c(cc21)c1ccccc1n3-c1nc(-c2ccc(-c3ccccc3)cc2)c2ccccc2n1] 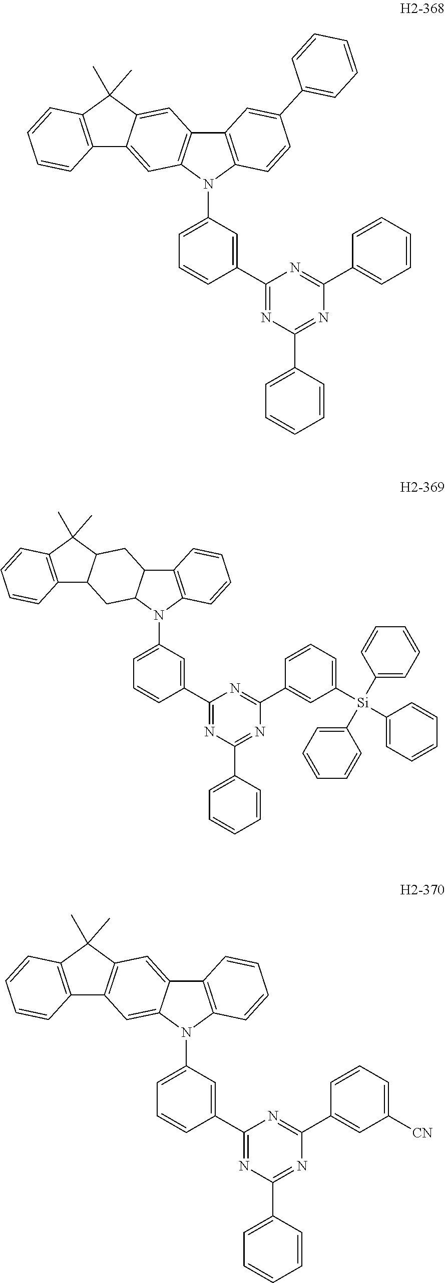 [SMILES: CC1(C)c2ccccc2-c2cc3c(cc21)c1cc(-c2ccccc2)ccc1n3-c1cccc(-c2nc(-c3ccccc3)nc(-c3ccccc3)n2)c1 ; CC1(C)c2ccccc2C2CC3C(CC21)c1ccccc1N3c1cccc(-c2nc(-c3ccccc3)nc(-c3cccc([Si](c4ccccc4)(c4ccccc4)c4ccccc4)c3)n2)c1 ; CC1(C)c2ccccc2-c2cc3c(cc21)c1ccccc1n3-c1cccc(-c2nc(-c3ccccc3)nc(-c3cccc(C#N)c3)n2)c1] 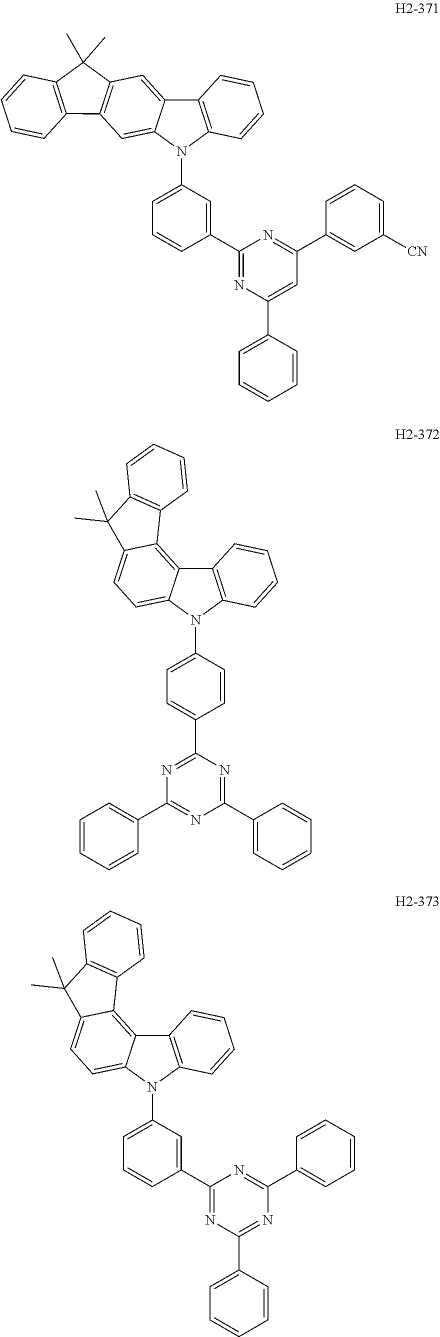 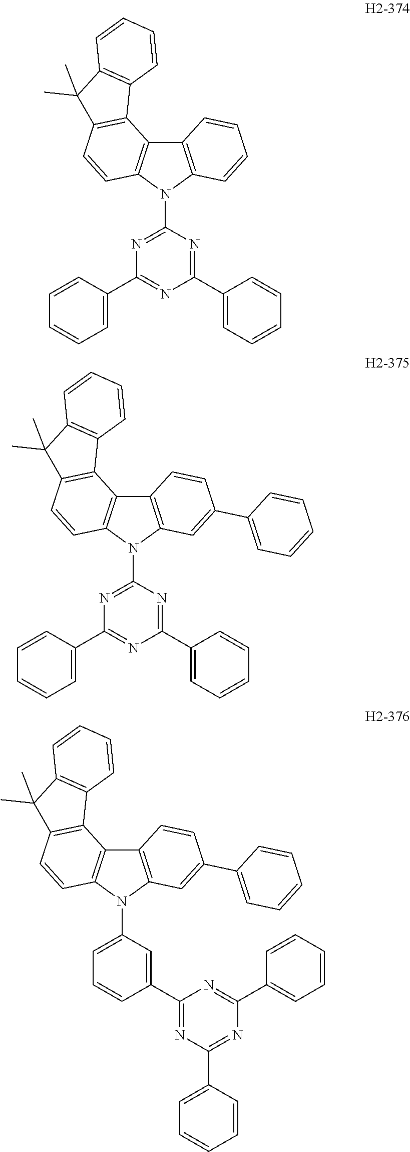 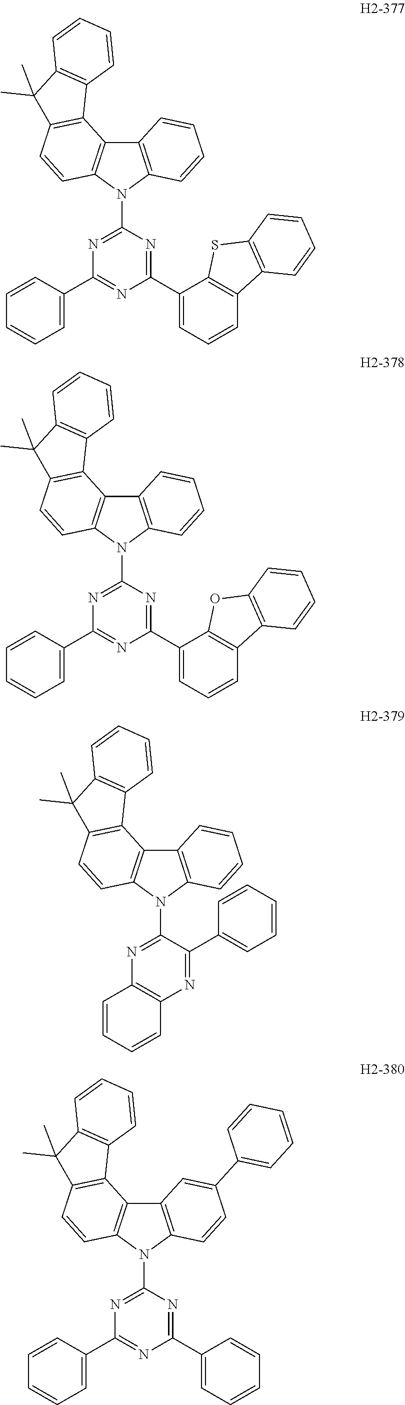 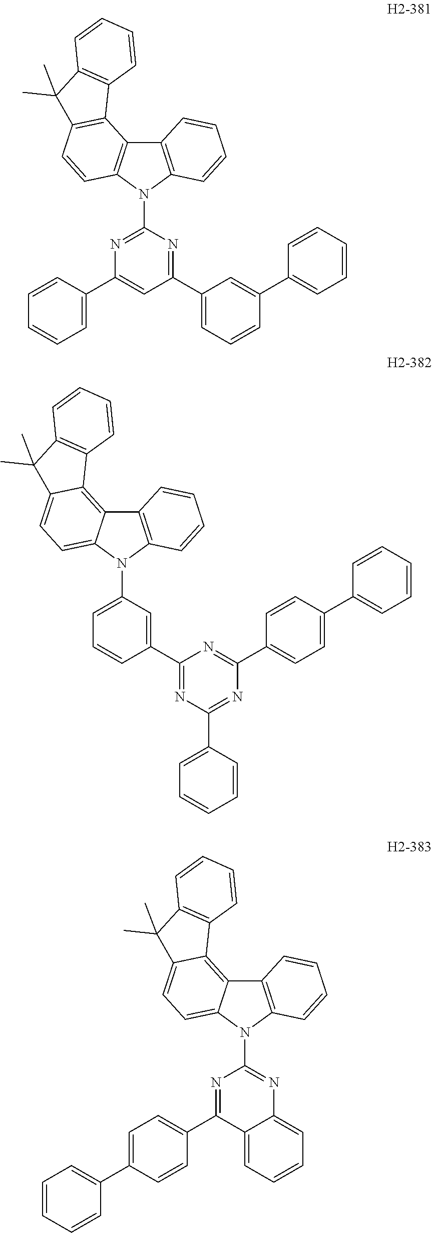 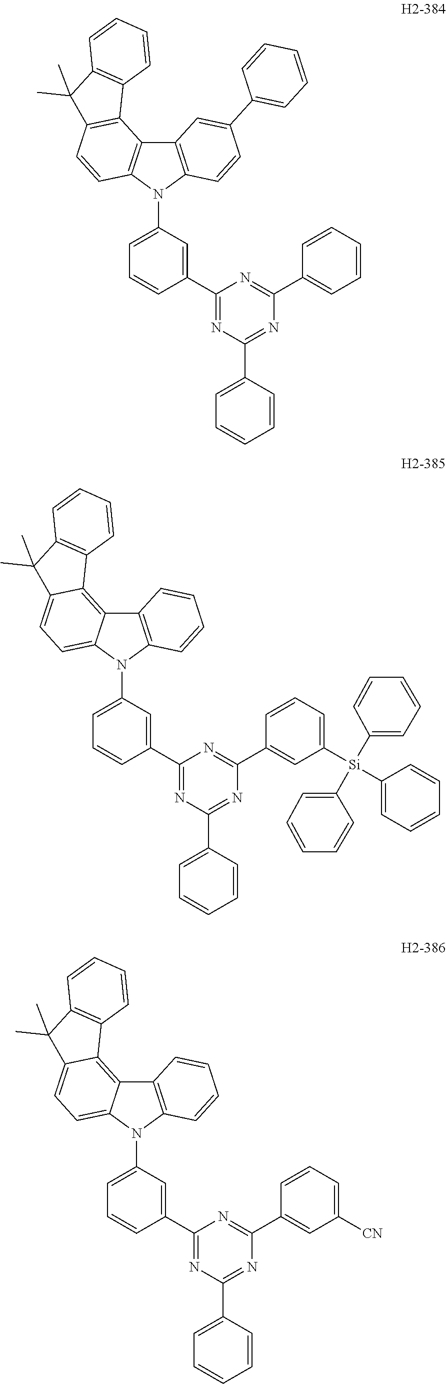 [SMILES: CC1(C)c2ccccc2-c2c1ccc1c2c2cc(-c3ccccc3)ccc2n1-c1cccc(-c2nc(-c3ccccc3)nc(-c3ccccc3)n2)c1 ; CC1(C)c2ccccc2-c2c1ccc1c2c2ccccc2n1-c1cccc(-c2nc(-c3ccccc3)nc(-c3cccc([Si](c4ccccc4)(c4ccccc4)c4ccccc4)c3)n2)c1 ; CC1(C)c2ccccc2-c2c1ccc1c2c2ccccc2n1-c1cccc(-c2nc(-c3ccccc3)nc(-c3cccc(C#N)c3)n2)c1] 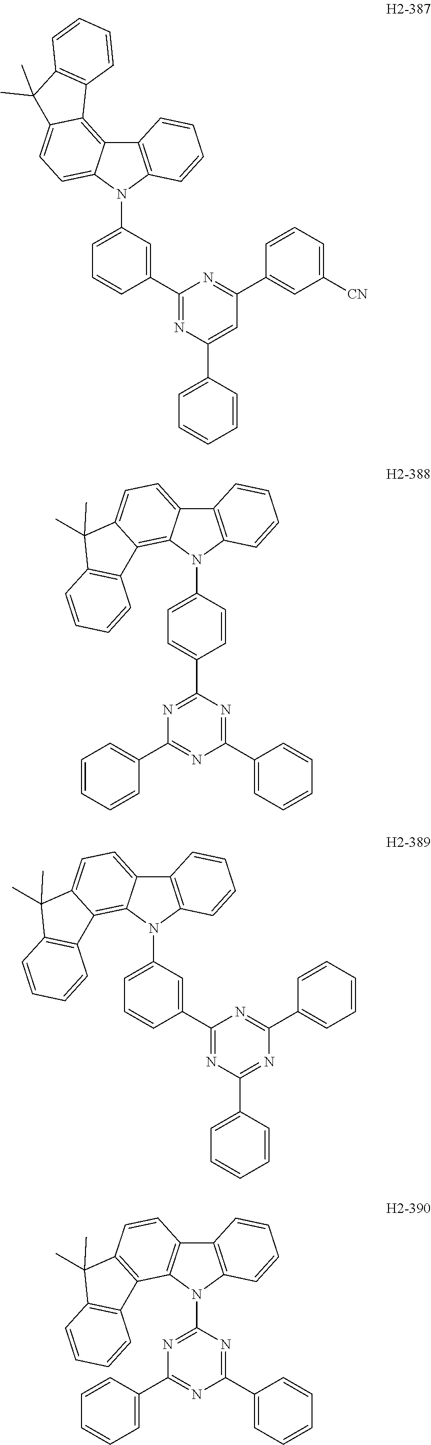 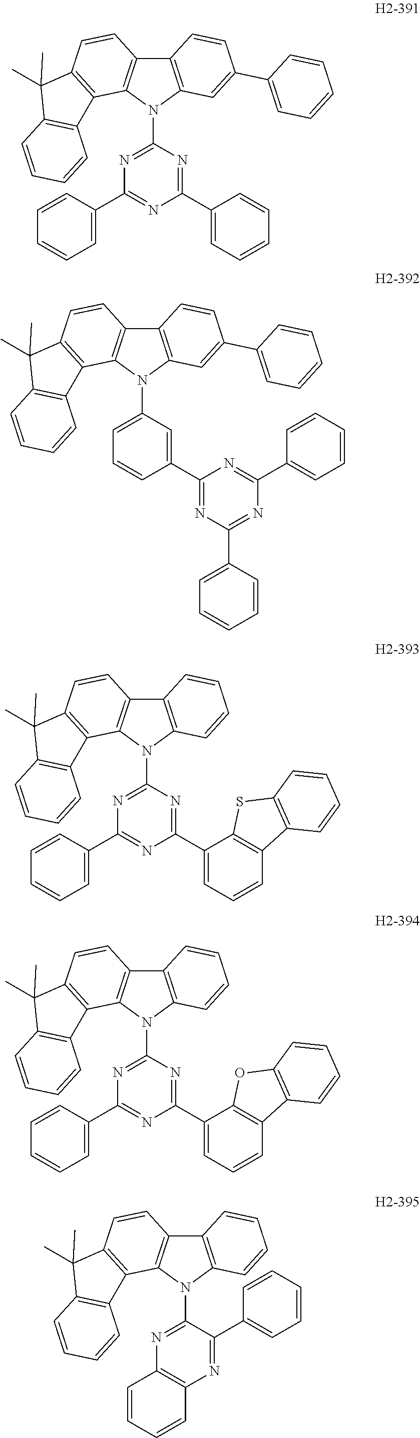 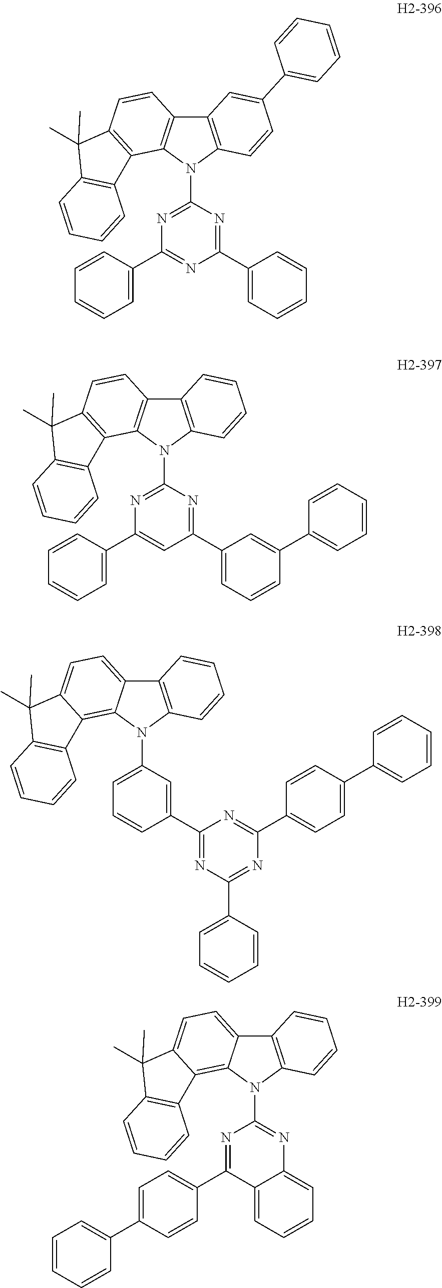 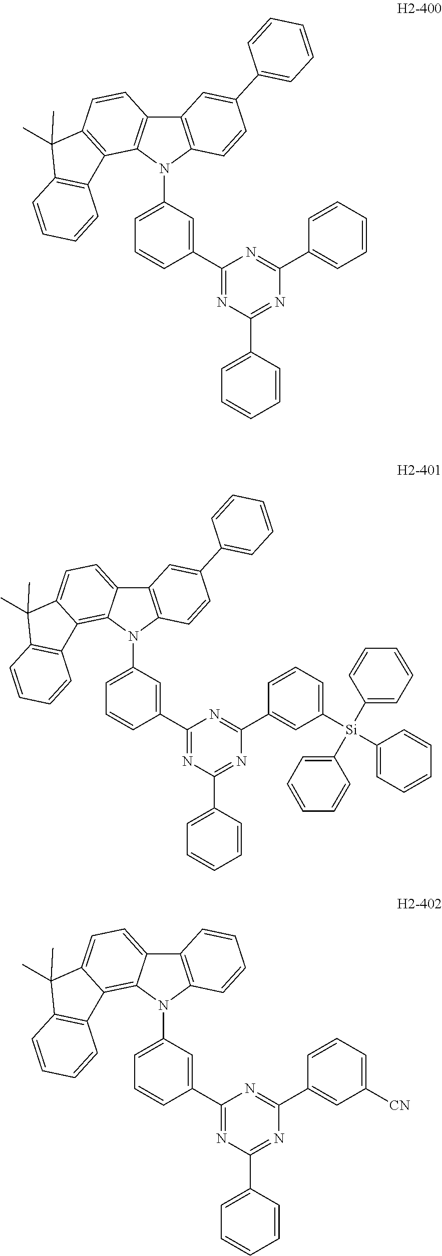 [SMILES: CC1(C)c2ccccc2-c2c1ccc1c3cc(-c4ccccc4)ccc3n(-c3cccc(-c4nc(-c5ccccc5)nc(-c5ccccc5)n4)c3)c21 ; CC1(C)c2ccccc2-c2c1ccc1c3cc(-c4ccccc4)ccc3n(-c3cccc(-c4nc(-c5ccccc5)nc(-c5cccc([Si](c6ccccc6)(c6ccccc6)c6ccccc6)c5)n4)c3)c21 ; CC1(C)c2ccccc2-c2c1ccc1c3ccccc3n(-c3cccc(-c4nc(-c5ccccc5)nc(-c5cccc(C#N)c5)n4)c3)c21] 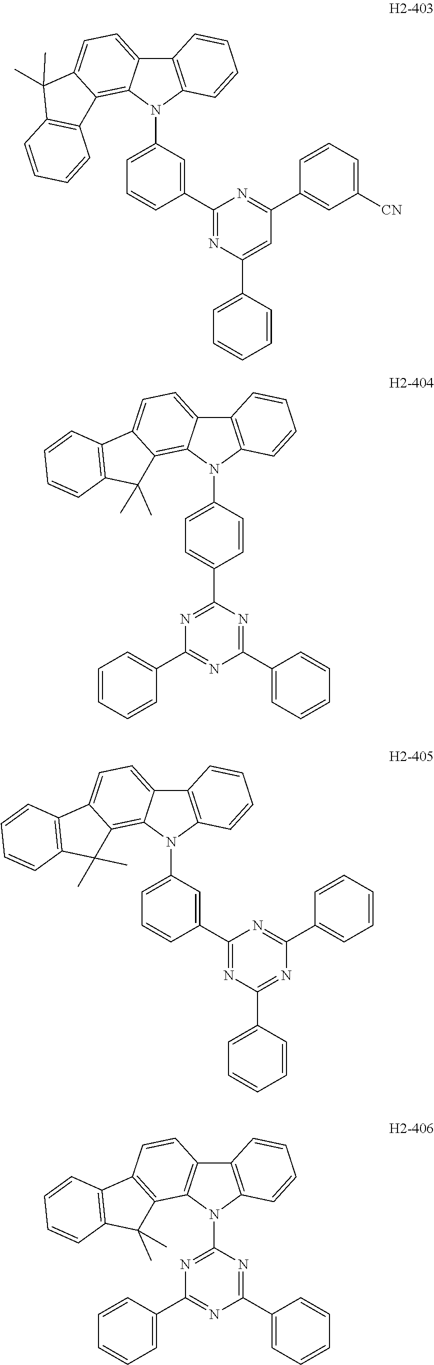 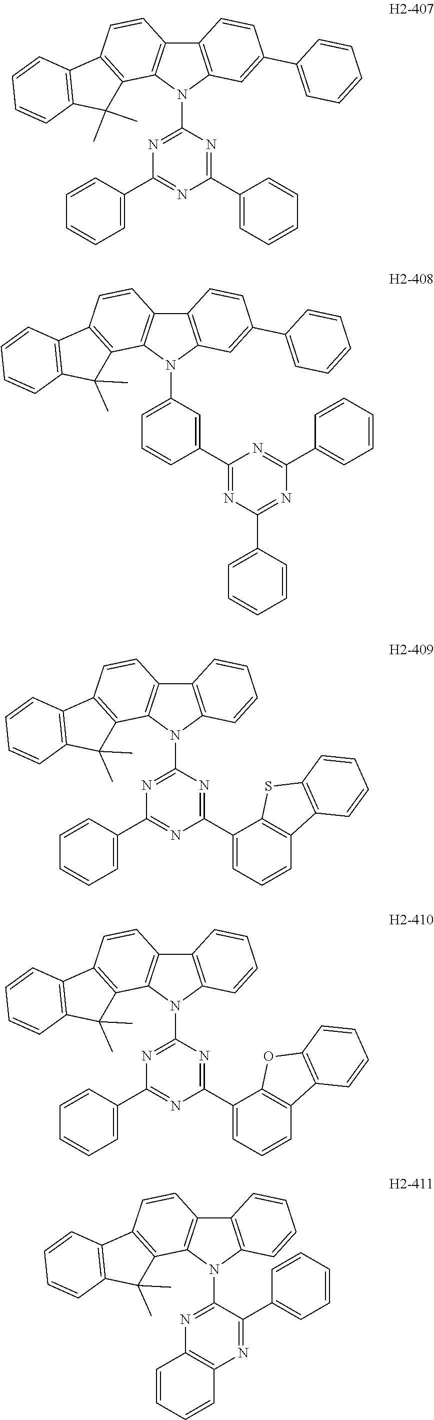 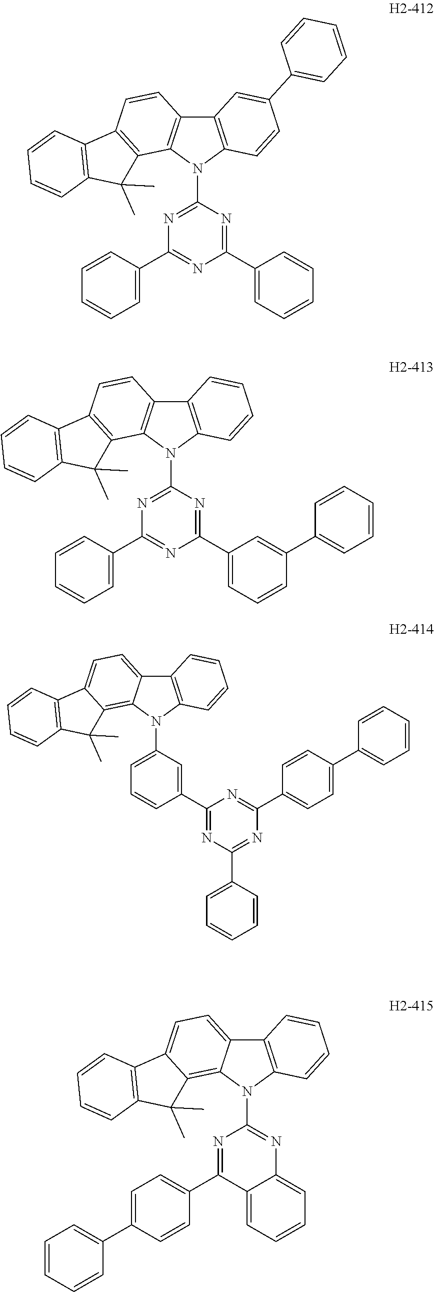 [SMILES: CC1(C)c2ccccc2-c2ccc3c4cc(-c5ccccc5)ccc4n(-c4nc(-c5ccccc5)nc(-c5ccccc5)n4)c3c21 ; CC1(C)c2ccccc2-c2ccc3c4ccccc4n(-c4nc(-c5ccccc5)nc(-c5cccc(-c6ccccc6)c5)n4)c3c21 ; CC1(C)c2ccccc2-c2ccc3c4ccccc4n(-c4cccc(-c5nc(-c6ccccc6)nc(-c6ccc(-c7ccccc7)cc6)n5)c4)c3c21 ; CC1(C)c2ccccc2-c2ccc3c4ccccc4n(-c4nc(-c5ccc(-c6ccccc6)cc5)c5ccccc5n4)c3c21] 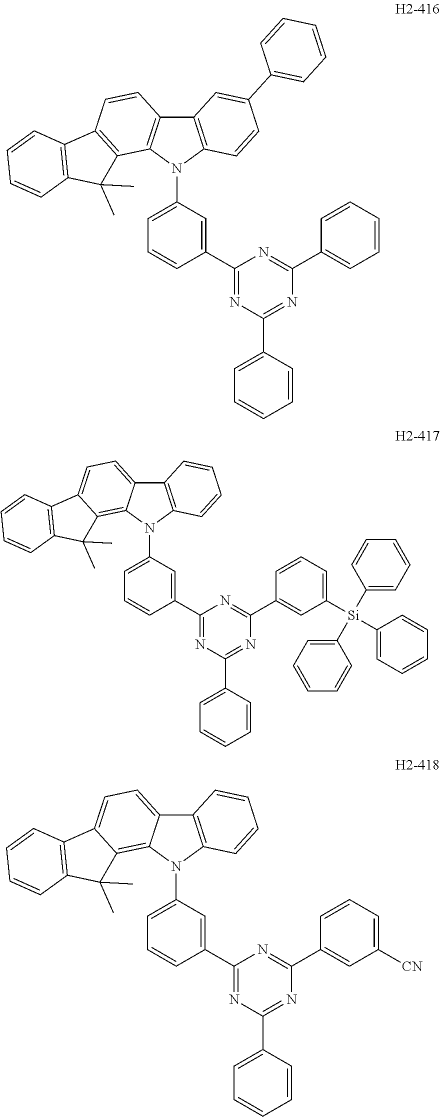 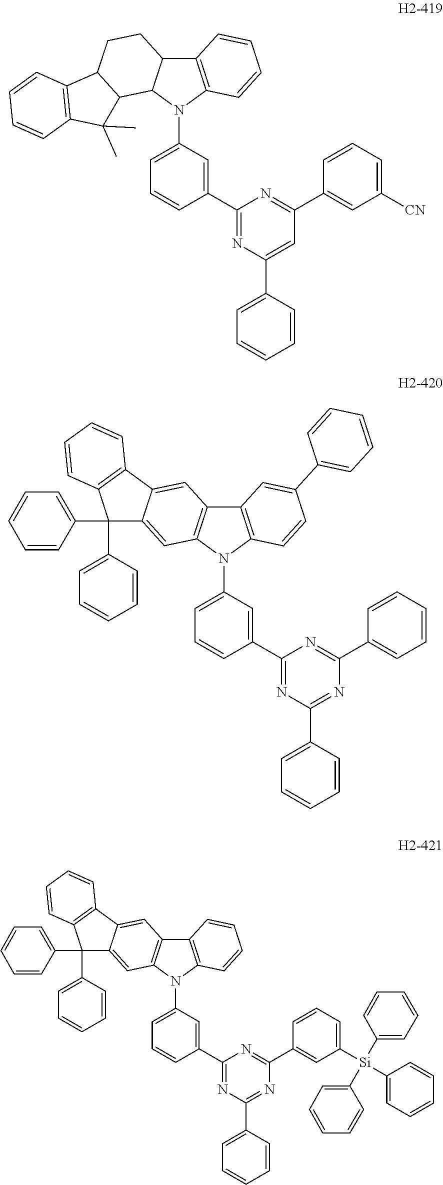 [SMILES: CC1(C)c2ccccc2C2CCC3c4ccccc4N(c4cccc(-c5nc(-c6ccccc6)cc(-c6cccc(C#N)c6)n5)c4)C3C21 ; c1ccc(-c2ccc3c(c2)c2cc4c(cc2n3-c2cccc(-c3nc(-c5ccccc5)nc(-c5ccccc5)n3)c2)C(c2ccccc2)(c2ccccc2)c2ccccc2-4)cc1 ; c1ccc(-c2nc(-c3cccc(-n4c5ccccc5c5cc6c(cc54)C(c4ccccc4)(c4ccccc4)c4ccccc4-6)c3)nc(-c3cccc([Si](c4ccccc4)(c4ccccc4)c4ccccc4)c3)n2)cc1] 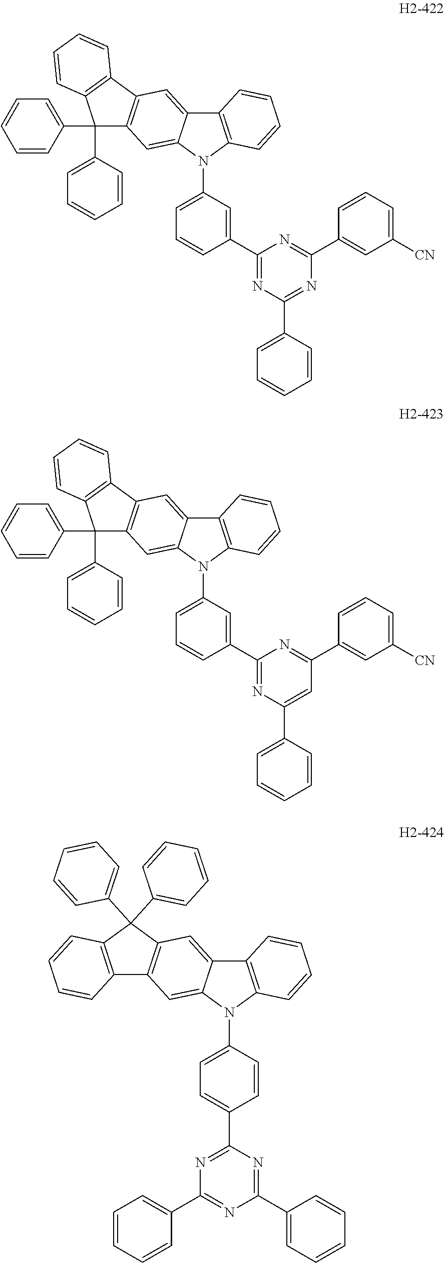 [SMILES: N#Cc1cccc(-c2nc(-c3ccccc3)nc(-c3cccc(-n4c5ccccc5c5cc6c(cc54)C(c4ccccc4)(c4ccccc4)c4ccccc4-6)c3)n2)c1 ; N#Cc1cccc(-c2cc(-c3ccccc3)nc(-c3cccc(-n4c5ccccc5c5cc6c(cc54)C(c4ccccc4)(c4ccccc4)c4ccccc4-6)c3)n2)c1 ; c1ccc(-c2nc(-c3ccccc3)nc(-c3ccc(-n4c5ccccc5c5cc6c(cc54)-c4ccccc4C6(c4ccccc4)c4ccccc4)cc3)n2)cc1] 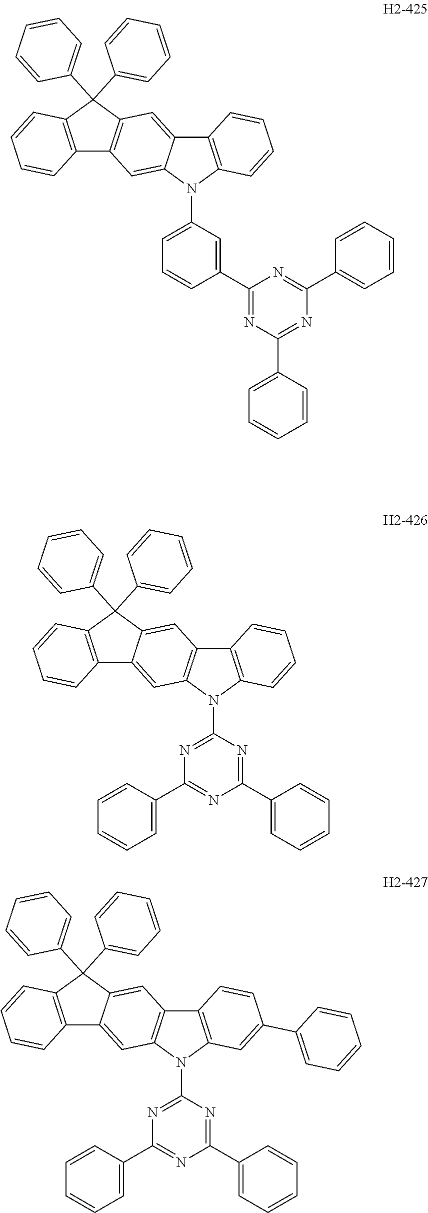 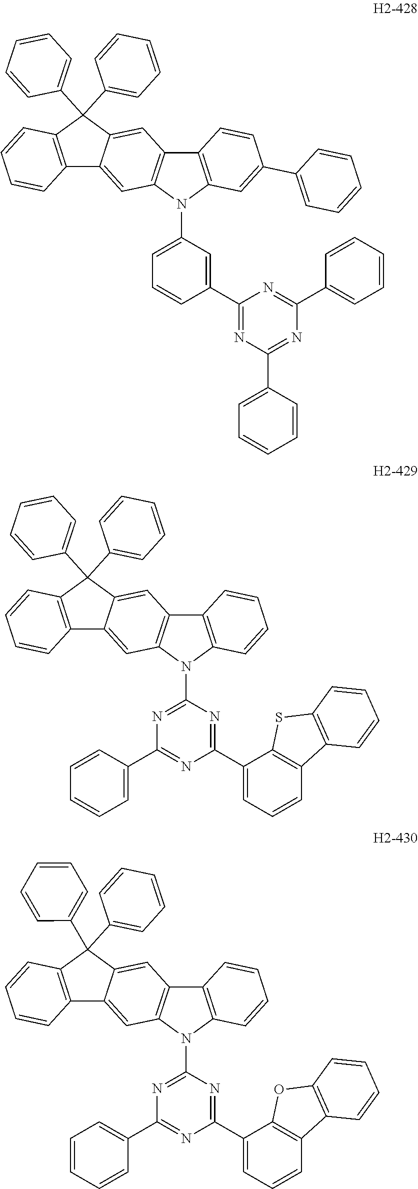 [SMILES: c1ccc(-c2ccc3c4cc5c(cc4n(-c4cccc(-c6nc(-c7ccccc7)nc(-c7ccccc7)n6)c4)c3c2)-c2ccccc2C5(c2ccccc2)c2ccccc2)cc1 ; c1ccc(-c2nc(-c3cccc4c3sc3ccccc34)nc(-n3c4ccccc4c4cc5c(cc43)-c3ccccc3C5(c3ccccc3)c3ccccc3)n2)cc1 ; c1ccc(-c2nc(-c3cccc4c3oc3ccccc34)nc(-n3c4ccccc4c4cc5c(cc43)-c3ccccc3C5(c3ccccc3)c3ccccc3)n2)cc1] 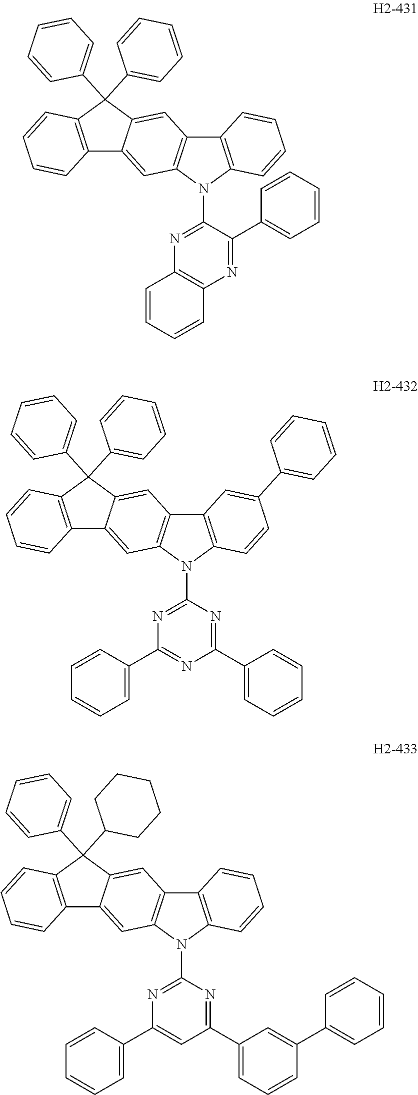 [SMILES: c1ccc(-c2nc3ccccc3nc2-n2c3ccccc3c3cc4c(cc32)-c2ccccc2C4(c2ccccc2)c2ccccc2)cc1 ; c1ccc(-c2ccc3c(c2)c2cc4c(cc2n3-c2nc(-c3ccccc3)nc(-c3ccccc3)n2)-c2ccccc2C4(c2ccccc2)c2ccccc2)cc1 ; c1ccc(-c2cccc(-c3cc(-c4ccccc4)nc(-n4c5ccccc5c5cc6c(cc54)-c4ccccc4C6(c4ccccc4)C4CCCCC4)n3)c2)cc1] 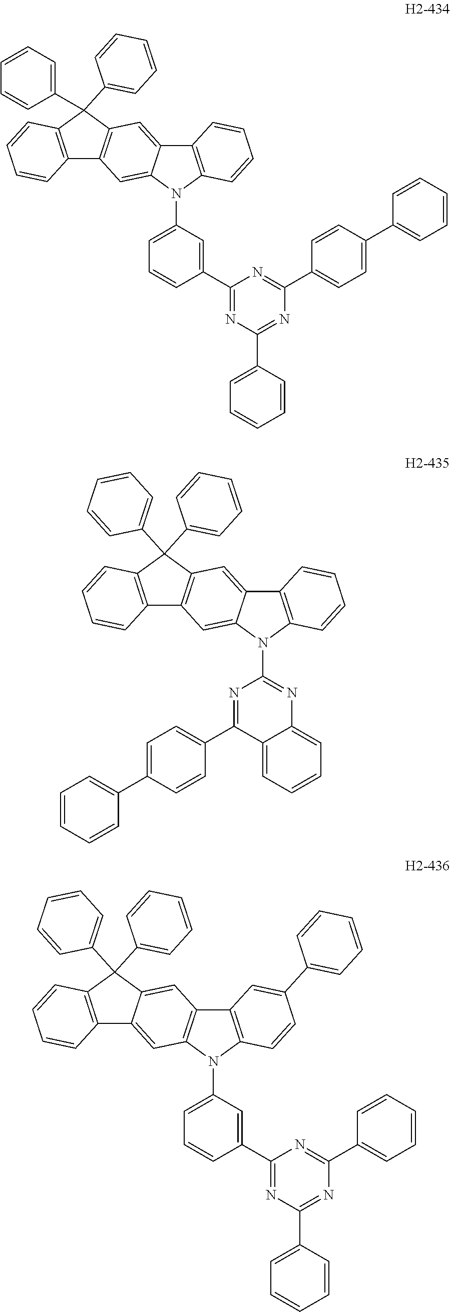 [SMILES: c1ccc(-c2ccc(-c3nc(-c4ccccc4)nc(-c4cccc(-n5c6ccccc6c6cc7c(cc65)-c5ccccc5C7(c5ccccc5)c5ccccc5)c4)n3)cc2)cc1 ; c1ccc(-c2ccc(-c3nc(-n4c5ccccc5c5cc6c(cc54)-c4ccccc4C6(c4ccccc4)c4ccccc4)nc4ccccc34)cc2)cc1 ; c1ccc(-c2ccc3c(c2)c2cc4c(cc2n3-c2cccc(-c3nc(-c5ccccc5)nc(-c5ccccc5)n3)c2)-c2ccccc2C4(c2ccccc2)c2ccccc2)cc1] 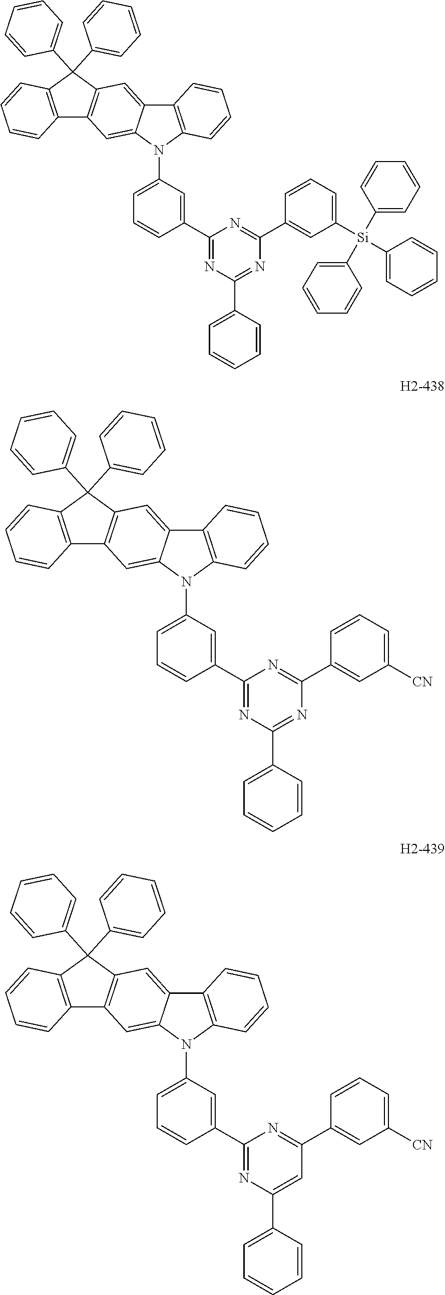 [SMILES: c1ccc(-c2nc(-c3cccc(-n4c5ccccc5c5cc6c(cc54)-c4ccccc4C6(c4ccccc4)c4ccccc4)c3)nc(-c3cccc([Si](c4ccccc4)(c4ccccc4)c4ccccc4)c3)n2)cc1 ; N#Cc1cccc(-c2nc(-c3ccccc3)nc(-c3cccc(-n4c5ccccc5c5cc6c(cc54)-c4ccccc4C6(c4ccccc4)c4ccccc4)c3)n2)c1 ; N#Cc1cccc(-c2cc(-c3ccccc3)nc(-c3cccc(-n4c5ccccc5c5cc6c(cc54)-c4ccccc4C6(c4ccccc4)c4ccccc4)c3)n2)c1] 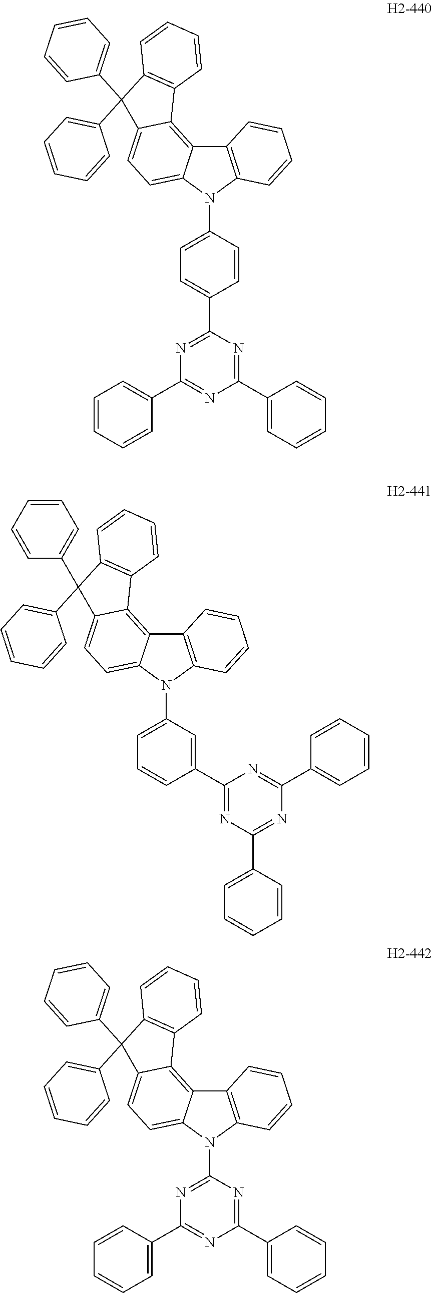 [SMILES: c1ccc(-c2nc(-c3ccccc3)nc(-c3ccc(-n4c5ccccc5c5c6c(ccc54)C(c4ccccc4)(c4ccccc4)c4ccccc4-6)cc3)n2)cc1 ; c1ccc(-c2nc(-c3ccccc3)nc(-c3cccc(-n4c5ccccc5c5c6c(ccc54)C(c4ccccc4)(c4ccccc4)c4ccccc4-6)c3)n2)cc1 ; c1ccc(-c2nc(-c3ccccc3)nc(-n3c4ccccc4c4c5c(ccc43)C(c3ccccc3)(c3ccccc3)c3ccccc3-5)n2)cc1] 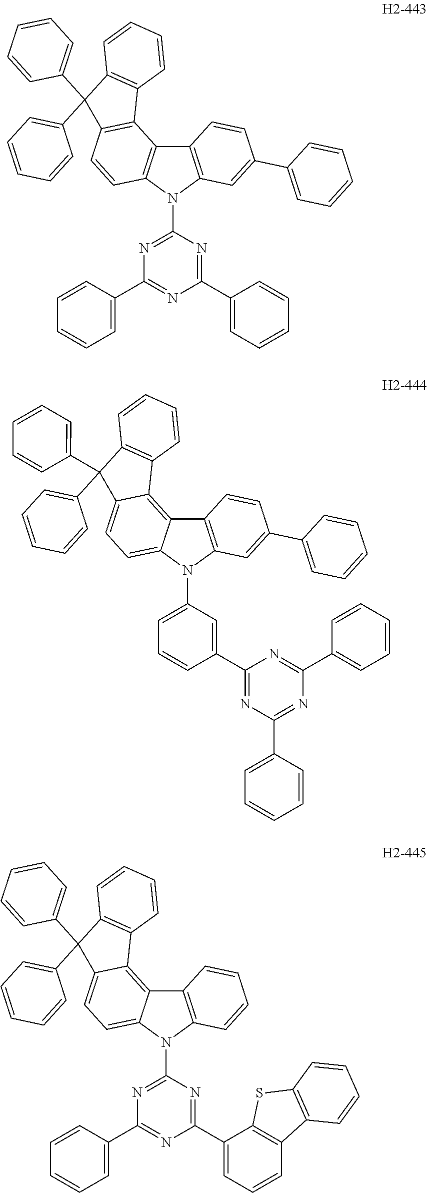 [SMILES: c1ccc(-c2ccc3c4c5c(ccc4n(-c4nc(-c6ccccc6)nc(-c6ccccc6)n4)c3c2)C(c2ccccc2)(c2ccccc2)c2ccccc2-5)cc1 ; c1ccc(-c2ccc3c4c5c(ccc4n(-c4cccc(-c6nc(-c7ccccc7)nc(-c7ccccc7)n6)c4)c3c2)C(c2ccccc2)(c2ccccc2)c2ccccc2-5)cc1 ; c1ccc(-c2nc(-c3cccc4c3sc3ccccc34)nc(-n3c4ccccc4c4c5c(ccc43)C(c3ccccc3)(c3ccccc3)c3ccccc3-5)n2)cc1] 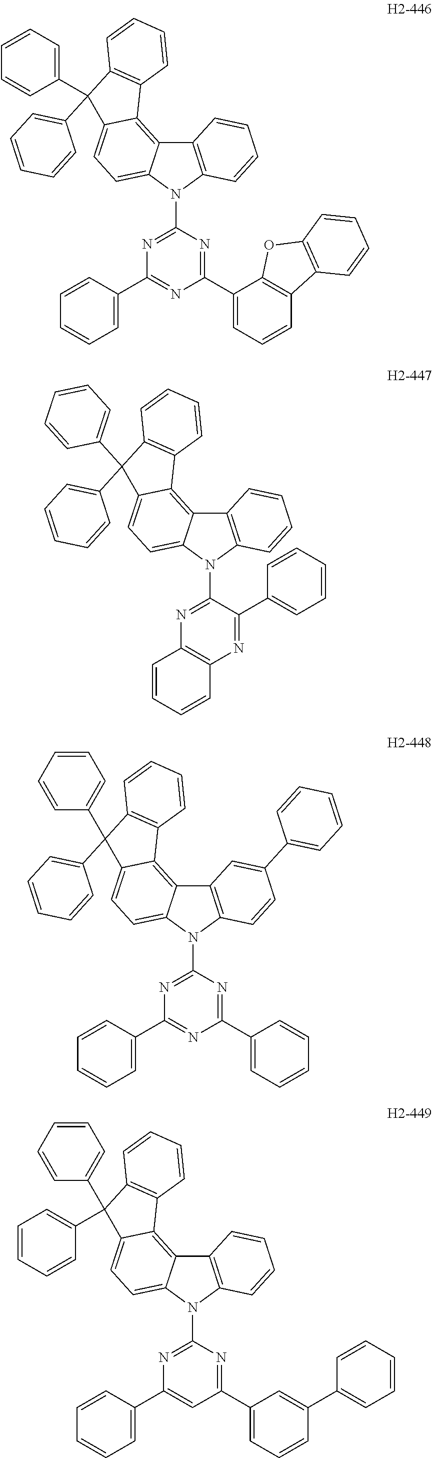 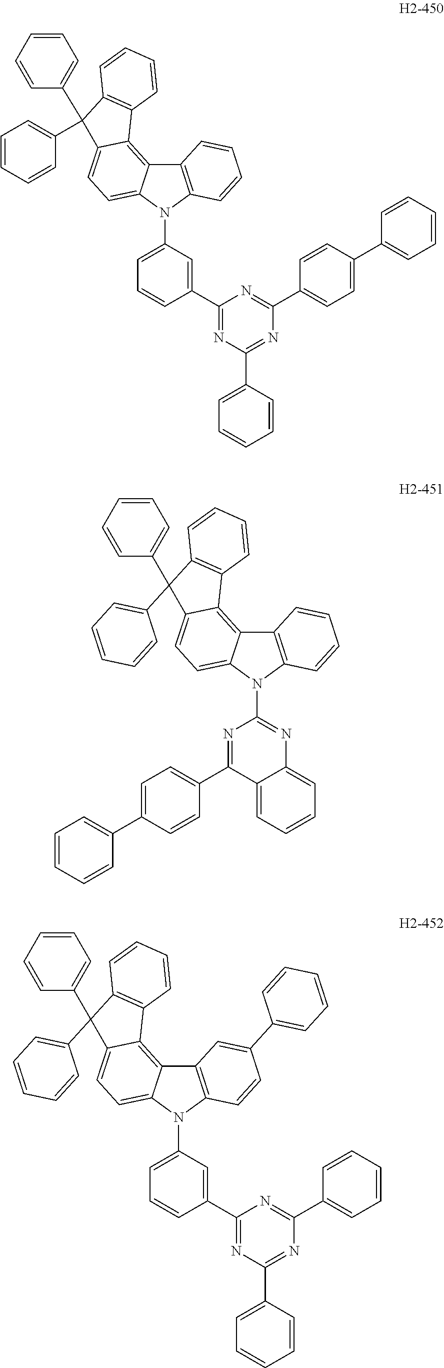 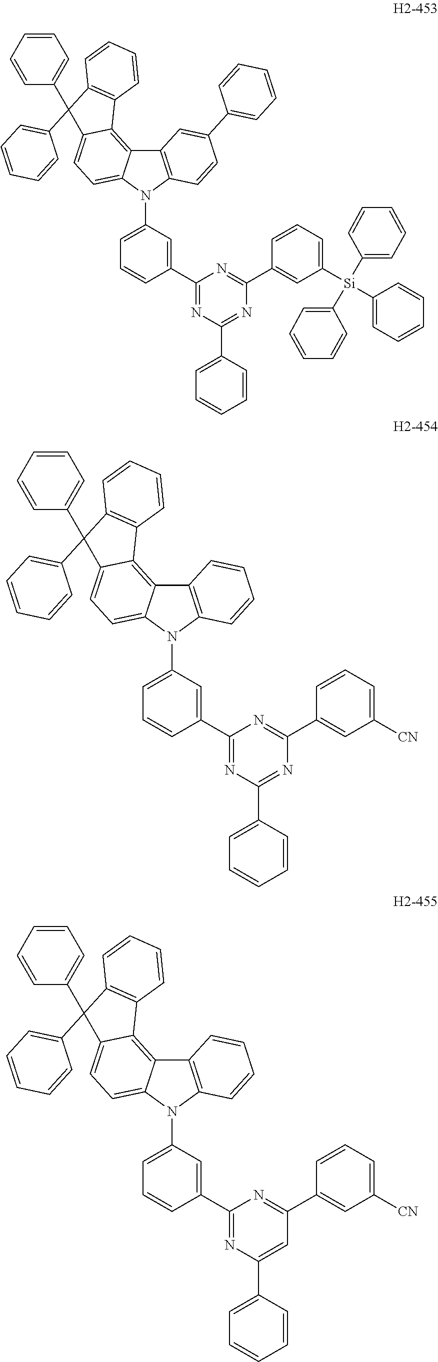 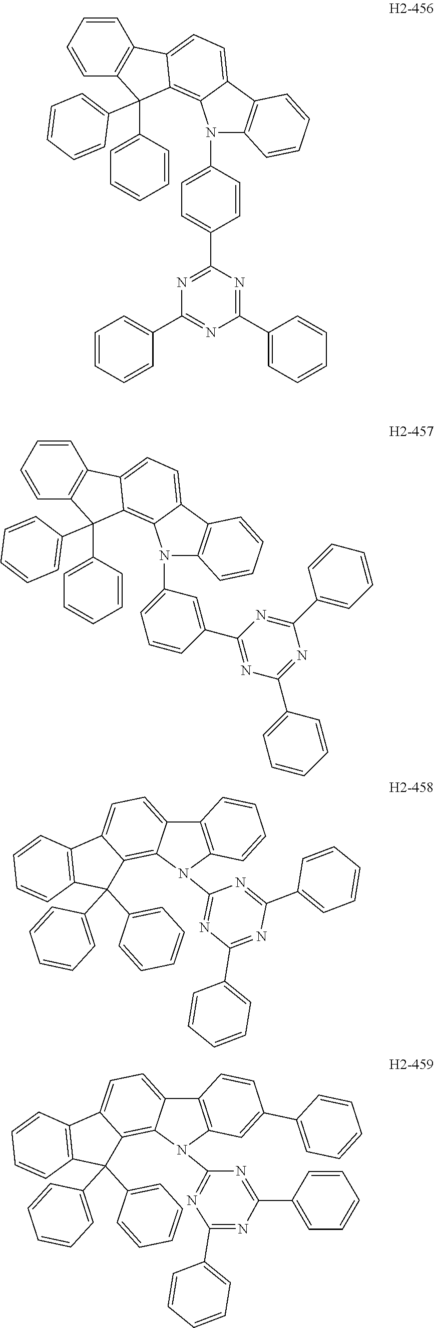 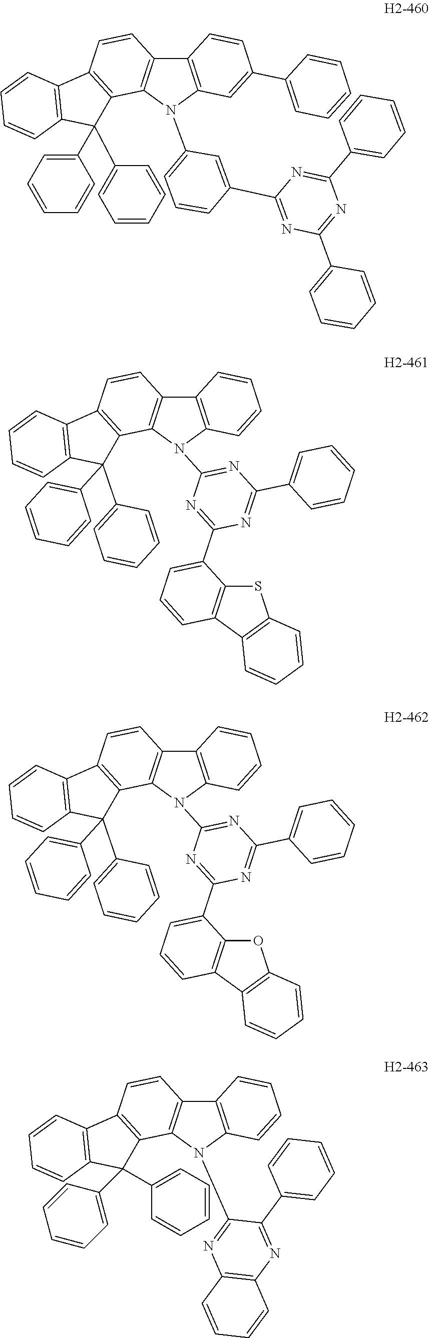 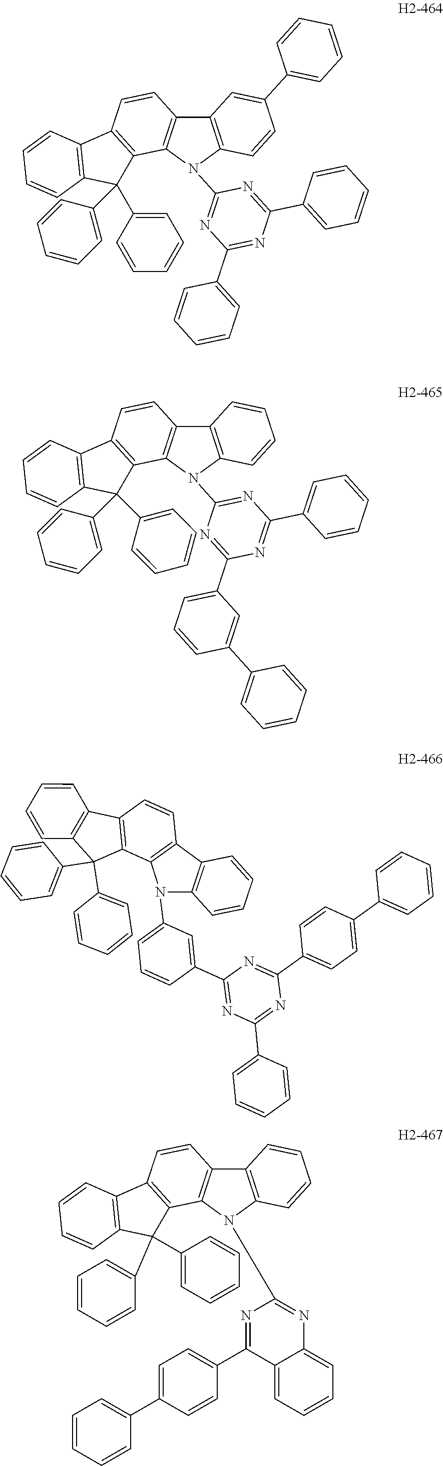 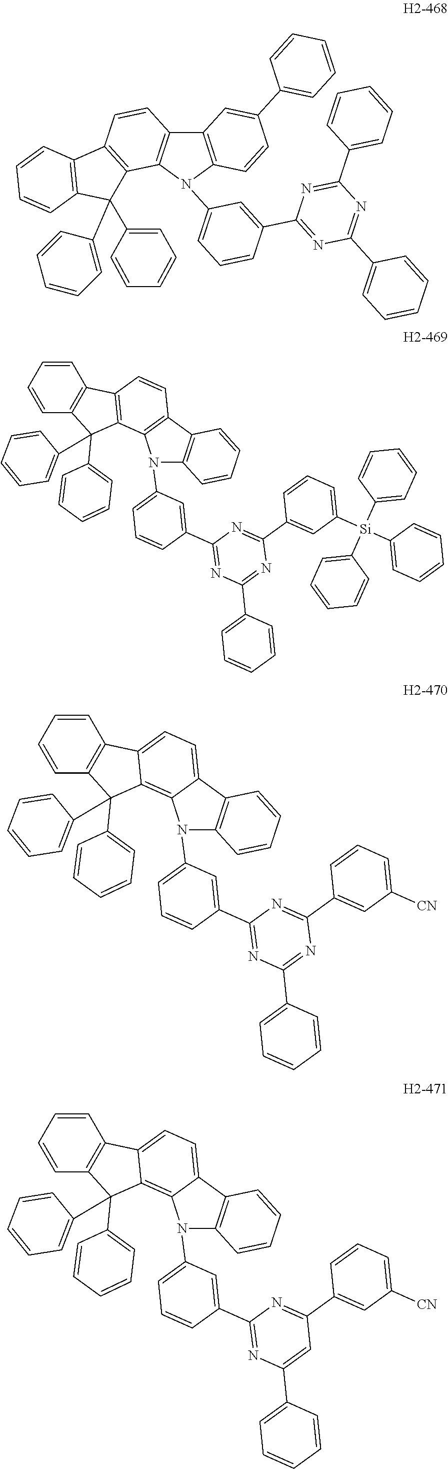 [SMILES: c1ccc(-c2ccc3c(c2)c2ccc4c(c2n3-c2cccc(-c3nc(-c5ccccc5)nc(-c5ccccc5)n3)c2)C(c2ccccc2)(c2ccccc2)c2ccccc2-4)cc1 ; c1ccc(-c2nc(-c3cccc(-n4c5ccccc5c5ccc6c(c54)C(c4ccccc4)(c4ccccc4)c4ccccc4-6)c3)nc(-c3cccc([Si](c4ccccc4)(c4ccccc4)c4ccccc4)c3)n2)cc1 ; N#Cc1cccc(-c2nc(-c3ccccc3)nc(-c3cccc(-n4c5ccccc5c5ccc6c(c54)C(c4ccccc4)(c4ccccc4)c4ccccc4-6)c3)n2)c1 ; N#Cc1cccc(-c2cc(-c3ccccc3)nc(-c3cccc(-n4c5ccccc5c5ccc6c(c54)C(c4ccccc4)(c4ccccc4)c4ccccc4-6)c3)n2)c1] 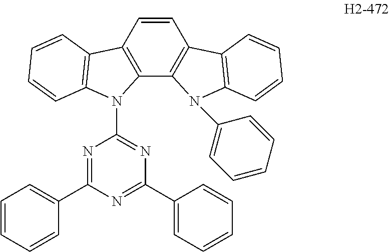 [SMILES: c1ccc(-c2nc(-c3ccccc3)nc(-n3c4ccccc4c4ccc5c6ccccc6n(-c6ccccc6)c5c43)n2)cc1] 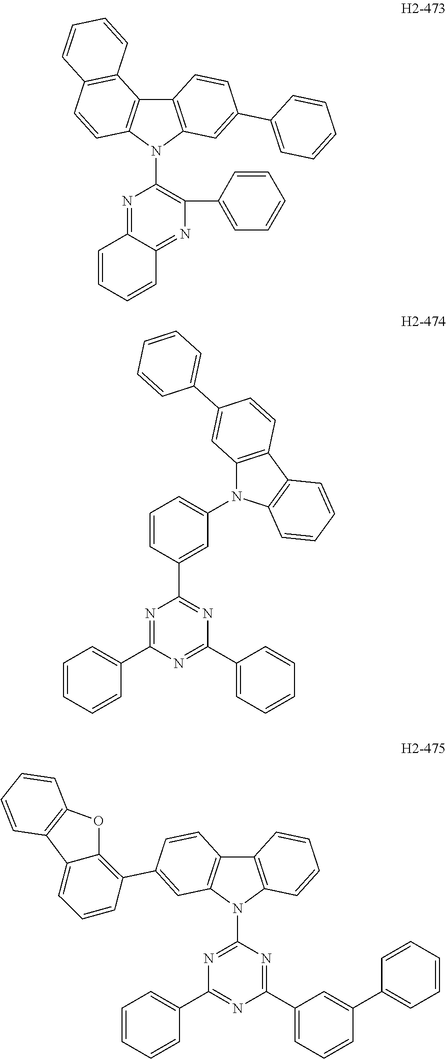 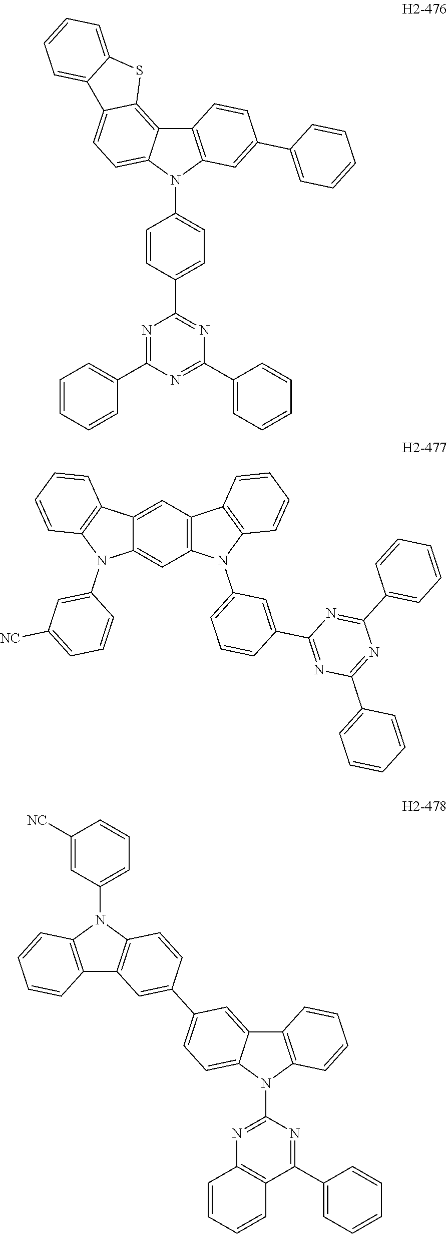 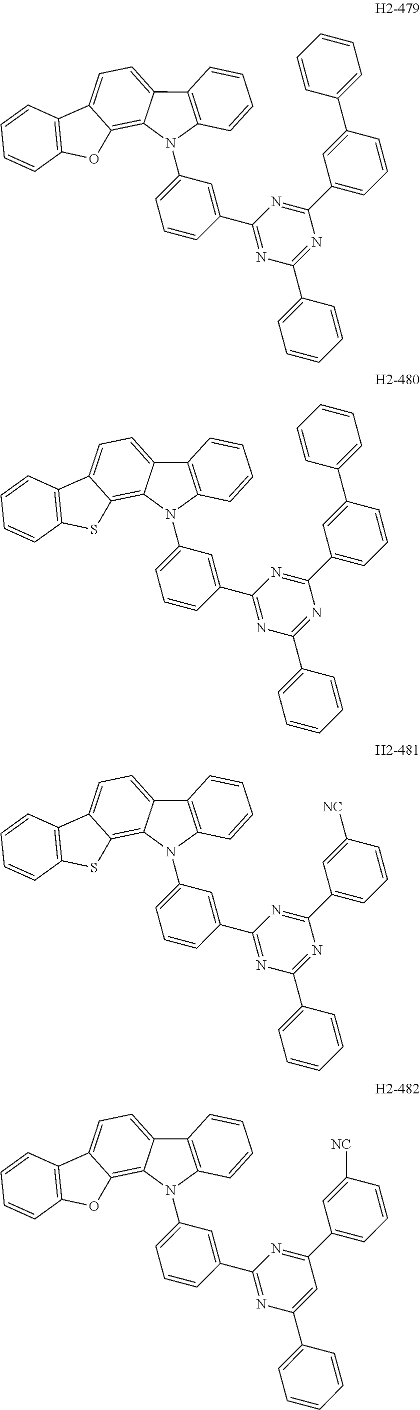 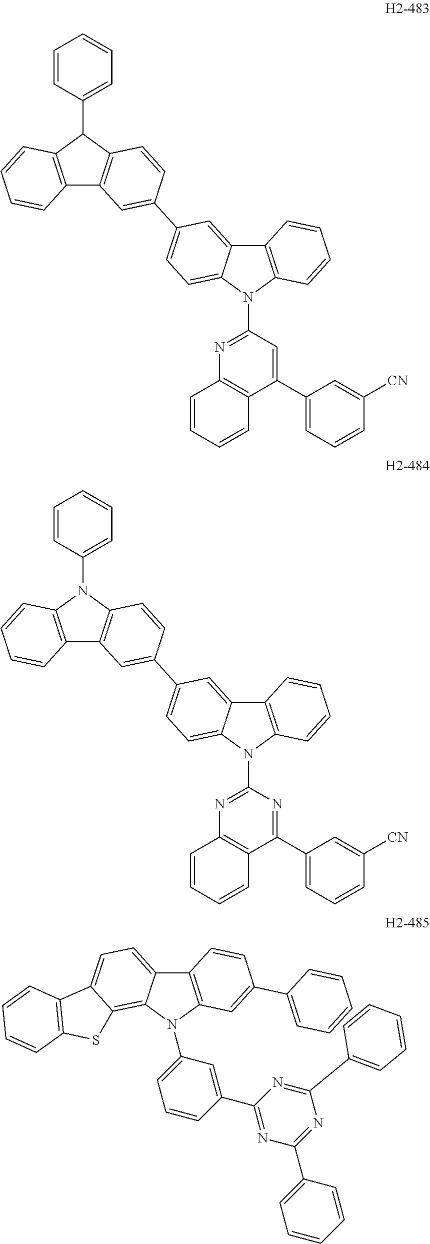 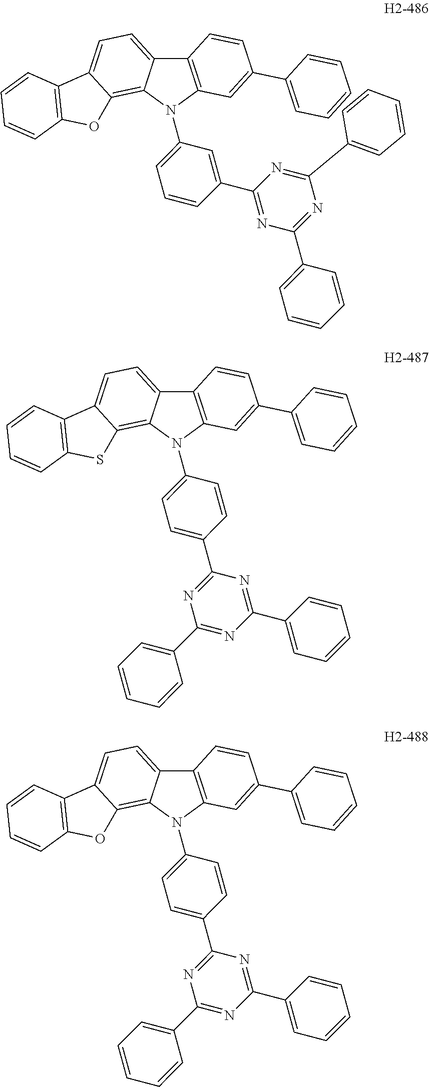 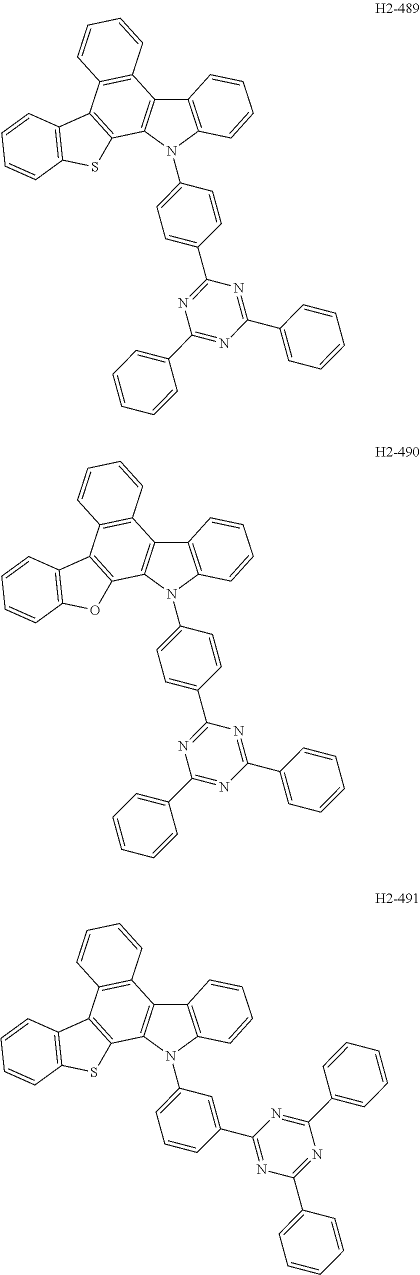 [SMILES: c1ccc(-c2nc(-c3ccccc3)nc(-c3ccc(-n4c5ccccc5c5c6ccccc6c6c7ccccc7sc6c54)cc3)n2)cc1 ; c1ccc(-c2nc(-c3ccccc3)nc(-c3ccc(-n4c5ccccc5c5c6ccccc6c6c7ccccc7oc6c54)cc3)n2)cc1 ; c1ccc(-c2nc(-c3ccccc3)nc(-c3cccc(-n4c5ccccc5c5c6ccccc6c6c7ccccc7sc6c54)c3)n2)cc1] 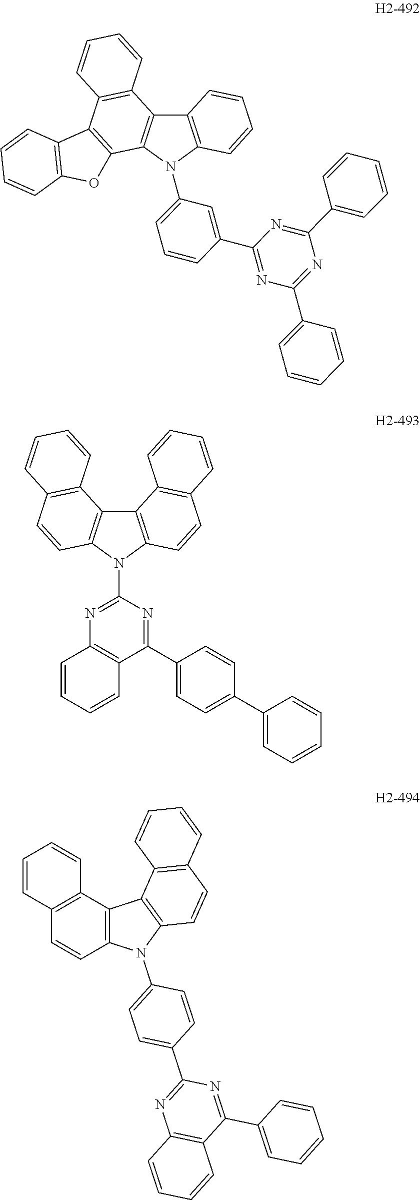 [SMILES: c1ccc(-c2nc(-c3ccccc3)nc(-c3cccc(-n4c5ccccc5c5c6ccccc6c6c7ccccc7oc6c54)c3)n2)cc1 ; c1ccc(-c2ccc(-c3nc(-n4c5ccc6ccccc6c5c5c6ccccc6ccc54)nc4ccccc34)cc2)cc1 ; c1ccc(-c2nc(-c3ccc(-n4c5ccc6ccccc6c5c5c6ccccc6ccc54)cc3)nc3ccccc23)cc1] 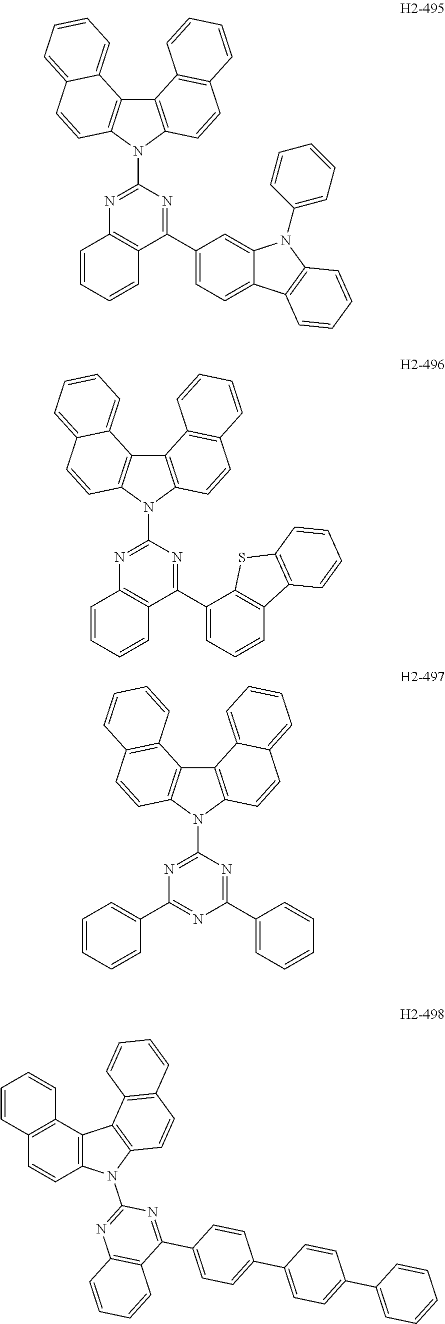 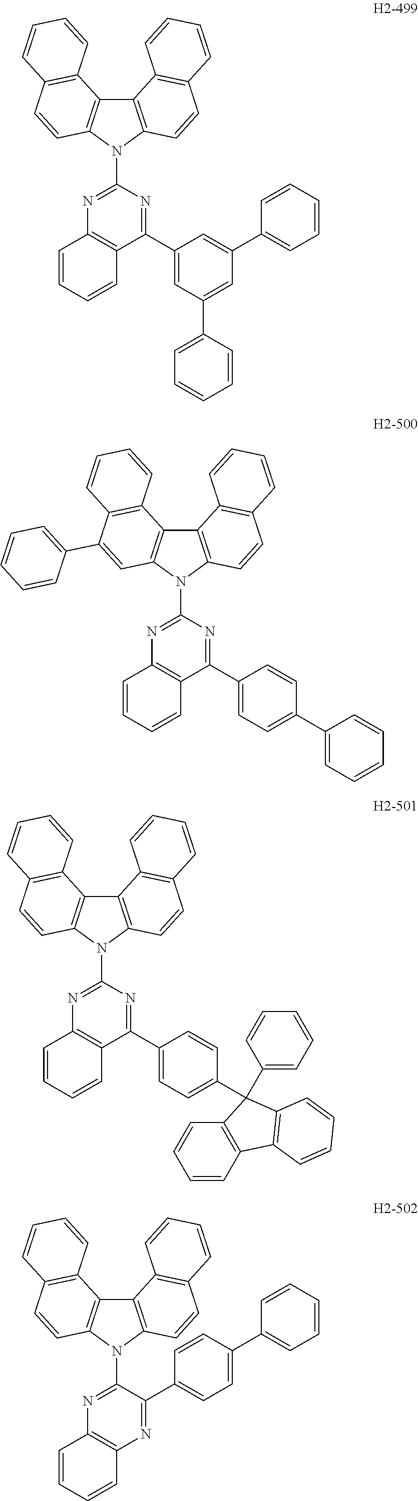 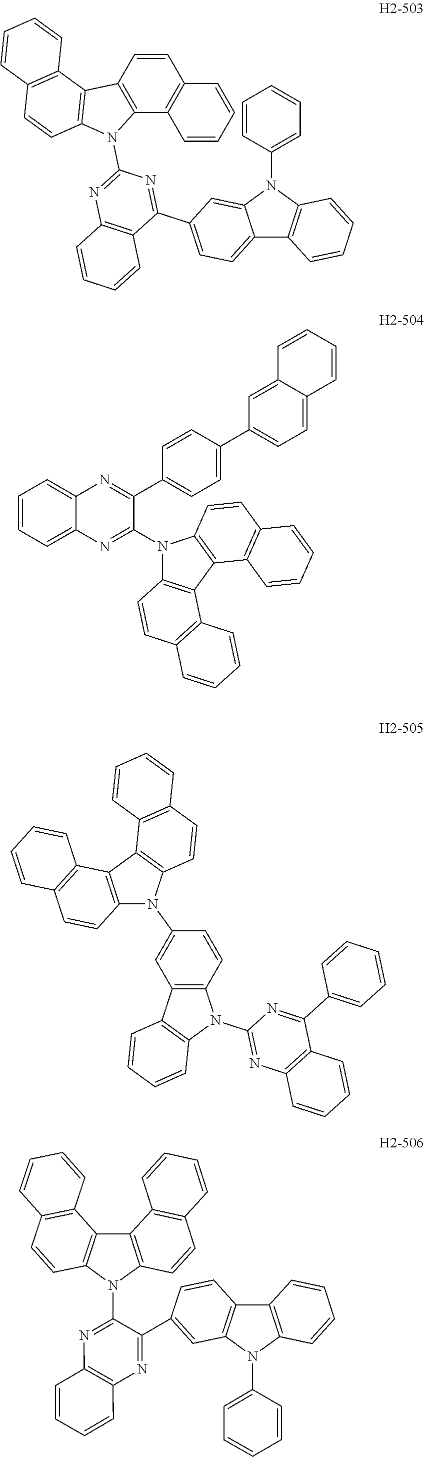 [SMILES: c1ccc(-n2c3ccccc3c3ccc(-c4nc(-n5c6ccc7ccccc7c6c6ccc7ccccc7c65)nc5ccccc45)cc32)cc1 ; c1ccc2cc(-c3ccc(-c4nc5ccccc5nc4-n4c5ccc6ccccc6c5c5c6ccccc6ccc54)cc3)ccc2c1 ; c1ccc(-c2nc(-n3c4ccccc4c4cc(-n5c6ccc7ccccc7c6c6c7ccccc7ccc65)ccc43)nc3ccccc23)cc1 ; c1ccc(-n2c3ccccc3c3ccc(-c4nc5ccccc5nc4-n4c5ccc6ccccc6c5c5c6ccccc6ccc54)cc32)cc1] 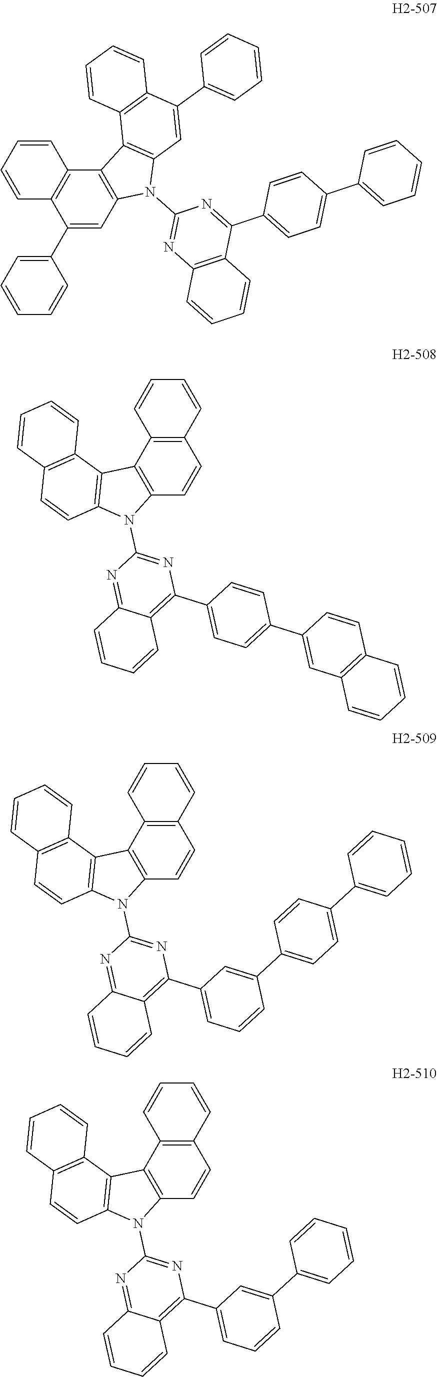 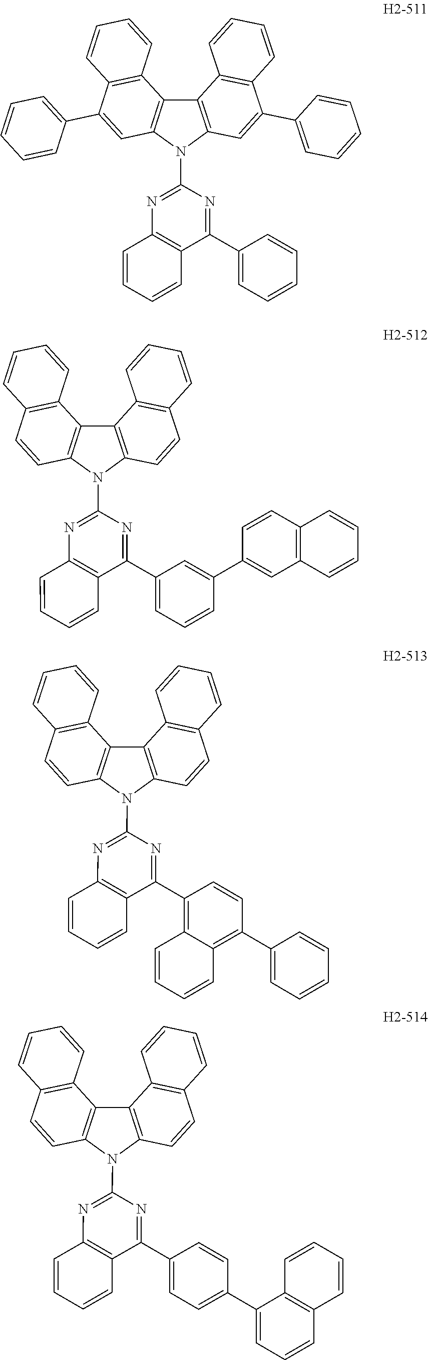 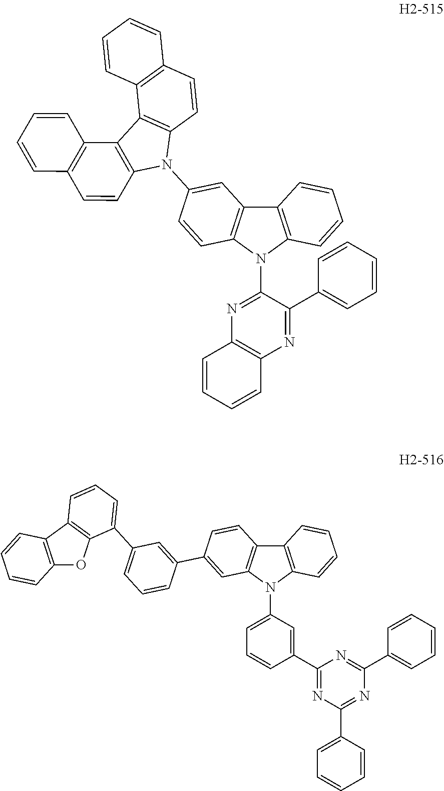 [SMILES: c1ccc(-c2nc3ccccc3nc2-n2c3ccccc3c3cc(-n4c5ccc6ccccc6c5c5c6ccccc6ccc54)ccc32)cc1 ; c1ccc(-c2nc(-c3ccccc3)nc(-c3cccc(-n4c5ccccc5c5ccc(-c6cccc(-c7cccc8c7oc7ccccc78)c6)cc54)c3)n2)cc1]
